(19) United States
(12) Patent Application Publication
(10) Pub. No.: US 2022/0025065 A1
(43) Pub. Date: Jan. 27, 2022

(54) LYSOSOMAL STORAGE DISORDER BIOMARKERS AND METHODS OF USE THEREOF

(71) Applicant: DENALI THERAPEUTICS INC., South San Francisco, CA (US)

(72) Inventors: Annie ARGUELLO, South San Francisco, CA (US); Giuseppe ASTARITA, South San Francisco, CA (US); Akhil BHALLA, South San Francisco, CA (US); Jessica R. BLUMENFELD, South San Francisco, CA (US); Gilbert DI PAOLO, South San Francisco, CA (US); Anastasia HENRY, South San Francisco, CA (US); Alicia A. NUGENT, South San Francisco, CA (US); Julie ULLMAN, South San Francisco, CA (US); Junhua WANG, South San Francisco, CA (US)

(73) Assignee: DENALI THERAPEUTICS INC., South San Francisco, CA (US)

(21) Appl. No.: 17/311,939

(22) PCT Filed: Dec. 10, 2019
(86) PCT No.: PCT/US2019/065485

§ 371 Date: Jun. 8, 2021

Related U.S. Application Data
(60) Provisional application No. 62/912,253, filed on Oct. 8, 2019.
(60) Provisional application No. 62/869,387, filed on Jul. 1, 2019.
(60) Provisional application No. 62/860,039, filed on Jun. 11, 2019.
(60) Provisional application No. 62/777,599, filed on Dec. 10, 2018.

(51) Int. Cl. C07K 16/28; C12N 9/16; G01N 33/68; A61P 43/00

(52) U.S. Cl. CPC C07K 16/2881; C12N 9/16; A61K 2039/505; A61P 43/00; C12Y 301/06013; G01N 33/6854

(57) ABSTRACT

Certain embodiments provide a method of detecting one or more biomarkers in a subject having a lysosomal storage disorder, the method comprising: 1) measuring the concentration of a combination of two or more lipids in a sample from the subject, wherein the combination of lipids is selected from the group consisting of: a) a bis(monoacylglycero)phosphate (BMP); b) a GM2 ganglioside and/or a GM3 ganglioside; c) a GD3 ganglioside; d) a GD1a/b ganglioside; and e) a glucosylceramide (GlcCer); 2) measuring the concentration of GlcCer in a sample from the subject; 3) measuring the concentration of neurofilament light chain (Nf-L) in a sample from the subject; and/or 4) measuring the concentration of soluble triggering receptor expressed on myeloid cells 2 (sTREM2) in a sample from the subject.

### CROSS-REFERENCE TO RELATED APPLICATIONS

This application claims priority to U.S. Provisional Application Ser. No. 62/777,599, filed Dec. 10, 2018, U.S. Provisional Application Ser. No. 62/860,039, filed Jun. 11, 2019, U.S. Provisional Application Ser. No. 62/869,387 filed Jul. 1, 2019 and U.S. Provisional Application Ser. No. 62/912,253, filed Oct. 8, 2019. The entire content of the applications referenced above are hereby incorporated by reference herein.

### BACKGROUND

Lysosomal storage disorders (LSDs) are relatively rare inherited metabolic diseases that result from defects in lysosomal function. LSDs are typically caused by the deficiency of a single enzyme that participates in the breakdown of metabolic products in the lysosome. The buildup of the product resulting from lack of the enzymatic activity affects various organ systems and can lead to severe symptoms and premature death. The majority of LSDs also have a significant neurological component, which ranges from progressive neurodegeneration and severe cognitive impairment to epileptic, behavioral, and psychiatric disorders. While much research has been done to investigate the molecular mechanisms underlying LSDs and to develop new treatments, additional work is still needed. In particular, there is a need for new LSD biomarkers for use in, e.g., evaluating patients and therapies, as well as for screening agents for therapeutic activity.

### SUMMARY

Accordingly, certain embodiments described herein provide a method of detecting one or more biomarkers in a subject having a lysosomal storage disorder (LSD), the method comprising:

1) measuring the concentration of a combination of two or more lipids in a sample from the subject, wherein the combination of lipids is selected from the group consisting of:
a) a bis(monoacylglycero)phosphate (BMP);
b) a GM2 ganglioside and/or a GM3 ganglioside;
c) a GD3 ganglioside;
d) a GD1a/b ganglioside; and
e) a glucosylceramide (GlcCer);

2) measuring the concentration of GlcCer in a sample from the subject, provided the LSD is a mucopolysaccharidosis (MPS) disorder;

3) measuring the concentration of neurofilament light chain (Nf-L) in a sample from the subject; and/or

4) measuring the concentration of soluble triggering receptor expressed on myeloid cells 2 (sTREM2) in a sample from the subject.

Certain other embodiments of the invention provide a method for treating an LSD in a subject, the method comprising:

1) administering an LSD treatment to the subject;

2) measuring the concentration of:
a) a combination of two or more lipids in a sample from the subject, wherein the combination of lipids is selected from the group consisting of:
   i) a BMP;
   ii) a GM2 ganglioside and/or a GM3 ganglioside;
   iii) a GD3;
   iv) a GD1a/b; and
   v) a GlcCer;
b) GlcCer in a sample from the subject, provided the LSD is an MPS disorder;
c) Nf-L in a sample from the subject; and/or
d) sTREM2 in a sample from the subject; and

3) adjusting the dosage of the LSD treatment based on the concentration of the selected lipid(s)/protein(s) in the sample from the subject as compared to a control value.

### BRIEF DESCRIPTION OF THE FIGURES

FIG. 1. Brain HS/DS (GAG) accumulation in IDS KO mice relative to age matched control. For each age grouping, wildtype (WT) is shown on the left and IDS KO is shown on the right.

FIGS. 2A-2D. Lysosomal lipids (GM2, GM3, BMP and GlcCer) accumulate in the brains of IDS KO mice relative to their age matched controls. In FIGS. 2B-2D, for each age grouping, the bar representing WT is shown on the left and bar representing IDS KO is shown on the right.

FIG. 3. Elevated levels of BMP observed in the serum of IDS KO mice relative to their age matched controls. For each age grouping, the bar representing wildtype (WT) is shown on the left and bar representing IDS KO is shown on the right.

FIG. 4. Elevated levels of lysosomal lipids (Gd1a/b, GM3, BMP and GlcCer) observed in the CSF of IDS KO mice relative to their age matched controls. For each age grouping, the bar representing wildtype (WT) is shown on the left, and bar representing IDS KO is shown on the right.

FIGS. 5A-5B. Peripheral administration of ETV:IDS (4 week treatment) corrects (FIG. 5A) brain and (FIG. 5B) CSF GAG accumulation in IDS KO mice. Graphs display mean±SEM and p values: one-way ANOVA with Dunnett multiple comparison test; * p≤0.05, ** p≤0.01, *** p≤0.001, and **** p≤0.0001.

FIG. 6. Peripheral administration of ETV:IDS (4 week treatment) corrects lysosomal lipid (gangliosides) accumulation in IDS KO brains. Graphs display mean±SEM and p values: one-way ANOVA with Dunnett multiple comparison test; * p≤0.05, ** p≤0.01, *** p≤0.001, and **** p≤0.0001.

FIGS. 7A-7B. Peripheral administration of ETV:IDS (4 week treatment) corrects lysosomal lipid (GlcCer) accumulation in IDS KO brains. Graphs display mean±SEM and p values: one-way ANOVA with Dunnett multiple comparison test; * p≤0.05, ** p≤0.01, *** p≤0.001, and **** p≤0.0001.

FIG. 8. Peripheral administration of ETV:IDS (4 week treatment) corrects lysosomal lipid (BMP) accumulation in IDS KO brains. The graph displays mean±SEM and p values: one-way ANOVA with Dunnett multiple comparison test; * p≤0.05, ** p≤0.01, *** p≤0.001, and **** p≤0.0001.

FIG. 9. Heat map illustrating lipid levels in brains of IDS KO mice treated with vehicle, ETV:IDS or Elaprase (idursulfase). Fold change as compared to WT.

FIG. 10. Peripheral administration of ETV:IDS corrects TREM2 accumulation in IDS KO brains. The graph displays mean±SEM and p values: one-way ANOVA with Dunnett multiple comparison test; **** p≤0.0001.

FIG. 11. Peripheral administration of ETV:IDS reduces accumulation of CSF soluble TREM2 (sTrem2) levels in IDS KO; TfR^{mu/hu} KI mice.

FIGS. 12A-12C. Serum (FIG. 12A), brain (FIG. 12B) and CSF (FIG. 12C) HS/DS (GAG) accumulation in IDS KO mice treated with vehicle or 3, 10, 20, or 40 mg/kg ETV:IDS.

FIGS. 13A-13C. Peripheral administration of ETV:IDS corrects GM3 (FIG. 13A), GlcCer (FIG. 13B) and BMP (FIG. 13C) accumulation in brains of IDS KO mice treated with 3, 10, 20, or 40 mg/kg ETV:IDS, as compared to IDS KO mice treated with vehicle. Graphs display mean±SEM and p values: one-way ANOVA with Dunnett multiple comparison test; * p≤0.05, ** p≤0.01, *** p≤0.001, and **** p≤0.0001.

FIGS. 14A-14B. Administration of ETV:IDS reduces the levels of individual GAG species (D0S0, D0A0 and D0a4) in brain (FIG. 14A) and CSF (FIG. 14B) from IDS KO mice, as compared to IDS KO mice treated with vehicle. Graphs display mean±SEM and p values: one-way ANOVA with Dunnett multiple comparison test; ** p≤0.01, *** p≤0.001, and **** p≤0.0001.

FIGS. 15A-15C. FIG. 15A shows a schematic of a CNS cell sorting protocol for isolation of enriched populations of neurons, astrocytes, and microglial cells. FIG. 15B shows a flowchart of a representative gating scheme used to isolate the enriched populations. FIG. 15C shows representative FACS gates for the sorting procedure described in FIG. 15B. Starting from top left to bottom right: Forward (FSC) and side (SSC) scatter determines cells from debris; live cells are positively gated; exclusion of CD31 positive endothelial cells is confirmed; EAAT2 positive astrocytes are subgated from CD11b microglia; Thy1 positive neurons are subgated from EAAT2 positive astrocytes; and finally removal of O1 oligodendrocytes cells in CD11b microglia, EAAT2 astrocytes, and Thy1 neurons cell populations determines the final sort criteria.

FIG. 16. Principle components analysis using log-transformed CPM expression values from the top 500 genes with the highest variance. Principal components 1 and 2 account for 48% and 26% of the variance between samples, respectively.

FIGS. 17A-17C. FIG. 17A illustrates expression of certain cell-specific markers in purified neurons, astrocytes, microglia, and the input cell suspension determined by RNA-Seq. Rows are gene groups specific to cell types: (endo.) endothelial, (oligo.) oligodendrocytes; expression values per gene are depicted as the number of standard deviations away from its mean (z-score); n=4 mice. FIG. 17B lists and illustrates expression of the top 20 enriched genes determined by fold enrichment >5.0 and FDR <0.01 of each cell type. Heatmap expression values are plotted as gene-level z-scores. Input cells are the single cell suspension from the dissociated brains. FIG. 17C shows qRT-PCR data generated from isolated cell populations of neurons, astrocytes, and microglia. Gene classes are grouped on the x axis: (Astro) astrocytes, (MG) microglia, (Neu) neurons, (Oligo) oligodendrocytes, and (Endo) endothelial cells; n=5 mice. Graphs display mean±SEM.

FIG. 18A. GAG levels in microglia, astrocytes, and neurons isolated from IDS KO; TfR^{mu/hu} KI mice relative to TfR^{mu/hu} KI controls (n=3-5 mice per group). All data are displayed as mean±SEM; unpaired student's t-test p≤0.05*, 0.001***.

FIG. 18B. Distribution of ETV:IDS in FACS-enriched neuron, astrocyte, and microglia cell populations at 2-hours post dose in vehicle-treated TfR^{mu/hu} KI and ETV:IDS (40 mg/kg) treated IDS KO; TfR^{mu/hu} KI mice; n=4 mice per group. Graph displays mean±SEM; two-way ANOVA with Sidak's test; ** p≤0.01, *** p≤0.001, and **** p≤0.0001.

FIG. 19. Reduction of GAG accumulation in neurons, astrocytes, and microglia from IDS KO; TfR^{mu/hu} KI mice dosed intravenously with 40 mg/kg ETV:IDS once a week for four weeks. Data are displayed as mean±SEM; one-way ANOVA with Tukey's multiple comparison test, * p≤0.05, ** p≤0.01, **** p≤0.0001.

FIG. 20. Reduction of ganglioside accumulation in neurons, astrocytes, and microglia from IDS KO; TfR^{mu/hu} KI mice dosed intravenously with 40 mg/kg ETV:IDS once a week for four weeks. Data are displayed as mean±SEM; one-way ANOVA with Tukey's multiple comparison test, * p≤0.05, ** p≤0.01, **** p≤0.0001.

FIG. 21. Reduction of glucosylceramide accumulation in neurons, astrocytes, and microglia from IDS KO; TfR^{mu/hu} KI mice dosed intravenously with 40 mg/kg ETV:IDS once a week for four weeks. Data are displayed as mean±SEM; one-way ANOVA with Tukey's multiple comparison test, * p≤0.05, ** p≤0.01, **** p≤0.0001.

FIG. 22. Rescue of BMP alterations in neurons, astrocytes, and microglia from IDS KO; TfR^{mu/hu} KI mice dosed intravenously with 40 mg/kg ETV:IDS once a week for four weeks. Data are displayed as mean±SEM; one-way ANOVA with Tukey's multiple comparison test, * p≤0.05, ** p≤0.01, **** p≤0.0001.

FIG. 23. H&E staining (top panel) and MALDI MS images (middle and bottom panels) of coronal brain sections of wild-type and IDS KO; TfR^{mu/hu} KI mice after four, weekly doses of vehicle, idursulfase or ETV:IDS. The middle panel shows the distribution of the signal at m/z 1382.816, corresponding to GM2 (d36:1); and the bottom panel shows the distribution of the signal at m/z 1179.738, corresponding to GM3 (d36:1). Images depict the relative intensity of each signal from 0-100%.

FIG. 24. Immunofluorescent images of brain tissue from TfR^{mu/hu} KI and IDS KO; TfR^{mu/hu} KI mice that were treated with vehicle, ETV:IDS or idursulfase. The brain tissue was stained for DAPI and CD68. Magnification is 20×, and the images represent hippocampus (top panel), cortex (middle panel), and striatum (bottom panel).

FIG. 25. A table of a cell-type specific enriched gene set; the information in the table corresponds to the heat map of FIG. 17B. Expression of the top 20 genes determined by fold enrichment >5.0 and FDR <0.01 for each cell type are listed in ascending order of p-value. Average log fold changes (log FC.avg) are relative to the other two “out-group” populations, and the far right three columns show the average expression of the gene within each cell population; n=4 mice.

FIGS. 26A-26C. Scatter plots of Nf-L concentrations in wild-type and IDS knockout mice at 3, 6, and 9 months of age. Nf-L levels in serum and CSF of 9-month old mice are illustrated in FIGS. 26A and 26B, respectively. Nf-L levels in CSF of mice cohorts at different ages are illustrated in FIG. 26C. Data are displayed as mean+/−SEM with p values obtained by unpaired t test analysis.

FIG. 27. Peripheral administration of ETV:IDS (13 weekly doses of 1 mg/kg or 3 mg/kg) reduces neurofilament light chain (Nf-L) levels in IDS knockout mice. Data are displayed as mean+/−SEM with p values obtained by unpaired t test analysis (*p<0.05; ***p<0.001; error bars=SEM).

FIG. 28. Peripheral administration of ETV:IDS (13 weekly doses of 1 mg/kg or 3 mg/kg) reduces accumulation of GAG levels in the liver and urine of IDS knockout mice. Urine GAG levels were normalized to creatinine. Data are displayed as mean+/−SEM with p values obtained by unpaired t test analysis (**** p<0.0001; error bars=SEM).

### DETAILED DESCRIPTION

As described herein, a series of biomarkers associated with LSDs have been identified. In particular, as described in the Examples, a series of secondary lysosomal lipids were shown to accumulate in brain, CSF and serum from an LSD murine model. Additionally, triggering receptor expressed on myeloid cells 2 (TREM2) and neurofilament light chain (Nf-L) were also shown to accumulate in brain tissue from these mice. Notably, this secondary lipid accumulation and TREM2 accumulation could be improved with the administration of an LSD treatment (e.g., ETV:IDS). Based on these discoveries, these lipids/proteins can be used as biomarkers for purposes including, but not limited to, evaluating subjects having such disorders, evaluating the efficacy of certain treatments, developing and/or modifying treatment regimens (e.g., adjusting dosing) and in methods for screening agents for therapeutic activity.

Accordingly, certain embodiments described herein provide a method of detecting one or more biomarkers in a subject having an LSD, the method comprising:

1) measuring the concentration of a combination of two or more lipids in a sample from the subject, wherein the combination of lipids is selected from the group consisting of:
a) a BMP;
b) a GM2 ganglioside and/or a GM3 ganglioside;
c) a GD3 ganglioside;
d) a GD1a/b ganglioside; and
e) a GlcCer;

2) measuring the concentration of GlcCer in a sample from the subject, provided the LSD is a MPS disorder; and/or

3) measuring the concentration of sTREM2 in a sample from the subject.

Certain embodiments described herein also provide a method of detecting one or more biomarkers in a subject having an LSD, the method comprising:

1) measuring the concentration of a combination of two or more lipids in a sample from the subject, wherein the combination of lipids is selected from the group consisting of:
a) a BMP;
b) a GM2 ganglioside and/or a GM3 ganglioside;
c) a GD3 ganglioside;
d) a GD1a/b ganglioside; and
e) a GlcCer;

2) measuring the concentration of GlcCer in a sample from the subject, provided the LSD is a NIPS disorder;

3) measuring the concentration of Nf-L in a sample from the subject; and/or

4) measuring the concentration of sTREM2 in a sample from the subject.

Certain embodiments described herein also provide a method of evaluating the efficacy of a treatment in a subject having an LSD, the method comprising:

1) measuring the concentration of a combination of two or more lipids in a sample from the subject (i.e., a sample obtained from the subject after administration of the treatment), wherein the combination of lipids is selected from the group consisting of:
a) a BMP;
b) a GM2 ganglioside and/or a GM3 ganglioside;
c) a GD3 ganglioside;
d) a GD1a/b ganglioside; and
e) a GlcCer;

2) measuring the concentration of GlcCer in a sample from the subject, provided the LSD is an MPS disorder; and/or

3) measuring the concentration of sTREM2 in a sample from the subject;

wherein a decrease in the concentration of the selected lipid(s)/protein in the sample from the subject as compared to the concentration of the lipid(s)/protein in a sample obtained from the subject prior to administration of the treatment correlates with treatment efficacy.

Certain embodiments described herein provide a method of evaluating the efficacy of a treatment in a subject having an LSD, the method comprising:

1) measuring the concentration of a combination of two or more lipids in a sample from the subject (i.e., a sample obtained from the subject after administration of the treatment), wherein the combination of lipids is selected from the group consisting of:
a) a BMP;
b) a GM2 ganglioside and/or a GM3 ganglioside;
c) a GD3 ganglioside;
d) a GD1a/b ganglioside; and
e) a GlcCer;

2) measuring the concentration of GlcCer in a sample from the subject, provided the LSD is an MPS disorder;

3) measuring the concentration of Nf-L in a sample from the subject; and/or

4) measuring the concentration of sTREM2 in a sample from the subject;

wherein a decrease in the concentration of the selected lipid(s)/protein(s) in the sample from the subject as compared to the concentration of the lipid(s)/protein(s) in a sample obtained from the subject prior to administration of the treatment correlates with treatment efficacy.

Certain embodiments described herein provide a method of identifying a subject having an LSD as a candidate for treatment, comprising:

1) measuring the concentration of a combination of two or more lipids in a sample from the subject, wherein the combination of lipids is selected from the group consisting of:
a) a BMP;
b) a GM2 ganglioside and/or a GM3 ganglioside;
c) a GD3 ganglioside;
d) a GD1a/b ganglioside; and
e) a GlcCer;

2) measuring the concentration of GlcCer in a sample from the subject, provided the LSD is an MPS disorder; and/or

3) measuring the concentration of sTREM2 in a sample from the subject;

wherein the subject is identified as a candidate or a non-candidate for treatment based on the concentration of the selected lipid(s)/protein in the sample as compared to a control value. For example, a concentration of the selected lipid(s)/protein in the sample from the subject that is at least as high as a control value identifies the subject as a candidate for treatment.

Certain embodiments described herein also provide a method of identifying a subject having an LSD as a candidate for treatment, comprising:

1) measuring the concentration of a combination of two or more lipids in a sample from the subject, wherein the combination of lipids is selected from the group consisting of:
a) a BMP;
b) a GM2 ganglioside and/or a GM3 ganglioside;
c) a GD3 ganglioside;
d) a GD1a/b ganglioside; and
e) a GlcCer;

2) measuring the concentration of GlcCer in a sample from the subject, provided the LSD is an MPS disorder;

3) measuring the concentration of Nf-L in a sample from the subject; and/or

4) measuring the concentration of sTREM2 in a sample from the subject;

wherein the subject is identified as a candidate or a non-candidate for treatment based on the concentration of the selected lipid(s)/protein(s) in the sample as compared to a control value. For example, a concentration of the selected lipid(s)/protein(s) in the sample from the subject that is at least as high as a control value identifies the subject as a candidate for treatment.

In certain embodiments, a method described herein further comprises administering an LSD treatment to a subject. In certain embodiments, a method described herein further comprises adjusting a subject's treatment regimen. For example, dosing may be increased or decreased, dosing frequency may be increased or decreased or an alternative therapy may be administered based on a comparison of the concentrations of each of the selected lipids/proteins to a control value.

Thus, certain embodiments described herein provide a method for treating an LSD in a subject, the method comprising:

1) administering an LSD treatment to the subject;

2) measuring the concentration of:
a) a combination of two or more lipids in a sample from the subject, wherein the combination of lipids is selected from the group consisting of:
   i) a BMP;
   ii) a GM2 ganglioside and/or a GM3 ganglioside;
   iii) a GD3 ganglioside;
   iv) a GD1a/b ganglioside; and
   v) a GlcCer;
b) GlcCer in a sample from the subject, provided the LSD is an MPS disorder; and/or
c) sTREM2 in a sample from the subject; and

3) adjusting the dosage of the LSD treatment based on the concentration of the selected lipid(s)/protein in the sample from the subject as compared to a control value. In certain embodiments, the method comprises administering to the subject an adjusted dosage of the LSD treatment, wherein the dosage adjustment is based on the concentration of the selected lipid(s)/protein as compared to a control value. In certain embodiments, the method comprises administering to the subject a dosage of the LSD treatment that is higher than the original LSD treatment dosage (step 1) when the concentration of the selected lipid(s)/protein is higher than a control value. In certain embodiments, the method comprises administering to the subject a dosage of the LSD treatment that is lower than the original LSD treatment dosage (step 1) when the concentration of the selected lipid(s)/protein is lower than a control value.

Certain embodiments described herein also provide a method for treating an LSD in a subject, the method comprising:

1) administering an LSD treatment to the subject;

2) measuring the concentration of:
a) a combination of two or more lipids in a sample from the subject, wherein the combination of lipids is selected from the group consisting of:
   i) a BMP;
   ii) a GM2 ganglioside and/or a GM3 ganglioside;
   iii) a GD3 ganglioside;
   iv) a GD1a/b ganglioside; and
   v) a GlcCer;
b) GlcCer in a sample from the subject, provided the LSD is an MPS disorder;
c) Nf-L in a sample from the subject; and/or
d) sTREM2 in a sample from the subject; and

3) adjusting the dosage of the LSD treatment based on the concentration of the selected lipid(s)/protein(s) in the sample from the subject as compared to a control value. In certain embodiments, the method comprises administering to the subject an adjusted dosage of the LSD treatment, wherein the dosage adjustment is based on the concentration of the selected lipid(s)/protein(s) as compared to a control value. In certain embodiments, the method comprises administering to the subject a dosage of the LSD treatment that is higher than the original LSD treatment dosage (step 1) when the concentration of the selected lipid(s)/protein(s) is higher than a control value. In certain embodiments, the method comprises administering to the subject a dosage of the LSD treatment that is lower than the original LSD treatment dosage (step 1) when the concentration of the selected lipid(s)/protein(s) is lower than a control value.

In certain embodiments, a method described herein comprises measuring, or having measured, the concentration of sTREM2 in a sample from a subject having an LSD.

In certain embodiments, a method described herein comprises measuring, or having measured, the concentration of Nf-L in a sample from a subject having an LSD.

As described herein, the concentration of GlcCer may be measured in a sample from a subject having an LSD. In such an embodiment, the LSD is an MPS. Thus, in certain embodiments, a method described herein comprises measuring, or having measured, the concentration of GlcCer in a sample from a subject having an LSD, provided the LSD is an MPS. In certain other embodiments, GlcCer is measured in combination with other lipids/proteins described herein. In such an embodiment, the LSD may be any LSD, such as an LSD described herein.

In certain embodiments, a method described herein comprises measuring, or having measured, the concentration of a combination of two or more lipids in a sample from a subject having an LSD.

In certain embodiments, a method described herein comprises measuring, or having measured, the concentration of a combination of one or more lipids and the concentration of sTREM2 in a sample from a subject having an LSD.

In certain embodiments, a method described herein comprises measuring, or having measured, the concentration of a combination of one or more lipids and the concentration of Nf-L in a sample from a subject having an LSD.

In certain embodiments, a method described herein comprises measuring, or having measured, the concentration of sTREM2 and the concentration of Nf-L in a sample from a subject having an LSD.

In certain embodiments, a method described herein comprises measuring, or having measured, the concentration of a combination of one or more lipids, the concentration of sTREM2 and the concentration of Nf-L in a sample from a subject having an LSD.

Certain embodiments described herein provide a method for treating an LSD in a subject, the method comprising administering an LSD treatment to the subject, wherein the subject has, or was determined to have:

1) an increased concentration of a combination of two or more lipids as compared to a control, wherein the combination of lipids is selected from the group consisting of:
a) a BMP;
b) a GM2 ganglioside and/or a GM3 ganglioside;
c) a GD3 ganglioside;
d) a GD1a/b ganglioside; and
e) a GlcCer;

2) an increased concentration of GlcCer, provided the LSD is an MPS disorder; and/or

3) an increased concentration of sTREM2.

Certain embodiments described herein provide a method for treating an LSD in a subject, the method comprising administering an LSD treatment to the subject, wherein the subject has, or was determined to have:

1) an increased concentration of a combination of two or more lipids as compared to a control, wherein the combination of lipids is selected from the group consisting of:
a) a BMP;
b) a GM2 ganglioside and/or a GM3 ganglioside;
c) a GD3 ganglioside;
d) a GD1a/b ganglioside; and
e) a GlcCer;

2) an increased concentration of GlcCer, provided the LSD is an MPS disorder;

3) an increased concentration of Nf-L; and/or

4) an increased concentration of sTREM2.

Certain embodiments described herein provide a method of treating an LSD in a subject, the method comprising:

1) obtaining or having obtained a sample from the subject;

2) detecting or having detected in the sample an increased concentration of:
a) a combination of two or more lipids in the sample as compared to a control, wherein the combination of lipids/protein is selected from the group consisting of:
   i) a BMP;
   ii) a GM2 ganglioside and/or a GM3 ganglioside;
   iii) a GD3 ganglioside;
   iv) a GD1a/b ganglioside; and
   v) a GlcCer;
b) GlcCer in the sample as compared to a control, provided the LSD is an MPS disorder; and/or
c) sTREM2 in the sample as compared to a control;

3) diagnosing the subject with an LSD when an increased concentration of the selected lipids/protein is detected; and

4) administering an effective amount of LSD treatment to the diagnosed subject.

Certain embodiments described herein provide a method of treating an LSD in a subject, the method comprising:

1) obtaining or having obtained a sample from the subject;

2) detecting or having detected in the sample an increased concentration of:
a) a combination of two or more lipids in the sample as compared to a control, wherein the combination of lipids/proteins is selected from the group consisting of:
   i) a BMP;
   ii) a GM2 ganglioside and/or a GM3 ganglioside;
   iii) a GD3 ganglioside;
   iv) a GD1a/b ganglioside; and
   v) a GlcCer;
b) GlcCer in the sample as compared to a control, provided the LSD is an MPS disorder;
c) Nf-L in the sample as compared to a control; and/or
d) sTREM2 in the sample as compared to a control.

3) diagnosing the subject with an LSD when an increased concentration of the selected lipids/proteins is detected; and

4) administering an effective amount of LSD treatment to the diagnosed subject.

In certain embodiments, the subject has, or was determined to have, an increased concentration of sTREM2.

In certain embodiments, the subject has, or was determined to have, an increased concentration of Nf-L.

In certain embodiments, the subject has, or was determined to have, an increased concentration of GlcCer.

In certain embodiments, the subject has, or was determined to have, an increased concentration of a combination of two or more lipids.

In certain embodiments, the subject has, or was determined to have, an increased concentration of one or more lipids and an increased concentration of sTREM2.

In certain embodiments, the subject has, or was determined to have, an increased concentration of one or more lipids and an increased concentration of Nf-L.

In certain embodiments, the subject has, or was determined to have, an increased concentration of Nf-L and an increased concentration of sTREM2.

In certain embodiments, the subject has, or was determined to have, an increased concentration of one or more lipids, an increased concentration of Nf-L and an increased concentration of sTREM2.

Certain embodiments also provide an LSD treatment for use in a method described herein.

Certain embodiments provide the use of an LSD treatment to prepare a medicament for use in a method described herein.

### Biomarkers for Lysosomal Storage Disorders

As described herein, a series of biomarkers for LSDs have been identified. In particular, these biomarkers include the accumulation of specific lipids (i.e., BMP, GlcCer, GD3, GD1a/b, GM2 and GM3) in a subject having an LSD, as well as the accumulation of TREM2, which may be measured based on sTREM2 levels, and the accumulation of Nf-L. Thus, these biomarkers may be evaluated by measuring the concentration of one or more of these lipids/proteins in a sample obtained from the subject.

As used herein, the phrase “sample” or “physiological sample” is meant to refer to a biological sample obtained from a subject that contains protein and/or lipid. Thus, the sample may be evaluated at the lipid or protein level. In certain embodiments, the physiological sample comprises tissue, cerebrospinal fluid (CSF), urine, blood, serum, or plasma. In certain embodiments, the sample comprises tissue, such as brain, liver, kidney, lung or spleen. The sample may include a fluid. In certain embodiments, the sample comprises CSF. In certain embodiments, the sample comprises blood and/or plasma. In certain embodiments, the sample comprises serum.

### TREM2

As used herein, the term “TREM2 protein” refers to a triggering receptor expressed on myeloid cells 2 protein that is encoded by the gene Trem2. As used herein, a “TREM2 protein” refers to a native (i.e., wild-type) TREM2 protein of any vertebrate, such as but not limited to human, non-human primates (e.g., cynomolgus monkey), rodents (e.g., mice, rat), and other mammals. In some embodiments, a TREM2 protein is a human TREM2 protein having the sequence identified in UniprotKB accession number Q9NZC2.

The TREM2 gene encodes a 230 amino acid-length protein that includes an extracellular domain, a transmembrane region and a short cytoplasmic tail (see, UniProtKB Q9NZC2; NCBI Reference Sequence: NP 061838.1). The extracellular region, encoded by exon 2, is composed of a single type V Ig-SF domain, containing three potential N-glycosylation sites. The putative transmembrane region contains a charged lysine residue. The cytoplasmic tail of TREM2 lacks signaling motifs and is thought to signal through the signaling adaptor molecule DAP12/TYROBP and through DAP10. TREM2 is found on the surface of osteoclasts, immature dendritic cells, and macrophages. In the central nervous system, TREM2 is exclusively expressed in microglia.

TREM2 may be cleaved by a disintegrin and metalloproteinase (ADAM) proteases (e.g., ADAM10 and ADAM17), which results in the release of soluble TREM2 (sTREM2) into the extracellular environment. As described herein, increased levels of TREM2 are indicative of downstream pathology in subjects having an LSD. Thus, TREM2 or sTREM2 levels can be measured using an assay known in the art or described herein. For example, assays for detecting and measuring levels of protein expression include, e.g., western blot analysis, immunofluorescence, immunohistochemistry (e.g., of tissue arrays), MesoScale Discovery (MSD) method, etc. In certain methods described herein, the concentration of TREM2 may be measured in a sample from a subject having, or suspected of having, an LSD. In certain methods described herein, the concentration of sTREM2 may be measured in a sample from a subject having, or suspected of having, an LSD.

In certain embodiments, the concentration of sTREM2 in a sample from a subject having an LSD is increased as compared to a control (e.g., a healthy control subject not having an LSD). In certain embodiments, the concentration of sTREM2 is increased at least about 1.25 fold, 1.5 fold, 1.75 fold, 2-fold, 3-fold, 4-fold, 5-fold, 6-fold, 7-fold, 8-fold, 9-fold, 10-fold or more as compared to a control. In certain embodiments, the increased sTREM2 concentration is observed in a tissue sample, such as brain. In certain embodiments, the increased sTREM2 concentration is observed in CSF. In certain embodiments, the increased sTREM2 concentration is observed in serum.

In certain other embodiments, a subject having an LSD is administered an effective LSD treatment, which decreases the concentration of sTREM2 in a sample from the subject as compared to a control (e.g., the same subject prior to receiving the treatment). In certain embodiments, the concentration of sTREM2 is decreased by at least about 5%, 10%, 15%, 20%, 25%, 30%, 35%, 40%, 45%, 50%, 55%, 60%, 65%, 70%, 75%, 80%, 85%, 90%, 95% or more as compared to a control. In certain embodiments, the decreased sTREM2 concentration is observed in a tissue sample, such as brain. In certain embodiments, the decreased sTREM2 concentration is observed in CSF. In certain embodiments, the decreased sTREM2 concentration is observed in serum.

### Nf-L

As used herein, the term “Nf-L” refers to neurofilament light chain (also referred to as neurofilament light chain polypeptide, neurofilament light polypeptide, and neurofilament light protein) that is encoded by the gene NEFL. As used herein, an “Nf-L protein” refers to a native (i.e., wild-type) Nf-L protein of any vertebrate, such as but not limited to human, non-human primates (e.g., cynomolgus monkey), rodents (e.g., mice, rat), and other mammals. In some embodiments, an Nf-L protein is a human Nf-L protein having the sequence identified in UniprotKB accession number P07196.

As described herein, increased levels of Nf-L are indicative of downstream pathology in subjects having an LSD. Thus, Nf-L levels can be measured using an assay known in the art or described herein. For example, assays for detecting and measuring levels of protein expression include, e.g., western blot analysis, immunofluorescence, immunohistochemistry (e.g., of tissue arrays), MesoScale Discovery (MSD) method, etc. In certain methods described herein, the concentration of Nf-L may be measured in a sample from a subject having, or suspected of having, an LSD.

In certain embodiments, the concentration of Nf-L in a sample from a subject having an LSD is increased as compared to a control (e.g., a healthy control subject not having an LSD). In certain embodiments, the concentration of Nf-L is increased at least about 1.25 fold, 1.5 fold, 1.75 fold, 2-fold, 3-fold, 4-fold, 5-fold, 6-fold, 7-fold, 8-fold, 9-fold, 10-fold or more as compared to a control. In certain embodiments, the increased Nf-L concentration is observed in a tissue sample, such as brain. In certain embodiments, the increased Nf-L concentration is observed in CSF. In certain embodiments, the increased Nf-L concentration is observed in serum.

In certain other embodiments, a subject having an LSD is administered an effective LSD treatment, which decreases the concentration of Nf-L in a sample from the subject as compared to a control (e.g., the same subject prior to receiving the treatment). In certain embodiments, the concentration of Nf-L is decreased by at least about 5%, 10%, 15%, 20%, 25%, 30%, 35%, 40%, 45%, 50%, 55%, 60%, 65%, 70%, 75%, 80%, 85%, 90%, 95% or more as compared to a control. In certain embodiments, the decreased Nf-L concentration is observed in a tissue sample, such as brain. In certain embodiments, the decreased Nf-L concentration is observed in CSF. In certain embodiments, the decreased Nf-L concentration is observed in serum.

### Lipids

As described herein, increased levels of a BMP, GlcCer, GD3, GD1a/b, GM2 and/or GM3 are indicative of downstream pathology in subjects having LSD. Thus, in certain methods described herein, the concentration of at least one BMP, GlcCer, GD3, GD1a/b, GM2 and/or GM3 may be measured in a sample from a subject having, or suspected of having, an LSD. The concentration of these lipids may be measured using an assay known in the art or described herein (e.g., by mass spectrometry).

Bis(monoacylglycero)phosphates (BMPs) refer to a class of an anionic phospholipids. BMPs are enriched in internal membranes of multivesicular endosomes and lysosomes and are thought to play a role in glycosphingolipid degradation and cholesterol transport (see, Kobayashi et al., Nat. Cell Biol. 1 (1999) 113-118). Particular BMP species are described herein (see, e.g., the Examples and Figures).

In certain embodiments, the concentration of at least one BMP species in a sample from a subject having an LSD is increased as compared to a control (e.g., a healthy control subject not having an LSD). In certain embodiments, the BMP is a BMP species described herein, such as in the Examples or Figures. For example, in certain embodiments, the BMP is BMP (44:12), BMP (36:2), BMP (di20:4), BMP (di22:6) or BMP (di18:1). In certain embodiments, the concentration of at least one BMP is increased by at least about 1.25 fold, 1.5 fold, 1.75 fold, 2-fold, 3-fold, 4-fold, 5-fold, 6-fold, 7-fold, 8-fold, 9-fold, 10-fold or more as compared to a control. In certain embodiments, the increased BMP concentration is observed in a tissue sample, such as brain. In certain embodiments, the increased BMP concentration is observed CSF. In certain embodiments, the increased BMP concentration is observed serum.

In certain other embodiments, a subject having an LSD is administered an effective LSD treatment, which decreases the concentration of at least one BMP species in a sample from the subject as compared to a control (e.g., the same subject prior to receiving the treatment). In certain embodiments, the concentration of at least one BMP is decreased by at least about 5%, 10%, 15%, 20%, 25%, 30%, 35%, 40%, 45%, 50%, 55%, 60%, 65%, 70%, 75%, 80%, 85%, 90%, 95% or more as compared to a control. In certain embodiments, the decreased BMP concentration is observed in a tissue sample, such as brain. In certain embodiments, the decreased BMP concentration is observed CSF. In certain embodiments, the decreased BMP concentration is observed serum.

Glucosylceramide (GlcCer) is a glycosphingolipid (ceramide and oligosaccharide) or oligoglycosylceramide with one or more sialic acids linked on the sugar chain. Particular GlcCer species are described herein (see, e.g., the Examples and Figures).

In certain embodiments, the concentration of at least one GlcCer species in a sample from a subject having an LSD is increased as compared to a control (e.g., a healthy control subject not having an LSD). In certain embodiments, the GlcCer is a GlcCer species described herein, such as in the Examples or Figures. For example, in certain embodiments, the GlcCer is GlcCer (d34:0), GlcCer (d34:1), GlcCer (d36:1), GlcCer (d42:1), GlcCer (d18:1, 16:0), GlcCer (d18:1, 18:0), GlcCer (d18:2, 18:0), GlcCer (d18:1, 20:0), GlcCer (d18:2, 20:0), GlcCer (d18:1, 22:0), GlcCer (d18:1, 22:1), GlcCer (d18:2, 22:0), GlcCer (d18:1, 24:1) or GlcCer (d18:1, 24:0). In certain embodiments, the GlcCer is GlcCer (d34:1), GlcCer (d36:1), GlcCer (d42:1), GlcCer (d18:1, 16:0) or GlcCer (d18:1, 22:0). In certain embodiments, the GlcCer is GlcCer (d34:0). In certain embodiments, the concentration of at least one GlcCer is increased by at least about 1.25 fold, 1.5 fold, 1.75 fold, 2-fold, 3-fold, 4-fold, 5-fold, 6-fold, 7-fold, 8-fold, 9-fold, 10-fold or more as compared to a control. In certain embodiments, the increased GlcCer concentration is observed in a tissue sample, such as brain. In certain embodiments, the increased GlcCer concentration is observed CSF. In certain embodiments, the increased GlcCer concentration is observed serum.

In certain other embodiments, a subject having an LSD is administered an effective LSD treatment, which decreases the concentration of at least one GlcCer species in a sample from the subject as compared to a control (e.g., the same subject prior to receiving the treatment). In certain embodiments, the concentration of at least one GlcCer is decreased by at least about 5%, 10%, 15%, 20%, 25%, 30%, 35%, 40%, 45%, 50%, 55%, 60%, 65%, 70%, 75%, 80%, 85%, 90%, 95% or more as compared to a control. In certain embodiments, the decreased GlcCer concentration is observed in a tissue sample, such as brain. In certain embodiments, the decreased GlcCer concentration is observed in CSF. In certain embodiments, the decreased GlcCer concentration is observed in serum.

Gangliosides are a type of glycosphingolipid. Particular GD3 species are described herein (see, e.g., the Examples and Figures).

In certain embodiments, the concentration of at least one GD3 species in a sample from a subject having an LSD is increased as compared to a control (e.g., a healthy control subject not having an LSD). In certain embodiments, the GD3 is a GD3 species described herein, such as in the Examples or Figures. For example, in certain embodiments, the GD3 is GD3 (d34:1), GD3 (d36:1), GD3 (d38:1), GD3 (d39:1), GD3 (d40:1), GD3 (d42:2) or GD3 (d42:1). In certain embodiments, the GD3 is GD3 (d34:1), GD3 (d36:1) or GD3 (d39:1). In certain embodiments, the GD3 is GD3 (d34:1) or GD3 (d36:1).

In certain embodiments, the concentration of at least one GD3 is increased by at least about 1.25 fold, 1.5 fold, 1.75 fold, 2-fold, 3-fold, 4-fold, 5-fold, 6-fold, 7-fold, 8-fold, 9-fold, 10-fold or more as compared to a control. In certain embodiments, the increased GD3 concentration is observed in a tissue sample, such as brain. In certain embodiments, the increased GD3 concentration is observed in CSF. In certain embodiments, the increased GD3 concentration is observed in serum.

In certain other embodiments, a subject having an LSD is administered an effective LSD treatment, which decreases the concentration of at least one GD3 species in a sample from the subject as compared to a control (e.g., the same subject prior to receiving the treatment). In certain embodiments, the concentration of at least one GD3 is decreased by at least about 5%, 10%, 15%, 20%, 25%, 30%, 35%, 40%, 45%, 50%, 55%, 60%, 65%, 70%, 75%, 80%, 85%, 90%, 95% or more as compared to a control. In certain embodiments, the decreased GD3 concentration is observed in a tissue sample, such as brain. In certain embodiments, the decreased GD3 concentration is observed in CSF. In certain embodiments, the decreased GD3 concentration is observed in serum.

Gangliosides GD1a and GD1b are glycosphingolipids. Particular GD1a/b species are described herein (see, e.g., the Examples and Figures).

In certain embodiments, the concentration of at least one GD1a/b species in a sample from a subject having an LSD is increased as compared to a control (e.g., a healthy control subject not having an LSD). In certain embodiments, the GD1a/b is a GD1a/b species described herein, such as in the Examples or Figures. For example, in certain embodiments, the GD1a/b is GD1a/b (d36:1) or GD1a/b (d38:1).

In certain embodiments, the concentration of at least one GD1a/b is increased by at least about 1.25 fold, 1.5 fold, 1.75 fold, 2-fold, 3-fold, 4-fold, 5-fold, 6-fold, 7-fold, 8-fold, 9-fold, 10-fold or more as compared to a control. In certain embodiments, the increased GD1a/b concentration is observed in a tissue sample, such as brain. In certain embodiments, the increased GD1a/b concentration is observed in CSF. In certain embodiments, the increased GD1a/b concentration is observed in serum.

In certain other embodiments, a subject having an LSD is administered an effective LSD treatment, which decreases the concentration of at least one GD1a/b species in a sample from the subject as compared to a control (e.g., the same subject prior to receiving the treatment). In certain embodiments, the concentration of at least one GD1a/b is decreased by at least 5%, 10%, 15%, 20%, 25%, 30%, 35%, 40%, 45%, 50%, 55%, 60%, 65%, 70%, 75%, 80%, 85%, 90%, 95% or more as compared to a control. In certain embodiments, the decreased GD1a/b concentration is observed in a tissue sample, such as brain. In certain embodiments, the decreased GD1a/b concentration is observed in CSF. In certain embodiments, the decreased GD1a/b concentration is observed in serum.

Ganglioside Monosialic 2 (GM2) is a glycosphingolipid. Particular GM2 species are described herein (see, e.g., the Examples and Figures).

In certain embodiments, the concentration of at least one GM2 species in a sample from a subject having an LSD is increased as compared to a control (e.g., a healthy control subject not having an LSD). In certain embodiments, the GM2 is a GM2 species described herein, such as in the Examples or Figures. For example, in certain embodiments the GM2 species is GM2 (d38:1) or GM2 (d36:1). In certain embodiments, the concentration of at least one GM2 is increased by at least about 1.25 fold, 1.5 fold, 1.75 fold, 2-fold, 3-fold, 4-fold, 5-fold, 6-fold, 7-fold, 8-fold, 9-fold, 10-fold or more as compared to a control. In certain embodiments, the increased GM2 concentration is observed in a tissue sample, such as brain. In certain embodiments, the increased GM2 concentration is observed in CSF. In certain embodiments, the increased GM2 concentration is observed in serum.

In certain other embodiments, a subject having an LSD is administered an effective LSD treatment, which decreases the concentration of at least one GM2 species in a sample from the subject as compared to a control (e.g., the same subject prior to receiving the treatment). In certain embodiments, the concentration of at least one GM2 is decreased by at least about 5%, 10%, 15%, 20%, 25%, 30%, 35%, 40%, 45%, 50%, 55%, 60%, 65%, 70%, 75%, 80%, 85%, 90%, 95% or more as compared to a control. In certain embodiments, the decreased GM2 concentration is observed in a tissue sample, such as brain. In certain embodiments, the decreased GM2 concentration is observed in CSF. In certain embodiments, the decreased GM2 concentration is observed in serum.

Similar to GM2, Ganglioside Monosialic 3 (GM3) is also a class of glycosphingolipids. Particular GM3 species are described herein (see, e.g., the Examples and Figures).

In certain embodiments, the concentration of at least one GM3 species in a sample from a subject having an LSD is increased as compared to a control (e.g., a healthy control subject not having an LSD). In certain embodiments, the GM3 is a GM3 species described herein, such as in the Examples or Figures. For example, in certain embodiments, the GM3 species is GM3 (d34:1), GM3 (d36:1), GM3 (d38:1), GM3 (d40:1), GM3 (d41:1), GM3 (d42:2), GM3 (d42:1), GM3 (d43:0), GM3 (d44:1) or GM3 (d44:2). In certain embodiments, the GM3 species is GM3 (d34:1), GM3 (d36:1) or GM3 (d38:1). In certain embodiments, the concentration of at least one GM3 is increased by at least about 1.25 fold, 1.5 fold, 1.75 fold, 2-fold, 3-fold, 4-fold, 5-fold, 6-fold, 7-fold, 8-fold, 9-fold, 10-fold or more as compared to a control. In certain embodiments, the increased GM3 concentration is observed in a tissue sample, such as brain. In certain embodiments, the increased GM3 concentration is observed CSF. In certain embodiments, the increased GM3 concentration is observed serum.

In certain other embodiments, a subject having an LSD is administered an effective LSD treatment, which decreases the concentration of at least one GM3 in a sample from the subject as compared to a control. In certain embodiments, the concentration of at least one GM3 is decreased by at least about 5%, 10%, 15%, 20%, 25%, 30%, 35%, 40%, 45%, 50%, 55%, 60%, 65%, 70%, 75%, 80%, 85%, 90%, 95% or more as compared to a control. In certain embodiments, the decreased GM3 concentration is observed in a tissue sample, such as brain. In certain embodiments, the decreased GM3 concentration is observed CSF. In certain embodiments, the decreased GM3 concentration is observed serum.

### Measurement of TREM2, Nf-L, Lipids, and Lipid Combinations

As used herein, the terms “combination of two or more lipids” refers to two or more lipids, wherein at least two lipids are from different classes, wherein the classes are selected from a) BMPs; b) GlcCers; c) GD3 gangliosides; d) GD1a/b gangliosides; and e) GM2 and/or GM3 gangliosides.

In certain embodiments, a combination of two or more lipids selected from the group consisting of a BMP, GlcCer, GD3, GD1a/b, GM2 and GM3 are evaluated or have been evaluated. In certain embodiments, the combination comprises a BMP. In certain embodiments, the combination comprises a GlcCer. In certain embodiments, the combination comprises a GD3. In certain embodiments, the combination comprises a GD1a/b. In certain embodiments, the combination comprises a GM2. In certain embodiments, the combination comprises a GM3. In certain embodiments, the combination comprises a BMP and a GlcCer. In certain embodiments, the combination comprises a BMP and a GD3. In certain embodiments, the combination comprises a BMP and a GD1a/b. In certain embodiments, the combination comprises a BMP and a GM2. In certain embodiments, the combination comprises a BMP and a GM3. In certain embodiments, the combination comprises a GlcCer and a GD3. In certain embodiments, the combination comprises a GlcCer and a GD1a/b. In certain embodiments, the combination comprises a GlcCer and a GM2. In certain embodiments, the combination comprises a GlcCer and a GM3. In certain embodiments, the combination comprises a GD3 and a GD1a/b. In certain embodiments, the combination comprises a GD3 and a GM2. In certain embodiments, the combination comprises a GD3 and a GM3. In certain embodiments, the combination comprises a GD1a/b and a GM2. In certain embodiments, the combination comprises a GD1a/b and a GM3. In certain embodiments, the combination does not consist of a GM2 and a GM3.

In certain embodiments, a combination of three or more lipids selected from the group consisting of a BMP, GlcCer, GD3, GD1a/b, GM2 and GM3 are evaluated or have been evaluated. In certain embodiments, the combination comprises a BMP, a GlcCer and a GD3. In certain embodiments, the combination comprises a BMP, a GlcCer and a GD1a/b. In certain embodiments, the combination comprises a BMP, a GlcCer and a GM2. In certain embodiments, the combination comprises a BMP, a GlcCer and a GM3. In certain embodiments, the combination comprises a BMP, a GD3 and a GD1a/b. In certain embodiments, the combination comprises a BMP, a GD3 and a GM2. In certain embodiments, the combination comprises a BMP, a GD3 and a GM3. In certain embodiments, the combination comprises a BMP, a GD1a/b and a GM2. In certain embodiments, the combination comprises a BMP, a GD1a/b and a GM3. In certain embodiments, the combination comprises a BMP, a GM2 and a GM3. In certain embodiments, the combination comprises a GlcCer, a GD3 and a GD1a/b. In certain embodiments, the combination comprises a GlcCer, a GD3 and a GM2. In certain embodiments, the combination comprises a GlcCer, a GD3 and a GM3. In certain embodiments, the combination comprises a GlcCer, a GD1a/b and a GM2. In certain embodiments, the combination comprises a GlcCer, a GD1a/b and a GM3. In certain embodiments, the combination comprises a GlcCer, a GM2 and a GM3. In certain embodiments, the combination comprises a GD3, a GD1a/b and a GM2. In certain embodiments, the combination comprises a GD3, a GD1a/b and a GM3. In certain embodiments, the combination comprises a GD3, GM2 and a GM3. In certain embodiments, the combination comprises a GD1a/b, a GM2 and a GM3.

In certain embodiments, a combination of four or more lipids selected from the group consisting of a BMP, GlcCer, GD3, GD1a/b, GM2 and GM3 are evaluated or have been evaluated. In certain embodiments, the combination comprises a BMP, a GlcCer, a GD3 and a GD1a/b. In certain embodiments, the combination comprises a BMP, a GlcCer, a GD3 and a GM2. In certain embodiments, the combination comprises a BMP, a GlcCer, a GD3 and a GM3. In certain embodiments, the combination comprises a BMP, a GlcCer, a GD1a/b and GM2. In certain embodiments, the combination comprises a BMP, a GlcCer, a GD1a/b and GM3. In certain embodiments, the combination comprises a BMP, a GlcCer, a GM2 and GM3. In certain embodiments, the combination comprises a BMP, a GD3, a GD1a/b and a GM2. In certain embodiments, the combination comprises a BMP, a GD3, a GD1a/b and a GM3. In certain embodiments, the combination comprises a BMP, a GD3, a GM2 and a GM3. In certain embodiments, the combination comprises a BMP, a GD1a/b, a GM2 and a GM3. In certain embodiments, the combination comprises a GlcCer, a GD3, a GD1a/b and a GM2. In certain embodiments, the combination comprises a GlcCer, a GD3, a GD1a/b and a GM3. In certain embodiments, the combination comprises a GlcCer, a GD3, a GM2 and a GM3. In certain embodiments, the combination comprises a GlcCer, a GD1a/b, a GM2 and a GM3. In certain embodiments, the combination comprises a GD3, a GD1a/b, a GM2 and a GM3.

In certain embodiments, a combination of five or more lipids selected from the group consisting of a BMP, GlcCer, GD3, GD1a/b, GM2 and GM3 are evaluated or have been evaluated. In certain embodiments, a combination comprises a BMP, a GlcCer, a GD3, a GD1a/b and a GM2. In certain embodiments, a combination comprises a BMP, a GlcCer, a GD3, a GD1a/b and a GM3. In certain embodiments, a combination comprises a BMP, a GD3, a GD1a/b, a GM2 and a GM3. In certain embodiments, a combination comprises a BMP, a GlcCer, a GD3, a GM2 and a GM3. In certain embodiments, a combination comprises a BMP, a GlcCer, a GD1a/b, a GM2 and a GM3. In certain embodiments, a combination comprises a GlcCer, a GD3, a GD1a/b, a GM2 and a GM3.

In certain embodiments, a combination of BMP, GlcCer, GD3, GD1a/b, GM2 and GM3 are evaluated or have been evaluated.

In certain embodiments, sTREM2 is evaluated or has been evaluated. In certain embodiments, sTREM2 and one or more lipids are evaluated or have been evaluated. In certain embodiments, the lipid is a BMP. In certain embodiments, the lipid is a GlcCer. In certain embodiments, the lipid is a GD3. In certain embodiments, the lipid is a GD1a/b. In certain embodiments, the lipid is a GM2. In certain embodiments, the lipid is a GM3.

In certain embodiments, sTREM2 and a combination of two or more lipids selected from the group consisting of a BMP, a GlcCer, a GD3, a GD1a/b, a GM2 and a GM3 are evaluated or have been evaluated. In certain embodiments, the combination of two or more lipids is a combination described herein.

In certain embodiments, sTREM2 and a combination of three or more lipids selected from the group consisting of a BMP, GlcCer, GD3, GD1a/b, GM2 and GM3 are evaluated or have been evaluated. In certain embodiments, the combination of three or more lipids is a combination described herein.

In certain embodiments, sTREM2 and a combination of four or more lipids selected from the group consisting of a BMP, GlcCer, GD3, GD1a/b, GM2 and GM3 are evaluated or have been evaluated. In certain embodiments, the combination of four or more lipids is a combination described herein.

In certain embodiments, sTREM2 and a combination of five or more lipids selected from the group consisting of a BMP, GlcCer, GD3, GD1a/b, GM2 and GM3 are evaluated or have been evaluated. In certain embodiments, the combination of five or more lipids is a combination described herein.

In certain embodiments, sTREM2, BMP, GlcCer, GD3, GD1a/b, GM2 and GM3 are evaluated or have been evaluated.

In certain embodiments, Nf-L is evaluated or has been evaluated.

In certain embodiments, Nf-L and sTREM2 are evaluated or have been evaluated.

In certain embodiments, Nf-L and one or more lipids are evaluated or have been evaluated. In certain embodiments, the lipid is a BMP. In certain embodiments, the lipid is a GlcCer. In certain embodiments, the lipid is a GD3. In certain embodiments, the lipid is a GD1a/b. In certain embodiments, the lipid is a GM2. In certain embodiments, the lipid is a GM3.

In certain embodiments, Nf-L and a combination of two or more lipids selected from the group consisting of a BMP, a GlcCer, a GD3, a GD1a/b, a GM2 and a GM3 are evaluated or have been evaluated. In certain embodiments, the combination of two or more lipids is a combination described herein.

In certain embodiments, Nf-L and a combination of three or more lipids selected from the group consisting of a BMP, GlcCer, GD3, GD1a/b, GM2 and GM3 are evaluated or have been evaluated. In certain embodiments, the combination of three or more lipids is a combination described herein.

In certain embodiments, Nf-L and a combination of four or more lipids selected from the group consisting of a BMP, GlcCer, GD3, GD1a/b, GM2 and GM3 are evaluated or have been evaluated. In certain embodiments, the combination of four or more lipids is a combination described herein.

In certain embodiments, Nf-L and a combination of five or more lipids selected from the group consisting of a BMP, GlcCer, GD3, GD1a/b, GM2 and GM3 are evaluated or have been evaluated. In certain embodiments, the combination of five or more lipids is a combination described herein.

In certain embodiments, Nf-L, BMP, GlcCer, GD3, GD1a/b, GM2 and GM3 are evaluated or have been evaluated.

In certain embodiments, sTREM2, Nf-L and one or more lipids are evaluated or have been evaluated. In certain embodiments, sTREM2, Nf-L, BMP, GlcCer, GD3, GD1a/b, GM2 and GM3 are evaluated or have been evaluated.

Thus, a sample obtained from a subject having, or suspected of having an LSD, may be evaluated for an accumulation of sTREM2, Nf-L and/or one or more lipids selected from a BMP, a GlcCer, a GD3, a GD1a/b, a GM2 and a GM3. Specifically, the concentration of the sTREM2 protein, Nf-L and/or the one or more lipids may be measured in a sample obtained from the subject using an assay known in the art or described herein (e.g., mass spectrometry).

In certain embodiments, the concentration of the protein and/or lipids is compared to the concentration of the corresponding protein and/or lipids in a sample from a control subject (e.g., a healthy subject that does not have LSD).

In some embodiments, the amount of each of the selected lipids/proteins in the sample from the subject is compared to a control value that is determined for a healthy control or population of healthy controls (i.e., not afflicted with an LSD). In some embodiments, the subject is identified as a candidate for treatment or for a treatment adjustment if the amount of each of the selected lipids/proteins in the sample from the subject is increased as compared to the control value. In some embodiments, the subject is identified as a candidate for treatment or for a treatment adjustment if the amount of each of the selected lipids/proteins in the sample from the subject is increased by at least 20%, at least 30%, at least 40%, at least 50%, at least 60%, at least 70%, at least 80%, at least 90% or more as compared to the control value. In some embodiments, the subject is identified as a candidate for treatment or for a treatment adjustment if the amount of each of the selected lipids/proteins in the sample from the subject is increased by at least 2-fold, 3-fold, 4-fold, 5-fold, 6-fold, 7-fold, 8-fold, 9-fold, 10-fold or more as compared to the control value. In some embodiments, the healthy control value for each of the selected lipids/proteins is determined by assessing the level of each lipid/protein in a subject or population of subjects (e.g., 10, 20, 50, 100, 200, 500, 1000 subjects or more) that all are known not to have an LSD.

In some embodiments, the amount of each of the selected lipids/proteins in the sample from the subject is compared to a control value that is determined for a disease control or population of disease controls (i.e., afflicted with an LSD). In some embodiments, the disease control value for each of the selected lipids/proteins is determined by assessing the level of the selected lipids/proteins in a subject or population of subjects (e.g., 10, 20, 50, 100, 200, 500, 1000 subjects or more) that all are known to have an LSD.

In some embodiments, the subject is identified as a candidate for treatment or for treatment adjustment (e.g., an increase in dosage or frequency) if the amount of each of the selected lipids/proteins in the sample from the subject is at least as high as an amount of each of the selected lipids/proteins in the disease control or population of disease controls. In some embodiments, the subject is identified as a candidate for treatment or for treatment adjustment if the amount of each of the selected lipids/proteins in the sample from the subject is comparable to (e.g., is within 20%, 10%, 5%, 4%, 3%, 2%, or 1%) the amount of each of the selected lipids/proteins in the disease control or population of disease controls.

In some embodiments, the subject having the LSD is a subject that has been administered a treatment for the LSD. The level in the subject after receiving the treatment is compared to the level in the same subject prior to treatment administration (e.g., prior to the first administration) of the treatment. The effectiveness of the treatment may be determined by the change (e.g., reduction) in the amount of the selected lipids/proteins.

In certain other embodiments, the subject is identified as a candidate for treatment adjustment (e.g., a decrease in dosage or frequency) if the amount of each of the selected lipids/proteins in the sample from the subject is less than amount of each of the selected lipids/proteins in the disease control or population of disease controls.

In some embodiments, the population of subjects is matched to a test subject according to one or more patient characteristics such as age, sex, ethnicity, or other criteria. In some embodiments, the control value is established using the same type of sample from the population of subjects (e.g., a sample comprising blood or PBMCs) as is used for assessing the level of lipids/proteins in the test subject.

### Lysosomal Storage Disorders

As described herein, certain biomarkers associated with LSDs have been identified. LSDs are inherited metabolic diseases characterized by the accumulation of undigested or partially digested macromolecules, which ultimately results in cellular dysfunction and clinical abnormalities. Classically, LSDs have been defined as deficiencies in lysosomal function generally classified by the accumulated substrate and include mucopolysaccharidoses. The classification of these disorders has recently been expanded to include other deficiencies or defects in proteins that result in accumulation of macromolecules, such as proteins necessary for normal post-translational modification of lysosomal enzymes, or proteins important for proper lysosomal trafficking.

In certain embodiments, the LSD is an MPS disorder (e.g., Hunter syndrome).

### Therapeutic Agents

Certain methods described herein comprise administering a lysosomal storage disorder treatment to a subject.

As used herein, a “lysosomal storage disorder treatment” may be any therapeutic agent or therapy capable of reducing one or more symptoms associated with an LSD (e.g., a neurological symptom). Certain lysosomal storage disorder treatments are known. For example, such treatments include, e.g., haematopoietic stein cell transplantation (HSCT), enzyme replacement therapies (ERT), substrate reduction therapies, chaperone therapy and gene therapy (e.g., in vivo or ex vivo).

In certain embodiments, an LSD treatment comprises ERT. In certain embodiments, the ERT may be a therapy that is designed to treat one or more neurological symptoms. As described below, certain ERT LSD therapies may be targeted to the brain using an enzyme transport vehicle (ETV). For example, certain fusion proteins comprising ERT enzymes, which may be used in a method described herein, are discussed below and are described in WO 2019/070577, which is incorporated by reference herein for all purposes.

### Certain Fusion Proteins Comprising ERT Enzymes

Described below are certain embodiments of fusion proteins that include an enzyme replacement therapy (ERT) enzyme linked to an Fc polypeptide; these fusion proteins may be used in certain methods described herein as an LSD treatment. In some cases, the protein includes a dimeric Fc polypeptide, where one of the Fc polypeptide monomers is linked to the ERT enzyme. The Fc polypeptides can increase enzyme half-life and, in some cases, can be modified to confer additional functional properties onto the protein. Also described herein are fusion proteins that facilitate delivery of an ERT enzyme across the blood-brain barrier (BBB). These proteins comprise an Fc polypeptide and a modified Fc polypeptide that form a dimer, and an ERT enzyme linked to the Fc region and/or the modified Fc region. The modified Fc region can specifically bind to a BBB receptor such as a transferrin receptor (TfR). In some embodiments, the ERT enzyme is iduronate 2-sulfatase (IDS), or a catalytically active variant or fragment of a wild-type IDS, e.g., a wild-type human IDS. Certain embodiments of these fusion proteins may be referenced herein as an enzyme transport vehicle (ETV) in conjunction with the particular enzyme, for example ETV:IDS.

### ERT Enzymes

In some aspects, a fusion protein described herein comprises: (i) an Fc polypeptide, which may contain modifications (e.g., one or more modifications that promote heterodimerization) or may be a wild-type Fc polypeptide; and an ERT enzyme; and (ii) an Fc polypeptide, which may contain modifications (e.g., one or more modifications that promote heterodimerization) or may be a wild-type Fc polypeptide; and optionally an ERT enzyme. In some embodiments, one or both Fc polypeptides may contain modifications that result in binding to a blood-brain barrier (BBB) receptor, e.g., a TfR. The ERT enzyme may be any enzyme that is deficient in an LSD. An ERT enzyme incorporated into the fusion protein is catalytically active, i.e., it retains the enzymatic activity that is deficient in the LSD. In some embodiments, the ERT enzyme is IDS, which is deficient in Hunter syndrome.

In some embodiments, a fusion protein comprising an ERT enzyme and optionally a modified Fc polypeptide that binds to a BBB receptor, e.g., a TfR-binding Fc polypeptide, comprises a catalytically active fragment or variant of a wild-type IDS. In some embodiments, the IDS enzyme is a variant or a catalytically active fragment of an IDS protein that comprises the amino acid sequence of any one of SEQ ID NOS:91, 92, 112, 192, and 196. In some embodiments, a catalytically active variant or fragment of an IDS enzyme has at least 50%, at least 55%, at least 60%, at least 65%, at least 70%, at least 75%, at least 80%, at least 85%, at least 90%, at least 95%, or greater of the activity of the wild-type IDS enzyme.

In some embodiments, an ERT enzyme (e.g., IDS), or a catalytically active variant or fragment thereof, that is present in a fusion protein described herein, retains at least 25% of its activity compared to its activity when not joined to an Fc polypeptide or a TfR-binding Fc polypeptide. In some embodiments, an ERT enzyme, or a catalytically active variant or fragment thereof, retains at least 10%, or at least 15%, 20%, 25%, 30%, 35%, 40%, 45%, 50%, 55%, 60%, 65%, 70%, 75%, 80%, 85%, 90%, or 95%, of its activity compared to its activity when not joined to an Fc polypeptide or a TfR-binding Fc polypeptide. In some embodiments, an ERT enzyme, or a catalytically active variant or fragment thereof, retains at least 80%, 85%, 90%, or 95% of its activity compared to its activity when not joined to an Fc polypeptide or a TfR-binding Fc polypeptide. In some embodiments, fusion to an Fc polypeptide does not decrease the activity of the ERT enzyme, e.g., IDS, or catalytically active variant or fragment thereof. In some embodiments, fusion to a TfR-binding Fc polypeptide does not decrease the activity of the ERT enzyme.

I. Fc Polypeptide Modifications for Blood-Brain Barrier (BBB) Receptor Binding

In some aspects, the fusion proteins are capable of being transported across the blood-brain barrier (BBB). Such a protein comprises a modified Fc polypeptide that binds to a BBB receptor. BBB receptors are expressed on BBB endothelia, as well as other cell and tissue types. In some embodiments, the BBB receptor is transferrin receptor (TfR).

Amino acid residues designated in various Fc modifications, including those introduced in a modified Fc polypeptide that binds to a BBB receptor, e.g., TfR, are numbered herein using EU index numbering. Any Fc polypeptide, e.g., an IgG1, IgG2, IgG3, or IgG4 Fc polypeptide, may have modifications, e.g., amino acid substitutions, in one or more positions as described herein.

A modified (e.g., enhancing heterodimerization and/or BBB receptor-binding) Fc polypeptide present in a fusion protein described herein can have at least 70% identity, at least 75% identity, at least 80% identity, at least 85% identity, at least 90% identity, or at least 95% identity to a native Fc region sequence or a fragment thereof, e.g., a fragment of at least 50 amino acids or at least 100 amino acids, or greater in length. In some embodiments, the native Fc amino acid sequence is the Fc region sequence of SEQ ID NO:1. In some embodiments, the modified Fc polypeptide has at least 70% identity, at least 75% identity, at least 80% identity, at least 85% identity, at least 90% identity, or at least 95% identity to amino acids 1-110 of SEQ ID NO:1, or to amino acids 111-217 of SEQ ID NO:1, or a fragment thereof, e.g., a fragment of at least 50 amino acids or at least 100 amino acids, or greater in length.

In some embodiments, a modified (e.g., enhancing heterodimerization and/or BBB receptor-binding) Fc polypeptide comprises at least 50 amino acids, or at least 60, 65, 70, 75, 80, 85, 90, or 95 or more, or at least 100 amino acids, or more, that correspond to a native Fc region amino acid sequence. In some embodiments, the modified Fc polypeptide comprises at least 25 contiguous amino acids, or at least 30, 35, 40, or 45 contiguous amino acids, or 50 contiguous amino acids, or at least 60, 65, 70, 75, 80 85, 90, or 95 or more contiguous amino acids, or 100 or more contiguous amino acids, that correspond to a native Fc region amino acid sequence, such as SEQ ID NO:1.

In some embodiments, the domain that is modified for BBB receptor-binding activity is a human Ig CH3 domain, such as an IgG1 CH3 domain. The CH3 domain can be of any IgG subtype, i.e., from IgG1, IgG2, IgG3, or IgG4. In the context of IgG1 antibodies, a CH3 domain refers to the segment of amino acids from about position 341 to about position 447 as numbered according to the EU numbering scheme.

In some embodiments, a modified (e.g., BBB receptor-binding) Fc polypeptide present in a fusion protein described herein comprises at least one, two, or three substitutions; and in some embodiments, at least four, five, six, seven, eight, or nine substitutions at amino acid positions 384, 386, 387, 388, 389, 390, 413, 416, and 421, according to the EU numbering scheme.

### FcRn Binding Sites

In certain aspects, modified (e.g., BBB receptor-binding) Fc polypeptides, or Fc polypeptides present in a fusion protein described herein that do not specifically bind to a BBB receptor, can also comprise an FcRn binding site. In some embodiments, the FcRn binding site is within the Fc polypeptide or a fragment thereof.

In some embodiments, the FcRn binding site comprises a native FcRn binding site. In some embodiments, the FcRn binding site does not comprise amino acid changes relative to the amino acid sequence of a native FcRn binding site. In some embodiments, the native FcRn binding site is an IgG binding site, e.g., a human IgG binding site. In some embodiments, the FcRn binding site comprises a modification that alters FcRn binding.

In some embodiments, an FcRn binding site has one or more amino acid residues that are mutated, e.g., substituted, wherein the mutation(s) increase serum half-life or do not substantially reduce serum half-life (i.e., reduce serum half-life by no more than 25% compared to a counterpart modified Fc polypeptide having the wild-type residues at the mutated positions when assayed under the same conditions). In some embodiments, an FcRn binding site has one or more amino acid residues that are substituted at positions 250-256, 307, 380, 428, and 433-436, according to the EU numbering scheme.

In some embodiments, one or more residues at or near an FcRn binding site are mutated, relative to a native human IgG sequence, to extend serum half-life of the modified polypeptide. In some embodiments, mutations are introduced into one, two, or three of positions 252, 254, and 256. In some embodiments, the mutations are M252Y, S254T, and T256E. In some embodiments, a modified Fc polypeptide further comprises the mutations M252Y, S254T, and T256E. In some embodiments, a modified Fc polypeptide comprises a substitution at one, two, or all three of positions T307, E380, and N434, according to the EU numbering scheme. In some embodiments, the mutations are T307Q and N434A. In some embodiments, a modified Fc polypeptide comprises mutations T307A, E380A, and N434A. In some embodiments, a modified Fc polypeptide comprises substitutions at positions T250 and M428, according to the EU numbering scheme. In some embodiments, the modified Fc polypeptide comprises mutations T250Q and/or M428L. In some embodiments, a modified Fc polypeptide comprises substitutions at positions M428 and N434, according to the EU numbering scheme. In some embodiments, the modified Fc polypeptide comprises mutations M428L and N434S. In some embodiments, a modified Fc polypeptide comprises an N434S or N434A mutation.

II. Transferrin Receptor-Binding Fc Polypeptides

This section describes generation of modified Fc polypeptides described herein that bind to transferrin receptor (TfR) and are capable of being transported across the blood-brain barrier (BBB).

### TfR-Binding Fc Polypeptides Comprising Mutations in the CH3 Domain

In some embodiments, a modified Fc polypeptide that specifically binds to TfR comprises substitutions in a CH3 domain. In some embodiments, a modified Fc polypeptide comprises a human Ig CH3 domain, such as an IgG CH3 domain, that is modified for TfR-binding activity. The CH3 domain can be of any IgG subtype, i.e., from IgG1, IgG2, IgG3, or IgG4. In the context of IgG antibodies, a CH3 domain refers to the segment of amino acids from about position 341 to about position 447 as numbered according to the EU numbering scheme.

In some embodiments, a modified Fc polypeptide that specifically binds to TfR binds to the apical domain of TfR and may bind to TfR without blocking or otherwise inhibiting binding of transferrin to TfR. In some embodiments, binding of transferrin to TfR is not substantially inhibited. In some embodiments, binding of transferrin to TfR is inhibited by less than about 50% (e.g., less than about 45%, 40%, 35%, 30%, 25%, 20%, 15%, 10%, or 5%). In some embodiments, binding of transferrin to TfR is inhibited by less than about 20% (e.g., less than about 19%, 18%, 17%, 16%, 15%, 14%, 13%, 12%, 11%, 10%, 9%, 8%, 7%, 6%, 5%, 4%, 3%, 2%, or 1%).

In some embodiments, a modified Fc polypeptide that specifically binds to TfR comprises at least two, three, four, five, six, seven, eight, or nine substitutions at positions 384, 386, 387, 388, 389, 390, 413, 416, and 421, according to the EU numbering scheme. Illustrative substitutions that may be introduced at these positions are shown in Tables 4 and 5. In some embodiments, the amino acid at position 388 and/or 421 is an aromatic amino acid, e.g., Trp, Phe, or Tyr. In some embodiments, the amino acid at position 388 is Trp. In some embodiments, the aromatic amino acid at position 421 is Trp or Phe.

In some embodiments, at least one position as follows is substituted: Leu, Tyr, Met, or Val at position 384; Leu, Thr, His, or Pro at position 386; Val, Pro, or an acidic amino acid at position 387; an aromatic amino acid, e.g., Trp at position 388; Val, Ser, or Ala at position 389; an acidic amino acid, Ala, Ser, Leu, Thr, or Pro at position 413; Thr or an acidic amino acid at position 416; or Trp, Tyr, His, or Phe at position 421. In some embodiments, the modified Fc polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set. Thus, for example, Ile may be present at position 384, 386, and/or position 413. In some embodiments, the acidic amino acid at position one, two, or each of positions 387, 413, and 416 is Glu. In other embodiments, the acidic amino acid at one, two or each of positions 387, 413, and 416 is Asp. In some embodiments, two, three, four, five, six, seven, or all eight of positions 384, 386, 387, 388, 389, 413, 416, and 421 have an amino acid substitution as specified in this paragraph.

In some embodiments, an Fc polypeptide that is modified as described in the preceding two paragraphs comprises a native Asn at position 390. In some embodiments, the modified Fc polypeptide comprises Gly, His, Gln, Leu, Lys, Val, Phe, Ser, Ala, or Asp at position 390. In some embodiments, the modified Fc polypeptide further comprises one, two, three, or four substitutions at positions comprising 380, 391, 392, and 415, according to the EU numbering scheme. In some embodiments, Trp, Tyr, Leu, or Gln may be present at position 380. In some embodiments, Ser, Thr, Gln, or Phe may be present at position 391. In some embodiments, Gln, Phe, or His may be present at position 392. In some embodiments, Glu may be present at position 415.

In certain embodiments, the modified Fc polypeptide comprises two, three, four, five, six, seven, eight, nine, ten, or eleven positions selected from the following: Trp, Leu, or Glu at position 380; Tyr or Phe at position 384; Thr at position 386; Glu at position 387; Trp at position 388; Ser, Ala, Val, or Asn at position 389; Ser or Asn at position 390; Thr or Ser at position 413; Glu or Ser at position 415; Glu at position 416; and/or Phe at position 421. In some embodiments, the modified Fc polypeptide comprises all eleven positions as follows: Trp, Leu, or Glu at position 380; Tyr or Phe at position 384; Thr at position 386; Glu at position 387; Trp at position 388; Ser, Ala, Val, or Asn at position 389; Ser or Asn at position 390; Thr or Ser at position 413; Glu or Ser at position 415; Glu at position 416; and/or Phe at position 421.

In certain embodiments, the modified Fc polypeptide comprises Leu or Met at position 384; Leu, His, or Pro at position 386; Val at position 387; Trp at position 388; Val or Ala at position 389; Pro at position 413; Thr at position 416; and/or Trp at position 421. In some embodiments, the modified Fc polypeptide further comprises Ser, Thr, Gln, or Phe at position 391. In some embodiments, the modified Fc polypeptide further comprises Trp, Tyr, Leu, or Gln at position 380 and/or Gln, Phe, or His at position 392. In some embodiments, Trp is present at position 380 and/or Gln is present at position 392. In some embodiments, the modified Fc polypeptide does not have a Trp at position 380.

In other embodiments, the modified Fc polypeptide comprises Tyr at position 384; Thr at position 386; Glu or Val and position 387; Trp at position 388; Ser at position 389; Ser or Thr at position 413; Glu at position 416; and/or Phe at position 421. In some embodiments, the modified Fc polypeptide comprises a native Asn at position 390. In certain embodiments, the modified Fc polypeptide further comprises Trp, Tyr, Leu, or Gln at position 380; and/or Glu at position 415. In some embodiments, the modified Fc polypeptide further comprises Trp at position 380 and/or Glu at position 415.

In additional embodiments, the modified Fc polypeptide further comprises one, two, or three substitutions at positions comprising 414, 424, and 426, according to the EU numbering scheme. In some embodiments, position 414 is Lys, Arg, Gly, or Pro; position 424 is Ser, Thr, Glu, or Lys; and/or position 426 is Ser, Trp, or Gly.

In some embodiments, the modified Fc polypeptide comprises one or more of the following substitutions: Trp at position 380; Thr at position 386; Trp at position 388; Val at position 389; Thr or Ser at position 413; Glu at position 415; and/or Phe at position 421, according to the EU numbering scheme.

In some embodiments, the modified Fc polypeptide has at least 70% identity, at least 75% identity, at least 80% identity, at least 85% identity, at least 90% identity, or at least 95% identity to amino acids 111-217 of any one of SEQ ID NOS:4-90, 95-98, and 103-106 (e.g., SEQ ID NOS:34-38, 58, and 60-90). In some embodiments, the modified Fc polypeptide has at least 70% identity, at least 75% identity, at least 80% identity, at least 85% identity, at least 90% identity, or at least 95% identity to any one of SEQ ID NOS:4-90, 95-98, and 103-106 (e.g., SEQ ID NOS:34-38, 58, and 60-90). In some embodiments, the modified Fc polypeptide comprises the amino acids at EU index positions 384-390 and/or 413-421 of any one of SEQ ID NOS:4-90, 95-98, and 103-106 (e.g., SEQ ID NOS:34-38, 58, and 60-90). In some embodiments, the modified Fc polypeptide comprises the amino acids at EU index positions 380-390 and/or 413-421 of any one of SEQ ID NOS:4-90, 95-98, and 103-106 (e.g., SEQ ID NOS:34-38, 58, and 60-90). In some embodiments, the modified Fc polypeptide comprises the amino acids at EU index positions 380-392 and/or 413-426 of any one of SEQ ID NOS:4-90, 95-98, and 103-106 (e.g.,

SEQ ID NOS:34-38, 58, and 60-90).

In some embodiments, the modified Fc polypeptide has at least 75% identity, at least 80% identity, at least 85% identity, at least 90% identity, or at least 95% identity to any one of SEQ ID NOS:4-90, 95-98, and 103-106 (e.g., SEQ ID NOS:34-38, 58, and 60-90), and further comprises at least five, six, seven, eight, nine, ten, eleven, twelve, thirteen, fourteen, fifteen, or sixteen of the positions, numbered according to the EU index, as follows: Trp, Tyr, Leu, Gln, or Glu at position 380; Leu, Tyr, Met, or Val at position 384; Leu, Thr, His, or Pro at position 386; Val, Pro, or an acidic amino acid at position 387; an aromatic amino acid, e.g., Trp, at position 388; Val, Ser, or Ala at position 389; Ser or Asn at position 390; Ser, Thr, Gln, or Phe at position 391; Gln, Phe, or His at position 392; an acidic amino acid, Ala, Ser, Leu, Thr, or Pro at position 413; Lys, Arg, Gly or Pro at position 414; Glu or Ser at position 415; Thr or an acidic amino acid at position 416; Trp, Tyr, His or Phe at position 421; Ser, Thr, Glu or Lys at position 424; and Ser, Trp, or Gly at position 426.

In some embodiments, the modified Fc polypeptide comprises the amino acid sequence of any one of SEQ ID NOS:34-38, 58, and 60-90. In other embodiments, the modified Fc polypeptide comprises the amino acid sequence of any one of SEQ ID NOS:34-38, 58, and 60-90, but in which one, two, or three amino acids are substituted.

In some embodiments, the modified Fc polypeptide comprises additional mutations such as the mutations described below, including, but not limited to, a knob mutation (e.g., T366W as numbered with reference to EU numbering), hole mutations (e.g., T366S, L368A, and Y407V as numbered with reference to EU numbering), mutations that modulate effector function (e.g., L234A, L235A, and/or P329G (e.g., L234A and L235A) as numbered with reference to EU numbering), and/or mutations that increase serum stability or serum half-life (e.g., (i) M252Y, S254T, and T256E as numbered with reference to EU numbering, or (ii) N434S with or without M428L as numbered according to the EU numbering scheme). By way of illustration, SEQ ID NOS:118-191 provide non-limiting examples of modified Fc polypeptides with mutations in the CH3 domain (e.g., clones CH3C.35.20.1, CH3C.35.23.2, CH3C.35.23.3, CH3C.35.23.4, CH3C.35.21.17.2, and CH3C.35.23) comprising one or more of these additional mutations.

In some embodiments, the modified Fc polypeptide comprises a knob mutation (e.g., T366W as numbered with reference to EU numbering) and has at least 85% identity, at least 90% identity, or at least 95% identity to the sequence of any one of SEQ ID NOS:118, 130, 142, 154, 166, and 178. In some embodiments, the modified Fc polypeptide comprises the sequence of any one of SEQ ID NOS:118, 130, 142, 154, 166, and 178.

In some embodiments, the modified Fc polypeptide comprises a knob mutation (e.g., T366W as numbered with reference to EU numbering) and mutations that modulate effector function (e.g., L234A, L235A, and/or P329G (e.g., L234A and L235A) as numbered with reference to EU numbering), and has at least 85% identity, at least 90% identity, or at least 95% identity to the sequence of any one of SEQ ID NOS:119, 120, 131, 132, 143, 144, 155, 156, 167, 168, 179, 180, 190, and 191. In some embodiments, the modified Fc polypeptide comprises the sequence of any one of SEQ ID NOS:119, 120, 131, 132, 143, 144, 155, 156, 167, 168, 179, and 180.

In some embodiments, the modified Fc polypeptide comprises a knob mutation (e.g., T366W as numbered with reference to EU numbering) and mutations that increase serum stability or serum half-life (e.g., (i) M252Y, S254T, and T256E as numbered with reference to EU numbering, or (ii) N434S with or without M428L as numbered according to the EU numbering scheme), and has at least 85% identity, at least 90% identity, or at least 95% identity to the sequence of any one of SEQ ID NOS:121, 133, 145, 157, 169, and 181. In some embodiments, the modified Fc polypeptide comprises the sequence of any one of SEQ ID NOS:121, 133, 145, 157, 169, and 181.

In some embodiments, the modified Fc polypeptide comprises a knob mutation (e.g., T366W as numbered with reference to EU numbering), mutations that modulate effector function (e.g., L234A, L235A, and/or P329G (e.g., L234A and L235A) as numbered with reference to EU numbering), and mutations that increase serum stability or serum half-life (e.g., (i) M252Y, S254T, and T256E as numbered with reference to EU numbering, or (ii) N434S with or without M428L as numbered according to the EU numbering scheme), and has at least 85% identity, at least 90% identity, or at least 95% identity to the sequence of any one of SEQ ID NOS:122, 123, 134, 135, 146, 147, 158, 159, 170, 171, 182, and 183. In some embodiments, the modified Fc polypeptide comprises the sequence of any one of SEQ ID NOS:122, 123, 134, 135, 146, 147, 158, 159, 170, 171, 182, and 183.

In some embodiments, the modified Fc polypeptide comprises hole mutations (e.g., T366S, L368A, and Y407V as numbered with reference to EU numbering) and has at least 85% identity, at least 90% identity, or at least 95% identity to the sequence of any one of SEQ ID NOS:124, 136, 148, 160, 172, and 184. In some embodiments, the modified Fc polypeptide comprises the sequence of any one of SEQ ID NOS:124, 136, 148, 160, 172, and 184.

In some embodiments, the modified Fc polypeptide comprises hole mutations (e.g., T366S, L368A, and Y407V as numbered with reference to EU numbering) and mutations that modulate effector function (e.g., L234A, L235A, and/or P329G (e.g., L234A and L235A) as numbered with reference to EU numbering), and has at least 85% identity, at least 90% identity, or at least 95% identity to the sequence of any one of SEQ ID NOS:125, 126, 137, 138, 149, 150, 161, 162, 173, 174, 185, and 186. In some embodiments, the modified Fc polypeptide comprises the sequence of any one of SEQ ID NOS:125, 126, 137, 138, 149, 150, 161, 162, 173, 174, 185, and 186.

In some embodiments, the modified Fc polypeptide comprises hole mutations (e.g., T366S, L368A, and Y407V as numbered with reference to EU numbering) and mutations that increase serum stability or serum half-life (e.g., (i) M252Y, S254T, and T256E as numbered with reference to EU numbering, or (ii) N434S with or without M428L as numbered according to the EU numbering scheme), and has at least 85% identity, at least 90% identity, or at least 95% identity to the sequence of any one of SEQ ID NOS:127, 139, 151, 163, 175, and 187. In some embodiments, the modified Fc polypeptide comprises the sequence of any one of SEQ ID NOS:127, 139, 151, 163, 175, and 187.

In some embodiments, the modified Fc polypeptide comprises hole mutations (e.g., T366S, L368A, and Y407V as numbered with reference to EU numbering), mutations that modulate effector function (e.g., L234A, L235A, and/or P329G (e.g., L234A and L235A) as numbered with reference to EU numbering), and mutations that increase serum stability or serum half-life (e.g., (i) M252Y, S254T, and T256E as numbered with reference to EU numbering, or (ii) N434S with or without M428L as numbered according to the EU numbering scheme), and has at least 85% identity, at least 90% identity, or at least 95% identity to the sequence of any one of SEQ ID NOS:128, 129, 140, 141, 152, 153, 164, 165, 176, 177, 188, and 189. In some embodiments, the modified Fc polypeptide comprises the sequence of any one of SEQ ID NOS:128, 129, 140, 141, 152, 153, 164, 165, 176, 177, 188, and 189.

In some embodiments, a modified Fc polypeptide that specifically binds to TfR comprises at least two, three, four, five, six, seven, or eight substitutions at positions 345, 346, 347, 349, 437, 438, 439, and 440, according to the EU numbering scheme. In some embodiments, the modified Fc polypeptide comprises Gly at position 437; Phe at position 438; and/or Asp at position 440. In some embodiments, Glu is present at position 440. In certain embodiments, the modified Fc polypeptide comprises at least one substitution at a position as follows: Phe or Ile at position 345; Asp, Glu, Gly, Ala, or Lys at position 346; Tyr, Met, Leu, Ile, or Asp at position 347; Thr or Ala at position 349; Gly at position 437; Phe at position 438; His Tyr, Ser, or Phe at position 439; or Asp at position 440. In some embodiments, two, three, four, five, six, seven, or all eight of positions 345, 346, 347, 349, 437, 438, 439, and 440 and have a substitution as specified in this paragraph. In some embodiments, the modified Fc polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set.

III. Additional Fc Polypeptide Mutations

In some aspects, a fusion protein described herein comprises two Fc polypeptides that may each comprise independently selected modifications or may be a wild-type Fc polypeptide, e.g., a human IgG1 Fc polypeptide. In some embodiments, one or both Fc polypeptides contains one or more modifications that confer binding to a blood-brain barrier (BBB) receptor, e.g., transferrin receptor (TfR). Non-limiting examples of other mutations that can be introduced into one or both Fc polypeptides include, e.g., mutations to increase serum stability or serum half-life, to modulate effector function, to influence glycosylation, to reduce immunogenicity in humans, and/or to provide for knob and hole heterodimerization of the Fc polypeptides.

In some embodiments, the Fc polypeptides present in the fusion protein independently have an amino acid sequence identity of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% to a corresponding wild-type Fc polypeptide (e.g., a human IgG1, IgG2, IgG3, or IgG4 Fc polypeptide).

In some embodiments, the Fc polypeptides present in the fusion protein include knob and hole mutations to promote heterodimer formation and hinder homodimer formation. Generally, the modifications introduce a protuberance (“knob”) at the interface of a first polypeptide and a corresponding cavity (“hole”) in the interface of a second polypeptide, such that the protuberance can be positioned in the cavity so as to promote heterodimer formation and thus hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine). In some embodiments, such additional mutations are at a position in the Fc polypeptide that does not have a negative effect on binding of the polypeptide to a BBB receptor, e.g., TfR.

In one illustrative embodiment of a knob and hole approach for dimerization, position 366 (numbered according to the EU numbering scheme) of one of the Fc polypeptides present in the fusion protein comprises a tryptophan in place of a native threonine. The other Fc polypeptide in the dimer has a valine at position 407 (numbered according to the EU numbering scheme) in place of the native tyrosine. The other Fc polypeptide may further comprise a substitution in which the native threonine at position 366 (numbered according to the EU numbering scheme) is substituted with a serine and a native leucine at position 368 (numbered according to the EU numbering scheme) is substituted with an alanine. Thus, one of the Fc polypeptides of a fusion protein described herein has the T366W knob mutation and the other Fc polypeptide has the Y407V mutation, which is typically accompanied by the T366S and L368A hole mutations.

In some embodiments, modifications to enhance serum half-life may be introduced. For example, in some embodiments, one or both Fc polypeptides present in a fusion protein described herein may comprise a tyrosine at position 252, a threonine at position 254, and a glutamic acid at position 256, as numbered according to the EU numbering scheme. Thus, one or both Fc polypeptides may have M252Y, S254T, and T256E substitutions. Alternatively, one or both Fc polypeptides may have M428L and N434S substitutions, as numbered according to the EU numbering scheme. Alternatively, one or both Fc polypeptides may have an N434S or N434A substitution.

In some embodiments, one or both Fc polypeptides present in a fusion protein described herein may comprise modifications that reduce effector function, i.e., having a reduced ability to induce certain biological functions upon binding to an Fc receptor expressed on an effector cell that mediates the effector function. Examples of antibody effector functions include, but are not limited to, C1q binding and complement dependent cytotoxicity (CDC), Fc receptor binding, antibody-dependent cell-mediated cytotoxicity (ADCC), antibody-dependent cell-mediated phagocytosis (ADCP), down-regulation of cell surface receptors (e.g., B cell receptor), and B-cell activation. Effector functions may vary with the antibody class. For example, native human IgG1 and IgG3 antibodies can elicit ADCC and CDC activities upon binding to an appropriate Fc receptor present on an immune system cell; and native human IgG1, IgG2, IgG3, and IgG4 can elicit ADCP functions upon binding to the appropriate Fc receptor present on an immune cell.

In some embodiments, one or both Fc polypeptides present in a fusion protein described herein may also be engineered to contain other modifications for heterodimerization, e.g., electrostatic engineering of contact residues within a CH3-CH3 interface that are naturally charged or hydrophobic patch modifications.

In some embodiments, one or both Fc polypeptides present in a fusion protein described herein may include additional modifications that modulate effector function.

In some embodiments, one or both Fc polypeptides present in a fusion protein described herein may comprise modifications that reduce or eliminate effector function. Illustrative Fc polypeptide mutations that reduce effector function include, but are not limited to, substitutions in a CH2 domain, e.g., at positions 234 and 235, according to the EU numbering scheme. For example, in some embodiments, one or both Fc polypeptides can comprise alanine residues at positions 234 and 235. Thus, one or both Fc polypeptides may have L234A and L235A (LALA) substitutions.

Additional Fc polypeptide mutations that modulate an effector function include, but are not limited to, the following: position 329 may have a mutation in which proline is substituted with a glycine or arginine or an amino acid residue large enough to destroy the Fc/Fcγ receptor interface that is formed between proline 329 of the Fc and tryptophan residues Trp 87 and Trp 110 of FcγRIII Additional illustrative substitutions include S228P, E233P, L235E, N297A, N297D, and P331S, according to the EU numbering scheme. Multiple substitutions may also be present, e.g., L234A and L235A of a human IgG1 Fc region; L234A, L235A, and P329G of a human IgG1 Fc region; S228P and L235E of a human IgG4 Fc region; L234A and G237A of a human IgG1 Fc region; L234A, L235A, and G237A of a human IgG1 Fc region; V234A and G237A of a human IgG2 Fc region; L235A, G237A, and E318A of a human IgG4 Fc region; and S228P and L236E of a human IgG4 Fc region, according to the EU numbering scheme. In some embodiments, one or both Fc polypeptides may have one or more amino acid substitutions that modulate ADCC, e.g., substitutions at positions 298, 333, and/or 334, according to the EU numbering scheme.

Illustrative Fc polypeptides comprising additional mutations

By way of non-limiting example, one or both Fc polypeptides present in a fusion protein described herein may comprise additional mutations including a knob mutation (e.g., T366W as numbered according to the EU numbering scheme), hole mutations (e.g., T366S, L368A, and Y407V as numbered according to the EU numbering scheme), mutations that modulate effector function (e.g., L234A, L235A, and/or P329G (e.g., L234A and L235A) as numbered according to the EU numbering scheme), and/or mutations that increase serum stability or serum half-life (e.g., (i) M252Y, S254T, and T256E as numbered with reference to EU numbering, or (ii) N434S with or without M428L as numbered according to the EU numbering scheme).

In some embodiments, an Fc polypeptide may have a knob mutation (e.g., T366W as numbered according to the EU numbering scheme) and at least 85% identity, at least 90% identity, or at least 95% identity to the sequence of any one of SEQ ID NOS:1 and 4-90. In some embodiments, an Fc polypeptide having the sequence of any one of SEQ ID NOS:1 and 4-90 may be modified to have a knob mutation.

In some embodiments, an Fc polypeptide may have a knob mutation (e.g., T366W as numbered according to the EU numbering scheme), mutations that modulate effector function (e.g., L234A, L235A, and/or P329G (e.g., L234A and L235A) as numbered according to the EU numbering scheme), and at least 85% identity, at least 90% identity, or at least 95% identity to the sequence of any one of SEQ ID NOS:1 and 4-90. In some embodiments, an Fc polypeptide having the sequence of any one of SEQ ID NOS:1 and 4-90 may be modified to have a knob mutation and mutations that modulate effector function.

In some embodiments, an Fc polypeptide may have a knob mutation (e.g., T366W as numbered according to the EU numbering scheme), mutations that increase serum stability or serum half-life (e.g., (i) M252Y, S254T, and T256E as numbered with reference to EU numbering, or (ii) N434S with or without M428L as numbered according to the EU numbering scheme), and at least 85% identity, at least 90% identity, or at least 95% identity to the sequence of any one of SEQ ID NOS:1 and 4-90. In some embodiments, an Fc polypeptide having the sequence of any one of SEQ ID NOS:1 and 4-90 may be modified to have a knob mutation and mutations that increase serum stability or serum half-life.

In some embodiments, an Fc polypeptide may have a knob mutation (e.g., T366W as numbered according to the EU numbering scheme), mutations that modulate effector function (e.g., L234A, L235A, and/or P329G (e.g., L234A and L235A) as numbered according to the EU numbering scheme), mutations that increase serum stability or serum half-life (e.g., (i) M252Y, S254T, and T256E as numbered with reference to EU numbering, or (ii) N434S with or without M428L as numbered according to the EU numbering scheme), and at least 85% identity, at least 90% identity, or at least 95% identity to the sequence of any one of SEQ ID NOS:1 and 4-90. In some embodiments, an Fc polypeptide having the sequence of any one of SEQ ID NOS:1 and 4-90 may be modified to have a knob mutation, mutations that modulate effector function, and mutations that increase serum stability or serum half-life.

In some embodiments, an Fc polypeptide may have hole mutations (e.g., T366S, L368A, and Y407V as numbered according to the EU numbering scheme) and at least 85% identity, at least 90% identity, or at least 95% identity to the sequence of any one of SEQ ID NOS:1 and 4-90. In some embodiments, an Fc polypeptide having the sequence of any one of SEQ ID NOS:1 and 4-90 may be modified to have hole mutations.

In some embodiments, an Fc polypeptide may have hole mutations (e.g., T366S, L368A, and Y407V as numbered according to the EU numbering scheme), mutations that modulate effector function (e.g., L234A, L235A, and/or P329G (e.g., L234A and L235A) as numbered according to the EU numbering scheme), and at least 85% identity, at least 90% identity, or at least 95% identity to the sequence of any one of SEQ ID NOS:1 and 4-90. In some embodiments, an Fc polypeptide having the sequence of any one of SEQ ID NOS:1 and 4-90 may be modified to have hole mutations and mutations that modulate effector function.

In some embodiments, an Fc polypeptide may have hole mutations (e.g., T366S, L368A, and Y407V as numbered according to the EU numbering scheme), mutations that increase serum or serum half-life (e.g., (i) M252Y, S254T, and T256E as numbered with reference to EU numbering, or (ii) N434S with or without M428L as numbered according to the EU numbering scheme), and at least 85% identity, at least 90% identity, or at least 95% identity to the sequence of any one of SEQ ID NOS:1 and 4-90. In some embodiments, an Fc polypeptide having sequence of any one of SEQ ID NOS:1 and 4-90 may be modified to have hole mutations and mutations that increase serum stability or serum half-life.

In some embodiments, an Fc polypeptide may have hole mutations (e.g., T366S,

L368A, and Y407V as numbered according to the EU numbering scheme), mutations that modulate effector function (e.g., L234A, L235A, and/or P329G (e.g., L234A and L235A) as numbered according to the EU numbering scheme), mutations that increase serum stability or serum half-life (e.g., (i) M252Y, S254T, and T256E as numbered with reference to EU numbering, or (ii) N434S with or without M428L as numbered according to the EU numbering scheme), and at least 85% identity, at least 90% identity, or at least 95% identity to the sequence of any one of SEQ ID NOS:1 and 4-90. In some embodiments, an Fc polypeptide having the sequence of any one of SEQ ID NOS:1 and 4-90 may be modified to have hole mutations, mutations that modulate effector function, and mutations that increase serum stability or serum half-life.

IV. Illustrative Fusion Proteins Comprising an ERT Enzyme

In some aspects, a fusion protein described herein comprises a first Fc polypeptide that is linked to an enzyme replacement therapy (ERT) enzyme, an ERT enzyme variant, or a catalytically active fragment thereof; and a second Fc polypeptide that forms an Fc dimer with the first Fc polypeptide. In some embodiments, the first Fc polypeptide and/or the second Fc polypeptide does not include an immunoglobulin heavy and/or light chain variable region sequence or an antigen-binding portion thereof. In some embodiments, the ERT enzyme is IDS. In some embodiments, the first Fc polypeptide is a modified Fc polypeptide and/or the second Fc polypeptide is a modified Fc polypeptide. In some embodiments, the second Fc polypeptide is a modified Fc polypeptide. In some embodiments, the modified Fc polypeptide contains one or more modifications that promote its heterodimerization to the other Fc polypeptide. In some embodiments, the modified Fc polypeptide contains one or more modifications that reduce effector function. In some embodiments, the modified Fc polypeptide contains one or more modifications that extend serum half-life. In some embodiments, the modified Fc polypeptide contains one or more modifications that confer binding to a blood-brain barrier (BBB) receptor, e.g., transferrin receptor (TfR).

In other aspects, a fusion protein described herein comprises a first polypeptide chain that comprises a modified Fc polypeptide that specifically binds to a BBB receptor, e.g., TfR, and a second polypeptide chain that comprises an Fc polypeptide which dimerizes with the modified Fc polypeptide to form an Fc dimer. An ERT enzyme may be linked to either the first or the second polypeptide chain. In some embodiments, the ERT enzyme is IDS. In some embodiments, the ERT enzyme is linked to the second polypeptide chain. In some embodiments, the protein comprises two ERT enzymes, each linked to one of the polypeptide chains. In some embodiments, the Fc polypeptide may be a BBB receptor-binding polypeptide that specifically binds to the same BBB receptor as the modified Fc polypeptide in the first polypeptide chain. In some embodiments, the Fc polypeptide does not specifically bind to a BBB receptor.

In some embodiments, a fusion protein described herein comprises a first polypeptide chain comprising a modified Fc polypeptide that specifically binds to TfR and a second polypeptide chain that comprises an Fc polypeptide, wherein the modified Fc polypeptide and the Fc polypeptide dimerize to from an Fc dimer. In some embodiments, the ERT enzyme is IDS. In some embodiments, the ERT enzyme is linked to the first polypeptide chain. In some embodiments, the ERT enzyme is linked to the second polypeptide chain. In some embodiments, the Fc polypeptide does not specifically bind to a BBB receptor, e.g., TfR.

In some embodiments, a fusion protein described herein comprises a first polypeptide chain that comprises a modified Fc polypeptide that binds to TfR and comprises a T366W (knob) substitution; and a second polypeptide chain that comprises an Fc polypeptide comprising T366S, L368A, and Y407V (hole) substitutions. In some embodiments, the modified Fc polypeptide and/or the Fc polypeptide further comprises L234A and L235A (LALA) substitutions. In some embodiments, the modified Fc polypeptide and/or the Fc polypeptide further comprises M252Y, S254T, and T256E (YTE) substitutions. In some embodiments, the modified Fc polypeptide and/or the Fc polypeptide further comprises L234A and L235A (LALA) substitutions and M252Y, S254T, and T256E (YTE) substitutions. In some embodiments, the modified Fc polypeptide and/or the Fc polypeptide comprises human IgG1 wild-type residues at positions 234, 235, 252, 254, 256, and 366.

In some embodiments, the modified Fc polypeptide comprises the knob, LALA, and YTE mutations as specified for any one of SEQ ID NOS:95-98, 117, 118-123, 130-135, 142-147, 154-159, 166-171, and 178-183, and has at least 85% identity, at least 90% identity, or at least 95% identity to the respective sequence; or comprises the sequence of any one of SEQ ID NOS:95-98, 117, 118-123, 130-135, 142-147, 154-159, 166-171, and 178-183. In some embodiments, the Fc polypeptide comprises the hole, LALA, and YTE mutations as specified for any one of SEQ ID NOS:99-102 and has at least 85% identity, at least 90% identity, or at least 95% identity to the respective sequence; or comprises the sequence of any one of SEQ ID NOS:99-102. In some embodiments, the modified Fc polypeptide comprises any one of SEQ ID NOS:95-98, 117, 118-123, 130-135, 142-147, 154-159, 166-171, and 178-183, and the Fc polypeptide comprises any one of SEQ ID NOS:99-102. In some embodiments, the N-terminus of the modified Fc polypeptide and/or the Fc polypeptide includes a portion of an IgG1 hinge region (e.g., DKTHTCPPCP; SEQ ID NO:111). In some embodiments, the modified Fc polypeptide has at least 85%, at least 90%, or at least 95% identity to any one of SEQ ID NOS:114, 190, and 191, or comprises the sequence of any one of SEQ ID NOS:114, 190, and 191.

In some embodiments, a fusion protein described herein comprises a first polypeptide chain that comprises a modified Fc polypeptide that binds to TfR and comprises T366S, L368A, and Y407V (hole) substitutions; and a second polypeptide chain that comprises an Fc polypeptide comprising a T366W (knob) substitution. In some embodiments, the modified Fc polypeptide and/or the Fc polypeptide further comprises L234A and L235A (LALA) substitutions. In some embodiments, the modified Fc polypeptide and/or the Fc polypeptide further comprises M252Y, S254T, and T256E (YTE) substitutions. In some embodiments, the modified Fc polypeptide and/or the Fc polypeptide further comprises L234A and L235A (LALA) substitutions and M252Y, S254T, and T256E (YTE) substitutions. In some embodiments, the modified Fc polypeptide and/or the Fc polypeptide comprises human IgG1 wild-type residues at positions 234, 235, 252, 254, 256, and 366.

In some embodiments, the modified Fc polypeptide comprises the hole, LALA, and YTE mutations as specified for any one of SEQ ID NOS:103-106, 124-129, 136-141, 148-153, 160-165, 172-177, and 184-189, and has at least 85% identity, at least 90% identity, or at least 95% identity to the respective sequence; or comprises the sequence of any one of SEQ ID NOS:103-106, 124-129, 136-141, 148-153, 160-165, 172-177, and 184-189. In some embodiments, the Fc polypeptide comprises the knob, LALA, and YTE mutations as specified for any one of SEQ ID NOS:107-110 and has at least 85% identity, at least 90% identity, or at least 95% identity to the respective sequence; or comprises the sequence of any one of SEQ ID NOS:107-110. In some embodiments, the modified Fc polypeptide comprises any one of SEQ ID NOS:103-106, 124-129, 136-141, 148-153, 160-165, 172-177, and 184-189, and the Fc polypeptide comprises any one of SEQ ID NOS:107-110. In some embodiments, the N-terminus of the modified Fc polypeptide and/or the Fc polypeptide includes a portion of an IgG1 hinge region (e.g., DKTHTCPPCP; SEQ ID NO:111).

In some embodiments, an ERT enzyme, e.g., IDS, present in a fusion protein described herein is linked to a polypeptide chain that comprises an Fc polypeptide having at least 85%, at least 90%, or at least 95% identity to any one of SEQ ID NOS:99-102, or comprises the sequence of any one of SEQ ID NOS:99-102 (e.g., as a fusion polypeptide). In some embodiments, the ERT enzyme, e.g., IDS, is linked to the Fc polypeptide by a linker, such as a flexible linker, and/or a hinge region or portion thereof (e.g., DKTHTCPPCP; SEQ ID NO:111). In some embodiments, the ERT enzyme comprises an IDS sequence having at least 85%, at least 90%, or at least 95% identity to any one of SEQ ID NOS:112, 192, and 196, or comprises the sequence of any one of SEQ ID NOS:112, 192, and 196. In some embodiments, the IDS sequence linked to the Fc polypeptide has at least 85%, at least 90%, or at least 95% identity to any one of SEQ ID NOS:113, 115, 193, 194, 197, and 198, or comprises the sequence of any one of SEQ ID NOS:113, 115, 193, 194, 197, and 198. In some embodiments, the fusion protein comprises a modified Fc polypeptide having at least 85%, at least 90%, or at least 95% identity to any one of SEQ ID NOS:95-98, 117, 118-123, 130-135, 142-147, 154-159, 166-171, and 178-183, or comprises the sequence of any one of SEQ ID NOS:95-98, 117, 118-123, 130-135, 142-147, 154-159, 166-171, and 178-183. In some embodiments, the N-terminus of the Fc polypeptide and/or the modified Fc polypeptide includes a portion of an IgG1 hinge region (e.g., DKTHTCPPCP; SEQ ID NO:111). In some embodiments, the modified Fc polypeptide has at least 85%, at least 90%, or at least 95% identity to any one of SEQ ID NOS:114, 190, and 191, or comprises the sequence of any one of SEQ ID NOS:114, 190, and 191.

In some embodiments, the fusion protein comprises an IDS-Fc fusion polypeptide comprising the sequence of SEQ ID NO:113, and a modified Fc polypeptide comprising the sequence of any one of SEQ ID NOS:167 and 190 (e.g., SEQ ID NO:190). In other embodiments, the fusion protein comprises an IDS-Fc fusion polypeptide comprising the sequence of SEQ ID NO:113, and a modified Fc polypeptide comprising the sequence of any one of SEQ ID NOS:131 and 191 (e.g., SEQ ID NO:191).

In some embodiments, the fusion protein comprises an IDS-Fc fusion polypeptide comprising the sequence of SEQ ID NO:193, and a modified Fc polypeptide comprising the sequence of any one of SEQ ID NOS:167 and 190 (e.g., SEQ ID NO:190). In other embodiments, the fusion protein comprises an IDS-Fc fusion polypeptide comprising the sequence of SEQ ID NO:193, and a modified Fc polypeptide comprising the sequence of any one of SEQ ID NOS:131 and 191 (e.g., SEQ ID NO:191).

In some embodiments, the fusion protein comprises an IDS-Fc fusion polypeptide comprising the sequence of SEQ ID NO:197, and a modified Fc polypeptide comprising the sequence of any one of SEQ ID NOS:167 and 190 (e.g., SEQ ID NO:190). In other embodiments, the fusion protein comprises an IDS-Fc fusion polypeptide comprising the sequence of SEQ ID NO:197, and a modified Fc polypeptide comprising the sequence of any one of SEQ ID NOS:131 and 191 (e.g., SEQ ID NO:191).

In some embodiments, an ERT enzyme, e.g., IDS, present in a fusion protein described herein is linked to a polypeptide chain that comprises an Fc polypeptide having at least 85%, at least 90%, or at least 95% identity to any one of SEQ ID NOS:107-110, or comprises the sequence of any one of SEQ ID NOS:107-110 (e.g., as a fusion polypeptide). In some embodiments, the ERT enzyme, e.g., IDS, is linked to the Fc polypeptide by a linker, such as a flexible linker, and/or a hinge region or portion thereof (e.g., DKTHTCPPCP; SEQ ID NO:111).

In some embodiments, the ERT enzyme comprises an IDS sequence having at least 85%, at least 90%, or at least 95% identity to any one of SEQ ID NOS:112, 192, and 196, or comprises the sequence of any one of SEQ ID NOS:112, 192, and 196. In some embodiments, the IDS sequence linked to the Fc polypeptide has at least 85%, at least 90%, or at least 95% identity to any one of SEQ ID NOS:116, 195, and 199, or comprises the sequence of any one of SEQ ID NOS:116, 195, and 199. In some embodiments, the fusion protein comprises a modified Fc polypeptide having at least 85%, at least 90%, or at least 95% identity to any one of SEQ ID NOS:103-106, 124-129, 136-141, 148-153, 160-165, 172-177, and 184-189, or comprises the sequence of any one of SEQ ID NOS:103-106, 124-129, 136-141, 148-153, 160-165, 172-177, and 184-189. In some embodiments, the N-terminus of the Fc polypeptide and/or the modified

Fc polypeptide includes a portion of an IgG1 hinge region (e.g., DKTHTCPPCP; SEQ ID NO:111).

In some embodiments, an ERT enzyme, e.g., IDS, present in a fusion protein described herein is linked to a polypeptide chain that comprises a modified Fc polypeptide having at least 85%, at least 90%, or at least 95% identity to any one of SEQ ID NOS:95-98, 117, 118-123, 130-135, 142-147, 154-159, 166-171, and 178-183, or comprises the sequence of any one of SEQ ID NOS:95-98, 117, 118-123, 130-135, 142-147, 154-159, 166-171, and 178-183 (e.g., as a fusion polypeptide). In some embodiments, the ERT enzyme, e.g., IDS, is linked to the modified Fc polypeptide by a linker, such as a flexible linker, and/or a hinge region or portion thereof (e.g., DKTHTCPPCP; SEQ ID NO:111). In some embodiments, the ERT enzyme comprises an IDS sequence having at least 85%, at least 90%, or at least 95% identity to any one of SEQ ID NOS:112, 192, and 196, or comprises the sequence of any one of SEQ ID NOS:112, 192, and 196. In some embodiments, the fusion protein comprises an Fc polypeptide having at least 85%, at least 90%, or at least 95% identity to any one of SEQ ID NOS:99-102, or comprises the sequence of any one of SEQ ID NOS:99-102. In some embodiments, the N-terminus of the modified Fc polypeptide and/or the Fc polypeptide includes a portion of an IgG1 hinge region (e.g., DKTHTCPPCP; SEQ ID NO:111).

In some embodiments, an ERT enzyme, e.g., IDS, present in a fusion protein described herein is linked to a polypeptide chain that comprises a modified Fc polypeptide having at least 85%, at least 90%, or at least 95% identity to any one of SEQ ID NOS:103-106, 124-129, 136-141, 148-153, 160-165, 172-177, and 184-189, or comprises the sequence of any one of SEQ ID NOS:103-106, 124-129, 136-141, 148-153, 160-165, 172-177, and 184-189 (e.g., as a fusion polypeptide). In some embodiments, the ERT enzyme, e.g., IDS, is linked to the modified Fc polypeptide by a linker, such as a flexible linker, and/or a hinge region or portion thereof (e.g., DKTHTCPPCP; SEQ ID NO:111). In some embodiments, the ERT enzyme comprises an IDS sequence having at least 85%, at least 90%, or at least 95% identity to any one of SEQ ID NOS:112, 192, and 196, or comprises the sequence of any one of SEQ ID NOS:112, 192, and 196. In some embodiments, the fusion protein comprises an Fc polypeptide having at least 85%, at least 90%, or at least 95% identity to any one of SEQ ID NOS:107-110, or comprises the sequence of any one of SEQ ID NOS:107-110. In some embodiments, the N-terminus of the modified Fc polypeptide and/or the Fc polypeptide includes a portion of an IgG1 hinge region (e.g., DKTHTCPPCP; SEQ ID NO:111).

V. ERT Enzymes Linked to Fc Polypeptides

In some embodiments, a fusion protein described herein comprises two Fc polypeptides as described herein and one or both of the Fc polypeptides may further comprise a partial or full hinge region. The hinge region can be from any immunoglobulin subclass or isotype. An illustrative immunoglobulin hinge is an IgG hinge region, such as an IgG1 hinge region, e.g., human IgG1 hinge amino acid sequence EPKSCDKTHTCPPCP (SEQ ID NO:93) or a portion thereof (e.g., DKTHTCPPCP; SEQ ID NO:111). In some embodiments, the hinge region is at the N-terminal region of the Fc polypeptide.

In some embodiments, an Fc polypeptide is joined to the ERT enzyme by a linker, e.g., a peptide linker. In some embodiments, the Fc polypeptide is joined to the ERT enzyme by a peptide bond or by a peptide linker, e.g., is a fusion polypeptide. The peptide linker may be configured such that it allows for the rotation of the ERT enzyme relative to the Fc polypeptide to which it is joined; and/or is resistant to digestion by proteases. Peptide linkers may contain natural amino acids, unnatural amino acids, or a combination thereof. In some embodiments, the peptide linker may be a flexible linker, e.g., containing amino acids such as Gly, Asn, Ser, Thr, Ala, and the like. Such linkers are designed using known parameters and may be of any length and contain any number of repeat units of any length (e.g., repeat units of Gly and Ser residues). For example, the linker may have repeats, such as two, three, four, five, or more Gly4-Ser (SEQ ID NO:201) repeats or a single Gly4-Ser (SEQ ID NO:201). In some embodiments, the peptide linker may include a protease cleavage site, e.g., that is cleavable by an enzyme present in the central nervous system.

In some embodiments, the ERT enzyme is joined to the N-terminus of the Fc polypeptide, e.g., by a Gly4-Ser linker (SEQ ID NO:201) or a (Gly₄-Ser)₂ linker (SEQ ID NO:202). In some embodiments, the Fc polypeptide may comprise a hinge sequence or partial hinge sequence at the N-terminus that is joined to the linker or directly joined to the ERT enzyme.

In some embodiments, the ERT enzyme is joined to the C-terminus of the Fc polypeptide, e.g., by a Gly4-Ser linker (SEQ ID NO:201) or a (Gly₄-Ser)₂ linker (SEQ ID NO:202). In some embodiments, the C-terminus of the Fc polypeptide is directly joined to the ERT enzyme.

In some embodiments, the ERT enzyme is joined to the Fc polypeptide by a chemical cross-linking agent. Such conjugates can be generated using well-known chemical cross-linking reagents and protocols. For example, there are a large number of chemical cross-linking agents that are known to those skilled in the art and useful for cross-linking the polypeptide with an agent of interest. For example, the cross-linking agents are heterobifunctional cross-linkers, which can be used to link molecules in a stepwise manner. Heterobifunctional cross-linkers provide the ability to design more specific coupling methods for conjugating proteins, thereby reducing the occurrences of unwanted side reactions such as homo-protein polymers. A wide variety of heterobifunctional cross-linkers are known in the art, including N-hydroxysuccinimide (NETS) or its water soluble analog N-hydroxysulfosuccinimide (sulfo-NHS), succinimidyl 4-(N-maleimidomethyl)cyclohexane-1-carboxylate (SMCC), m-maleimidobenzoyl-N-hydroxysuccinimide ester (MBS); N-succinimidyl (4-iodoacetyl) aminobenzoate (STAB), succinimidyl 4-(p-maleimidophenyl)butyrate (SMPB), 1-ethyl-3-(3-dimethylaminopropyl)carbodiimide hydrochloride (EDC); 4-succinimidyloxycarbonyl-a-methyl-a-(2-pyridyldithio)-toluene (SMPT), N-succinimidyl 3-(2-pyridyldithio)propionate (SPDP), and succinimidyl 6-[3-(2-pyridyldithio)propionate]hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo. In addition to the heterobifunctional cross-linkers, there exist a number of other cross-linking agents including homobifunctional and photoreactive cross-linkers. Disuccinimidyl subcrate (DSS), bismaleimidohexane (BMH) and dimethylpimelimidate. 2HCl (DMP) are examples of useful homobifunctional cross-linking agents, and bis-[B-(4-azidosalicylamido)ethyl]disulfide (BASED) and N-succinimidyl-6(4′-azido-2′-nitrophenylamino)hexanoate (SANPAH) are examples of useful photoreactive cross-linkers.

### Screening Methods

Certain embodiments described herein also provide a method of screening a test agent for activity as an LSD treatment, the method comprising:

1) contacting a cell with the test agent, wherein the cell has impaired lysosomal storage; and

2) measuring the concentration of:
a) a combination of two or more lipids in the cell, wherein the combination of lipids is selected from the group consisting of:
   i) a BMP;
   ii) a GM2 ganglioside and/or a GM3 ganglioside;
   iii) GD3;
   iv) GD1a/b; and
   v) a GlcCer;
b) GlcCer in the cell, provided the test agent is being screened for activity as an MPS treatment; and/or
c) sTREM2 in the cell,

wherein a decrease in the concentration of the selected lipid(s)/protein in the cell as compared to the concentration of the corresponding lipid(s)/protein in a control cell (e.g., a healthy cell, such as a cell that does not have an LSD mutation) indicates the test agent has activity as an LSD treatment.

Certain embodiments described herein provide a method of screening a test agent for activity as an LSD treatment, the method comprising:

1) contacting a cell with the test agent, wherein the cell has impaired lysosomal storage; and

2) measuring the concentration of:
a) a combination of two or more lipids in the cell, wherein the combination of lipids is selected from the group consisting of:
   i) a BMP;
   ii) a GM2 ganglioside and/or a GM3 ganglioside;
   iii) GD3;
   iv) GD1a/b; and
   v) a GlcCer;
b) GlcCer in the cell, provided the test agent is being screened for activity as an MPS treatment;
c) Nf-L in the cell; and/or
d) sTREM2 in the cell,

wherein a decrease in the concentration of the selected lipid(s)/protein(s) in the cell as compared to the concentration of the corresponding lipid(s)/protein(s) in a control cell (e.g., a healthy cell, such as a cell that does not have an LSD mutation) indicates the test agent has activity as an LSD treatment.

In certain embodiments, the method comprises measuring the concentration of sTREM2.

In certain embodiments, the method comprises measuring the concentration of Nf-L.

In certain embodiments, the method comprises measuring the concentration of GlcCer, wherein the test agent is being screened for activity as an MPS treatment.

In certain embodiments, the method comprises measuring the concentration of a combination of two or more lipids, such as a combination described herein.

In certain embodiments, the method comprises measuring the concentration of one or more lipids and the concentration of sTREM2.

In certain embodiments, the method comprises measuring the concentration of one or more lipids and the concentration of Nf-L.

In certain embodiments, the method comprises measuring the concentration of sTREM2 and the concentration of Nf-L.

In certain embodiments, the method comprises measuring the concentration of one or more lipids, the concentration of sTREM2 and the concentration of Nf-L.

In certain embodiments, the cell is from tissue. For example, in certain embodiments, the cell is a cell from the brain, liver, kidney, lung or spleen. In certain embodiments, the cell is a brain cell or a cell derived from a brain cell. In embodiments, the cell is a cell obtained from CSF. In embodiments, the cell is a cell obtained from serum.

In certain embodiments, the concentrations of the lipids/proteins are measured using an assay described herein (e.g., mass spectrometry).

### Methods of Isolating Enriched CNS Cell Populations

Methods of isolating enriched populations of CNS cell types from brain tissue are provided herein (e.g., enriched populations of neurons, astrocytes or microglial cells).

Thus, certain embodiments provide a method of sorting populations of CNS cells from a tissue sample, comprising:

(a) contacting the tissue sample with a neuronal marker primary antibody, an astrocyte marker primary antibody, a microglial marker primary antibody, an endothelial marker primary antibody, and an oligodendrocyte marker primary antibody, wherein each primary antibody is uniquely labeled, to provide a labeled tissue sample; and

(b) sorting the cells in the labeled tissue sample by flow cytometry, wherein the method provides distinct cell populations of neurons, astrocytes, and microglial cells.

As used herein, the term “distinct cell population” refers to a physically separate population of cells that is enriched for a particular CNS cell type (e.g., neuronal, astrocytic, microglial).

As used herein, the term “neuronal marker” refers to a protein or peptide that is preferentially expressed in the CNS by neurons. In certain embodiments, the neuronal marker is expressed by at least about 50%, 60%, 70%, 80%, 90%, 95%, 99%, or 99.5% of neuronal cells present in the CNS. In certain embodiments, the neuronal marker is expressed by less than about 35%, 30%, 25%, 20%, 15%, 10%, 9%, 8%, 7%, 6%, 5%, 4%, 3%, 2%, 1% or 0.5% of non-neuronal cells present in the CNS. In certain embodiments, the neuronal marker is not expressed by non-neuronal cell types that are present in the CNS. In certain embodiments, the neuronal marker is Thy1 (see, e.g., UniProtKB P01831 (mouse)).

As used herein, the term “neuronal marker primary antibody” or an “anti-neuronal marker antibody” refers to an antibody that is capable of binding to a neuronal marker with sufficient affinity such that the antibody is useful to sort neurons from a mixed population of cells using flow cytometry. In one embodiment, the extent of binding of an anti-neuronal marker antibody to an unrelated protein is less than about 10% of the binding of the antibody to the neuronal marker as measured, e.g., by a radioimmunoassay (RIA). In certain embodiments, an antibody that binds to the neuronal marker has a dissociation constant (Kd) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸M to 10⁻¹³ M, e.g., from 10⁻⁹M to 10⁻¹³M). In certain embodiments, the neuronal marker primary antibody is an anti-Thy1 antibody (e.g., an anti-Thy1 antibody used herein).

As used herein, the term “an astrocyte marker” refers to a protein or peptide that is preferentially expressed in the CNS by astrocytes. In certain embodiments, the astrocyte marker is expressed by at least about 50%, 60%, 70%, 80%, 90%, 95%, 99%, or 99.5% of astrocytes present in the CNS. In certain embodiments, the astrocyte marker is expressed by less than about 35%, 30%, 25%, 20%, 15%, 10%, 9%, 8%, 7%, 6%, 5%, 4%, 3%, 2%, 1% or 0.5% of non-astrocytic cells present in the CNS. In certain embodiments, the astrocyte marker is not expressed by non-astrocytic cell types that are present in the CNS. In certain embodiments, the astrocyte marker is excitatory amino acid transporter 2 (EAAT2) (see, e.g., UniProtKB P43006 (mouse)). In certain embodiments, the astrocyte marker is a glycosylated surface molecule recognized by an anti-astrocyte cell surface antigen-2 (ACSA-2) antibody (see, e.g., Kantzer et al., 2017, Glia, 65:990-1004).

As used herein, the term “astrocyte marker primary antibody” or an “anti-astrocyte marker antibody” refers to an antibody that is capable of binding to an astrocyte marker with sufficient affinity such that the antibody is useful to sort astrocytes from a mixed population of cells using flow cytometry. In one embodiment, the extent of binding of an anti-astrocyte marker antibody to an unrelated protein is less than about 10% of the binding of the antibody to the astrocyte marker as measured, e.g., by a radioimmunoassay (MA). In certain embodiments, an antibody that binds to the astrocyte marker has a dissociation constant (Kd) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸ M to 10⁻¹³ M, e.g., from 10⁻⁹M to 10⁻¹³M). In certain embodiments, the astrocyte marker primary antibody is an anti-EAAT2 antibody (e.g., an anti-EAAT2 antibody used herein). In certain embodiments, the astrocyte marker primary antibody is an anti-ACSA-2 antibody (e.g., ACSA-2-PE (Miltenyi Biotec 130-102-365)).

As used herein, the term “microglial marker” refers to a protein or peptide that is preferentially expressed within the CNS by microglial cells. In certain embodiments, the microglial marker is expressed by at least about 50%, 60%, 70%, 80%, 90%, 95%, 99%, or 99.5% of microglial cells present in the CNS. In certain embodiments, the microglial marker is expressed by less than about 35%, 30%, 25%, 20%, 15%, 10%, 9%, 8%, 7%, 6%, 5%, 4%, 3%, 2%, 1% or 0.5% of non-microglial cells present in the CNS. In certain embodiments, the microglial marker is not expressed by non-microglial cell types that are present in the CNS. In certain embodiments, the microglial marker is CD11b (see, e.g., UniProtKB P05555 (mouse)).

As used herein, the term “microglial marker primary antibody” or an “anti-microglial marker antibody” refers to an antibody that is capable of binding to a microglial marker with sufficient affinity such that the antibody is useful to sort microglial cells from a mixed population of cells using flow cytometry. In one embodiment, the extent of binding of an anti-microglial marker antibody to an unrelated protein is less than about 10% of the binding of the antibody to the microglial marker as measured, e.g., by a radioimmunoassay (MA). In certain embodiments, an antibody that binds to the microglial marker has a dissociation constant (Kd) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸ M to 10⁻¹³M, e.g., from 10⁻⁹M to 10⁻¹³M). In certain embodiments, the microglial marker primary antibody is an anti-CD11b antibody (e.g., an anti-CD11b antibody used herein).

As used herein, the term “endothelial marker” refers to a protein or peptide that is preferentially expressed within the CNS by endothelial cells. In certain embodiments, the endothelial cell marker is expressed by at least about 50%, 60%, 70%, 80%, 90%, 95%, 99%, or 99.5% of endothelial cells present in the CNS. In certain embodiments, the endothelial cell marker is expressed by less than about 35%, 30%, 25%, 20%, 15%, 10%, 9%, 8%, 7%, 6%, 5%, 4%, 3%, 2%, 1% or 0.5% of non-endothelial cells present in the CNS. In certain embodiments, the endothelial cell marker is not expressed by non-endothelial cell types that are present in the CNS. In certain embodiments, the endothelial cell marker is CD31 (see, e.g., UniProtKB Q08481 (mouse)).

As used herein, the term “endothelial marker primary antibody” or an “anti-endothelial cell marker antibody” refers to an antibody that is capable of binding to an endothelial cell marker with sufficient affinity such that the antibody is useful to sort endothelial cells from a mixed population of cells using flow cytometry. In one embodiment, the extent of binding of an anti-endothelial cell marker antibody to an unrelated protein is less than about 10% of the binding of the antibody to the endothelial cell marker as measured, e.g., by a radioimmunoassay (MA). In certain embodiments, an antibody that binds to the endothelial cell marker has a dissociation constant (Kd) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸M to 10⁻¹³M, e.g., from 10⁻⁹M to 10⁻¹³M). In certain embodiments, the endothelial cell marker primary antibody is an anti-CD31 antibody (e.g., an anti-CD-31 antibody used herein).

As used herein, the term “oligodendrocyte marker” refers to a protein or peptide that is preferentially expressed within the CNS by oligodendrocyte cells. In certain embodiments, the oligodendrocyte marker is expressed by at least about 50%, 60%, 70%, 80%, 90%, 95%, 99%, or 99.5% of oligodendrocyte cells present in the CNS. In certain embodiments, the oligodendrocyte marker is expressed by less than about 35%, 30%, 25%, 20%, 15%, 10%, 9%, 8%, 7%, 6%, 5%, 4%, 3%, 2%, 1% or 0.5% of non-oligodendrocyte cells present in the CNS. In certain embodiments, the oligodendrocyte marker is not expressed by non-oligodendrocyte cell types that are present in the CNS. In certain embodiments, the oligodendrocyte marker is a membrane lipid marker. In certain embodiments, the oligodendrocyte marker is a lipid that is enriched on a mature oligodendrocyte, such as, e.g., a galactocerebroside (GalCer).

As used herein, the term “oligodendrocyte marker primary antibody” or an “anti-oligodendrocyte marker antibody” refers to an antibody that is capable of binding to an oligodendrocyte marker with sufficient affinity such that the antibody is useful to sort oligodendrocyte cells from a mixed population of cells using flow cytometry. In one embodiment, the extent of binding of an anti-oligodendrocyte marker antibody to an unrelated protein is less than about 10% of the binding of the antibody to the oligodendrocyte marker as measured, e.g., by a radioimmunoassay (MA). In certain embodiments, an antibody that binds to the oligodendrocyte marker has a dissociation constant (Kd) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸M to 10⁻¹³M, e.g., from 10⁻⁹ M to 10⁻¹³M). In certain embodiments, the oligodendrocyte marker primary antibody is an anti-O1 antibody, which reacts to a membrane lipid marker (e.g., a GalCer) (e.g., an anti-O1 antibody used herein).

In certain embodiments, the tissue sample is contacted with an anti-Thy1 antibody, an anti-EAAT2 antibody, an anti-CD11b antibody, an anti-CD31 antibody and an anti-O1 antibody. In certain embodiments, the tissue sample is contacted with an anti-Thy1 antibody, an anti-ACSA-2 antibody, an anti-CD11b antibody, an anti-CD31 antibody and an anti-O1 antibody. In certain embodiments, the primary antibodies are comprised within a composition, and the tissue sample is contacted with the composition. In certain embodiments, each primary antibody is uniquely labeled (i.e., each antibody comprises a different label) with a label suitable for sorting by flow cytometry (e.g., a fluorescent label). In certain embodiments, the tissue sample is further contacted with a viability dye, which may be used to distinguish viable and non-viable cells by flow cytometry (e.g., Fixable Viability Stain BV510). In certain embodiments, the tissue sample is contacted with the viability dye simultaneously or sequentially with the primary antibodies. In certain other embodiments, the viability dye is comprised within the composition comprising the primary antibodies, and the tissue sample is contacted with composition comprising the primary antibodies and the viability dye.

In certain embodiments, the cells present within the tissue sample are dissociated prior to being contacted with the viability dye, the primary antibodies, and/or the composition comprising the primary antibodies with or without the viability dye.

In certain embodiments, the tissue sample is contacted with the primary antibodies under conditions suitable for the antibodies to bind to its corresponding marker and label the cells. In certain embodiments, the labeled tissue sample prior to being sorted by flow cytometry comprises labeled Thy1⁺ cells, labeled EAAT2⁺ cells, labeled CD11b⁺ cells, labeled CD31⁺ cells and labeled O1⁺ cells. In certain embodiments, the labeled tissue sample prior to being sorted by flow cytometry comprises labeled Thy1⁺ cells, labeled ACSA-2⁺ cells, labeled CD11b⁺ cells, labeled CD31⁺ cells and labeled O1⁺ cells. In certain embodiments, the cells are further labeled with a viability dye.

In certain embodiments, the cells present within the tissue sample are sorted by flow cytometry into a population of non-viable cells and a population of viable cells (e.g., with a viability dye).

In certain embodiments, the cells present within the tissue sample are sorted by flow cytometry into a population of O1⁺ cells and a population of O1⁻ cells.

In certain embodiments, the cells present within the tissue sample are sorted by flow cytometry into a population of CD31⁺ cells and a population of CD31⁻ cells.

In certain embodiments, the cells present within the tissue sample are sorted by flow cytometry into a population of Thy⁺ cells and a population of Thy⁻ cells.

In certain embodiments, the cells present within the tissue sample are sorted by flow cytometry into a population of EAAT2⁺ cells and a population of EAAT2⁻ cells.

In certain embodiments, the cells present within the tissue sample are sorted by flow cytometry into a population of ACSA-2⁺ cells and a population of ACSA-2⁻ cells.

In certain embodiments, the cells present within the tissue sample are sorted by flow cytometry into a population of CD11b⁺ cells and a population of CD11b⁻ cells.

In certain embodiments, the cells present within the tissue sample are sorted by flow cytometry into a population of viable, O1⁻, CD31⁻, Thy1⁺, EAAT2⁻ cells (i.e., neuronal cells).

In certain embodiments, the cells present within the tissue sample are sorted by flow cytometry into a population of viable, O1⁻, CD31⁻, Thy1⁻, EAAT2⁺ cells (i.e., astrocytic cells).

In certain embodiments, the cells present within the tissue sample are sorted by flow cytometry into a population of viable, O1⁻, CD31⁻, CD11b⁺ cells (i.e., microglial cells).

In certain embodiments, the cells present within the tissue sample are sorted by flow cytometry into a population of viable, O1⁻, CD31⁻, Thy1⁺, ACSA-2⁻ cells (i.e., neuronal cells).

In certain embodiments, the cells present within the tissue sample are sorted by flow cytometry into a population of viable, O1⁻, CD31⁻, Thy1⁻, ACSA-2⁺ cells (i.e., astrocytic cells).

In certain embodiments, the cells present within the tissue sample are sorted by flow cytometry into a population of viable, O1⁻, CD31⁻, CD11b⁺ cells (i.e., microglial cells).

In certain embodiments, the cells present within the tissue sample are sorted by flow cytometry into a population of non-viable cells and a population of viable cells (e.g., with a viability dye). In certain embodiments, the population of viable cells is further sorted into a population of O1⁺, CD31⁺ cells (i.e., oligodendrocytes and endothelial cells) and a population of O1⁻, CD31⁻ cells. In certain embodiments, the population of O1⁻, CD31⁻ cells are further sorted into a population of viable, O1⁻, CD31⁻, Thy1⁺, EAAT2⁻ cells (i.e., neuronal cells); a population of viable, O1⁻, CD31⁻, Thy1⁻, EAAT2⁺ cells (i.e., astrocytic cells); and a population of viable, O1⁻, CD31⁻, CD11b⁺ cells (i.e., microglial cells).

In certain embodiments, the cells present within the tissue sample are sorted by flow cytometry into a population of non-viable cells and a population of viable cells (e.g., with a viability dye). In certain embodiments, the population of viable cells is further sorted into a population of O1⁺, CD31⁺ cells (i.e., oligodendrocytes and endothelial cells) and a population of O1⁻, CD31⁻ cells. In certain embodiments, the population of O1⁻, CD31⁻ cells are further sorted into a population of viable, O1⁻, CD31⁻, Thy1⁺, ACSA-2⁻ cells (i.e., neuronal cells); a population of viable, O1⁻, CD31⁻, Thy1⁻, ACSA-2⁺ cells (i.e., astrocytic cells); and a population of viable, O1⁻, CD3 CD11b⁺ cells (i.e., microglial cells).

In certain embodiments, the cells present within the tissue sample are sorted by flow cytometry into a population of non-viable cells and a population of viable cells (e.g., with a viability dye). In certain embodiments, the population of viable cells is further sorted into a population of CD31⁺ cells (i.e., endothelial cells) and a population of CD31⁻ cells. In certain embodiments, the population of CD31⁻ cells are further sorted into a population of CD3 Thy1⁻, EAAT2⁻ cells; a population of CD31⁻, Thy1⁻, EAAT2⁺ cells; and a population of CD31⁻, CD11b⁺ cells. In certain embodiments, the populations of CD31⁻, Thy1⁺, EAAT2⁻ cells; CD31⁻, Thy1⁻, EAAT2⁺ cells; and CD3 CD11b⁺ cells are further sorted to remove O1⁺ cells (i.e., oligodendrocytes), to provide a population of viable, O1⁻, CD31⁻, Thy1⁻, EAAT2⁻ cells (i.e., neuronal cells); a population of viable, O1⁻, CD31⁻, Thy1⁻, EAAT2⁺ cells (i.e., astrocytic cells); and a population of viable, O1⁻, CD3 CD11b⁺ cells (i.e., microglial cells).

In certain embodiments, the cells present within the tissue sample are sorted by flow cytometry into a population of non-viable cells and a population of viable cells (e.g., with a viability dye). In certain embodiments, the population of viable cells is further sorted into a population of CD31⁺ cells (i.e., endothelial cells) and a population of CD31⁻ cells. In certain embodiments, the population of CD31⁻ cells are further sorted into a population of CD3 Thy1⁺, ACSA-2⁻ cells; a population of CD31⁻, Thy1⁻, ACSA-2⁺ cells; and a population of CD3 CD11b⁺ cells. In certain embodiments, the populations of CD31⁻, Thy1⁺, ACSA-2⁻ cells; CD31⁻, Thy1⁻, ACSA-2⁺ cells; and CD3 CD11b⁺ cells are further sorted to remove O1⁺ cells (i.e., oligodendrocytes), to provide a population of viable, O1⁻, CD31⁻, Thy1⁺, ACSA-2⁻ cells (i.e., neuronal cells); a population of viable, O1⁻, CD31⁻, Thy1⁻, ACSA-2⁺ cells (i.e., astrocytic cells); and a population of viable, O1⁻, CD3 CD11b⁺ cells (i.e., microglial cells).

In certain embodiments, a microglial cell population is sorted based on the marker profile O1⁻/CD31⁻/CD11b⁺.

In certain embodiments, the astrocyte population is sorted based on the marker profile O1⁻/CD31⁻/Thy1⁻/EAAT2⁺. In certain embodiments, the astrocyte population is sorted based on the marker profile O1⁻/CD31⁻/Thy1⁻/ACSA-2⁺.

In certain embodiments, the neuronal population is sorted based on the marker profile O1⁻/CD31⁻/Thy1⁺/EAAT2⁻. In certain embodiments, the neuronal population is sorted based on the marker profile O1⁻/CD31⁻/Thy1⁺/ACSA-2⁻.

In certain embodiments, the sorted population of enriched neuronal cells (e.g., viable, O1⁻, CD31⁻, Thy1⁺, EAAT2⁻ cells or viable, O1⁻, CD31⁻, Thy1⁺, ACSA-2⁻ cells) comprises less than about 25%, 20%, 19%, 18%, 17%, 16%, 15%, 14%, 13%, 12%, 11%, 10%, 9%, 8%, 7%, 6%, 5%, 4%, 3%, 2%, 1% or less of non-neuronal cells. In certain embodiments, the sorted population of enriched neuronal cells (e.g., viable, O1⁻, CD31⁻, Thy1⁺, EAAT2⁻ cells or viable, O1⁻, CD31⁻, Thy1⁺, ACSA-2⁻ cells) does not contain non-neuronal cells.

In certain embodiments, the sorted population of enriched astrocytic cells (e.g., viable, O1⁻, CD31⁻, Thy1⁻, EAAT2⁺ cells or viable, O1⁻, CD31⁻, Thy1⁻, ACSA-2⁺ cells) comprises less than about 25%, 20%, 19%, 18%, 17%, 16%, 15%, 14%, 13%, 12%, 11%, 10%, 9%, 8%, 7%, 6%, 5%, 4%, 3%, 2%, 1% or less of non-astrocytic cells. In certain embodiments, the sorted population of enriched astrocytic cells (e.g., viable, O1⁻, CD31⁻, Thy1⁻, EAAT2⁺ cells or viable, O1⁻, CD31⁻, Thy1⁻, ACSA-2⁺ cells) does not contain non-astrocytic cells.

In certain embodiments, the sorted population of enriched microglial cells (e.g., viable, O1⁻, CD31⁻, CD11b⁺ cells) comprises less than about 25%, 20%, 19%, 18%, 17%, 16%, 15%, 14%, 13%, 12%, 11%, 10%, 9%, 8%, 7%, 6%, 5%, 4%, 3%, 2%, 1% or less of non-microglial cells. In certain embodiments, the sorted population of enriched microglial cells (e.g., viable, O1⁻, CD31⁻, CD11b⁺ cells) does not contain non-microglial cells.

In certain embodiments, one or more of the enriched cell populations (e.g., 1, 2 or 3 of the enriched cell populations) are analyzed for quantification of a metabolic or nucleic acid species. In certain embodiments, the enriched neuronal cell population is analyzed for quantification of a metabolic or nucleic acid species. In certain embodiments, the enriched astrocytic cell population is analyzed for quantification of a metabolic or nucleic acid species. In certain embodiments, the enriched microglial cell population is analyzed for quantification of a metabolic or nucleic acid species. In certain embodiments, the enriched neuronal, astrocytic and microglial cell populations are analyzed for quantification of a metabolic or nucleic acid species.

In certain embodiments, the one or more enriched cell populations are analyzed for quantification of a metabolic species. In certain embodiments, the one or more enriched cell populations are analyzed for quantification of more than one metabolic species (e.g., 2, 3, 4, 5, 10, 25, 50 or more). In certain embodiments, the one or more enriched cell populations are analyzed for quantification of a nucleic acid species. In certain embodiments, the one or more enriched cell populations are analyzed for quantification of more than one nucleic acid species (e.g., 2, 3, 4, 5, 10, 25, 50 or more). In certain embodiments, the one or more enriched cell populations are analyzed for quantification of a metabolic and a nucleic acid species. In certain embodiments, the one or more enriched cell populations are analyzed for quantification of more than one metabolic species and more than one nucleic acid species.

As used herein, the term metabolic species includes macromolecules that are normally broken down by the lysosome. For example, in certain embodiments, the metabolic species is a glycosaminoglycan (GAG) species, such as a GAG species described herein (e.g., D0S0, D0A0 or D0a4). In certain embodiments, the metabolic species is a lipid species, such as a ganglioside, glycosylceramide (e.g., glucosylceramide), galactosylceramide or bis(monoacylglycerol)phosphate (BMP) species (e.g., a species described herein). In certain embodiments, the metabolic species is a BMP, GlcCer, GD3, GD1a/b, GM2 and/or GM3 species (e.g., as described herein). In certain embodiments, a combination of metabolic species are quantified, such as a combination of lipids described herein. Metabolic species may be quantified using methods known in the art. For example, a metabolic species may be quantified using a liquid chromatography mass spectrometry (LCMS) assay (see, e.g., the Examples).

A nucleic acid, may be e.g., RNA or DNA, such as genomic DNA, RNA transcribed from genomic DNA, or cDNA generated from RNA. In certain embodiments, the nucleic acid species is RNA. In certain embodiments, the nucleic acid species is DNA. In certain embodiments, the nucleic acid species is genomic DNA. Methods of quantifying nucleic acid species are known in the art. For example, such methods include, but are not limited to, polymerase chain reaction (PCR), including quantitative PCR (qPCR) and Real-Time Quantitative Reverse Transcription PCR (qRT-PCR); RNAseq; Northern blot analysis, expression microarray analysis; next generation sequencing (NGS); and fluorescence in situ hybridization (FISH). In certain embodiments, a nucleic acid species is quantified using an assay described herein.

In certain embodiments, one or more enriched cell populations are analyzed for quantification of sTREM2. In certain embodiments, the enriched microglial cell population is analyzed for quantification of sTREM2. sTREM2 may be quantified using methods known in the art. For example, sTREM2 may be quantified using an assay described in the Examples.

In certain embodiments, one or more enriched cell populations are analyzed for quantification of Nf-L. In certain embodiments, the enriched neuronal cell population is analyzed for quantification of Nf-L. Nf-L may be quantified using methods known in the art. For example, Nf-L may be quantified using an assay described in the Examples.

In certain embodiments, one or more enriched cell populations are analyzed for quantification of an administered therapeutic agent. In certain embodiments, the enriched neuronal cell population is analyzed for quantification of an administered therapeutic agent. In certain embodiments, the enriched astrocytic cell population is analyzed for quantification of an administered therapeutic agent. In certain embodiments, the enriched microglial cell population is analyzed for quantification of an administered therapeutic agent. In certain embodiments, the enriched neuronal, astrocytic and microglial cell populations are analyzed for quantification of an administered therapeutic agent. In certain embodiments, the therapeutic agent is an agent that is capable of reducing one or more symptoms associated with an LSD. In certain embodiments, the therapeutic agent is ETV:IDS. Methods for quantifying a therapeutic agent are known in the art and are described herein. For example, a therapeutic agent could be quantified by an assay described in the Examples.

Certain embodiments provide a collection of CNS cells comprising three physically separate cell populations, wherein:

1) the first cell population comprises an enriched population of O1⁻/CD31⁻/CD11b⁺ cells;

2) the second cell population comprises an enriched population O1⁻/CD31⁻/Thy1⁻/EAAT2⁺ cells or an enriched population of O1⁻/CD31⁻/Thy1⁻/ACSA-2⁻ cells; and

3) the third cell population comprises an enriched population of O1⁻/CD31⁻/Thy1⁺/EAAT2⁻ or an enriched population of O1⁻/CD31⁻/Thy1⁺/ACSA-2⁻ cells.

### Corrected Cells and Associated Methods

As described herein, LSDs are caused by deficiencies in certain lysosomal enzymes, which result in the accumulation of metabolic species within certain cell types (e.g., certain CNS cells). This accumulation may be corrected by contacting the affected cells with certain therapeutic agents (e.g., a therapeutic agent described herein). For example, the accumulation of a metabolic species within a cell (e.g., a CNS cell) having a lysosomal enzyme deficiency may be reduced by contacting the cell with a fusion protein that includes an enzyme replacement therapy (ERT) enzyme linked to an Fc polypeptide.

Accordingly, certain embodiments provide a corrected CNS cell comprising a deficiency in a lysosomal enzyme that causes accumulation of a metabolic species within the cell, wherein the cell is corrected by contact with a protein comprising (i) a first Fc polypeptide linked to the lysosomal enzyme, and (ii) a second Fc polypeptide that forms an Fc dimer with the first Fc polypeptide, wherein the protein is capable of binding to the transferrin receptor (TfR), and wherein the correction is a reduction in the accumulation of the metabolic species. In certain embodiments, the corrected CNS cell is a human cell. In certain embodiments, the corrected CNS cell is a non-human cell. In certain embodiments, the cell is an isolated or purified corrected CNS cell.

Certain embodiments also provide a corrected CNS cell comprising reduced accumulation of a metabolic species, wherein a CNS cell comprising a deficiency in a lysosomal enzyme that causes accumulation of the metabolic species within the cell was contacted with a protein comprising:

(i) a first Fc polypeptide linked to the lysosomal enzyme; and

(ii) a second Fc polypeptide that forms an Fc dimer with the first Fc polypeptide, wherein the protein is capable of binding to the transferrin receptor (TfR), to provide the corrected CNS cell comprising the reduced accumulation of the metabolic species.

Certain embodiments also provide a corrected CNS cell produced by a method described herein, such as a method described below.

Certain embodiments also provide a method of correcting a CNS cell having a deficiency in a lysosomal enzyme that causes accumulation of a metabolic species in the cell, the method comprising contacting the cell with a protein comprising (i) a first Fc polypeptide linked to the lysosomal enzyme, and (ii) a second Fc polypeptide that forms an Fc dimer with the first Fc polypeptide, wherein the protein is capable of binding to the transferrin receptor (TfR), to provide a corrected CNS cell having reduced accumulation of the metabolic species.

Certain embodiments also provide a method of reducing accumulation of a metabolic species in a CNS cell having a lysosomal enzyme deficiency, the method comprising contacting the cell with a protein comprising (i) a first Fc polypeptide linked to the lysosomal enzyme, and (ii) a second Fc polypeptide that forms an Fc dimer with the first Fc polypeptide, wherein the protein is capable of binding to the transferrin receptor (TfR).

In certain embodiments, the cell is contacted with the protein in vitro, ex vivo or in vivo. In certain embodiments, the cell is contacted with the protein in vitro. In certain embodiments, the cell is contacted with the protein ex vivo. In certain embodiments, the cell is contacted with the protein in vivo (i.e., via administration of the protein). In certain embodiments, the cell is from a tissue sample and has been sorted by a method described herein. In certain embodiments, the cell is contacted with the protein in vivo and prior to being sorted. In certain other embodiments, the cell is contacted with the protein in vitro, before or after being sorted.

In certain embodiments, the first Fc polypeptide linked to the lysosomal enzyme is an Fc polypeptide described herein. In certain embodiments, the lysosomal enzyme is iduronate 2-sulfatase (IDS), or a catalytically active variant or fragment of a wild-type IDS, e.g., a wild-type human IDS. In certain embodiments, the second Fc polypeptide is a polypeptide described herein. In certain embodiments, the protein is ETV:IDS.

In certain embodiments, the protein has at least 5-fold, 10-fold, 50-fold, 100-fold, 1,000-fold, 10,000-fold, or greater affinity for TfR as compared to an unrelated target, when assayed under the same affinity assay conditions. In certain embodiments, the protein binds to TfR with an affinity of from about 50 nM to about 350 nM. In certain embodiments, the protein binds to TfR with an affinity of about 50 nM, about 60 nM, about 70 nM, about 80 nM, about 90 nM, about 100 nM, about 110 nM, about 120 nM, about 130 nM, about 140 nM, about 150 nM, about 160 nM, about 170 nM, about 180 nM, about 190 nM, about 200 nM, about 210 nM, about 220 nM, about 230 nM, about 240 nM, about 250 nM, about 275 nM, about 300 nM, about 325 nM, or about 350 nM.

In certain embodiments, the metabolic species is a GAG species, such as a GAG species described herein (e.g., D0S0, D0A0 or D0a4). In certain embodiments, the metabolic species is a lipid species, such as a ganglioside, glycosylceramide (e.g., glucosylceramide), galactosylceramide or bis(monoacylglycerol)phosphate (BMP) species (e.g., a species described herein). In certain embodiments, the metabolic species is a BMP, GlcCer, GD3, GD1a/b, GM2 and/or GM3 species (e.g., as described herein). In certain embodiments, the accumulation of a combination of metabolic species is reduced, such as a combination of lipids described herein. Quantifying the amount of accumulation of a metabolic species in a cell may be performed using methods known in the art. For example, a metabolic species may be quantified using a liquid chromatography mass spectrometry (LCMS) assay (see, e.g., the Examples).

In certain embodiments, the accumulation of at least one metabolic species in the cell is reduced by at least about 10%, 15%, 20%, 25%, 30%, 40%, 50%, 60%, 70%, 80%, 90%, 95% or more. In certain embodiments, the accumulation of at least one metabolic species in the cell is reduced to levels in a control cell (e.g., a corresponding cell that does not comprise a lysosomal enzyme deficiency). In certain embodiments, the accumulation of a plurality of metabolic species is reduced (e.g., 2 or more metabolic species, such as 2, 3, 4, 5, 6, 7, 8, 9, 10, 25, 50 or more).

In certain embodiments, the CNS cell is selected from the group consisting of: a neuron, an astrocyte, and a microglial cell. In certain embodiments, the CNS cell is a neuron. In certain embodiments, the CNS cell is an astrocyte. In certain embodiments, the CNS cell is a microglial cell.

### CERTAIN EMBODIMENTS

Embodiment 1. A method of detecting one or more biomarkers in a subject having a lysosomal storage disorder (LSD), the method comprising:

1) measuring the concentration of a combination of two or more lipids in a sample from the subject, wherein the combination of lipids is selected from the group consisting of:
a) a bis(monoacylglycero)phosphate (BMP);
b) a GM2 ganglioside and/or a GM3 ganglioside;
c) a GD3 ganglioside;
d) a GD1a/b ganglioside; and
e) a glucosylceramide (GlcCer);

2) measuring the concentration of GlcCer in a sample from the subject, provided the LSD is a mucopolysaccharidosis (MPS) disorder;

3) measuring the concentration of neurofilament light chain (Nf-L) in a sample from the subject; and/or

4) measuring the concentration of soluble triggering receptor expressed on myeloid cells 2 (sTREM2) in a sample from the subject.

Embodiment 2. A method of evaluating the efficacy of a treatment in a subject having an LSD, the method comprising:

1) measuring the concentration of a combination of two or more lipids in a sample obtained from the subject after administration of the treatment, wherein the combination of lipids is selected from the group consisting of:
a) a BMP;
b) a GM2 ganglioside and/or a GM3 ganglioside;
c) a GD3;
d) a GD1a/b; and
e) a GlcCer;

2) measuring the concentration of GlcCer in a sample obtained from the subject after administration of the treatment, provided the LSD is an MPS disorder;

3) measuring the concentration of Nf-L in a sample obtained from the subject after administration of the treatment; and/or

4) measuring the concentration of sTREM2 in a sample obtained from the subject after administration of the treatment;

wherein a decrease in the concentration of the selected lipid(s)/protein(s) in the sample from obtained the subject after administration of the treatment as compared to the concentration of the lipid(s)/protein(s) in a sample obtained from the subject prior to administration of the treatment correlates with treatment efficacy.

Embodiment 3. A method of identifying a subject having an LSD as a candidate for treatment, comprising:

1) measuring the concentration of a combination of two or more lipids in a sample from the subject, wherein the combination of lipids is selected from the group consisting of:
a) a BMP;
b) a GM2 ganglioside and/or a GM3 ganglioside;
c) a GD3;
d) a GD1a/b; and
e) a GlcCer;

2) measuring the concentration of GlcCer in a sample from the subject, provided the LSD is an MPS disorder;

3) measuring the concentration of Nf-L in a sample from the subject; and/or

4) measuring the concentration of sTREM2 in a sample from the subject;

wherein a concentration of the selected lipid(s)/protein(s) in the sample from the subject that is at least as high as a control value identifies the subject as a candidate for treatment.

Embodiment 4. The method of any one of embodiments 1-3, further comprising administering an LSD treatment to the subject.

Embodiment 5. The method of any one of embodiments 1-4, further comprising adjusting a treatment regimen for the subject.

Embodiment 6. A method for treating an LSD in a subject, the method comprising:

1) administering an LSD treatment to the subject;

2) measuring the concentration of:
a) a combination of two or more lipids in a sample from the subject, wherein the combination of lipids is selected from the group consisting of:
   i) a BMP;
   ii) a GM2 ganglioside and/or a GM3 ganglioside;
   iii) a GD3;
   iv) a GD1a/b; and
   v) a GlcCer;
b) GlcCer in a sample from the subject, provided the LSD is an MPS disorder;
c) Nf-L in a sample from the subject; and/or
d) sTREM2 in a sample from the subject; and

3) adjusting the dosage of the LSD treatment based on the concentration of the selected lipid(s)/protein(s) in the sample from the subject as compared to a control value.

Embodiment 7. The method of any one of embodiments 1-6, comprising measuring the concentration of sTREM2.

Embodiment 8. The method of any one of embodiments 1-6, comprising measuring the concentration of Nf-L.

Embodiment 9. The method of any one of embodiments 1-6, comprising measuring the concentration of GlcCer, wherein the LSD is an MPS disorder.

Embodiment 10. The method of any one of embodiment 1-6, comprising measuring the concentration of a combination of two or more lipids.

Embodiment 11. The method of any one of embodiments 1-6, comprising measuring the concentration of one or more lipids and the concentration of sTREM2.

Embodiment 12. The method of any one of embodiments 1-6, comprising measuring the concentration of one or more lipids and the concentration of Nf-L.

Embodiment 13. The method of any one of embodiments 1-12, wherein the sample is a tissue sample, a serum sample or a cerebrospinal fluid sample.

Embodiment 14. The method of embodiment 13, wherein the sample is a tissue sample.

Embodiment 15. The method of embodiment 14, wherein the tissue is brain, liver, kidney, lung or spleen.

Embodiment 16. The method of embodiment 13, wherein the sample is a serum sample.

Embodiment 17. The method of embodiment 13, wherein the sample is a cerebrospinal fluid sample.

Embodiment 18. A method for treating an LSD in a subject, the method comprising administering an LSD treatment to the subject, wherein the subject has, or was determined to have:

1) an increased concentration of a combination of two or more lipids as compared to a control, wherein the combination of lipids is selected from the group consisting of:
a) a BMP;
b) a GM2 ganglioside and/or a GM3 ganglioside;
c) a GD3;
d) a GD1a/b; and
e) a GlcCer;

2) an increased concentration of GlcCer, provided the LSD is an MPS disorder;

3) an increased concentration of Nf-L; and/or

4) an increased concentration of sTREM2.

Embodiment 19. The method of embodiment 18, wherein the subject has, or was determined to have, an increased concentration of sTREM2.

Embodiment 20. The method of embodiment 18, wherein the subject has, or was determined to have, an increased concentration of Nf-L.

Embodiment 21. The method of embodiment 18, wherein the subject has, or was determined to have, an increased concentration of GlcCer, and wherein the LSD is an MPS disorder.

Embodiment 22. The method of embodiment 18, wherein the subject has, or was determined to have, an increased concentration of a combination of two or more lipids.

Embodiment 23. The method of embodiment 18, wherein the subject has, or was determined to have, an increased concentration of one or more lipids and an increased concentration of sTREM2.

Embodiment 24. The method of embodiment 18, wherein the subject has, or was determined to have, an increased concentration of one or more lipids and an increased concentration of Nf-L.

Embodiment 25. The method of any one of embodiments 1-24, wherein the combination comprises a BMP.

Embodiment 26. The method of any one of embodiments 1-25, wherein the combination comprises a GlcCer.

Embodiment 27. The method of any one of embodiments 1-26, wherein the combination comprises a GD3.

Embodiment 28. The method of any one of embodiments 1-27, wherein the combination comprises a GD1a/b Embodiment 29. The method of any one of embodiments 1-28, wherein the combination comprises a GM2.

Embodiment 30. The method of any one of embodiments 1-29, wherein the combination comprises a GM3.

Embodiment 31. The method of any one of embodiments 1-24, wherein the combination comprises: a BMP and a GlcCer; a BMP and a GD3; a BMP and a GD1a/b; a BMP and a GM2; a BMP and a GM3; a GlcCer and a GD3; a GlcCer and a GD1a/b; a GlcCer and a GM2; a GlcCer and a GM3; a GD3 and a GD1a/b; a GD3 and a GM2; a GD3 and a GM3; a GD1a/b and a GM2; a GD1a/b and a GM3; a BMP, a GlcCer and a GD3; a BMP, a GlcCer and a GD1a/b; a BMP, a GlcCer and a GM2; a BMP, a GlcCer and a GM3; a BMP, a GD3 and a GD1a/b; a BMP, a GD3 and a GM2; a BMP, a GD3 and a GM3; a BMP, a GD1a/b and a GM2; a BMP, a GD1a/b and a GM3; a BMP, a GM2 and a GM3; a GlcCer, a GD3 and a GD1a/b; a GlcCer, a GD3 and a GM2; a GlcCer, a GD3 and a GM3; a GlcCer, a GD1a/b and a GM2; a GlcCer, a GD1a/b and a GM3; a GlcCer, a GM2 and a GM3; a GD3, a GD1a/b and a GM2; a GD3, a GD1a/b and a GM3; a GD3, GM2 and a GM3; a GD1a/b, a GM2 and a GM3; a BMP, a GlcCer, a GD3 and a GD1a/b; a BMP, a GlcCer, a GD3 and a GM2; a BMP, a GlcCer, a GD3 and a GM3; a BMP, a GlcCer, a GD1a/b and GM2; a BMP, a GlcCer, a GD1a/b and GM3; a BMP, a GlcCer, a GM2 and GM3; a BMP, a GD3, a GD1a/b and a GM2; a BMP, a GD3, a GD1a/b and a GM3; a BMP, a GD3, a GM2 and a GM3; a BMP, a GD1a/b, a GM2 and a GM3; a GlcCer, a GD3, a GD1a/b and a GM2; a GlcCer, a GD3, a GD1a/b and a GM3; a GlcCer, a GD3, a GM2 and a GM3; a GlcCer, a GD1a/b, a GM2 and a GM3; a GD3, a GD1a/b, a GM2 and a GM3; a BMP, a GlcCer, a GD3, a GD1a/b and a GM2; a BMP, a GlcCer, a GD3, a GD1a/b and a GM3; a BMP, a GD3, a GD1a/b, a GM2 and a GM3; a BMP, a GlcCer, a GD3, a GM2 and a GM3; a BMP, a GlcCer, a GD1a/b, a GM2 and a GM3; a GlcCer, a GD3, a GD1a/b, a GM2 and a GM3; or a BMP, a GlcCer, a GD3, a GD1/b, a GM2 and a GM3.

Embodiment 32. The method of any one of embodiments 1-24, wherein the combination comprises: a BMP and a GlcCer; a BMP and a GD3; a BMP and a GD1a/b; a BMP and a GM2; a BMP and a GM3; a GlcCer and a GD3; a GlcCer and a GD1a/b; a GlcCer and a GM2; a GlcCer and a GM3; a GD3 and a GD1a/b; a GD3 and a GM2; a GD3 and a GM3; a GD1a/b and a GM2; or a GD1a/b and a GM3.

Embodiment 33. The method of any one of embodiments 1-24, wherein the combination comprises: a BMP, a GlcCer and a GD3; a BMP, a GlcCer and a GD1a/b; a BMP, a GlcCer and a GM2; a BMP, a GlcCer and a GM3; a BMP, a GD3 and a GD1a/b; a BMP, a GD3 and a GM2; a BMP, a GD3 and a GM3; a BMP, a GD1a/b and a GM2; a BMP, a GD1a/b and a GM3; a BMP, a GM2 and a GM3; a GlcCer, a GD3 and a GD1a/b; a GlcCer, a GD3 and a GM2; a GlcCer, a GD3 and a GM3; a GlcCer, a GD1a/b and a GM2; a GlcCer, a GD1a/b and a GM3; a GlcCer, a GM2 and a GM3; a GD3, a GD1a/b and a GM2; a GD3, a GD1a/b and a GM3; a GD3, GM2 and a GM3; or a GD1a/b, a GM2 and a GM3.

Embodiment 34. The method of any one of embodiments 1-24, wherein the combination comprises: a BMP, a GlcCer, a GD3 and a GD1a/b; a BMP, a GlcCer, a GD3 and a GM2; a BMP, a GlcCer, a GD3 and a GM3; a BMP, a GlcCer, a GD1a/b and GM2; a BMP, a GlcCer, a GD1a/b and GM3; a BMP, a GlcCer, a GM2 and GM3; a BMP, a GD3, a GD1a/b and a GM2; a BMP, a GD3, a GD1a/b and a GM3; a BMP, a GD3, a GM2 and a GM3; a BMP, a GD1a/b, a GM2 and a GM3; a GlcCer, a GD3, a GD1a/b and a GM2; a GlcCer, a GD3, a GD1a/b and a GM3; a GlcCer, a GD3, a GM2 and a GM3; a GlcCer, a GD1a/b, a GM2 and a GM3; or a GD3, a GD1a/b, a GM2 and a GM3.

Embodiment 35. The method of any one of embodiments 1-24, wherein the combination comprises: a BMP, a GlcCer, a GD3, a GD1a/b and a GM2; a BMP, a GlcCer, a GD3, a GD1a/b and a GM3; a BMP, a GD3, a GD1a/b, a GM2 and a GM3; a BMP, a GlcCer, a GD3, a GM2 and a GM3; a BMP, a GlcCer, a GD1a/b, a GM2 and a GM3; or a GlcCer, a GD3, a GD1a/b, a GM2 and a GM3.

Embodiment 36. The method of any one of embodiments 1-24, wherein the combination comprises: a BMP, a GlcCer, a GD3, a GD1/b, a GM2 and a GM3.

Embodiment 37. The method of embodiment any one of embodiments 1-36, wherein the LSD is an MPS disorder.

Embodiment 38. The method of embodiment 37, wherein the MPS disorder is Hunter's syndrome.

Embodiment 39. The method of any one of embodiments 2-38, wherein the LSD treatment comprises haematopoietic stem cell transplantation (HSCT), enzyme replacement therapy (ERT), substrate reduction therapy, chaperone therapy and/or gene therapy.

Embodiment 40. The method of embodiment 39, wherein the LSD treatment comprises ERT.

Embodiment 41. The method of embodiment 40, wherein the ERT is targeted to the brain.

Embodiment 42. The method of embodiment 40, wherein the LSD treatment is a protein comprising:
(a) a first Fc polypeptide that is linked to an enzyme replacement therapy (ERT) enzyme, an ERT enzyme variant, or a catalytically active fragment thereof; and
(b) a second Fc polypeptide that forms an Fc dimer with the first Fc polypeptide,
wherein the first Fc polypeptide and/or the second Fc polypeptide does not include an immunoglobulin heavy and/or light chain variable region sequence or an antigen-binding portion thereof.

Embodiment 43. The method of embodiment 42, wherein the ERT enzyme is iduronate 2-sulfatase (IDS), an IDS variant, or a catalytically active fragment thereof.

Embodiment 44. The method of embodiment 42, wherein the first Fc polypeptide comprises the amino acid sequence of any one of SEQ ID NOS:113, 193, and 197, and the second Fc polypeptide comprises the amino acid sequence SEQ ID NO:114.

Embodiment 45. The method of embodiment 42, wherein the first Fc polypeptide comprises the amino acid sequence of any one of SEQ ID NOS: 113, 193, and 197, and the second Fc polypeptide comprises the amino acid sequence of SEQ ID NO:131.

Embodiment 46. The method of embodiment 42, wherein the first Fc polypeptide comprises the amino acid sequence of any one of SEQ ID NOS: 113, 193, and 197, and the second Fc polypeptide comprises the amino acid sequence SEQ ID NO:167.

Embodiment 47. The method of embodiment 42, wherein the first Fc polypeptide comprises the amino acid sequence of any one of SEQ ID NOS: 113, 193, and 197, and the second Fc polypeptide comprises the amino acid sequence SEQ ID NO:190.

Embodiment 48. The method of embodiment 42, wherein the first Fc polypeptide comprises the amino acid sequence of any one of SEQ ID NOS: 113, 193, and 197, and the second Fc polypeptide comprises the amino acid sequence SEQ ID NO:191.

Embodiment 49. The method of embodiment 42, wherein the first Fc polypeptide comprises the amino acid sequence of any one of SEQ ID NOS: 113, 193, and 197, and the second Fc polypeptide comprises the amino acid sequence SEQ ID NO:117.

Embodiment 50. The method of embodiment 42, wherein the first Fc polypeptide comprises the amino acid sequence of any one of SEQ ID NOS: 113, 193, and 197, and the second Fc polypeptide comprises the amino acid sequence SEQ ID NO:130.

Embodiment 51. The method of embodiment 42, wherein the first Fc polypeptide comprises the amino acid sequence of any one of SEQ ID NOS: 113, 193, and 197, and the second Fc polypeptide comprises the amino acid sequence SEQ ID NO:132.

Embodiment 52. The method of embodiment 42, wherein the first Fc polypeptide comprises the amino acid sequence of any one of SEQ ID NOS: 113, 193, and 197, and the second Fc polypeptide comprises the amino acid sequence SEQ ID NO:166.

Embodiment 53. The method of embodiment 42, wherein the first Fc polypeptide comprises the amino acid sequence of any one of SEQ ID NOS: 113, 193, and 197, and the second Fc polypeptide comprises the amino acid sequence SEQ ID NO:168.

Embodiment 54. A method of screening a test agent for activity as an LSD treatment, the method comprising:

1) contacting a cell with the test agent, wherein the cell has impaired lysosomal storage; and

2) measuring the concentration of:
a) a combination of two or more lipids in the cell, wherein the combination of lipids is selected from the group consisting of:
   i) a BMP;
   ii) a GM2 ganglioside and/or a GM3 ganglioside;
   iii) a GD3;
   iv) a GD1a/b; and
   v) a GlcCer;
b) GlcCer in the cell, provided the test agent is screened for activity as an MPS treatment;
c) Nf-L in the cell; and/or
d) sTREM2 in the cell;

wherein a decrease in the concentration of the selected lipid(s)/protein(s) in the cell as compared to the concentration of corresponding lipid(s)/protein(s) in a control cell indicates the test agent has activity as an LSD treatment.

Embodiment 55. The method of embodiment 54, comprising measuring the concentration of sTREM2.

Embodiment 56. The method of embodiment 54, comprising measuring the concentration of Nf-L.

Embodiment 57. The method of embodiment 54, comprising measuring the concentration of GlcCer.

Embodiment 58. The method of embodiment 54, comprising measuring the concentration of a combination of two or more lipids.

Embodiment 59. The method of embodiment 54, comprising measuring the concentration of one or more lipids and the concentration of sTREM2.

Embodiment 60. The method of embodiment 54, comprising measuring the concentration of one or more lipids and the concentration of Nf-L.

Embodiment 61. The method of any one of embodiments 54-60, wherein the cell is a brain cell.

Embodiment 62. A corrected CNS cell comprising a deficiency in a lysosomal enzyme that causes accumulation of a metabolic species within the cell, wherein the cell is contacted with a protein comprising (i) a first Fc polypeptide linked to the lysosomal enzyme, and (ii) a second Fc polypeptide that forms an Fc dimer with the first Fc polypeptide, wherein the protein is capable of binding to the transferrin receptor (TfR), and wherein the correction is a reduction in the accumulation of the metabolic species.

Embodiment 63. A corrected CNS cell comprising reduced accumulation of a metabolic species, wherein a CNS cell comprising a deficiency in a lysosomal enzyme that causes accumulation of the metabolic species within the cell was contacted with a protein comprising:
(i) a first Fc polypeptide linked to the lysosomal enzyme; and
(ii) a second Fc polypeptide that forms an Fc dimer with the first Fc polypeptide, wherein the protein is capable of binding to the transferrin receptor (TfR), to provide the corrected CNS cell comprising the reduced accumulation of the metabolic species.

Embodiment 64. The CNS cell of embodiment 62 or 63, wherein the protein binds to TfR with an affinity of from about 50 nM to about 350 nM.

Embodiment 65. The CNS cell of any one of embodiments 62-64, wherein the enzyme is iduronate 2-sulfatase (IDS), or an enzymatically active variant thereof.

Embodiment 66. The CNS cell of any one of embodiments 62-65, wherein the metabolic species is a glycosaminoglycan (GAG) and/or a lysosomal lipid.

Embodiment 67. The CNS cell of any one of embodiments 62-65, wherein the metabolic species is a glycosaminoglycan (GAG).

Embodiment 68. The CNS cell of any one of embodiments 62-65, wherein the metabolic species is a lysosomal lipid.

Embodiment 69. The CNS cell of embodiment 68, wherein the lysosomal lipid is selected from the group consisting of: a ganglioside, a glucosylceramide, a galactosylceramide, and a bis(monoacylglycerol)phosphate (BMP).

Embodiment 70. The CNS cell of any one of embodiments 62-69, wherein the CNS cell is selected from the group consisting of: a neuron, an astrocyte, and a microglial cell.

Embodiment 71. A method of sorting populations of CNS cells from a tissue sample, comprising:

(a) contacting the tissue sample with a neuronal marker primary antibody, an astrocyte marker primary antibody, a microglial marker primary antibody, an endothelial marker primary antibody, and an oligodendrocyte marker primary antibody, wherein each primary antibody is uniquely labeled, to provide a labeled tissue sample; and

(b) sorting the cells in the labeled tissue sample by flow cytometry,

wherein the method provides distinct cell populations of neurons, astrocytes, and microglial cells.

Embodiment 72. The method of embodiment 71, wherein the neuronal marker primary antibody is an anti-Thy1 antibody.

Embodiment 73. The method of embodiment 71 or 72, wherein the microglial marker primary antibody is an anti-CD11b antibody.

Embodiment 74. The method of any one of embodiments 71-73, wherein the astrocyte marker primary antibody is selected from the group consisting of: an anti-EAAT2 antibody and an anti-astrocyte cell surface antigen-2 (ACSA-2) antibody.

Embodiment 75. The method of any one of embodiments 71-74, wherein the endothelial marker primary antibody is an anti-CD31 antibody.

Embodiment 76. The method of any one of embodiments 71-75, wherein the oligodendrocyte marker primary antibody is an anti-O1 antibody.

Embodiment 77. The method of any one of embodiments 71-76, further comprising contacting the tissue sample with a viability dye.

Embodiment 78. The method of any one of embodiments 71-77, which provides a distinct population of microglial cells comprising less than about 20% non-microglial cells, a distinct population of astrocytes comprising less than about 20% non-astrocytic cells and/or a distinct population of neurons comprising less than about 20% non-neuronal cells.

Embodiment 79. The method of embodiment 78, which provides a distinct population of microglial cells comprising less than about 20% non-microglial cells.

Embodiment 80. The method of embodiment 78 or 79, which provides a distinct population of astrocytes comprising less than about 20% non-astrocytic cells.

Embodiment 81. The method of any one of embodiments 78-80, which provides a distinct population of neurons comprising less than about 20% non-neuronal cells.

Embodiment 82. The method of any one of embodiments 71-81, wherein the microglial cell population is sorted based on the marker profile O1⁻/CD31⁻/CD11b⁺; the astrocyte population is sorted based on the marker profile O1⁻/CD31⁻/Thy1⁻/EAAT2⁺ or O1⁻/CD31⁻/Thy1⁻/ACSA-2⁺; and/or the neuron population is sorted based on the marker profile O1⁻/CD31⁻/Thy1⁺/EAAT2⁻ or O1⁻/CD31⁻/Thy1⁺/ACSA-2⁻.

Embodiment 83. The method of embodiment 82, wherein the microglial cell population is sorted based on the marker profile O1⁻/CD31⁻/CD11b⁺.

Embodiment 84. The method of embodiment 82 or 83, wherein the astrocyte population is sorted based on the marker profile O1⁻/CD31⁻/Thy1⁻/EAAT2⁺ or O1⁻/CD31⁻/Thy1⁻/ACSA-2⁺.

Embodiment 85. The method of any one of embodiments 82-84, wherein the neuron population is sorted based on the marker profile O1⁻/CD31⁻/Thy1⁺/EAAT2⁻ or O1⁻/CD31⁻/Thy1⁺/ACSA-2⁻.

Embodiment 86. The method of any one of embodiments 71-85, wherein the enriched cell populations are analyzed for quantification of sTREM2, Nf-L, a metabolic and/or a nucleic acid species.

Embodiment 87. The method of any one of embodiments 71-85, wherein the enriched cell populations are analyzed for quantification of a metabolic or nucleic acid species.

Embodiment 88. The method of embodiment 87, wherein the metabolic species is a glycosaminoglycan (GAG) species.

Embodiment 89. The method of embodiment 87, wherein the metabolic species is a lipid species.

Embodiment 90. The method of embodiment 89, wherein the lipid species is selected from the group consisting of: a ganglioside, a glucosylceramide, a galactosylceramide, and a bis(monoacylglycerol)phosphate (BMP).

Embodiment 91. The method of embodiment 87, wherein the nucleic acid species is selected from RNA, DNA, and genomic DNA.

Embodiment 92. The method of any one of embodiments 71-91, wherein the enriched cell populations are analyzed for quantification of sTREM2.

Embodiment 93. The method of any one of embodiments 71-92, wherein the enriched cell populations are analyzed for quantification of Nf-L.

Embodiment 94. The method of any one of embodiments 71-93, wherein the enriched cell populations are analyzed for quantification of an administered therapeutic agent.

Embodiment 95. The method of embodiment 94, wherein the administered therapeutic agent is ETV:IDS.

Certain Definitions

The terms “control” or “control sample” refer to any sample appropriate to the detection technique employed. The control sample may contain the products of the detection technique employed or the material to be tested. Further, the controls may be positive or negative controls.

The term “subject,” “individual,” and “patient,” as used interchangeably herein, refer to a mammal, including but not limited to humans, non-human primates, rodents (e.g., rats, mice, and guinea pigs), rabbits, cows, pigs, horses, and other mammalian species. In one embodiment, the patient is a human.

The term “pharmaceutically acceptable excipient” refers to a non-active pharmaceutical ingredient that is biologically or pharmacologically compatible for use in humans or animals, such as but not limited to a buffer, carrier, or preservative.

The term “administer” refers to a method of delivering agents (e.g., an LSD therapeutic agent, such as an ETV therapy described herein), compounds, or compositions (e.g, pharmaceutical composition) to the desired site of biological action. These methods include, but are not limited to, oral, topical delivery, parenteral delivery, intravenous delivery, intradermal delivery, intramuscular delivery, intrathecal delivery, colonic delivery, rectal delivery, or intraperitoneal delivery. In one embodiment, the polypeptides described herein are administered intravenously.

As used herein, “treatment” (and grammatical variations thereof such as “treat” or “treating”) refers to clinical intervention to alter the natural course of the individual being treated, and can be performed either for prophylaxis or during the course of clinical pathology.

Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis.

The phrase “effective amount” means an amount of a compound described herein that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

A “therapeutically effective amount” of a substance/molecule disclosed herein may vary according to factors such as the disease state, age, sex, and weight of the individual, and the ability of the substance/molecule, to elicit a desired response in the individual. A therapeutically effective amount encompasses an amount in which any toxic or detrimental effects of the substance/molecule are outweighed by the therapeutically beneficial effects. A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount.

The terms “obtaining a sample from a patient”, “obtained from a patient” and similar phrasing, is used to refer to obtaining the sample directly from the patient, as well as obtaining the sample indirectly from the patient through an intermediary individual (e.g., obtaining the sample from a courier who obtained the sample from a nurse who obtained the sample from the patient).

An “enzyme replacement therapy enzyme” or “ERT enzyme” refers to an enzyme that is deficient in a lysosomal storage disorder. An “ERT enzyme variant” refers to a functional variant, including allelic and splice variants, of a wild-type ERT enzyme or a fragment thereof, where the ERT enzyme variant has at least 50%, at least 55%, at least 60%, at least 65%, at least 70%, at least 75%, at least 80%, at least 85%, at least 90%, or at least 95% of the activity of the corresponding wild-type ERT enzyme or fragment thereof, e.g., when assayed under identical conditions. A “catalytically active fragment” of an ERT enzyme refers to a portion of a full-length ERT enzyme or a variant thereof, where the catalytically active fragment has at least 50%, at least 55%, at least 60%, at least 65%, at least 70%, at least 75%, at least 80%, at least 85%, at least 90%, or at least 95% of the activity of the corresponding full-length ERT enzyme or variant thereof, e.g., when assayed under identical conditions.

An “iduronate sulfatase,” “iduronate-2-sulfatase,” or “IDS” as used herein refers to iduronate 2-sulfatase (EC 3.1.6.13), which is an enzyme involved in the lysosomal degradation of the glycosaminoglycans heparan sulfate and dermatan sulfate. Deficiency of IDS is associated with Mucopolysaccharidosis II, also known as Hunter syndrome. The term “IDS” as used herein as a component of a protein that comprises an Fc polypeptide is catalytically active and encompasses functional variants, including allelic and splice variants, of a wild-type IDS or a fragment thereof. The sequence of human IDS isoform I, which is the human sequence designated as the canonical sequence, is available under UniProt entry P22304 and is encoded by the human IDS gene at Xq28. The full-length sequence is provided as SEQ ID NO:91. A “mature” IDS sequence as used herein refers to a form of a polypeptide chain that lacks the signal and propeptide sequences of the naturally occurring full-length polypeptide chain. The amino acid sequence of a mature human IDS polypeptide is provided as SEQ ID NO:92, which corresponds to amino acids 34-550 of the full-length human sequence. A “truncated” IDS sequence as used herein refers to a catalytically active fragment of the naturally occurring full-length polypeptide chain. The amino acid sequence of an exemplary truncated human IDS polypeptide is provided as SEQ ID NO:112, which corresponds to amino acids 26-550 of the full-length human sequence. The structure of human IDS has been well-characterized. An illustrative structure is available under PDB accession code 5FQL. The structure is also described in Nat. Comm. 8:15786 doi: 10.1038/ncomms15786, 2017. Non-human primate IDS sequences have also been described, including chimpanzee (UniProt entry K7BKV4) and rhesus macaque (UniProt entry H9FTX2). A mouse IDS sequence is available under Uniprot entry Q08890. An IDS variant has at least 50%, at least 55%, at least 60%, at least 65%, at least 70%, at least 75%, at least 80%, at least 85%, at least 90%, or at least 95% of the activity of the corresponding wild-type IDS or fragment thereof, e.g., when assayed under identical conditions. A catalytically active IDS fragment has at least 50%, at least 55%, at least 60%, at least 65%, at least 70%, at least 75%, at least 80%, at least 85%, at least 90%, or at least 95% of the activity of the corresponding full-length IDS or variant thereof, e.g., when assayed under identical conditions.

A “transferrin receptor” or “TfR” as used herein refers to transferrin receptor protein 1. The human transferrin receptor 1 polypeptide sequence is set forth in SEQ ID NO:94. Transferrin receptor protein 1 sequences from other species are also known (e.g., chimpanzee, accession number XP_003310238.1; rhesus monkey, NP_001244232.1; dog, NP_001003111.1; cattle, NP_001193506.1; mouse, NP_035768.1; rat, NP_073203.1; and chicken, NP_990587.1). The term “transferrin receptor” also encompasses allelic variants of exemplary reference sequences, e.g., human sequences, that are encoded by a gene at a transferrin receptor protein 1 chromosomal locus. Full-length transferrin receptor protein includes a short N-terminal intracellular region, a transmembrane region, and a large extracellular domain. The extracellular domain is characterized by three domains: a protease-like domain, a helical domain, and an apical domain. The apical domain sequence of human transferrin receptor 1 is set forth in SEQ ID NO:200.

A “fusion protein” or “[ERT enzyme]-Fc fusion protein” as used herein refers to a dimeric protein comprising a first Fc polypeptide that is linked (e.g., fused) to an ERT enzyme, an ERT enzyme variant, or a catalytically active fragment thereof (i.e., an “[ERT]-Fc fusion polypeptide”); and a second Fc polypeptide that forms an Fc dimer with the first Fc polypeptide. The second Fc polypeptide may also be linked (e.g., fused) to an ERT enzyme, an ERT enzyme variant, or a catalytically active fragment thereof. The first Fc polypeptide and/or the second Fc polypeptide may be linked to the ERT enzyme, ERT enzyme variant, or catalytically active fragment thereof by a peptide bond or by a polypeptide linker. The first Fc polypeptide and/or the second Fc polypeptide may be a modified Fc polypeptide that contains one or more modifications that promote its heterodimerization to the other Fc polypeptide. The first Fc polypeptide and/or the second Fc polypeptide may be a modified Fc polypeptide that contains one or more modifications that confer binding to a transferrin receptor. The first Fc polypeptide and/or the second Fc polypeptide may be a modified Fc polypeptide that contains one or more modifications that reduce effector function. The first Fc polypeptide and/or the second Fc polypeptide may be a modified Fc polypeptide that contains one or more modifications that extend serum half-life.

A “fusion polypeptide” or “[ERT enzyme]-Fc fusion polypeptide” as used herein refers to an Fc polypeptide that is linked (e.g., fused) to an ERT enzyme, an ERT enzyme variant, or a catalytically active fragment thereof. The Fc polypeptide may be linked to the ERT enzyme, ERT enzyme variant, or catalytically active fragment thereof by a peptide bond or by a polypeptide linker. The Fc polypeptide may be a modified Fc polypeptide that contains one or more modifications that promote its heterodimerization to another Fc polypeptide. The Fc polypeptide may be a modified Fc polypeptide that contains one or more modifications that confer binding to a transferrin receptor. The Fc polypeptide may be a modified Fc polypeptide that contains one or more modifications that reduce effector function. The Fc polypeptide may be a modified Fc polypeptide that contains one or more modifications that extend serum half-life.

As used herein, the term “Fc polypeptide” refers to the C-terminal region of a naturally occurring immunoglobulin heavy chain polypeptide that is characterized by an Ig fold as a structural domain. An Fc polypeptide contains constant region sequences including at least the CH2 domain and/or the CH3 domain and may contain at least part of the hinge region. In general, an Fc polypeptide does not contain a variable region.

A “modified Fc polypeptide” refers to an Fc polypeptide that has at least one mutation, e.g., a substitution, deletion or insertion, as compared to a wild-type immunoglobulin heavy chain Fc polypeptide sequence, but retains the overall Ig fold or structure of the native Fc polypeptide.

The term “FcRn” refers to the neonatal Fc receptor. Binding of Fc polypeptides to FcRn reduces clearance and increases serum half-life of the Fc polypeptide. The human FcRn protein is a heterodimer that is composed of a protein of about 50 kDa in size that is similar to a major histocompatibility (MHC) class I protein and a β2-microglobulin of about 15 kDa in size.

As used herein, an “FcRn binding site” refers to the region of an Fc polypeptide that binds to FcRn. In human IgG, the FcRn binding site, as numbered using the EU index, includes T250, L251, M252, 1253, S254, R255, T256, T307, E380, M428, H433, N434, H435, and Y436. These positions correspond to positions 20 to 26, 77, 150, 198, and 203 to 206 of SEQ ID NO:1.

As used herein, a “native FcRn binding site” refers to a region of an Fc polypeptide that binds to FcRn and that has the same amino acid sequence as the region of a naturally occurring Fc polypeptide that binds to FcRn.

The terms “CH3 domain” and “CH2 domain” as used herein refer to immunoglobulin constant region domain polypeptides. For purposes of this application, a CH3 domain polypeptide refers to the segment of amino acids from about position 341 to about position 447 as numbered according to EU, and a CH2 domain polypeptide refers to the segment of amino acids from about position 231 to about position 340 as numbered according to the EU numbering scheme and does not include hinge region sequences. CH2 and CH3 domain polypeptides may also be numbered by the IMGT (ImMunoGeneTics) numbering scheme in which the CH2 domain numbering is 1-110 and the CH3 domain numbering is 1-107, according to the IMGT Scientific chart numbering (IMGT website). CH2 and CH3 domains are part of the Fc region of an immunoglobulin. An Fc region refers to the segment of amino acids from about position 231 to about position 447 as numbered according to the EU numbering scheme, but as used herein, can include at least a part of a hinge region of an antibody. An illustrative hinge region sequence is the human IgG1 hinge sequence EPKSCDKTHTCPPCP (SEQ ID NO:93).

“Naturally occurring,” “native” or “wild type” is used to describe an object that can be found in nature as distinct from being artificially produced. For example, a nucleotide sequence present in an organism (including a virus), which can be isolated from a source in nature and which has not been intentionally modified in the laboratory, is naturally occurring. Furthermore, “wild-type” refers to the normal gene, or organism found in nature without any known mutation. For example, the terms “wild-type,” “native,” and “naturally occurring” with respect to a CH3 or CH2 domain are used herein to refer to a domain that has a sequence that occurs in nature. As used herein, the term “mutant” with respect to a mutant polypeptide or mutant polynucleotide is used interchangeably with “variant.” A variant with respect to a given wild-type CH3 or CH2 domain reference sequence can include naturally occurring allelic variants. A “non-naturally” occurring CH3 or CH2 domain refers to a variant or mutant domain that is not present in a cell in nature and that is produced by genetic modification, e.g., using genetic engineering technology or mutagenesis techniques, of a native CH3 domain or CH2 domain polynucleotide or polypeptide. A “variant” includes any domain comprising at least one amino acid mutation with respect to wild-type. Mutations may include substitutions, insertions, and deletions.

The term “amino acid” refers to naturally occurring and synthetic amino acids, as well as amino acid analogs and amino acid mimetics that function in a manner similar to the naturally occurring amino acids.

Naturally occurring amino acids are those encoded by the genetic code, as well as those amino acids that are later modified, e.g., hydroxyproline, γ-carboxyglutamate and 0-phosphoserine. “Amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that function in a manner similar to a naturally occurring amino acid.

Naturally occurring a-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring a-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Amino acids may be referred to herein by either their commonly known three letter symbols or by the one-letter symbols recommended by the IUPAC-IUB Biochemical Nomenclature Commission.

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids.

The term “protein” as used herein refers to either a polypeptide or a dimer (i.e, two) or multimer (i.e., three or more) of single chain polypeptides. The single chain polypeptides of a protein may be joined by a covalent bond, e.g., a disulfide bond, or non-covalent interactions.

The term “conservative substitution,” “conservative mutation,” or “conservatively modified variant” refers to an alteration that results in the substitution of an amino acid with another amino acid that can be categorized as having a similar feature. Examples of categories of conservative amino acid groups defined in this manner can include: a “charged/polar group” including Glu (Glutamic acid or E), Asp (Aspartic acid or D), Asn (Asparagine or N), Gln (Glutamine or Q), Lys (Lysine or K), Arg (Arginine or R), and His (Histidine or H); an “aromatic group” including Phe (Phenylalanine or F), Tyr (Tyrosine or Y), Trp (Tryptophan or W), and (Histidine or H); and an “aliphatic group” including Gly (Glycine or G), Ala (Alanine or A), Val (Valine or V), Leu (Leucine or L), Ile (Isoleucine or I), Met (Methionine or M), Ser (Serine or S), Thr (Threonine or T), and Cys (Cysteine or C). Within each group, subgroups can also be identified. For example, the group of charged or polar amino acids can be sub-divided into sub-groups including: a “positively-charged sub-group” comprising Lys, Arg and His; a “negatively-charged sub-group” comprising Glu and Asp; and a “polar sub-group” comprising Asn and Gln. In another example, the aromatic or cyclic group can be sub-divided into sub-groups including: a “nitrogen ring sub-group” comprising Pro, His and Trp; and a “phenyl sub-group” comprising Phe and Tyr. In another further example, the aliphatic group can be sub-divided into sub-groups, e.g., an “aliphatic non-polar sub-group” comprising Val, Leu, Gly, and Ala; and an “aliphatic slightly-polar sub-group” comprising Met, Ser, Thr, and Cys. Examples of categories of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn or vice versa, such that a free —NH₂ can be maintained. In some embodiments, hydrophobic amino acids are substituted for naturally occurring hydrophobic amino acid, e.g., in the active site, to preserve hydrophobicity.

The terms “identical” or percent “identity,” in the context of two or more polypeptide sequences, refer to two or more sequences or subsequences that are the same or have a specified percentage of amino acid residues, e.g., at least 60% identity, at least 65%, at least 70%, at least 75%, at least 80%, at least 85%, at least 90%, or at least 95% or greater, that are identical over a specified region when compared and aligned for maximum correspondence over a comparison window, or designated region, as measured using a sequence comparison algorithm or by manual alignment and visual inspection.

For sequence comparison of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 (Genentech, South San Francisco, Calif.) or Megalign (DNASTAR). The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. Thus, for example, an amino acid residue in a modified Fc polypeptide “corresponds to” an amino acid in SEQ ID NO:1, when the residue aligns with the amino acid in SEQ ID NO:1 when optimally aligned to SEQ ID NO:1. The polypeptide that is aligned to the reference sequence need not be the same length as the reference sequence.

The term “polynucleotide” and “nucleic acid” interchangeably refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. Examples of polynucleotides contemplated herein include single- and double-stranded DNA, single- and double-stranded RNA, and hybrid molecules having mixtures of single- and double-stranded DNA and RNA.

A “binding affinity” as used herein refers to the strength of the non-covalent interaction between two molecules, e.g., a single binding site on a polypeptide and a target, e.g., transferrin receptor, to which it binds. Thus, for example, the term may refer to 1:1 interactions between a polypeptide and its target, unless otherwise indicated or clear from context. Binding affinity may be quantified by measuring an equilibrium dissociation constant (KD), which refers to the dissociation rate constant (k_{d}, time⁻¹) divided by the association rate constant (kₐ, time⁻¹W). KD can be determined by measurement of the kinetics of complex formation and dissociation, e.g., using Surface Plasmon Resonance (SPR) methods, e.g., a Biacore™ system; kinetic exclusion assays such as KinExA®; and BioLayer interferometry (e.g., using the ForteBio® Octet® platform). As used herein, “binding affinity” includes not only formal binding affinities, such as those reflecting 1:1 interactions between a polypeptide and its target, but also apparent affinities for which Kg's are calculated that may reflect avid binding.

As used herein, the term “specifically binds” or “selectively binds” to a target, e.g., TfR, when referring to an engineered TfR-binding polypeptide, TfR-binding peptide, or TfR-binding antibody as described herein, refers to a binding reaction whereby the engineered TfR-binding polypeptide, TfR-binding peptide, or TfR-binding antibody binds to the target with greater affinity, greater avidity, and/or greater duration than it binds to a structurally different target. In typical embodiments, the engineered TfR-binding polypeptide, TfR-binding peptide, or TfR-binding antibody has at least 5-fold, 10-fold, 50-fold, 100-fold, 1,000-fold, 10,000-fold, or greater affinity for a specific target, e.g., TfR, compared to an unrelated target when assayed under the same affinity assay conditions. The term “specific binding,” “specifically binds to,” or “is specific for” a particular target (e.g., TfR), as used herein, can be exhibited, for example, by a molecule having an equilibrium dissociation constant KD for the target to which it binds of, e.g., 10⁻⁴ M or smaller, e.g., 10⁻⁵ M, 10⁻⁶ M, 10⁻⁷ M, 10⁻⁸ M, 10⁻⁹M, 10⁻¹⁰M, 10⁻¹¹ M, or 10⁻¹²M. In some embodiments, an engineered TfR-binding polypeptide, TfR-binding peptide, or TfR-binding antibody specifically binds to an epitope on TfR that is conserved among species, (e.g., structurally conserved among species), e.g., conserved between non-human primate and human species (e.g., structurally conserved between non-human primate and human species). In some embodiments, an engineered TfR-binding polypeptide, TfR-binding peptide, or TfR-binding antibody may bind exclusively to a human TfR.

The term “variable region” or “variable domain” refers to a domain in an antibody heavy chain or light chain that is derived from a germline Variable (V) gene, Diversity (D) gene, or Joining (J) gene (and not derived from a Constant (Cμ and Cδ) gene segment), and that gives an antibody its specificity for binding to an antigen. Typically, an antibody variable region comprises four conserved “framework” regions interspersed with three hypervariable “complementarity determining regions.”

The terms “antigen-binding portion” and “antigen-binding fragment” are used interchangeably herein and refer to one or more fragments of an antibody that retains the ability to specifically bind to an antigen via its variable region. Examples of antigen-binding fragments include, but are not limited to, a Fab fragment (a monovalent fragment consisting of the VL, VH, CL, and CH1 domains), a F(ab′)2 fragment (a bivalent fragment comprising two Fab fragments linked by a disulfide bridge at the hinge region), a single chain Fv (scFv), a disulfide-linked Fv (dsFv), complementarity determining regions (CDRs), a VL (light chain variable region), and a VH (heavy chain variable region).

The following Examples are intended to be non-limiting.

### Example 1: Effect of Peripheral Administration of ETV:IDS on Brain GAG and Lysosomal Lipids in IDS KO x TfR^{muhu} Mice

As described below, the effects of peripheral administration of ETV:IDS on brain GAG and lysosomal lipids in IDS KO x TfR^{muhu} mice was investigated.

### Materials and Methods

### Animal Care

Mice were housed under a 12-hour light/dark cycle and had access to water and standard rodent diet (LabDiet® #25502, Irradiated) ad libitum.

### Mouse Strains

A previously described, IDS KO mice on a B6N background were obtained from The Jackson Laboratories (JAX strain 024744). Development and characterization of the TfR^{mu/hu} KI mouse line harboring the human TfR apical domain knocked into the mouse receptor was previously described (U.S. Pat. No. 10,143,187). TfR^{mu/hu} male mice were bred to female IDS heterozygous mice to generate IDS KO x TfR^{hu/mu} mice. All mice used in this study were males.

Administration and tissue collection 2 month old IDS KO x TfR^{mu/hu} mice were injected i.v. with idursulfase (14.2 mg/kg body weight), or ETV:IDS (40 mg/kg body weight) once every week for 4 weeks (n=8). 2 month-old littermate TfR^{hu/mu} mice, injected i.v. with saline once every week for 4 weeks (n=5) were used as controls. For the 7-day cohort, animals were sacrificed 7 days following the first. For the 28-day cohort, animals were sacrificed 7 days following fourth weekly dose.

For terminal sample collection, animals were deeply anesthetized via intraperitoneal (i.p.) injection of 2.5% Avertin. For CSF collection, a sagittal incision was made at the back of the animal's skull, subcutaneous tissue and muscle was separated to expose the cisterna magna and a pre-pulled glass capillary tube was used to puncture the cisterna magna to collect CSF. CSF was transferred to a Low Protein LoBind Eppendorf tube and centrifuged at 12,700 rpm for 10 minutes at 4° C. CSF was transferred to a fresh tube and snap frozen on dry ice. Lack of blood contamination in mouse CSF was confirmed by measuring the absorbance of the samples at 420 nm. Blood was collected via cardiac puncture for serum collection. For serum collection, blood was allowed to clot at room temperature for at least 30 minutes. Tubes were then centrifuged at 12,700 rpm for 7 minutes at 4° C. Serum was transferred to a fresh tube and flash-frozen on dry ice. Animals were transcardially perfused with ice-cold PBS using a peristaltic pump (Gilson Inc. Minipuls Evolution). The brain and liver were dissected and flash-frozen on dry ice.

### Tissue Preparation: GAG Extraction and Processing

Brain and liver tissue samples and CSF were collected as described above. Brain and liver tissue were homogenized using a TissueLyser from Qiagen. Tissue homogenate was then transferred to a 96-well deep plate and sonicated using a 96-tip sonicator (Q Sonica). A BCA protein assay was performed to quantify total protein, and 20 μg liver and 100 μg brain per sample were subjected to LC-MS/MS sample preparation. 3 μL was used per sample for CSF. Briefly, heparan and dermatan sulfate derived disaccharides were generated by digesting tissue homogenates using a combination of Heparinases I, II, III and Chondriotinase B. Digests were mixed with acetonitrile and subjected to LC-MS/MS as described below.

### LCMS Assay for GAG

Quantification of GAG was performed by liquid chromatography (Shimadzu Nexera X2 system, Shimadzu Scientific Instrument, Columbia, Md., USA) coupled to electrospray mass spectrometry (Sciex QTRAP 6500+, Sciex, Framingham, Mass., USA). For each analysis, sample was injected on a ACQUITY UPLC BEH Amide 1.7 mm, 2.1×150 mm column (Waters Corporation) using a flow rate of 0.55 mL/minute with a column temperature of 55° C. Mobile phases A and B consisted of water with 10 mM ammonium formate and 0.1% formic acid, and acetonitrile with 0.1% formic acid, respectively. A gradient was programmed as follows: 0.0-0.5 minutes at 80% B, 0.5-3.5 minutes from 80% B to 50% B, 3.5-4.0 minutes 50% B to 80% B, 4.0-4.5 minutes hold at 80% B. Electrospray ionization was performed in the negative-ion mode applying the following settings: curtain gas at 25; collision gas was set at medium; ion spray voltage at −4500; temperature at 600° C.; ion source Gas 1 at 50; ion source Gas 2 at 60. Data acquisition was performed using Analyst 1.6.3 (Sciex) in multiple reaction monitoring mode (MRM), with dwell time 50 (msec) for each species. collision energy (CE) was set at −30; declustering potential (DP) at −80; entrance potential (EP) at −10; collision cell exit potential (CXP) at −10. GAGs were detected as [M−H]− using the following MRM transitions: D0A0 at m/z 378.1>87.0; D0S0 at m/z 416.1>138.0; D0a4 at m/z 458.1>300.0; D4UA-2S-G1cNCOEt-6S (Iduron Ltd, Manchester, UK) at m/z 472.0 (in source fragment ion) >97.0 was used as internal standard. Individual disaccharide species were identified based on their retention times and MRM transitions using commercially available reference standards (Iduron Ltd). GAGs were quantified by the peak area ratio of D0A0, D0S0 and D0a4 to the internal standard using MultiQuant 3.0.2 (Sciex). Reported GAG amounts were normalized to total protein levels as measured by a BCA assay (Pierce).

Tissue Preparation: Lipid extraction from brain tissue

Frozen brain tissues (20±2 mg) were transferred into dry ice 2 mL Safe-Lock Eppendorf tube (Eppendorf Cat #022600044) and kept in dry ice containing a 5 mm stainless steel bead (QIAGEN Cat #69989) and 400 μl of MS-grade methanol containing internal standards. The tissues were homogenized with Tissuelyser for 30 sec at 25 Hz (in the cold room). The samples were then centrifuged for 20 min at 21,000×g at 4° C. The methanol supernatant was transferred into new eppendorf vials and were left at −20° C. for 1 hour to allow for further precipitation of proteins. The samples were then centrifuged for 10 min at 21,000×g at 4° C. 200 uL of the methanol supernatant was transferred into a LCMS 96 well-plate and dry down under nitrogen and then resuspended in 100 uL of ACN/IPA/H2O (92.5/5/2.5) with 5 mM ammonium formate and 0.5% formic acid for GlcCer analysis. The rest of the supernatant was transferred, without disturbing pellet, into a separate LCMS 96 well-plate for analysis of BMP and ganglioside species. The samples were either directly run on LCMS or stored at −80° C.

LCMS Assay Fin. BMP and Gangliosides

BMP and gangliosides analyses were performed by liquid chromatography (Shimadzu Nexera X2 system, Shimadzu Scientific instrument, Columbia, Md., USA) coupled to electrospray mass spectrometry (Sciex QTRAP 6500+, Sciex, Framingham, Mass., USA). For each analysis, 5 μL of sample was injected on a BEH C18 1.7 μm, 2.1×100 mm column (Waters Corporation, Milford, Mass., USA) using a flow rate of 0.25 mL/min at 55° C. Mobile phase A consisted of 60:40 acetonitrile/water (v/v) with 10 mM ammonium acetate Mobile phase B consisted of 90:10 isopropyl alcohol/acetonitrile (v/v) with 10 mM ammonium acetate. The gradient was programmed as follows: 0.0-0.01 min from 45% B to 99% B, 0.1-3.0 min at 99% B, 3.0-3.01 min to 45% B, and 3.01-3.50 min at 45% B. Electrospray ionization was performed in negative ion mode applying the following settings: curtain gas at 30; collision gas was set at medium; ion spray voltage at −4500; temperature at 600° C.; ion source Gas I at 50; ion source Gas 2 at 60. Data acquisition was performed using Analyst 1.6.3 (Sciex) in multiple reaction monitoring mode (MRM), with the following parameters: dwell time (msec) for each species reported in the Table A, collision energy (CE) at −50, declustering potential (DP) at −80; entrance potential (EP) at −10; and collision cell exit (CSP) potential at −15. BMP and gangliosides species were quantified using the non endogenous internal standards BMP di14:0 and GM3 (d36:1 (d5)). Quantification was performed using MultiQuant 3.02. (Sciex). BMP and gangliosides concentration were normalized to either total protein amount, tissue weight or volume. Protein concentration was measured using the bicinchoninic acid (BCA) assay (Pierce, Rockford, Ill. USA),

TABLE A
Acquisition parameters information for the BMP and gangliosides assay.

| | | | Q1 | Q2 | Time | | |
|---|---|---|---|---|---|---|---|
| Lipid | Internal standard | Group | mass | mass | (msec) | DP | CE |
| BMP (14:0/14:0) | Internal standard | BMP | 665.3 | 227.2 | 20 | −80 | −50 |
| BMP (20:4/20:4) | BMP (14:0/14:0) | BMP | 817.5 | 303.3 | 200 | −80 | −50 |
| BMP (22:6/22:6) | BMP (14:0/14:0) | BMP | 865.5 | 327.3 | 800 | −80 | −50 |
| BMP (18:1/18:1) | BMP (14:0/14:0) | BMP | 773.5 | 281.3 | 50 | −80 | −50 |
| GM3(d18:1/18:0(d5)) | Internal standard | GM | 1184.8 | 290.1 | 10 | −60 | −65 |
| GM3(d34:1) | GM3(d18:1/18:0(d5)) | GM3 | 1151.7 | 290.1 | 10 | −60 | −65 |
| GM3(d36:1) | GM3(d18:1/18:0(d5)) | GM3 | 1179.8 | 290.1 | 10 | −60 | −65 |
| GM3(d38:1) | GM3(d18:1/18:0(d5)) | GM3 | 1207.8 | 290.1 | 10 | −60 | −65 |
| GM3(d40:1) | GM3(d18:1/18:0(d5)) | GM3 | 1235.8 | 290.1 | 10 | −60 | −65 |
| GM3(d41:1) | GM3(d18:1/18:0(d5)) | GM3 | 1249.8 | 290.1 | 10 | −60 | −65 |
| GM3(d42:2) | GM3(d18:1/18:0(d5)) | GM3 | 1261.8 | 290.1 | 10 | −60 | −65 |
| GM3(d42:1) | GM3(d18:1/18:0(d5)) | GM3 | 1263.8 | 290.1 | 10 | −60 | −65 |
| GM3(d43:0) | GM3(d18:1/18:0(d5)) | GM3 | 1279.8 | 290.1 | 10 | −60 | −65 |
| GM3(d44:1) | GM3(d18:1/18:0(d5)) | GM3 | 1291.8 | 290.1 | 10 | −60 | −65 |
| GM3(d44:2) | GM3(d18:1/18:0(d5)) | GM3 | 1289.8 | 290.1 | 10 | −60 | −65 |
| GM1(d36:1) | GM3(d18:1/18:0(d5)) | GM1 | 1544.9 | 290.1 | 10 | −275 | −75 |
| GM1(d38:1) | GM3(d18:1/18:0(d5)) | GM1 | 1572.9 | 290.1 | 10 | −275 | −75 |
| GM2(d38:1) | GM3(d18:1/18:0(d5)) | GM2 | 1410.7 | 290.1 | 10 | −60 | −65 |
| GM2(d36:1) | GM3(d18:1/18:0(d5)) | GM2 | 1382.8 | 290.1 | 10 | −60 | −65 |
| GD3(d34:1) | GM3(d18:1/18:0(d5)) | GM3 | 720.9 | 290.1 | 8 | −60 | −40 |
| GD3(d36:1) | GM3(d18:1/18:0(d5)) | GM3 | 734.9 | 290.1 | 8 | −60 | −40 |
| GD3(d38:1) | GM3(d18:1/18:0(d5)) | GM3 | 748.9 | 290.1 | 8 | −60 | −40 |
| GD3(d40:1) | GM3(d18:1/18:0(d5)) | GM3 | 762.9 | 290.1 | 8 | −60 | −40 |
| GD3(d42:2) | GM3(d18:1/18:0(d5)) | GM3 | 775.9 | 290.1 | 8 | −60 | −40 |
| GD3(d42:1) | GM3(d18:1/18:0(d5)) | GM3 | 776 | 290.1 | 8 | −60 | −40 |
| GD1a/b(d36:1) | GM3(d18:1/18:0(d5)) | GM3 | 917.5 | 290.1 | 8 | −60 | −52 |
| GD1a/b(d38:1) | GM3(d18:1/18:0(d5)) | GM3 | 931.5 | 290.1 | 8 | −60 | −52 |
| GT1b(d36:1) | GM3(d18:1/18:0(d5)) | GM3 | 1063 | 290.1 | 8 | −60 | −35 |
| GT1b(d38:1) | GM3(d18:1/18:0(d5)) | GM3 | 1077 | 290.1 | 8 | −60 | −35 |
| GQ1b(d36:1) | GM3(d18:1/18:0(d5)) | GM3 | 1208.6 | 290.1 | 8 | −60 | −55 |
| GQ1b(d38:1) | GM3(d18:1/18:0(d5)) | GM3 | 1222.6 | 290.1 | 8 | −60 | −55 |

LCMS assay for GlcCer and GalCer

Glucosylceramide and galactosylceramide analyses were performed by liquid chromatography (Shimadzu Nexera X2 system, Shimadzu Scientific instrument, Columbia, Md., USA) coupled to electrospray mass spectrometry (Sciex QTRAP 6500+ Sciex, Framingham, Mass., USA). For each analysis, 10 μL of sample was injected on a HALO HILIC 2.0 μm, 3.0×150) mm column (Advanced Materials Technology, PN 91813-701) using a flow rate of 0.45 mL/min at 45° C. Mobile phase A consisted of 92.5/5/2.5 ACN/IPA/H2O with 5 mM ammonium formate and 0.5% formic Acid. Mobile phase B consisted of 92.5/5/2.5 H2O/IPA/ACN with 5 mM ammonium formate and 0.5% formic Acid. The gradient was programmed as follows: 0.0-3.1 min at 100% B, 3.2 min at 95% B, 5.7 min at 85% B, hold to 7.1 min at 85% B, drop to 0% B at 7.25 min and hold to 8.75 min, ramp back to 100% at 10.65 min and hold to 11 min. Electrospray ionization was performed in the positive-ion mode applying the following settings: curtain gas at 25; collision gas was set at medium; ion spray voltage at 5500; temperature at 350° C.; ion source Gas I at 55; ion source Gas 2 at 60. Data acquisition was performed using Analyst 1.6 (Sciex) in multiple reaction monitoring mode (MRM) with the following parameters: dwell time (msec) and collision energy (CE) for each species reported in Table B; declustering potential (DP) at 45; entrance potential (EP) at 10; and collision cell exit potential (CXP) at 12.5. Lipids were quantified using a mixture of isotope labeled internal standards as reported in Table B. Glucosylceramide and Galactosylceramide were identified based on their retention times and MRM properties of commercially available reference standards (Avanti Polar Lipids, Birmingham, Ala., USA). Quantification was performed using MultiQuant 3.02 (Sciex). Metabolites were normalized to either total protein amount, tissue weight or volume.

TABLE B
Acquisition parameters information for GlcCer and GalCer Assay.

| | | | Q1 | Q2 | Time | | |
|---|---|---|---|---|---|---|---|
| Lipid | Internal Standard | Group | mass | mass | (msec) | DP | CE |
| GlcCer(d18:1/16:0) | GlcCer (d18:1(d5)/18:0) | GlcCer | 700.6 | 264.3 | 50 | 45 | 45 |
| GlcCer(d18:1/18:0) | GlcCer (d18:1(d5)/18:0) | GlcCer | 728.6 | 264.3 | 50 | 45 | 45 |
| GlcCer(d18:2/18:0) | GlcCer (d18:1(d5)/18:0) | GlcCer | 726.6 | 262.3 | 50 | 45 | 45 |
| GlcCer(d18:1/20:0) | GlcCer (d18:1(d5)/18:0) | GlcCer | 756.6 | 264.3 | 50 | 45 | 50 |
| GlcCer (d18:2/20:0) | GlcCer (d18:1(d5)/18:0) | GlcCer | 754.6 | 262.3 | 50 | 45 | 50 |
| GlcCer (d18:1/22:0) | GlcCer (d18:1(d5)/18:0) | GlcCer | 784.6 | 264.3 | 50 | 45 | 50 |
| GlcCer (d18:1/22:1) | GlcCer (d18:1(d5)/18:0) | GlcCer | 782.6 | 264.3 | 50 | 45 | 50 |
| GlcCer (d18:2/22:0) | GlcCer (d18:1(d5)/18:0) | GlcCer | 782.6 | 262.3 | 50 | 45 | 50 |
| GlcCer (d18:1/24:1) | GlcCer (d18:1(d5)/18:0) | GlcCer | 810.7 | 264.3 | 50 | 45 | 50 |
| GlcCer (d18:1/24:0) | GlcCer (d18:1(d5)/18:0) | GlcCer | 812.7 | 264.3 | 50 | 45 | 50 |
| Glucosyl Sphingosine | Glucosyl Sphingosine-d5 | GlcCer | 462.2 | 264.3 | 200 | 45 | 16 |
| GlcCer (d18:1/18:0)-d5 | Internal standard | GlcCer | 733.6 | 269.3 | 7 | 45 | 45 |
| Glucosyl Sphingosine-d5 | Internal standard | GlcCer | 467.2 | 269.3 | 15 | 16 | 30 |
| GalCer (d18:1/16:0) | GlcCer (d18:1(d5)/18:0) | GalCer | 700.6 | 264.3 | 50 | 45 | 45 |
| GalCer (d18:1/18:0) | GlcCer (d18:1(d5)/18:0) | GalCer | 728.6 | 264.3 | 50 | 45 | 45 |
| GalCer (d18:2/18:0) | GlcCer (d18:1(d5)/18:0) | GalCer | 726.6 | 262.3 | 50 | 45 | 45 |
| GalCer (d18:1/20:0) | GlcCer (d18:1(d5)/18:0) | GalCer | 756.6 | 264.3 | 50 | 45 | 50 |
| GalCer (d18:2/20:0) | GlcCer (d18:1(d5)/18:0) | GalCer | 754.6 | 262.3 | 50 | 45 | 50 |
| GalCer (d18:1/22:0) | GlcCer (d18:1(d5)/18:0) | GalCer | 784.6 | 264.3 | 50 | 45 | 50 |
| GalCer (d18:1/22:1) | GlcCer (d18:1(d5)/18:0) | GalCer | 782.6 | 264.3 | 50 | 45 | 50 |
| GalCer (d18:2/22:0) | GlcCer (d18:1(d5)/18:0) | GalCer | 782.6 | 262.3 | 50 | 45 | 50 |
| GalCer (d18:1/24:1) | GlcCer (d18:1(d5)/18:0) | GalCer | 810.7 | 264.3 | 50 | 45 | 50 |
| GalCer (d18:1/24:0) | GlcCer (d18:1(d5)/18:0) | GalCer | 812.7 | 264.3 | 50 | 45 | 50 |
| Gal-Sph 18:1 | Glucosyl Sphingosine-d5 | GalCer | 462.2 | 282.3 | 200 | 45 | 30 |

### Mass Spectrometry Analysis of Eicosanoids

Eicosanoid analyses were performed by liquid chromatography (Shimadzu Nexera X2 system, Shimadzu Scientific Instrument, Columbia, Md., USA) coupled to electrospray mass spectrometry (Sciex QTRAP 6500+, Sciex, Framingham, Mass., USA). For each analysis, 5 μL of sample was injected on a BEH C18 1.7 μm, 2.1×100 mm column (Waters Corporation, Milford, Mass., USA) using a flow rate of 0.6 mL/′min at 40° C. Mobile phases were composed as follows: A=water+0.1% acetic acid, and B=90:10 acetonitrile/isopropyl alcohol (v/v). The gradient was programmed as follows: 0.0-1.0 min at 25% B; 0-8.5 min to 95% B; 8.50-8.51 min at 95% B; 8.51-10.00 min at 25% B. Electrospray ionization was performed in negative ion mode applying the following settings: curtain gas at 30; collision gas was set at medium; ion spray voltage at −4500; temperature at 600° C.; ion source Gas 1 at 50; ion source Gas 2 at 60. Data acquisition was performed using Analyst 1.6.3 (Sciex) in multiple reaction monitoring mode (MRM), with the following parameters: dwell time (msec), collision energy (CE), and declustering potential (DP) for each species reported in Table C; entrance potential (EP) at −10; and collision cell exit potential (CXP) at −12. Eicosanoids were quantified using a mixture of non endogenous, deuterated internal standards as reported in Table C. Eicosanoids were identified based on their retention times and MRM properties of commercially available reference standards (Avanti Polar Lipids, Birmingham, Ala., USA). Quantification was performed using MultiQuant 3.02 (Sciex) and Skyline. Metabolites were normalized to either total protein amount, tissue weight or volume. Protein concentration was measured using the bicinchoninic acid (BCA) assay (Pierce, Rockford, Ill., USA).

TABLE C
Acquisition parameters information for Eicosanoid Assay.

| | | Q1 | Q2 | Time | | |
|---|---|---|---|---|---|---|
| Lipid | Internal Standard | mass | mass | (msec) | DP | CE |
| 12-HETE-d8 | N/A | 327.3 | 184.1 | 20 | −65 | −20 |
| 15-HETE-d8 | N/A | 327.3 | 226.1 | 20 | −65 | −20 |
| 5-HETE-d8 | N/A | 327.3 | 116.1 | 20 | −65 | −20 |
| (d4) PGE2 | N/A | 355.31 | 275.1 | 20 | −35 | −23 |
| PGF2alpha-d4 | N/A | 357.2 | 197.1 | 20 | −60 | −35 |
| 6-k-PGF1alpha-d4 | N/A | 373.2 | 249.1 | 20 | −45 | −35 |
| Arachidonic Acid-d8 | N/A | 311.2 | 267.1 | 10 | −110 | −18 |
| 5-HETE | 5-HETE-d8 | 319.2 | 115.1 | 20 | −50 | −18 |
| 12-HETE | 12-HETE-d8 | 319.2 | 179.1 | 20 | −50 | −18 |
| 15-HETE | 15-HETE-d8 | 319.2 | 219.1 | 20 | −50 | −18 |
| 6k PGF1α | 6-k-PGF1alpha-d4 | 369.2 | 245.1 | 20 | −45 | −34 |
| TxB2 | 15-HETE-d8 | 369.2 | 169.1 | 20 | −50 | −22 |
| PGF2a/8-iso PGF2a | PGF2alpha-d4 | 353.2 | 193.1 | 20 | −50 | −35 |
| PGE2/D2 | (d4) PGE2 | 351.2 | 271.1 | 40 | −30 | −28 |
| Arachidonic Acid | Arachidonic Acid-d8 | 303.2 | 259.1 | 10 | −110 | −18 |
| 5,15-diHETE | 15-HETE-d8 | 335.2 | 115.1 | 20 | −35 | −18 |
| 17 HDoHE | 15-HETE-d8 | 343.2 | 229.1 | 20 | −35 | −15 |
| 5-iso PGF2a | PGF2alpha-d4 | 353.2 | 115.1 | 20 | −60 | −28 |
| PGE2 | (d4) PGE2 | 351.2 | 175.1 | 20 | −30 | −28 |
| DHA | Arachidonic Acid-d8 | 327.2 | 229.1 | 10 | −110 | −17 |
| PGD2 | (d4) PGE2 | 351.2 | 233.2 | 20 | −30 | −28 |
| (d4) PGE2 | N/A | 355.31 | 193.1 | 20 | −35 | −23 |
| LTB4 | 15-HETE-d8 | 335.3 | 195.2 | 20 | −45 | −21 |
| EPA | (d8) Arachidonic Acid | 301.3 | 257.1 | 20 | −110 | −18 |
| LTE4 | 15-HETE-d8 | 438 | 235 | 20 | −60 | −28 |
| 9-HOTrE | 15-HETE-d9 | 293 | 171 | 20 | −45 | −15 |
| 13-HOTrE | 15-HETE-d10 | 293 | 195 | 20 | −45 | −15 |
| 9-oxoODE | 15-HETE-d11 | 293 | 185 | 20 | −70 | −20 |
| 13-oxoODE | 15-HETE-d12 | 293 | 113 | 20 | −70 | −20 |
| 9,10 EpOME | 15-HETE-d13 | 295 | 171.1 | 20 | −70 | −20 |
| 9-HODE | 15-HETE-d14 | 295 | 171 | 20 | −115 | −23 |
| 13-HODE | 15-HETE-d15 | 295 | 195 | 20 | −115 | −23 |
| Leukotriene E4 (LTE4) | 15-HETE-d16 | 438 | 333 | 20 | −80 | −25 |
| 8,15 diHETE | 15-HETE-d8 | 335 | 127 | 20 | −45 | −15 |

### Lipidomics Analysis

Lipid analyses were performed by liquid chromatography (Shimadzu Nexera X2 system, Shimadzu Scientific Instrument, Columbia, Md., USA) coupled to electrospray mass spectrometry (QTRAP 6500+, Sciex, Framingham, Mass., USA). For each analysis, 5 μL of sample was injected on a BEH C18 1.7 μm, 2.1×100 mm column (Waters Corporation, Milford, Mass., USA) using a flow rate of 0.25 ml/min at 55° C. For positive ionization mode, mobile phase A consisted of 60:40 acetonitrile/water (v/v) with 10 mM ammonium formate+0.1% formic acid; mobile phase B consisted of 90:10 isopropyl alcohol/acetonitrile (v/v) with 10 mM ammonium formate+0.1% formic acid. For negative ionization mode, mobile phase A consisted of 60:40 acetonitrile/water (v/v) with 10 mM ammonium acetate; mobile phase 13 consisted of 90:10 isopropyl alcohol/acetonitrile (v/v) with 10 μM ammonium acetate. The gradient was programmed as follows: 0.0-8.0 min from 45% B to 99% B, 8.0-9.0 min at 99% B, 9.0-9.1 min to 45% B, and 9.1-10.0 min at 45% B. Electrospray ionization was performed in either positive or negative ion mode applying the following settings: curtain gas at 30; collision gas was set at medium; ion spray voltage at 5500 (positive mode) or 4500 (negative mode); temperature at 250° C. (positive mode) or 600° C. (negative mode); ion source Gas 1 at 50; ion source Gas 2 at 60. Data acquisition was performed using Analyst 1.6.3 (Sciex) in multiple reaction monitoring mode (MRM), with the following parameters: dwell time (msec) and collision energy (CE) for each species reported in Table D (negative mode) or Table E (positive mode); declustering potential (DP) at 80 (positive mode) and at −80 (negative mode); entrance potential (EP) at 10 (positive mode) or −10 (negative mode); and collision cell exit potential (CM)) at 12.5 (positive mode) or −12.5 (negative mode). Lipids were quantified using a mixture of non-endogenous internal standards as reported in Tables D and E. Lipids were identified based on their retention times and MRM properties of commercially available reference standards (Avanti Polar Lipids, Birmingham, Ala., USA). Quantification was performed using MultiQuant 3.02 (Sciex). Metabolites were normalized to either total protein amount or cell number.

TABLE D
Acquisition parameters information for Lipidomics Assay in negative mode.

| | | Q1 | Q2 | Time | |
|---|---|---|---|---|---|
| Lipid | Internal Standard | mass | mass | (msec) | CE |
| PA(15:0/18:1(d7)) | N/A | 666.52 | 241.3 | 5 | −50 |
| PA(16:0_18:1) | PA(15:0/18:1(d7)) | 673.5 | 255.3 | 5 | −50 |
| PA(18:0_18:1) | PA(15:0/18:1(d7)) | 701.5 | 283.3 | 5 | −50 |
| PA(18:1_18:1) | PA(15:0/18:1(d7)) | 699.5 | 281.3 | 5 | −50 |
| PA(18:0_20:4) | PA(15:0/18:1(d7)) | 723.5 | 283.3 | 5 | −50 |
| PA(18:1_22:6) | PA(15:0/18:1(d7)) | 745.5 | 281.3 | 5 | −50 |
| PA(18:0_22:6) | PA(15:0/18:1(d7)) | 747.5 | 283.3 | 5 | −50 |
| PE(15:0/18:1(d7)) | N/A | 709.56 | 241.3 | 5 | −50 |
| PE(P-18:0/18:1) | PE(15:0/18:1(d7)) | 728.6 | 283.3 | 5 | −50 |
| PE(P-18:0/18:2) | PE(15:0/18:1(d7)) | 726.6 | 281.3 | 5 | −50 |
| PE(P-16:0/20:4) | PE(15:0/18:1(d7)) | 722.6 | 303.3 | 5 | −50 |
| PE(P-18:0/20:4) | PE(15:0/18:1(d7)) | 750.6 | 303.3 | 5 | −50 |
| PE(P-16:0/22:6) | PE(15:0/18:1(d7)) | 746.6 | 327.3 | 5 | −50 |
| PE(P-18:0/22:6) | PE(15:0/18:1(d7)) | 774.6 | 327.3 | 5 | −50 |
| (3-O-sulfo)Gal- | N/A | 722.5 | 97 | 8 | −150 |
| Cer(d18:1/12:0) | | | | | |
| (3-O-sulfo)Gal- | (3-O-sulfo)Gal- | 778.5 | 97 | 8 | −150 |
| Cer(d18:1/16:0) | Cer(d18:1/12:0) | | | | |
| (3-O-sulfo)Gal- | (3-O-sulfo)Gal- | 806.6 | 97 | 8 | −150 |
| Cer(d18:1/18:0) | Cer(d18:1/12:0) | | | | |
| (3-O-sulfo)Gal- | (3-O-sulfo)Gal- | 822.6 | 97 | 8 | −150 |
| Cer(d18:1/18:0(2OH) | Cer(d18:1/12:0) | | | | |
| (3-O-sulfo)Gal- | (3-O-sulfo)Gal- | 890.7 | 97 | 8 | −150 |
| Cer(d18:1/24:0) | Cer(d18:1/12:0) | | | | |
| (3-O-sulfo)Gal- | (3-O-sulfo)Gal- | 906.7 | 97 | 8 | −150 |
| Cer(d18:1/24:0(2OH)) | Cer(d18:1/12:0) | | | | |
| (3-O-sulfo)Gal- | (3-O-sulfo)Gal- | 888.7 | 97 | 8 | −150 |
| Cer(d18:1/24:1) | Cer(d18:1/12:0) | | | | |
| (3-O-sulfo)Gal- | (3-O-sulfo)Gal- | 904.7 | 97 | 8 | −150 |
| Cer(d18:1/24:1(2OH)) | Cer(d18:1/12:0) | | | | |
| LPE(P-16:0) | LPE(18:1(d7)) | 436.3 | 196.1 | 5 | −50 |
| LPE(P-18:0) | LPE(18:1(d7)) | 464.3 | 196.1 | 5 | −50 |
| LPE(P-18:1) | LPE(18:1(d7)) | 462.3 | 196.1 | 5 | −50 |
| LPE(18:1(d7)) | N/A | 485.3 | 288.3 | 5 | −50 |
| LPE(16:0) | LPE(18:1(d7)) | 452.278 | 255.3 | 5 | −50 |
| LPE(18:0) | LPE(18:1(d7)) | 480.31 | 283.3 | 5 | −50 |
| LPE(18:1) | LPE(18:1(d7)) | 478.3 | 281.3 | 5 | −50 |
| LPI(16:0) | LPE(18:1(d7)) | 571.3 | 241.1 | 5 | −50 |
| LPI(18:0) | LPE(18:1(d7)) | 599.3 | 241.1 | 5 | −50 |
| LPI(20:4) | LPE(18:1(d7)) | 619.3 | 241.1 | 5 | −50 |
| LPG(16:0) | LPE(18:1(d7)) | 483.3 | 255.3 | 5 | −50 |
| LPG(18:0) | LPE(18:1(d7)) | 511.3 | 283.3 | 5 | −50 |
| LPG(18:1) | LPE(18:1(d7)) | 509.3 | 281.3 | 5 | −50 |
| LPG(20:4) | LPE(18:1(d7)) | 531.3 | 303.3 | 5 | −50 |
| CL(14:0/14:0/14:0/14:0) | N/A | 555.3 | 327.3 | 5 | −50 |
| CL(72:8) | CL(14:0/14:0/14:0/14:0) | 619.5 | 227.2 | 5 | −50 |
| Cholesterol Sulfate | N/A | 465.3 | 96.7 | 5 | −80 |
| PG(15:0/18:1(d7)) | N/A | 740.55 | 241.3 | 5 | −50 |
| PG(16:0_18:1) | PG(15:0/18:1(d7)) | 747.5 | 255.3 | 5 | −50 |
| PG(18:0_18:1) | PG(15:0/18:1(d7)) | 775.5 | 283.3 | 5 | −50 |
| PG(18:1/18:1) | PG(15:0/18:1(d7)) | 773.5 | 281.3 | 5 | −50 |
| PG(18:0_20:4) | PG(15:0/18:1(d7)) | 797.6 | 283.3 | 5 | −50 |
| PI(15:0/18:1(d7)) | N/A | 828.6 | 241.3 | 5 | −50 |
| PI(18:0_18:1) | PI(15:0/18:1(d7)) | 863.6 | 283.3 | 5 | −50 |
| PI(18:1/18:1) | PI(15:0/18:1(d7)) | 861.6 | 281.3 | 5 | −50 |
| PI(16:0_20:4) | PI(15:0/18:1(d7)) | 857.6 | 255.3 | 5 | −50 |
| PI(18:0_20:4) | PI(15:0/18:1(d7)) | 885.6 | 283.3 | 5 | −50 |
| PI(16:0_22:6) | PI(15:0/18:1(d7)) | 881.6 | 255.3 | 5 | −50 |
| PI(18:0_22:6) | PI(15:0/18:1(d7)) | 909.6 | 283.3 | 5 | −50 |
| PI(20:4/20:4) | PI(15:0/18:1(d7)) | 905.6 | 303.3 | 5 | −50 |
| PS(15:0/18:1(d7)) | N/A | 753.55 | 241.3 | 5 | −50 |
| PS(18:0_18:1) | PS(15:0/18:1(d7)) | 788.6 | 283.3 | 5 | −50 |
| PS(18:0_20:4) | PS(15:0/18:1(d7)) | 810.6 | 283.3 | 5 | −50 |
| PS(16:0_22:6) | PS(15:0/18:1(d7)) | 806.6 | 255.3 | 5 | −50 |
| PS(18:1_22:6) | PS(15:0/18:1(d7)) | 832.6 | 281.3 | 5 | −50 |
| PS(18:0_22:6) | PS(15:0/18:1(d7)) | 834.6 | 283.3 | 5 | −50 |
| PS(22:6/22:6) | PS(15:0/18:1(d7)) | 878.5 | 327.3 | 5 | −50 |
| Arachidonic acid(d8) | N/A | 311.3 | 311.3 | 5 | −10 |
| Palmitic acid | Arachidonic acid(d8) | 255.1 | 255.1 | 5 | −10 |
| Palmitoleic acid | Arachidonic acid(d8) | 253.1 | 253.1 | 5 | −10 |
| Stearic acid | Arachidonic acid(d8) | 283.2 | 283.2 | 5 | −10 |
| Oleic acid | Arachidonic acid(d8) | 281.2 | 281.2 | 5 | −10 |
| Linoleic acid | Arachidonic acid(d8) | 279.2 | 279.2 | 5 | −10 |
| Linolenic acid | Arachidonic acid(d8) | 277.2 | 277.2 | 5 | −10 |
| Arachidonic acid | Arachidonic acid(d8) | 303.2 | 303.2 | 5 | −10 |
| EPA | Arachidonic acid(d8) | 301.2 | 301.2 | 5 | −10 |
| DHA | Arachidonic acid(d8) | 327.2 | 327.2 | 5 | −10 |

TABLE E
Acquisition parameters information for Lipidomics Assay in positive mode.

| | | Q1 | Q3 | Time | |
|---|---|---|---|---|---|
| Lipid | Internal Standard | mass | mass | (msec) | CE |
| Sphingosine(d17:1) | N/A | 286.2 | 268.3 | 5 | 20 |
| Sphingosine | Sphingosine(d17:1) | 300.2 | 282.2 | 5 | 20 |
| Sphinganine | Sphingosine(d17:1) | 302.2 | 284.2 | 5 | 20 |
| Glucosyl | N/A | 467.2 | 269.3 | 5 | 16 |
| sphingosine(d5) | | | | | |
| Hexosyl sphingosine | Glucosyl sphingosine | 462.3 | 282.2 | 5 | 16 |
| | (d5) | | | | |
| Lactosyl sphingosine | Glucosyl sphingosine | 624.4 | 282.3 | 5 | 16 |
| | (d5) | | | | |
| Cer(d18:1/17:0) | N/A | 552.4 | 264.3 | 5 | 40 |
| Cer(d18:1/16:0) | Cer(d18:1/17:0) | 538.5 | 264.4 | 5 | 40 |
| Cer(d18:1/18:0) | Cer(d18:1/17:0) | 566.6 | 264.4 | 5 | 40 |
| Cer(d18:1/24:0) | Cer(d18:1/17:0) | 650.6 | 264.4 | 5 | 40 |
| Cer(d18:1/24:1) | Cer(d18:1/17:0) | 648.6 | 264.4 | 5 | 40 |
| SM(d18:1(d9)/18:1) | N/A | 738.7 | 184.1 | 5 | 40 |
| SM(d18:1/16:0) | SM(d18:1(d9)/18:1) | 703.6 | 184.1 | 5 | 40 |
| SM(d18:1/18:0) | SM(d18:1(d9)/18:1) | 731.6 | 184.1 | 5 | 40 |
| SM(d18:1/24:0) | SM(d18:1(d9)/18:1) | 815.7 | 184.1 | 5 | 40 |
| SM(d18:1/24:1) | SM(d18:1(d9)/18:1) | 813.7 | 184.1 | 5 | 40 |
| HexCer(d18:1/12:0) | N/A | 644.5 | 264.3 | 5 | 40 |
| HexCer(d18:1/16:0) | HexCer(d18:1/12:0) | 700.6 | 264.6 | 5 | 40 |
| HexCer(d18:1/18:0) | HexCer(d18:1/12:0) | 728.6 | 264.4 | 5 | 40 |
| HexCer(d18:1/24:0) | HexCer(d18:1/12:0) | 812.7 | 264.4 | 5 | 40 |
| HexCer(d18:1/24:1) | HexCer(d18:1/12:0) | 810.7 | 264.4 | 5 | 40 |
| LacCer(d18:1/16:0) | HexCer(d18:1/12:0) | 862.6 | 264.6 | 5 | 40 |
| LacCer(d18:1/18:0) | HexCer(d18:1/12:0) | 890.7 | 264.4 | 5 | 40 |
| LacCer(d18:1/24:0) | HexCer(d18:1/12:0) | 974.8 | 264.4 | 5 | 40 |
| LacCer(d18:1/24:1) | HexCer(d18:1/12:0) | 972.7 | 264.4 | 5 | 40 |
| LPC(18:1(d7)) | N/A | 529.3 | 184.1 | 5 | 40 |
| LPC(16:1) | LPC(18:1(d7)) | 494.5 | 184.1 | 5 | 40 |
| LPC(16:0) | LPC(18:1(d7)) | 496.3 | 184.1 | 5 | 40 |
| LPC(18:0) | LPC(18:1(d7)) | 524.3 | 184.1 | 5 | 40 |
| LPC(18:1) | LPC(18:1(d7)) | 522.3 | 184.1 | 5 | 40 |
| LPC(20:4) | LPC(18:1(d7)) | 544.3 | 184.1 | 5 | 40 |
| LPC(22:6) | LPC(18:1(d7)) | 568.3 | 184.1 | 5 | 40 |
| LPC(24:1) | LPC(18:1(d7)) | 606.5 | 184.1 | 5 | 40 |
| LPC(24:0) | LPC(18:1(d7)) | 608.5 | 184.1 | 5 | 40 |
| LPC(26:1) | LPC(18:1(d7)) | 634.5 | 104.1 | 5 | 40 |
| LPC(26:0) | LPC(18:1(d7)) | 636.5 | 104.1 | 5 | 40 |
| LSM | LPC(18:1(d7)) | 465.5 | 184.1 | 5 | 40 |
| PC(15:0/18:1(d7)) | N/A | 754.6 | 184.1 | 5 | 40 |
| PC(36:1) | PC(15:0/18:1(d7)) | 788.6 | 184.1 | 5 | 40 |
| PC(36:2) | PC(15:0/18:1(d7)) | 786.6 | 184.1 | 5 | 40 |
| PC(36:4) | PC(15:0/18:1(d7)) | 782.6 | 184.1 | 5 | 40 |
| PC(38:4) | PC(15:0/18:1(d7)) | 810.6 | 184.1 | 5 | 40 |
| PC(38:6) | PC(15:0/18:1(d7)) | 806.6 | 184.1 | 5 | 40 |
| PC(40:6) | PC(15:0/18:1(d7)) | 834.6 | 184.1 | 5 | 40 |
| PC(O-18:0/2:0) | LPC(18:1(d7)) | 524.3 | 184.1 | 5 | 40 |
| PE(15:0/18:1(d7)) | N/A | 711.6 | 570.5 | 5 | 40 |
| PE(36:1) | PE(15:0/18:1(d7)) | 746.6 | 605.5 | 5 | 40 |
| PE(36:2) | PE(15:0/18:1(d7)) | 744.6 | 603.5 | 5 | 40 |
| PE(36:4) | PE(15:0/18:1(d7)) | 740.6 | 599.5 | 5 | 40 |
| PE(38:4) | PE(15:0/18:1(d7)) | 768.6 | 627.5 | 5 | 40 |
| PE(38:6) | PE(15:0/18:1(d7)) | 764.6 | 623.5 | 5 | 40 |
| PE(40:6) | PE(15:0/18:1(d7)) | 792.6 | 651.5 | 5 | 40 |
| Cholesterol(d7) | N/A | 376.2 | 376.2 | 5 | 10 |
| Cholesterol | Cholesterol(d7) | 369.3 | 369.3 | 5 | 10 |
| CE(18:1(d7)) | N/A | 675.2 | 369.4 | 5 | 26 |
| CE(16:1) | CE(18:1(d7)) | 640.6 | 369.3 | 5 | 26 |
| CE(18:1) | CE(18:1(d7)) | 668.6 | 369.3 | 5 | 26 |
| CE(18:2) | CE(18:1(d7)) | 666.6 | 369.3 | 5 | 26 |
| CE(20:4) | CE(18:1(d7)) | 690.6 | 369.3 | 5 | 26 |
| CE(20:5) | CE(18:1(d7)) | 688.6 | 369.3 | 5 | 26 |
| CE(22:6) | CE(18:1(d7)) | 714.6 | 369.3 | 5 | 26 |
| TG(15:0/18:1(d7)/15:0) | N/A | 829.4 | 523.5 | 5 | 40 |
| TG 52:4/18:1 | TG(15:0/18:1(d7)/15:0) | 872.7 | 573.4 | 5 | 40 |
| TG 52:3/18:1 | TG(15:0/18:1(d7)/15:0) | 874.7 | 575.4 | 5 | 40 |
| TG 54:2/18:0 | TG(15:0/18:1(d7)/15:0) | 904.7 | 603.4 | 5 | 40 |
| TG(52:5/20:4) | TG(15:0/18:1(d7)/15:0) | 870.6 | 549.3 | 5 | 40 |
| TG 54:6/20:4 | TG(15:0/18:1(d7)/15:0) | 896.6 | 575.3 | 5 | 40 |
| TG 54:7/20:4 | TG(15:0/18:1(d7)/15:0) | 894.6 | 573.3 | 5 | 40 |
| TG 56:4/20:4 | TG(15:0/18:1(d7)/15:0) | 928.8 | 607.5 | 5 | 40 |
| TG 56:6/20:4 | TG(15:0/18:1(d7)/15:0) | 924.7 | 603.4 | 5 | 40 |
| TG 56:7/20:4 | TG(15:0/18:1(d7)/15:0) | 922.7 | 601.4 | 5 | 40 |
| TG(58:5/20:4) | TG(15:0/18:1(d7)/15:0) | 954.7 | 633.4 | 5 | 40 |
| TG(58:7/20:4) | TG(15:0/18:1(d7)/15:0) | 950.7 | 629.4 | 5 | 40 |
| TG 58:8/22:6 | TG(15:0/18:1(d7)/15:0) | 948.7 | 603.4 | 5 | 40 |
| TG 60:7/22:6 | TG(15:0/18:1(d7)/15:0) | 978.7 | 633.4 | 5 | 40 |
| TG 60:8/22:6 | TG(15:0/18:1(d7)/15:0) | 976.7 | 631.4 | 5 | 40 |
| Sphingosine-1- | N/A | 366.3 | 250.3 | 5 | 25 |
| phosphate(d17:1) | | | | | |
| Sphingosine-1- | Sphingosine(d17:1) | 380.3 | 264.3 | 5 | 25 |
| phosphate | | | | | |
| Sphinganine-1- | Sphingosine(d17:1) | 382.3 | 266.3 | 5 | 18 |
| phosphate | | | | | |
| GB3(d18:1/16:0) | HexCer(d18:1/12:0) | 1025 | 520.5 | 5 | 40 |
| GB3(d18:1/18:0) | HexCer(d18:1/12:0) | 1053 | 548.6 | 5 | 40 |
| GB3(d18:1/24:0) | HexCer(d18:1/12:0) | 1137 | 632.6 | 5 | 40 |
| GB3(d18:1/24:1) | HexCer(d18:1/12:0) | 1135 | 630.6 | 5 | 40 |
| GlcCer(d18:1/18:0(d5)) | N/A | 733.6 | 269.3 | 5 | 45 |
| Cer(d18:1/16:0(d7)) | N/A | 545.5 | 271.4 | 5 | 40 |
| LPC(26:0) | LPC(18:1(d7)) | 636.5 | 104.1 | 5 | 40 |
| LPC(24:0) | LPC(18:1(d7)) | 608.5 | 184.1 | 5 | 40 |
| LPC(26:1) | LPC(18:1(d7)) | 634.5 | 104.1 | 5 | 40 |
| LPC(24:1) | LPC(18:1(d7)) | 606.5 | 184.1 | 5 | 40 |
| LPC(16:1) | LPC(18:1(d7)) | 494.5 | 184.1 | 5 | 40 |
| Cer(d18:0/16:0) | Cer(d18:1/17:0) | 540.6 | 522.3 | 5 | 40 |
| Cer(d18:0/18:0) | Cer(d18:1/17:0) | 568.7 | 550.4 | 5 | 40 |
| Cer(d18:0/24:0) | Cer(d18:1/17:0) | 652.9 | 634.4 | 5 | 40 |
| Cer(d18:0/24:1) | Cer(d18:1/17:0) | 650.9 | 632.4 | 5 | 40 |
| Glc-Cholesterol | CE(18:1(d7)) | 566.6 | 369.3 | 5 | 17 |
| Glc-Sitosterol | CE(18:1(d7)) | 594.6 | 397.4 | 5 | 17 |
| 7-HOCA | CE(18:1(d7)) | 431.3 | 367.3 | 5 | 25 |
| 24-hydroxy- | N/A | 392.4 | 374.4 | 5 | 15 |
| cholesterol(d7) | | | | | |
| hydroxy-cholesterol | CE(18:1(d7)) | 385.3 | 367.3 | 5 | 30 |
| DG(16:0_18:1) | DG(15:0/18:1(d7)) | 614.4 | 313.4 | 5 | 30 |
| DG(18:0_18:1) | DG(15:0/18:1(d7)) | 640.4 | 341.3 | 5 | 30 |
| DG(18:1/18:1) | DG(15:0/18:1(d7)) | 638.4 | 339.3 | 5 | 30 |
| DG(16:0_20:4) | DG(15:0/18:1(d7)) | 634.5 | 313.3 | 5 | 30 |
| DG(18:0_20:4) | DG(15:0/18:1(d7)) | 662.5 | 341.3 | 5 | 30 |
| DG(18:0_22:6) | DG(15:0/18:1(d7)) | 686.6 | 341.3 | 5 | 30 |
| DG(18:1_20:4) | DG(15:0/18:1(d7)) | 660.5 | 339.3 | 5 | 30 |
| DG(15:0/18:1(d7)) | N/A | 605.6 | 346.5 | 5 | 30 |
| MG(18:1(d7)) | N/A | 381.3 | 272.5 | 5 | 22 |
| MG(16:0) | MG(18:1(d7)) | 348.3 | 239.3 | 5 | 22 |
| MG(20:4) | MG(18:1(d7)) | 396.3 | 287.3 | 5 | 22 |
| MG(18:0) | MG(18:1(d7)) | 376.3 | 267.3 | 5 | 22 |
| MG(18:1) | MG(18:1(d7)) | 374.3 | 265.3 | 5 | 22 |
| MG(22:6) | MG(18:1(d7)) | 403.3 | 311.3 | 5 | 22 |
| MG(16:1) | MG(18:1(d7)) | 346.3 | 237.3 | 5 | 22 |
| 7-keto-cholesterol | CE(18:1(d7)) | 401.3 | 383.3 | 5 | 15 |
| 4-beta- | CE(18:1(d7)) | 420.3 | 385.3 | 5 | 15 |
| hydroxycholesterol | | | | | |
| CE oxoODE | CE(18:1(d7)) | 680.6 | 369.2 | 5 | 25 |
| CE HODE | CE(18:1(d7)) | 682.6 | 369.2 | 5 | 25 |
| CE HpODE | CE(18:1(d7)) | 698.6 | 369.2 | 5 | 25 |
| Glc Sphingosine(d5) | N/A | 467.2 | 269.3 | 5 | 16 |
| PC(15:0/18:1(d7)) | N/A | 754.6 | 184.1 | 5 | 40 |
| PC(36:1) | PC(15:0/18:1(d7)) | 788.6 | 184.1 | 5 | 40 |
| PC(36:2) | PC(15:0/18:1(d7)) | 786.6 | 184.1 | 5 | 40 |
| PC(36:4) | PC(15:0/18:1(d7)) | 782.6 | 184.1 | 5 | 40 |
| PC(38:4) | PC(15:0/18:1(d7)) | 810.6 | 184.1 | 5 | 40 |
| PC(38:6) | PC(15:0/18:1(d7)) | 806.6 | 184.1 | 5 | 40 |
| PC(40:4) | PC(15:0/18:1(d7)) | 838.6 | 184.1 | 5 | 40 |
| PC(40:6) | PC(15:0/18:1(d7)) | 834.6 | 184.1 | 5 | 40 |
| PC(O-18:0/2:0) | LPC(18:1(d7)) | 524.3 | 184.1 | 5 | 40 |
| PE(15:0/18:1(d7)) | N/A | 711.6 | 570.5 | 5 | 40 |
| PE(36:1) | PE(15:0/18:1(d7)) | 746.6 | 605.5 | 5 | 40 |
| PE(36:2) | PE(15:0/18:1(d7)) | 744.6 | 603.5 | 5 | 40 |
| PE(36:4) | PE(15:0/18:1(d7)) | 740.6 | 599.5 | 5 | 40 |
| PE(38:4) | PE(15:0/18:1(d7)) | 768.6 | 627.5 | 5 | 40 |
| PE(38:6) | PE(15:0/18:1(d7)) | 764.6 | 623.5 | 5 | 40 |
| PE(40:4) | PE(15:0/18:1(d7)) | 796.6 | 655.5 | 5 | 40 |
| PE(40:6) | PE(15:0/18:1(d7)) | 792.6 | 651.5 | 5 | 40 |
| POV-PC | PC(15:0/18:1(d7)) | 594.5 | 184.1 | 5 | 40 |
| PC(16:0/9:0(CHO)) | PC(15:0/18:1(d7)) | 650.4 | 184.1 | 5 | 40 |
| PC(16:0/9:0(COOH)) | PC(15:0/18:1(d7)) | 666.4 | 184.1 | 5 | 40 |
| LysoPAF(16:0) | PC(15:0/18:1(d7)) | 482.3 | 184.1 | 5 | 40 |
| PC(O-16:0/2:0) | PC(15:0/18:1(d7)) | 524.3 | 184.1 | 5 | 40 |
| PC(18:0/20:4(OH[S])) | PC(15:0/18:1(d7)) | 826.6 | 184.1 | 5 | 40 |
| PC(18:0/20:4(OOH[S])) | PC(15:0/18:1(d7)) | 842.6 | 184.1 | 5 | 40 |
| PE(18:0/20:4(OH[S])) | PE(15:0/18:1(d7)) | 784.5 | 643.4 | 5 | 40 |
| PE(18:0/20:4(OOH[S])) | PE(15:0/18:1(d7)) | 800.5 | 659.4 | 5 | 40 |
| Coenzyme Q10 | TG(15:0/18:1(d7)/15:0) | 863.3 | 197.2 | 5 | 35 |

Homogenization of ETV:IDS brain tissue and Trem2 analysis of ETV:IDS brain tissue and CSF

50 mg tissue was homogenized in 500 μL 1×CST buffer (Cell Signaling Technology 9803S) made with complete Protease Inhibitor (Roche #04693132001) and PhosStop (Roche 04906837001) using the Qiagen TissueLyzer II (Cat No./ID: 85300) for 2 rounds of 3 minutes at 30 Hz. Homogenate was incubated on ice for 20 minutes and spun at 21,100 g for 30 minutes at 4° C. Subsequent lysate was transferred to a clean 96-well deep plate, and a BCA was performed to quantify total protein amounts. Samples were then stored at −80° C. until assay use.

For Trem2 analysis in brain tissue and soluble Trem2 (sTrem2) analysis in CSF, an MSD GOLD 96w small spot streptavidin plate (MSD L45SA) was prepared for Trem2 assay by coating with 1 ug/mL biotinylated sheep anti-mouse antibody (R&D Systems BAF1729) overnight at 4° C. The next day, the MSD plate was rinsed with tris buffered saline with triton (TBST) and blocked for two hours using 3% bovine serum albumin in TBST, while shaking at 600 rpm. The MSD plate was again rinsed again with TBST, and brain lysates were diluted 5× in blocking solution and added to the MSD plate to incubate for 1 hour at 600 rpm. Following the next TBST rinse, sulfotagged sheep anti-mouse antibody (R&D Systems AF1729) was added to the plate and incubated for 1 hour, again at 600 rpm, and a final rinse was conducted before adding 2× MSD read buffer diluted in water. The plate was then read using the MSD Meso Sector S600. The Trem2 signal was normalized to the protein concentration and plotted with GraphPad Prism.

### ABBREVIATIONS

BMP=bis(monoacylglycerol)phosphate; ETV:IDS=enzyme transport vehicle: iduronate 2-sulfatase; GlcCer=glucosylceramide; GalCer=galactosyl ceramide; IDS=iduronate 2-sulfatase; KI=knock-in; KO=knockout; TfR^{mu/hu}=chimeric human/mouse transferrin receptor.

### Results

To determine whether the robust GAG reduction observed in brain translated to correction of downstream disease-relevant pathology, the ability of ETV:IDS to correct secondary lysosomal storage was assessed. Four weekly activity-equivalent doses of ETV:IDS or idursulfase were intravenously administered to IDS KO; TfR^{mu/hu} KI mice, and the levels of a panel of lysosomal lipids, including gangliosides, glucosylceramide, and bis(monoacylglycerol)phosphate, were measured using liquid chromatography-tandem mass spectrometry (LCMS). Significant accumulation of lysosomal lipids was observed in the brains of IDS KO; TfR^{mu/hu} KI mice compared with wild-type controls. Following 4 weekly doses of 40 mg/kg, ETV:IDS was highly effective in lowering lysosomal lipids in the brain, completely reducing levels of these lipids to that seen in wild-type mice. Treatment with an activity-equivalent dose of idursulfase, however, failed to reduce levels of these lysosomal lipids in the brain. Together, these data demonstrate that ETV:IDS effectively corrects secondary lysosomal storage in addition to its proximal effects on GAG accumulation.

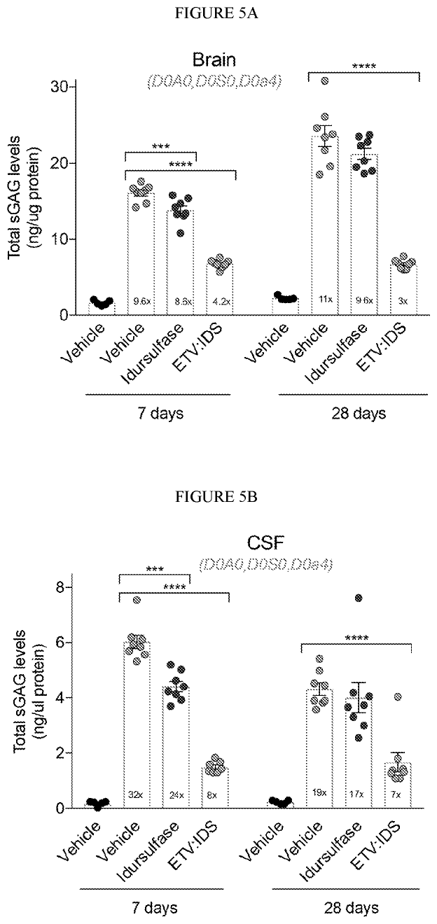

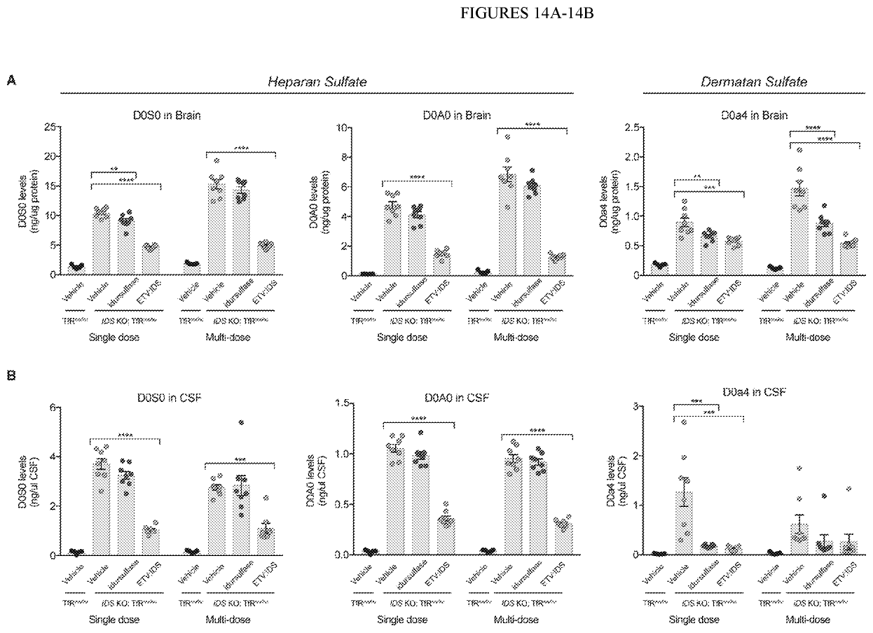

In particular FIGS. 5A-5B show peripheral administration of ETV:IDS (4 week treatment) corrects (FIG. 5A) brain and (FIG. 5B) CSF GAG accumulation in IDS KO mice. A 58% reduction in brain GAG was observed with peripheral administration of ETV:IDS compared to a 14% reduction with an activity equivalent dose of Elaprase. Additionally, a 66% reduction in CSF GAG was observed with peripheral administration of ETV:IDS compared to a 27% reduction with an activity equivalent dose of Elaprase (one outlier omitted for ETV:IDS group). FIGS. 14A-14B illustrate the reduction of individual GAG species (D00S0, D00A0, D00a4) in the brain and CSF. In a follow-up study, CSF GAGs were reduced three times over WT at a dosing of 40 mg/kg of ETV:IDS.

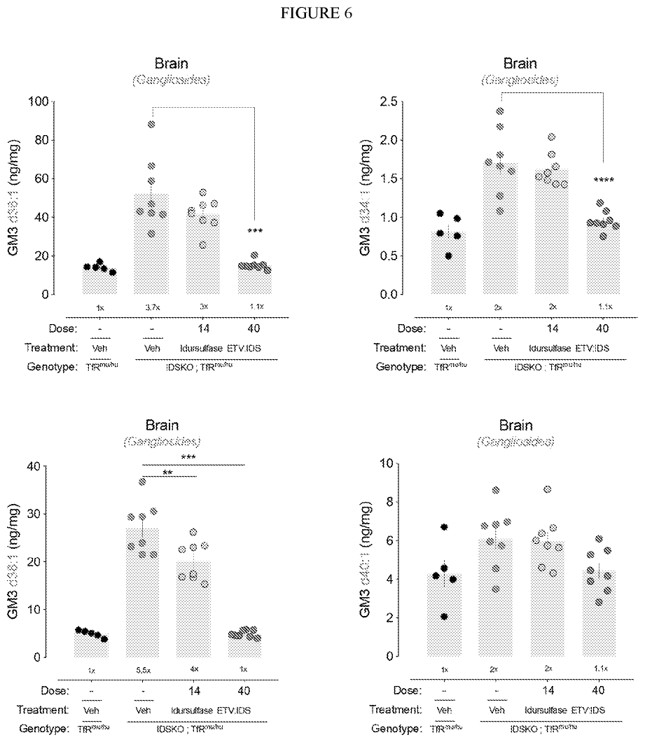

FIG. 6 shows peripheral administration of ETV:IDS (4 week treatment) corrected lysosomal lipid (gangliosides) accumulation in IDS KO brains.

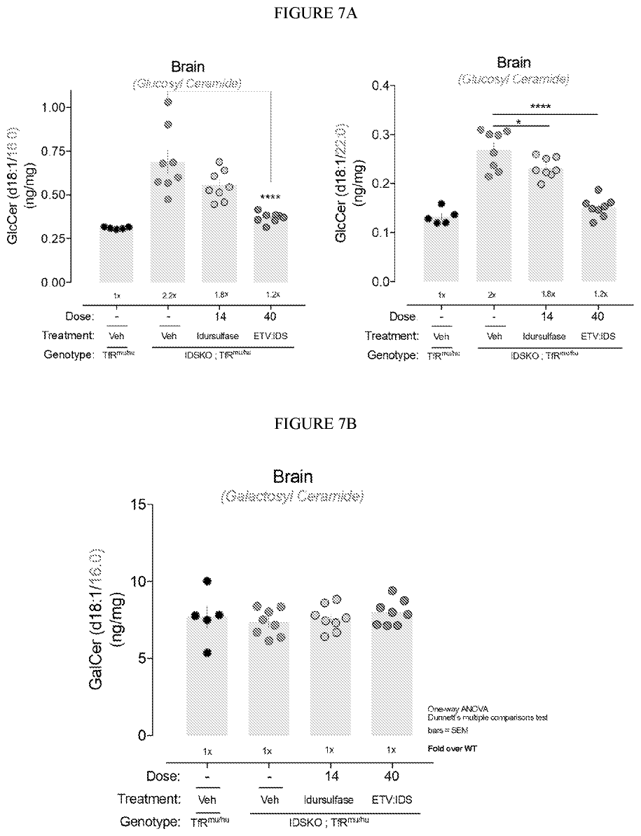

FIGS. 7A-7B show that peripheral administration of ETV:IDS (4 week treatment) corrected lysosomal lipid (GlcCer) accumulation in IDS KO brains. Correction of brain GlcCer accumulation with peripheral administration of ETV:IDS was observed, while no change was observed in galactosyl ceramides (GalCer).

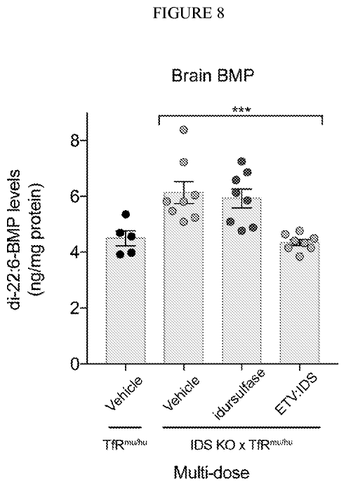

FIG. 8 shows that peripheral administration of ETV:IDS (4 week treatment) corrected lysosomal lipid (BMP) accumulation in IDS KO brains.

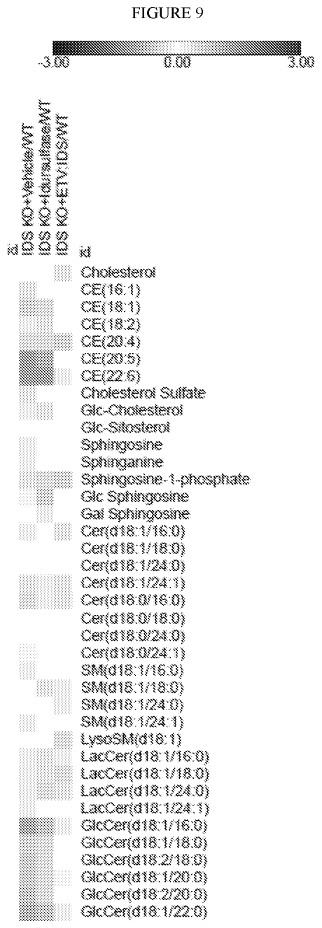

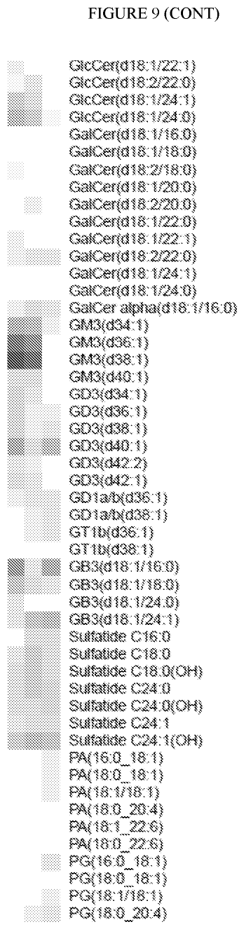

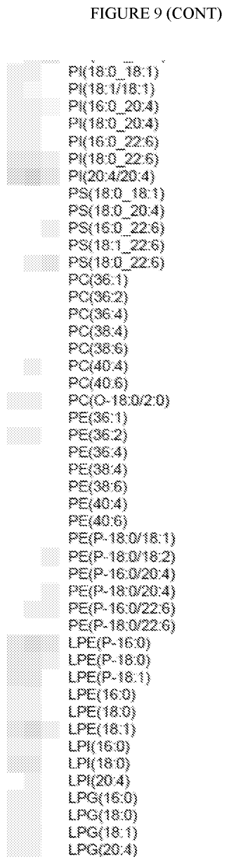

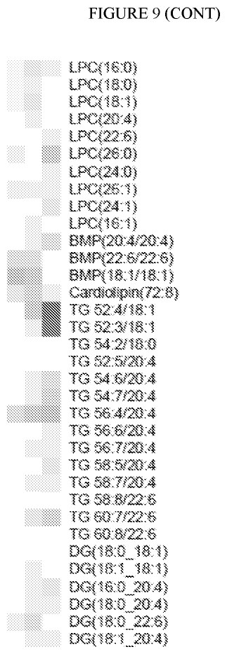

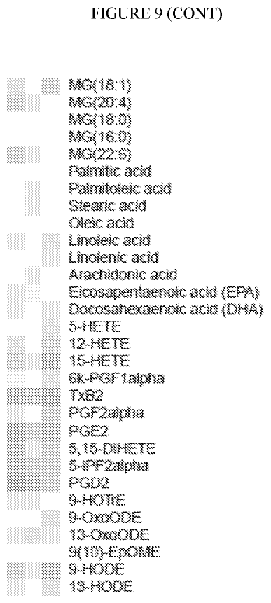

FIG. 9 shows a heat map illustrating lipid levels in brains of IDS KO mice treated with vehicle, ETV:IDS or Elaprase (idursulfase). This heat map was generated using modified data from Table 2. Specifically, the data in Table 2 was modified with a cutoff, converting values between 0.9 and 1.1 to 1, in order to show differences greater than 10%.

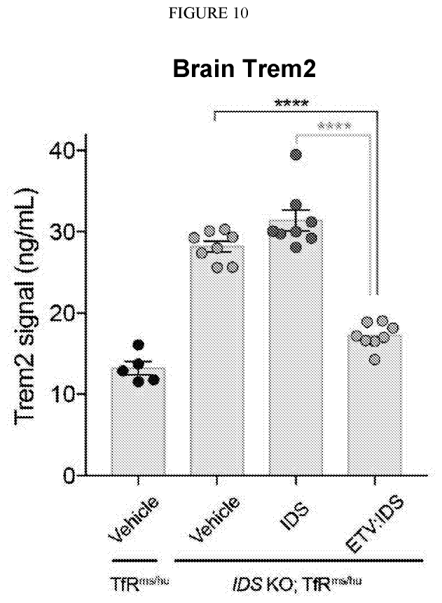

FIG. 10 shows that peripheral administration of ETV:IDS corrected TREM2 accumulation in IDS KO brains.

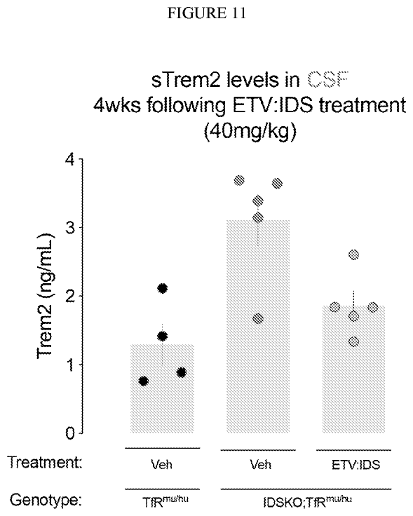

FIG. 11 illustrates that an increase in CSF sTrem2 levels was observed in IDS KO; TfR^{mu/hu} KI mice relative to a TfR^{mu/hu} KI mice cohort. Treatment with ETV:IDS reduced the accumulation of CSF sTrem2 levels in the IDS KO; TfR^{mu/hu} KI mice.

Table 1 provides a summary of the GlcCer species analyzed, with the levels represented as fold over WT.

Table 2 provides data for lipid levels in brains of IDS KO mice treated with vehicle, ETV:IDS or Elaprase (fold changes over WT).

### Example 2. Measurement of Brain GAG, Lysosomal Lipids, and Neurofilament Light Chain (Nf-L) in IDS KO Mice at 3, 6 and 9 Months of Age

As described below, brain GAG, lysosomal lipid, and neurofilament light chain (Nf-L) levels were investigated in WT and IDS KO mice at 3, 6 and 9 months of age.

### Materials and Methods

Animals used in this study were cared for as described herein (Example 1). Tissues were sampled, and lipid and GAG levels were measured using methods described herein (Example 1). Nf-L levels were measured as described below.

### Methods for CSF and Serum Analysis of Nf-L

Using Quanterix Simoa Neurofilament-Light (NF-L) Sample Diluent (Quanterix 102252), cerebrospinal fluid (CSF) was diluted 100× and serum was diluted 4× before being added to Simoa 96-well microplate (Quanterix 101457). NF-light assay was carried out according to Simoa NF-Light Advantage Kit (Quanterix 1031086) instructions using Simoa detector reagent and bead reagent (Quanterix 103159 and 102246, respectively). After incubation of samples with detector and bead reagent at 30° C., 800 rpm for 30 minutes, the sample plate was washed with Simoa Wash Buffer A (Quanterix 103078) on Simoa Microplate Washer according to Quanterix two step protocol. SBG reagent (Quanterix 102250) was subsequently added, and samples were incubated at 30° C., 800 rpm for 10 minutes. The 2-step washer protocol was continued, with the sample beads being twice resuspended in Simoa Wash Buffer B (Quanterix 103079) before final aspiration of buffer. Sample NF-L levels were measured using the NF-Light analysis protocol on the Quanterix SR-X instrument and interpolated against a calibration curve provided with the Quanterix assay kit.

### Results

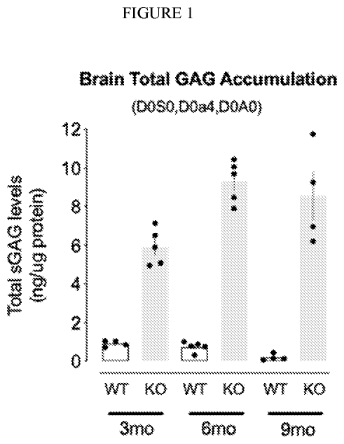

Brain HS/DS (GAG) accumulation at ages 3, 6, and 9 months in IDS KO mice relative to age-matched WT controls was measured (FIG. 1). GAG accumulation was significantly higher in the IDS KO mice at all ages tested.

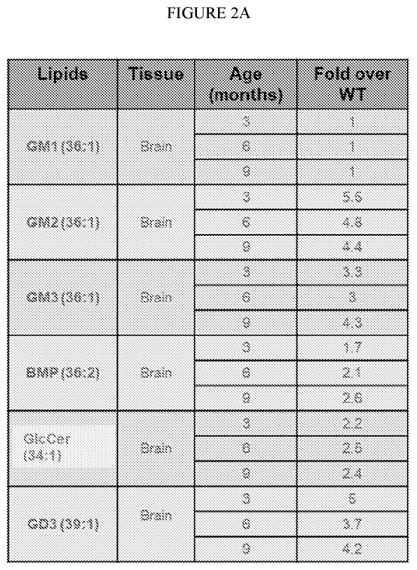

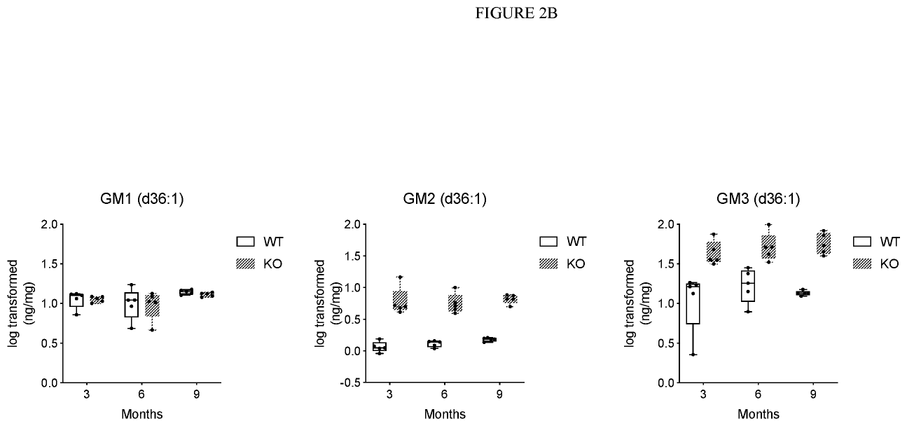

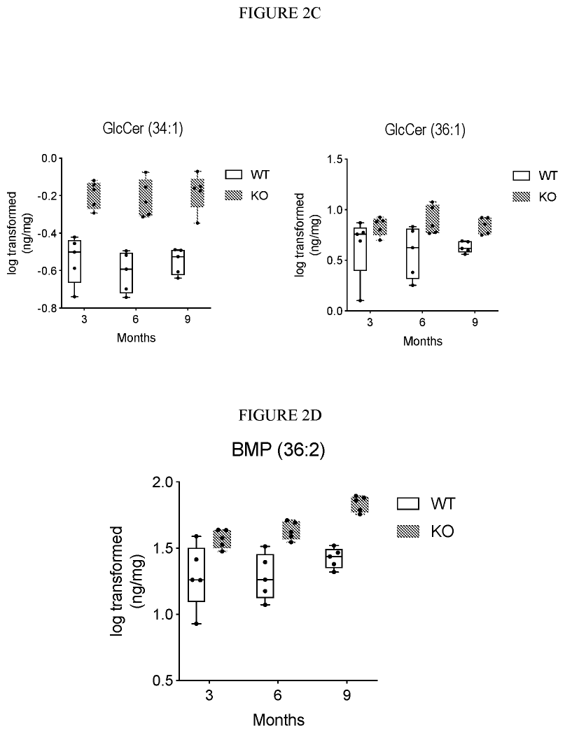

Lysosomal lipid accumulation (GM1, GM2, GM3, BMP, GlcCer and GD3) in the brains of IDS KO mice relative to age-matched WT controls was also measured (FIGS. 2A-2D). GM1 (d36:1) showed similar levels in both IDS KO and WT mice (FIGS. 2A and 2B), whereas levels of GM2 (36:1), GM3 (36:1), BMP (36:2), GlcCer (34:1), and GD (39:1) all showed between 1.7 and 5.5 fold increases over WT animals (FIGS. 2A-2D). Similar to GD3 (39:1), GD3 (36:1) also is increased in the IDS KO (2 fold on average).

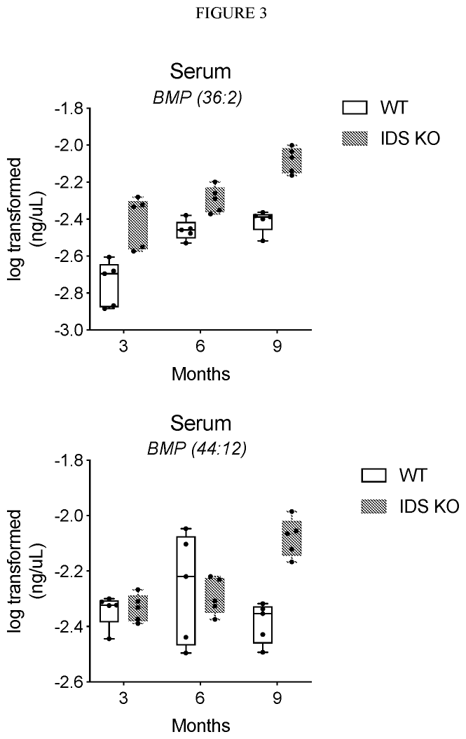

Elevated levels of BMP in the serum of IDS KO mice relative to their age-matched controls were also observed. In particular, BMP (36:2) and BMP (44:12) were increased particularly in the 9 month serum samples taken in IDS KO mice as compared to WT controls (FIG. 3).

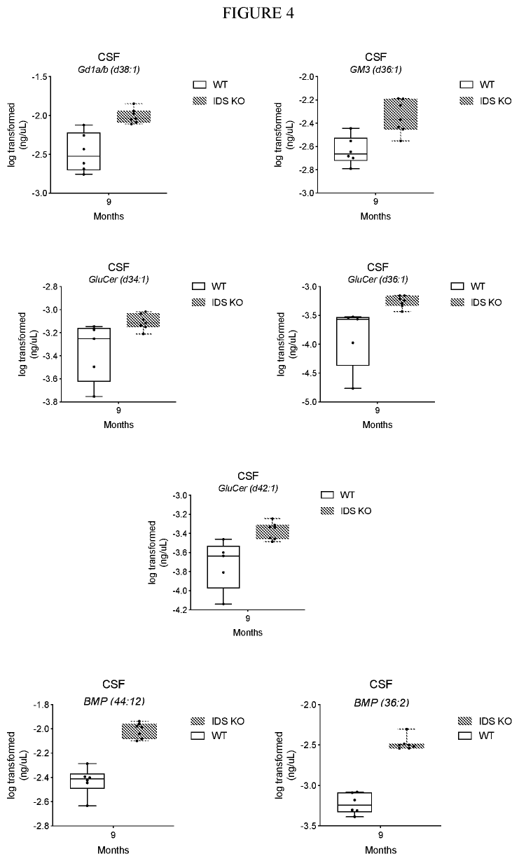

Elevated levels of lysosomal lipids (Gd1a/b, GM3, BMP and GlcCer) were observed in the CSF of 9 month old IDS KO mice relative to WT age-matched controls (FIG. 4).

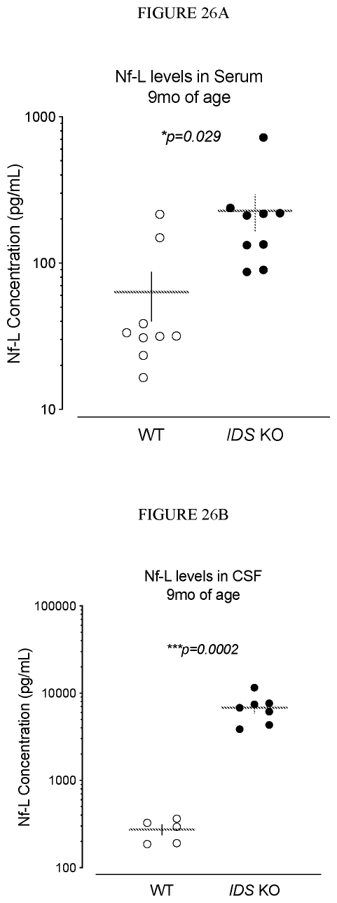

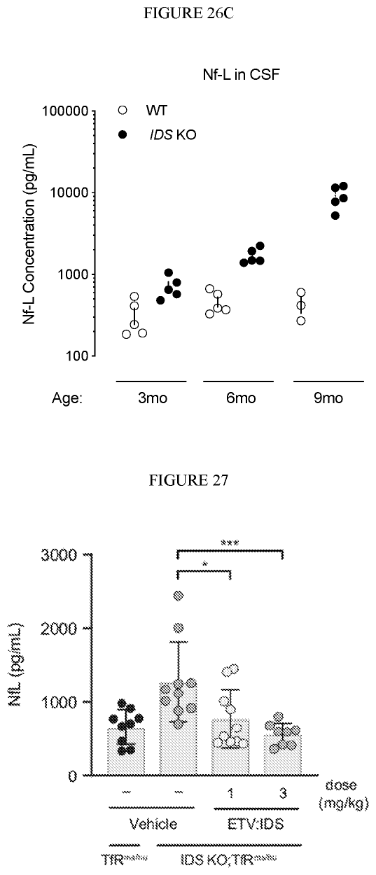

Nf-L is a useful marker for neurodegeneration (Norgren et al. 2003. Brain Research 987(1):25-31), but it has not previously been associated with Hunter syndrome/MPSII. Nf-L concentrations in serum and CSF of 9-month old IDS KO mice were elevated relative to a same-age cohort of wild-type mice (FIGS. 26A, 26B). The relative difference in Nf-L levels in CSF increased with the age of the mice cohorts, with the largest difference in Nf-L levels observed in the 9-month old mice cohort groups (FIG. 26C). These results indicate that Nf-L can also be useful as a disease-related and treatment-responsive biomarker in mouse models of Hunter syndrome.

### Example 3. Effect of Peripheral Administration of ETV:IDS on GAG and Lysosomal Lipids in IDS KO x TfR^{muhu} Mice

The effect of varying doses of ETV:IDS on GAG and lysosomal lipids in IDS KO x TfR^{muhu} mice was examined.

### Materials and Methods

### Animal Care

Mice were housed under a 12-hour light/dark cycle and had access to water and standard rodent diet (LabDiet® #25502, Irradiated) ad libitum.

### Mouse Strains

The IDS KO x TfR^{mu/hu} mice used in this study are described in Example 1 above. All mice used in this study were males.

### Administration and Sample Collection

2-3 month old IDS KO x TfR^{mu/hu} mice were injected i.v. with saline, idursulfase (14.2 mg/kg body weight), or ETV:IDS (3, 10, 20, or 40 mg/kg body weight) once every week for 4 weeks (n=5-8). 2-3 month-old littermate TfR^{mu/hu} mice, injected i.v. with saline once every week for 4 weeks (n=5) were used as controls. Animals were sacrificed 7 days following last 4-week dose.

For terminal sample collection, animals were deeply anesthetized via intraperitoneal (i.p.) injection of 2.5% Avertin. For CSF collection, a sagittal incision was made at the back of the animal's skull, subcutaneous tissue and muscle was separated to expose the cisterna magna and a pre-pulled glass capillary tube was used to puncture the cisterna magna to collect CSF. CSF was transferred to a Low Protein LoBind Eppendorf tube and centrifuged at 12,700 rpm for 10 minutes at 4° C. CSF was transferred to a fresh tube and snap frozen on dry ice. Lack of blood contamination in mouse CSF was confirmed by measuring the absorbance of the samples at 420 nm. Blood was collected via cardiac puncture for serum collection. For serum collection, blood was allowed to clot at room temperature for at least 30 minutes. Tubes were then centrifuged at 12,700 rpm for 7 minutes at 4° C. Serum was transferred to a fresh tube and flash-frozen on dry ice. Animals were transcardially perfused with ice-cold PBS using a peristaltic pump (Gilson Inc. Minipuls Evolution). The brain was dissected and flash-frozen on dry ice.

### Tissue Preparation and LCMS Assay

Tissue preparation and LCMS assays were performed using methods similar to those described in Example 1.

### Results

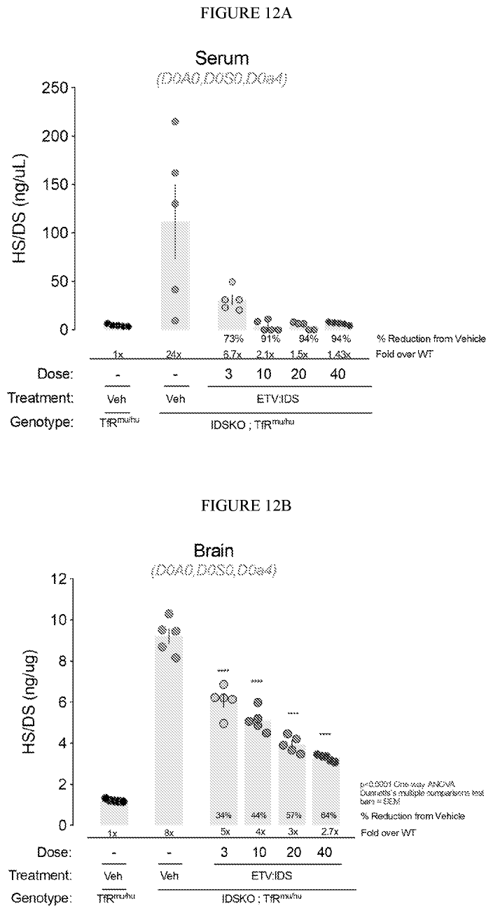

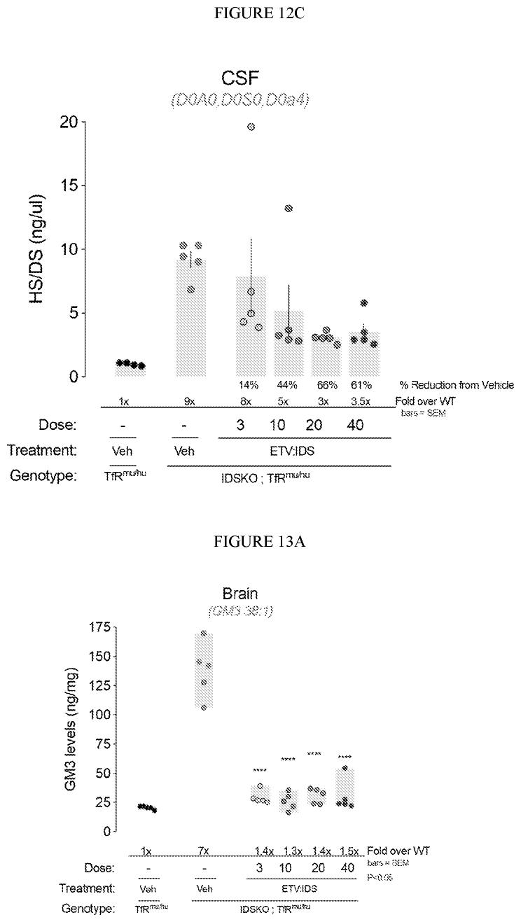

A dose-dependent decrease in serum brain and CSF GAGs was observed (FIGS. 12A-12C) for mice receiving ETV:IDS. Notably, serum GAGs were corrected down to wildtype levels.

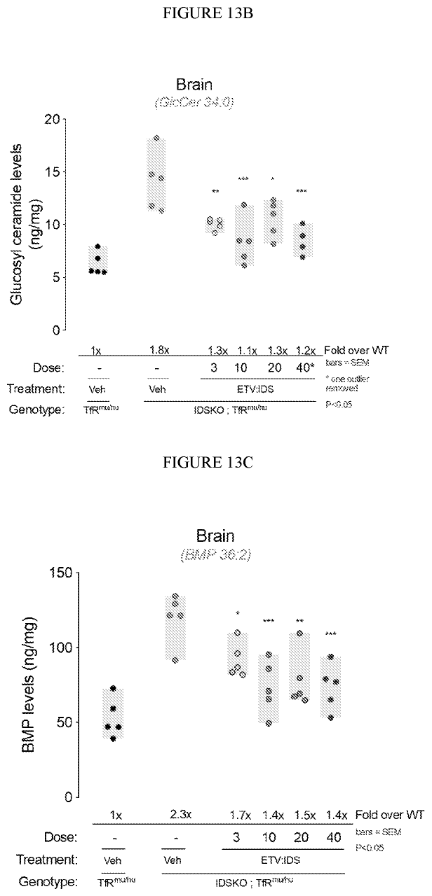

Importantly, there was a substantial reduction in brain lysosomal lipids (GM3, GlcCer and BMP), even at the lowest ETV:IDS dose (3 mg/kg) tested (FIGS. 13A-13C). Because lipid accumulation in IDS-deficient mice is believed to be a functional result of the GAG accumulation, these results may suggest that functional restoration of lysosomal function in the brain can be achieved even with GAG levels that are elevated relative to WT mice.

### Example 4. Sorting of Specific CNS Cell Types from Brain Tissue

A protocol was developed to isolate enriched populations of neurons, astrocytes, and microglial cells from brain tissue. The enriched populations were then used to investigate the effects of administering ETV:IDS in IDS KO x TfR^{hu/mu} mice (Example 5).

### Materials and Methods Animal Care

Mice were housed under a 12-hour light/dark cycle and had access to water and standard rodent diet (LabDiet® #25502, Irradiated) ad libitum.

### CNS Cell Type Isolation

To prepare a single cell suspension for sorting CNS cells, mice were perfused with PBS, brains dissected and processed into a single cell suspension according to the manufacturers' protocol using the adult brain dissociation kit (Miltenyi Biotec 130-107-677). Cells were Fc blocked (Biolegend #101320, 1:100) and stained for flow cytometric analysis with Fixable Viability Stain BV510 (BD Biosciences #564406, 1:100) to exclude dead cells, CD11b-BV421 (BD Biosciences 562605, 1:100), CD31-PerCP Cy5.5 (BD Biosciences #562861, 1:100), 01-488 (Thermo/eBio #14-6506-82, 1:37.5), Thy1-PE (R&D #FAB7335P, 1:100), and EAAT2-633 (Alomone #AGC-022-FR, 1:50). Cells were washed with PBS/1% BSA and strained through a 100 μm filter before sorting CD11b+ microglia, EAAT2+ astrocytes, and Thy1+ neurons on a FACS Aria III (BD Biosciences) with a 100 μm nozzle. In order to achieve pure populations of astrocytes, microglia, and neurons negative gates were set to remove 01+ and CD31+ cells which are predominantly oligodendrocytes and endothelial cells respectively. Sorted cells were either pelleted or collected directly into lysis buffers in preparation for downstream analysis, including qRT-PCR, RNAseq, or glycomics as described in the relevant methods disclosed herein. Cell numbers were used to calculate pg GAG/cell.

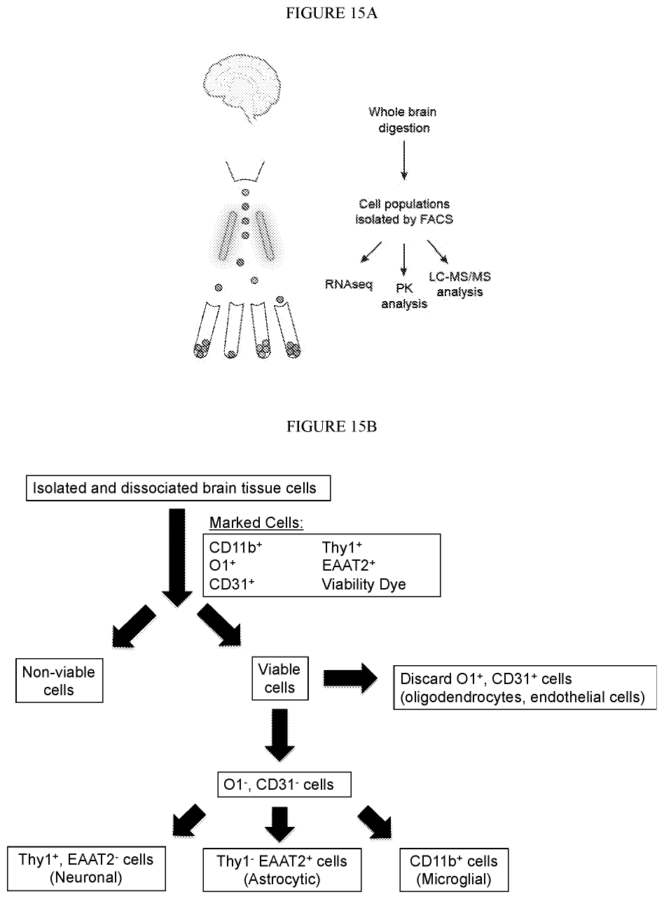

FIG. 15A is a schematic of the CNS cell sorting protocol for isolation of pure populations of neurons, astrocytes, and microglial cells as well as downstream endpoints analyzed for administration of ETV:IDS to mice (Example 5). FIG. 15B is a flowchart of the gating scheme used to isolate the enriched populations of cells. FIG. 15C includes representative FACS gates for the sorting procedure. Starting from top left to bottom right: Forward (FSC) and side (SSC) scatter determines cells from debris, live cells are positively gated, exclusion of CD31 positive endothelial cells is confirmed, EAAT2 positive astrocytes are subgated from CD11b microglia, Thy1 positive neurons are subgated from EAAT2 positive astrocytes, and finally removal of O1 oligodendrocytes cells in CD11b microglia, EAAT2 astrocytes, and Thy1 neurons cell populations determines final sort criteria.

RNAseq and qPCR Analysis of Gene Expression Isolated CNS Cell Types

To validate the sorting method, sorted cell populations were analyzed for expression of neuronal, astrocytic, and microglial genes by RNAseq and qPCR. Live cells were sorted directly in 350 μL RLT-plus buffer (Qiagen, Hilden, Germany) with 1:100 beta-mercaptoethanol. RNA was extracted using the RNeasy Plus Micro Kit (Qiagen, 74034) and resuspended in 14 μL nucleasefree water. RNA quantity and quality were assessed with a RNA 6000 Pico chip (Agilent 5067-1513) on a 2100 Bioanalyzer (Agilent). For qPCR validation, 1-2 μL RNA was transcribed into cDNA using SuperScript IV (Invitrogen). Gene expression was assessed using Taqman probes for target genes on a QuantStudio 6 Flex (Applied Biosystems) and normalized to Gapdh. For QuantSeq library prep, RNA was processed using the QuantSeq 3′ mRNAseq Library Prep Kit FWD for Illumina (Lexogen) with the UMI second strand synthesis module in order to identify and remove PCR duplicates, following the ‘low-input’ protocol defined by the manufacturer. Barcoded samples were quantified using the NEBNext Library Quant Kit for Illumina (NEB, E7630S). All samples were pooled in equimolar ratios into one sequencing library, which was quantified on a Bioanalyzer with a High Sensitivity DNA chip (Agilent, 5067-4626). 50 bp single end reads were generated in on an Illumina HiSeq 4000 lane at the UCSF Center for Advanced Technology.

For RNAseq raw data processing, UMIs were extracted from raw sequencing reads using umi2index (Lexogen) and sequencing adapters were trimmed with skewer (Jiang et al. 2014. BMC Bioinformatics 15:182). Reads were aligned to the mouse genome version GRCm38_p6. A STAR index (version 2.5.3a) was built with the -sjdbOverhang=50 argument (Dobin et al. 2013. Bioinformatics 29:15-21). Splice junctions from Gencode gene models (release M17) were provided via the -sjdbGTFfile argument. STAR alignments were generated with the following parameters: -outFilterType BySJout, -quantMode TranscriptomeSAM, -outFilterIntronMotifs RemoveNoncanonicalUnannotated, -outSAMstrandField intronMotif, -outSAMattributes NH HI AS nM MD XS and -outSAMunmapped Within. Alignments were obtained with the following parameters: -readFilesCommand zcat -outFilterType BySJout -outFilterMultimapNmax 20 -alignSJoverhangMin 8 -alignSJDBoverhangMin 1 -outFilterMismatchNmax 999 -outFilterMismatchNoverLmax 0.6 -alignIntronMin 20 -alignIntronMax 1000000 -alignMatesGapMax 1000000 -quantMode GeneCounts -outSAMunmapped Within -outSAMattributes NH HI AS nM MD XS -outSAMstrandField intronMotif -outSAMtype BAM SortedByCoordinate -outBAMcompression 6. Alignments mapped to the same genomic location that shared the same UMI were collapsed using the collapse_UMI_bam tool (Lexogen). Gene level counts were obtained using feature Counts from the subread package (version 1.6.2) (Liao et al. 2013. Bioinformatics 30:923-930). Gene symbols and biotype information were extracted from the Gencode GTF file.

All RNA-seq expression analyses were performed with R (R Core Team 2018; version 3.2), using the voom analysis framework (Law et al. 2014. Genome biology 15:R29) from the limma package (Ritchie et al. 2015. Nucleic Acids Research 43:e47). Gene expression profiles were TMM normalized (Robinson and Oshlack. 2010. Genome Biology 11:R25) and low abundance genes were identified and removed prior to downstream analysis. Low abundance genes were defined as those which were not expressed higher than 10 ten counts per million (CPM) in at least four samples.

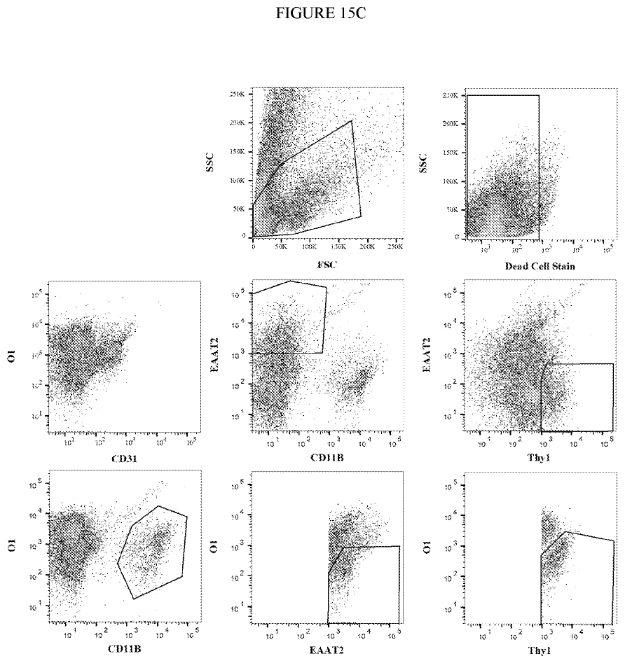

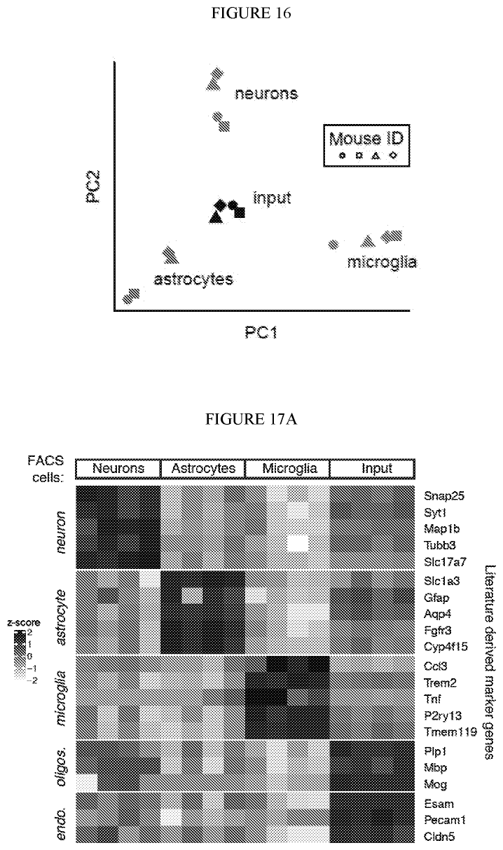

For principal components analysis to determine what variables account for primary differences between samples, log-transformed CPM expression values from the top 500 genes with the highest variance were used for principal components analysis. The projection of the samples on the first two principal components are shown in FIG. 16. Principal components 1 and 2 account for 48% and 26% of the variance in the data, respectively.

Marker genes were identified for each cell type by combining the results of individual pairwise differential expression tests between itself and the other two cell types. Marker gene p-values for each gene were calculated by combining the nominal p-values from the two “out-group” differential expression tests using Simes' method (Simes, R. J. 1986. Biometrika 73:751-754). The false discovery rate (FDR) was calculated from the combined p-values using the Benjamini-Hochberg method (Benjamini and Hochberg. 1995. Journal of the Royal Statistical Society. Series B (Methodological) 57:289-300). Genes were sorted by decreasing average log fold change vs. the other two cell types and the top 20 genes with an FDR <0.01 were used as the cell's marker genes. The individual pairwise differential expression tests were performed using limma/voom. To identify genes with strong enrichment of expression in the target cell type vs the rest, limma's treat framework was used to test statistical significance relative to a five-fold change threshold (Robinson and Oshlack, supra).

### Results

To confirm the enrichment and purity of isolated cell populations, gene expression profiles for each of the sorted populations were analyzed using RNA-Seq and qRT-PCR and compared to known profiles (FIGS. 16, 17A-17C, and 25). A strong enrichment of corresponding cell-type specific genes in isolated populations and a reduction in gene markers of endothelial cells and oligodendrocytes was observed. This collective data demonstrates that highly pure populations of neurons, astrocytes, and microglia were obtained.

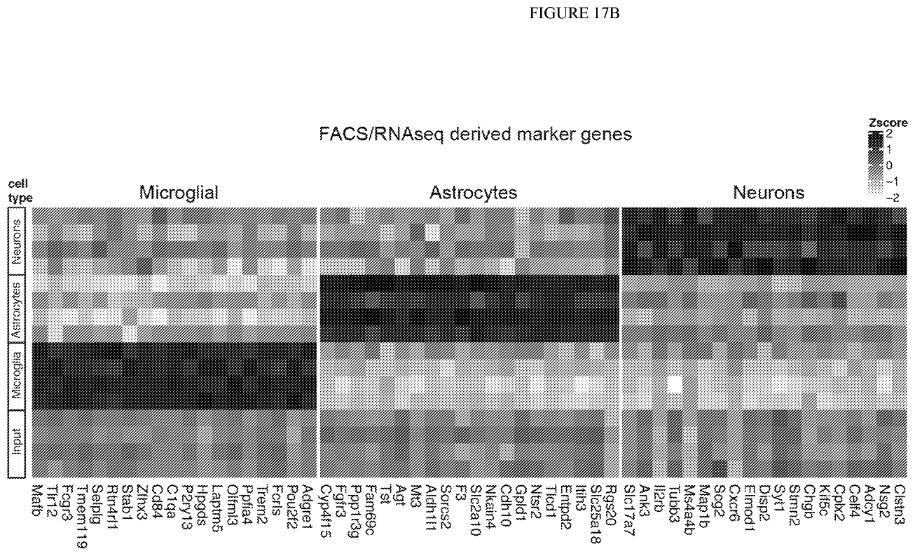

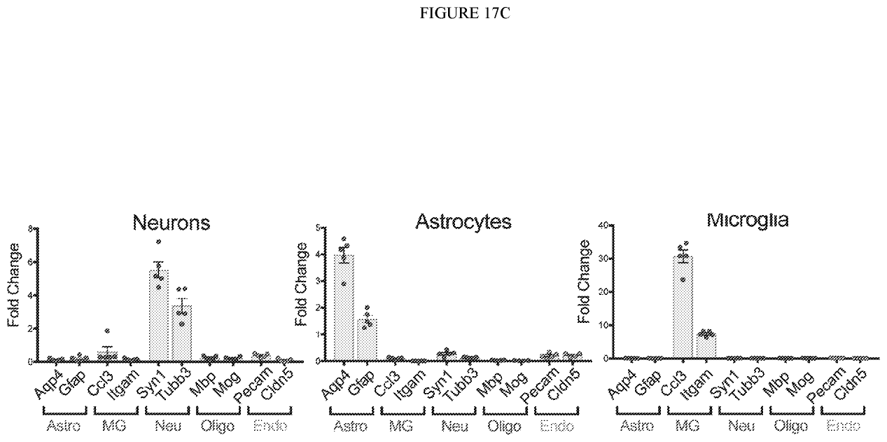

FIG. 17A illustrates expression of classic cell-specific markers identified from the literature in purified neurons, astrocytes, microglia, and the input cell suspension determined by RNA-Seq. Rows are gene groups specific to cell types: (endo.) endothelial, (oligo.) oligodendrocytes; expression values per gene are depicted as the number of standard deviations away from its mean (z-score); n=4 mice.

FIG. 17B lists and illustrates expression of the top 20 enriched genes determined by fold enrichment >5.0 and FDR <0.01 of each cell type. Heatmap expression values are plotted as gene-level z-scores. Input cells are the single cell suspension from the dissociated brains.

FIG. 17C includes representative qRT-PCR data generated from isolated cell populations confirming that the populations were successfully enriched for neurons, astrocytes, and microglia. Gene classes are grouped on the x axis: (Astro) astrocytes, (MG) microglia, (Neu) neurons, (Oligo) oligodendrocytes, and (Endo) endothelial cells; n=5 mice. Graphs display mean±SEM.

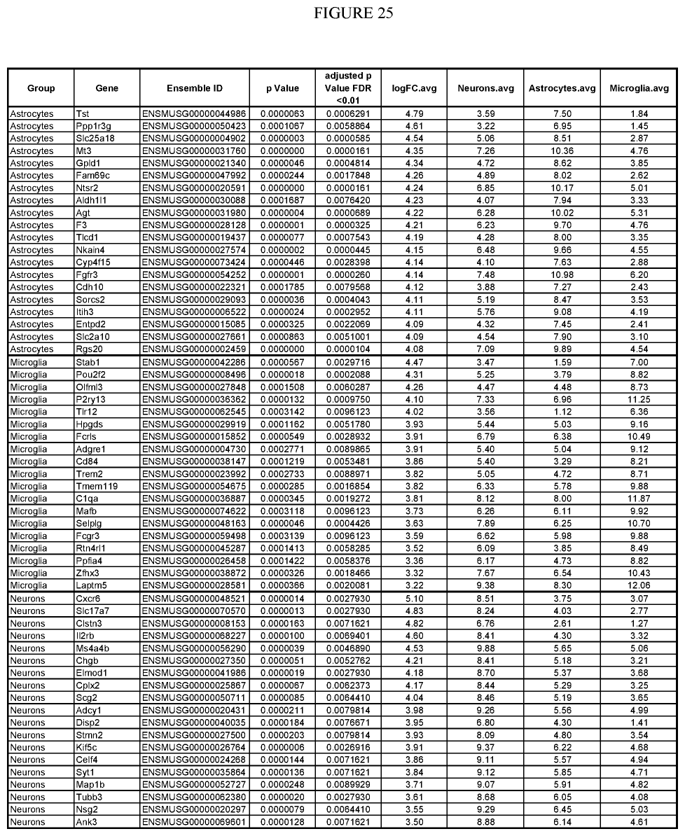

FIG. 25 is a table of a cell-type specific enriched gene set; the information in the table which corresponds to the heat map of FIG. 17B. Expression of the top 20 genes determined by fold enrichment >5.0 and FDR <0.01 for each cell type are listed in ascending order of p-value. Average log fold changes (log FC.avg) are relative to the other two “out-group” populations, and the far right three columns show the average expression of the gene within each cell population; n=4 mice.

### Example 5. Effect of Peripheral Administration of ETV:IDS on GAG and Lysosomal Lipids in Specific CNS Cell Types of IDS KO x TfR^{muhu} Mice

The effect of varying doses of ETV:IDS on GAG and lysosomal lipids in specific CNS cell types of IDS KO x TfR^{muhu} mice was examined.

### Materials and Methods

### Animal Care

Mice were housed under a 12-hour light/dark cycle and had access to water and standard rodent diet (LabDiet® #25502, Irradiated) ad libitum.

### Mouse Strains

The TfR^{hu/mu} mice and IDS KO x TfR^{mu/hu} mice used in this study are described in Example 1 above. All mice used in this study were males.

### Administration and Sample Collection

2-3 month old IDS KO x TfR^{mu/hu} mice were injected i.v. with saline or ETV:IDS (40 mg/kg body weight) once every week for 4 weeks (n=4-6 per treatment). 2-3 month-old littermate TfR^{hu/mu} mice, injected i.v. with saline once every week for 4 weeks (n=4-6) were used as controls. Animals were sacrificed 7 days following last 4-week dose.

For terminal sample collection, animals were deeply anesthetized via intraperitoneal (i.p.) injection of 2.5% Avertin. For CSF collection, a sagittal incision was made at the back of the animal's skull, subcutaneous tissue and muscle was separated to expose the cisterna magna and a pre-pulled glass capillary tube was used to puncture the cisterna magna to collect CSF. CSF was transferred to a Low Protein LoBind Eppendorf tube and centrifuged at 12,700 rpm for 10 minutes at 4° C. CSF was transferred to a fresh tube and snap frozen on dry ice. Lack of blood contamination in mouse CSF was confirmed by measuring the absorbance of the samples at 420 nm. Blood was collected via cardiac puncture for serum collection. For serum collection, blood was allowed to clot at room temperature for at least 30 minutes. Tubes were then centrifuged at 12,700 rpm for 7 minutes at 4° C. Serum was transferred to a fresh tube and flash-frozen on dry ice. Animals were transcardially perfused with ice-cold PBS using a peristaltic pump (Gilson Inc. Minipuls Evolution). The brain was dissected and flash-frozen on dry ice.

### CNS Cell Type Isolation

CNS cells were sorted as described to achieve pure populations of astrocytes, microglia, and neurons (Example 4). Sorted cells were either pelleted or collected directly into lysis buffers, and then processed for downstream analysis including qRT-PCR, RNAseq, or glycomics as described in the relevant methods. Cell numbers were used to calculate pg GAG/cell.

Cell lysate preparation and LCMS assays for measurement of GAGs, BMPs, gangliosides, GlcCer, and GalCer were performed using methods similar to those described in Example 1.

### Distribution Analysis of ETV:IDS Across CNS Cell Types

Live cells in sheath fluid (˜1.5 ml) were sorted directly into 150 μL 5% CHAPS buffer for lysis, final concentration of CHAPS 0.5%. Samples were concentrated with Amicon Ultra 30KDa filters. Five (5) μL of sample or recombinant ETV:IDS dilution series was run with an IgG (human) AlphaLISA Detection Kit (PerkinElmer #AL205C) per the manufacturer's instructions and read on an EnVision™ plate reader. Sample concentrations were interpolated from the standard curve generated using ETV:IDS and normalized to total cell input number.

### Results

The cell-type specific distribution and efficacy of ETV:IDS in the brains of IDS KO; TfR^{mu/hu} KI mice were assessed.

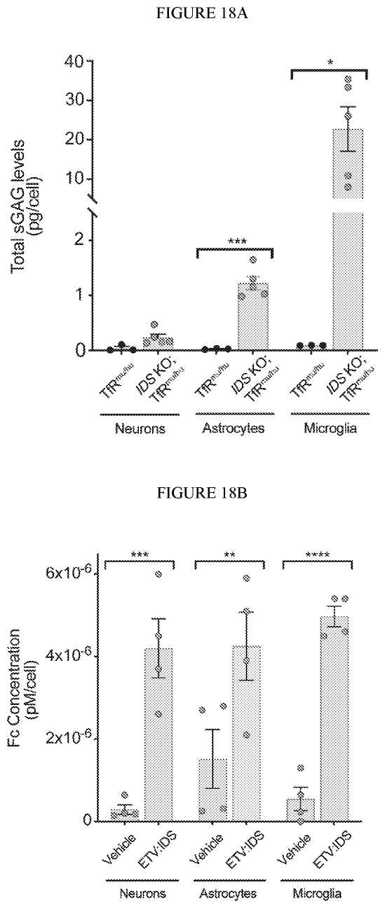

GAG levels in enriched CNS cell populations were quantified as described by LC-MS/MS. FIG. 18A illustrates that GAG levels were elevated in microglia, astrocytes, and neurons isolated from IDS KO; TfR^{mu/hu} KI mice relative to TfR^{mu/hu} KI controls, demonstrating substrate accumulation across all three CNS cell types when IDS expression was knocked-out (n=3-5 mice per group). The IDS KO; TfR^{mu/hu} KI mice (n=4 mice per group) were dosed intravenously with 40 mg/kg ETV:IDS, and enzyme concentration was assessed across CNS cell populations two hours post-dose. FIG. 18B illustrates that significant accumulation of ETV:IDS was observed in neurons, astrocytes, and microglia, demonstrating that ETV:IDS effectively distributes to the brain parenchyma and is taken up by key CNS cell types. All data are displayed as mean±SEM; unpaired student's t-test p≤0.05*, 0.001***.

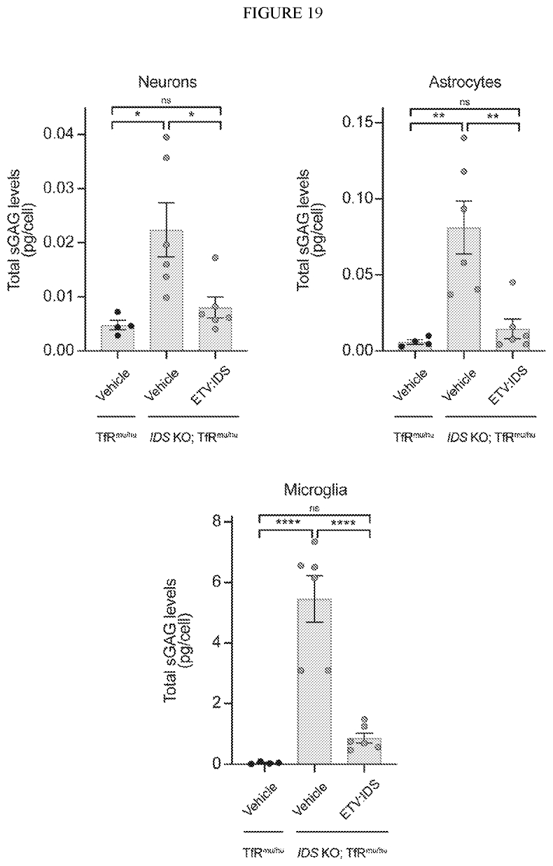

Next, IDS KO; TfR^{mu/hu} KI mice were dosed with 40 mg/kg ETV:IDS intravenously once a week for four weeks, and LC-MS/MS was used to assess the ability of ETV:IDS to reduce GAG accumulation in all three CNS cell types. FIG. 19 illustrates that ETV:IDS treatment reduced GAG levels in neurons, astrocytes, and microglia to levels comparable to that seen in wild-type mice following repeated administration. Data are displayed as mean±SEM; one-way ANOVA with Tukey's multiple comparison test, * p≤0.05, ** p≤0.01. The data demonstrates that ETV:IDS enables the delivery of IDS past the cerebral endothelium to brain cells and that this delivery is sufficient for efficacy across key CNS cell types.

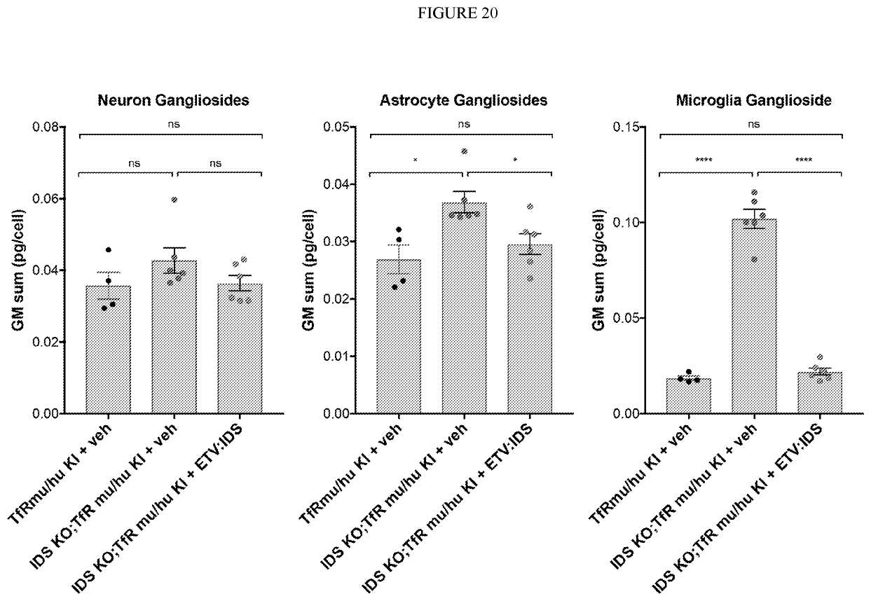

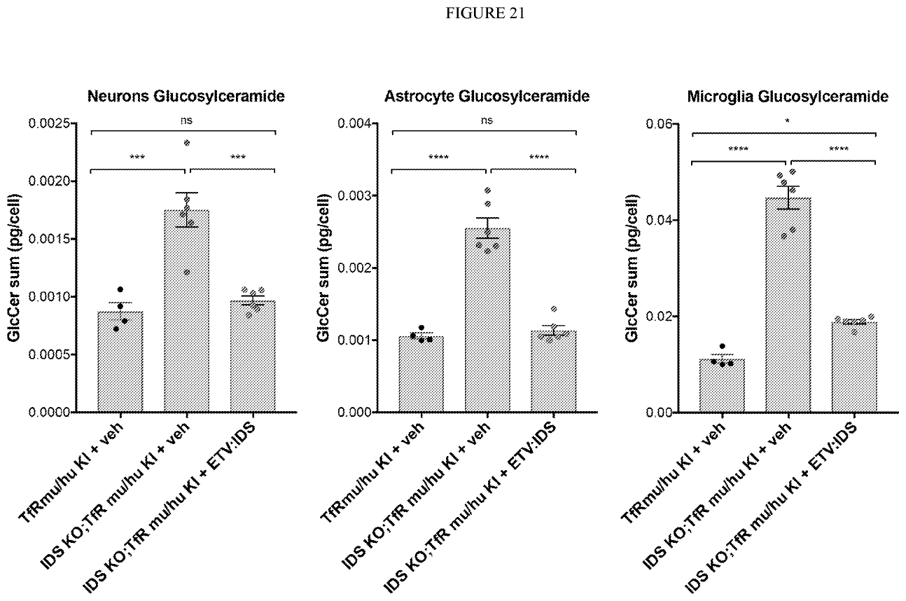

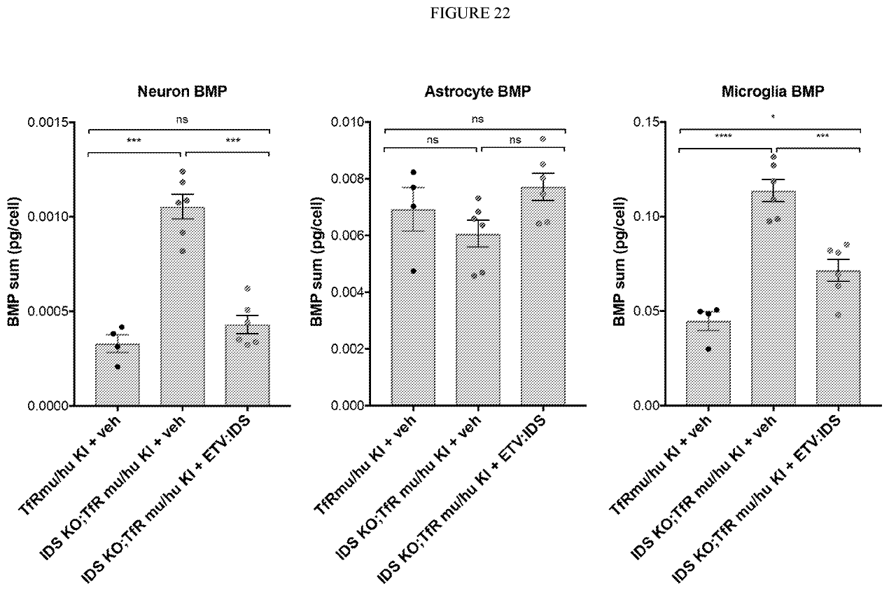

ETV:IDS treatment also reduced the secondary accumulation of the lysosomal lipids including gangliosides (FIG. 20), glucosylceramides (FIG. 21), and bis(monoacylglycerol)phosphate (BMP) (FIG. 22) in CNS cell types of interest (e.g., neurons, astrocytes, microglial cells). These data illustrate that treatment with ETV:IDS corrects secondary lysosomal dysfunction in addition to primary GAG storage in CNS cell types.

### Example 6. Spatial Distribution of Accumulated Lipid Species and Correction of Lipid Accumulation by ETV:IDS

The effect of varying doses of ETV:IDS on the spatial distribution of lysosomal lipids and on microglial activation in IDS KO x TfR^{my/hu} mice was examined.

### Mouse Strains and Administration

The TfR^{hu/mu} mice and IDS KO x TfR^{mu/hu} mice used in this study are described in Example 1 above. All mice used in this study were males. The mice were administered saline, idursulfase, or ETV:IDS (40 mg/kg body weight) and sacrificed for tissue analysis as described in Example 1.

### Mass-Spectrometry Based Imaging of Ganglioside Levels

Brain tissue was flash frozen on aluminum foil that was slowly lowered into liquid nitrogen for approximately 10 seconds. Frozen brains were stored at −80° C. until ready for use. Prior to sectioning, the brains were placed in a cryostat chamber to equilibrate the tissues to −20° C. Brains were cut on a cryostat (Leica Biosystems, Buffalo Grove, Ill.) into 12 μm thick sections and thaw-mounted onto indium-tix oxide (ITO) coated slides (Delta Technologies, Loveland, Colo.). Two brain levels were collected at approximately +0.72 mm and −1.82 mm from Bregma. Plates with sections designated for IMS were washed three times with chilled (about 4° C.) 50 mM ammonium formate and allowed to dry at room temperature prior to matrix application. Additional sections were obtained for H&E staining. After staining, digital micrographs were obtained via a slide scanner (Leica Biosystems, Buffalo Grove, Ill.). For matrix application, the plates were coated with 1,5-diaminonaphthalene (DAN) MALDI matrix via sublimation (Hankin et al. 2007. Journal of the American Society for Mass Spectrometry 18:1646-1652; Thomas et al. 2012. Analytical Chemistry 84:2048-2054). Briefly, 100 mg of recrystallized DAN was placed in the bottom of a glass sublimation apparatus (Chemglass Life Sciences, Vineland, N.J.). The apparatus was placed on a metal heating block set to 130° C., and DAN was sublimated onto the tissue surface for 4 minutes at a pressure of less than 25 mTorr. Approximately 1.8 mg of DAN was applied to each slide, determined by weighing the slide before and after matrix application. The coated plates were then placed in a Petri dish, flushed with nitrogen gas, and stored at −80° C. for two days prior to MS analysis (Yang et al. 2019. International Journal of Mass Spectrometry 437:3-9).

For imaging mass spectrometry, the plates were allowed to equilibrate to room temperature prior to removal from the sealed Petri dish. The brain sections were imaged on a Solarix 15T FT-ICR MS (Bruker Daltonics, Billerica, Mass.), equipped with a SmartBeam II 2 kHz frequency tripled Nd:YAG laser (355 nm). Images were acquired at 100 μm spatial resolution in negative ion mode. Each pixel is the average of 1000 laser shots using the small laser focus setting and random-walking within the 100 μm pixel. The mass spectrometer was externally calibrated with a series of phosphorus clusters (Sládková et al. 2009. Rapid Communications in Mass Spectrometry 23:3114-3118). Data were collected from m/z 600-3,000 with a time-domain file size of 1 M (FID length=1.3631 sec), resulting in a resolving power of 153,000 at m/z 1041. Images were generated using FlexImaging 3.0 (Bruker Daltonics, Billerica, Mass.). Gangliosides were identified by accurate mass, with the mass accuracies typically better than 1 ppm.

### Immunohistochemistry

Fresh frozen mouse brain tissue was sectioned coronally at 10 micron thickness using a Leica Cryostat (Leica CM 1950). Sections were directly mounted onto Fisherbrand Superfrost Plus microscope slides and stored at −80° C. until processed for immunohistochemistry. Sections were rinsed in 1×PBS for 3 rounds of 5 minutes then fixed in 4% Paraformaldehyde for 15 minutes. Sections were then rinsed in 1×PBS for 3 rounds of 5 minutes and incubated in Blocking Solution (1×PBS/5% normal goat serum/0.3% Triton X-100) for 1 hour at room temperature. Sections were then incubated in primary antibody (BioRad: Rat anti-Cd68, 1:500) prepared in Blocking Solution for 2 hours at room temperature. Sections were rinsed in 1×PBS/0.3% Triton X-100 for 3 rounds of 5 minutes followed by incubation in secondary antibody (Invitrogen: Goat anti-rat Alexa Fluor 488, 1:500) and DAPI (Invitrogen Molecular Probes D1306: 1:10,000 from 5 mg/mL stock) prepared in Blocking Solution for 1 hour at room temperature in the dark. Sections were then rinsed in 1×PBS/0.3% Triton X-100 for 3 rounds of 5 minutes, quickly rinsed in 1×PBS, and cover slipped with polyvinyl alcohol mounting medium with DABCO antifading (Sigma 10981). Fluorescent images were taken at 20× magnification using a Zeiss Axio Scan Z1. Each fluorophore was individually imaged with appropriate single-channel filter sets, using identical exposure times per fluorophore across all tissue samples imaged. Individual images were then tiled and stitched with shading correction using Zeiss Zen software.

### Results

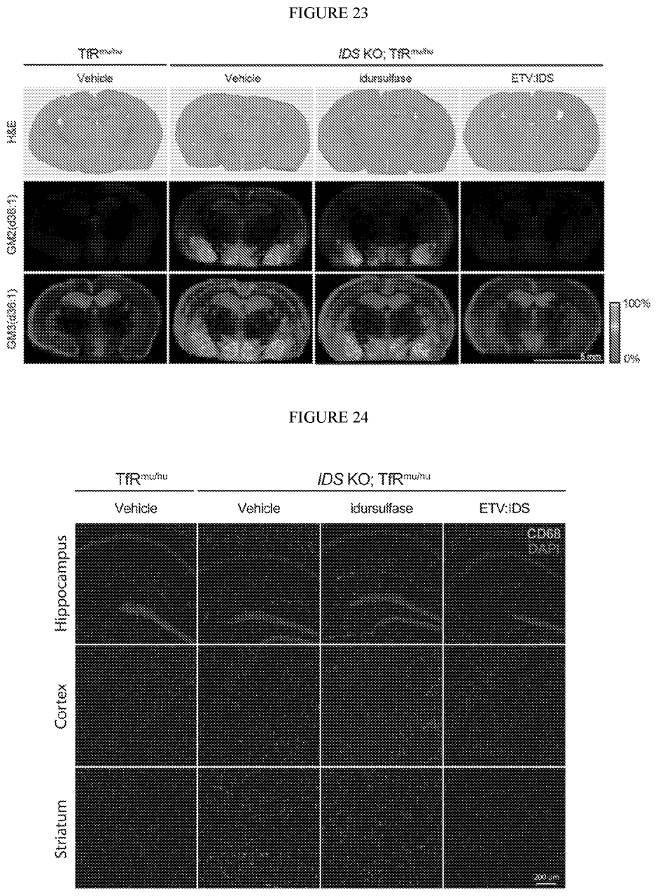

Imaging mass-spectrometry (IMS) was carried out to determine the spatial distribution of lipid accumulation in brains of in IDS KO; TfR^{mu/hu} KI mice and to assess whether ETV:IDS administration could correct lysosomal lipid accumulation throughout the brain. MALDI MS images were acquired from coronal brain sections of wild-type and IDS KO; TfR^{mu/hu} KI mice after four, weekly doses of vehicle, idursulfase or ETV:IDS. Representative images for select ganglioside species are illustrated in FIG. 23. The middle panel shows the distribution of the signal at m/z 1382.816, corresponding to GM2 (d36:1), while the bottom panel shows the distribution of the signal at m/z 1179.738, corresponding to GM3 (d36:1). Images depict the relative intensity of each signal from 0-100%.

As illustrated in FIG. 23, an enrichment of several species of gangliosides in the brains of IDS KO; TfR^{mu/hu} KI mice was observed compared with wild-type controls. This was similar to results observed in analysis of homogenized tissues (FIG. 6). Ganglioside accumulation was region specific and was concentrated in the hypothalamus and amygdala brain regions. Four weekly doses of ETV:IDS reduced the accumulation of these ganglioside species throughout all brain regions assessed to levels seen in wild-type mice, while only modest reductions in gangliosides were observed with idursulfase treatment. Taken together, these data demonstrate that ETV:IDS can correct lysosomal dysfunction downstream of GAG accumulation in the CNS.

Neuroinflammation represents a common hallmark of many neuronopathic LSDs, and there is an emerging consensus that glial activation, commonly reported in mouse models of MPS II disease as well as in MPS patients, may contribute to progressive degeneration throughout the brain in MPS disorders. The levels of two markers of microglia activity, CD68 and triggering receptor expressed on myeloid cells 2 (Trem2) were assessed through immunohistochemical analysis of brain tissue sections or biochemical analysis of brain lysates, respectively. FIG. 24 is a representative image of an immunofluorescent stain for DAPI and CD68 from the brains of TfR^{mu/hu} KI and IDS KO; TfR^{mu/hu} KI mice treated with vehicle, ETV:IDS or idursulfase. Magnification is 20×, and the images represent hippocampus (top panel), cortex (middle panel), and striatum (bottom panel). FIG. 10 illustrates levels of Trem2 in the treated mice.

The levels of CD68 and Trem2 were both elevated in the brains of IDS KO; TfR^{mu/hu} KI mice compared to TfR^{mu/hu} KI controls (FIGS. 10, 24). Four weekly doses of 40 mg/kg ETV:IDS effectively reduced the CD68 signal throughout the brains of IDS KO; TfR^{mu/hu} KI mice (FIG. 21). Levels of Trem2 in brain were completely reduced to levels seen in wild-type mice following repeated systemic administration of ETV:IDS (FIG. 10). Four weekly activity-equivalent doses of idursulfase, however, failed to lower levels of CD68 and Trem2. Administration of ETV:IDS also reduced accumulation of soluble Trem2 (sTrem2) in the CSF of treated IDS KO; TfR^{mu/hu} KI mice (FIG. 11). These data demonstrate that ETV:IDS can attenuate microglia activity in addition to GAG and secondary lysosomal lipid accumulation in the brain.

### Example 7. Construction of Fusion Proteins Comprising IDS

### Design and Cloning

IDS-Fc fusion proteins were designed that contain (i) a fusion polypeptide where a mature, human IDS enzyme is fused to a human IgG1 fragment that includes the Fc region (an “IDS-Fc fusion polypeptide”), and (ii) a modified human IgG1 fragment which contains mutations in the Fc region that confer transferrin receptor (TfR) binding (a “modified Fc polypeptide”). In particular, IDS-Fc fusion polypeptides were created in which IDS fragments were fused to either the N- or C-terminus of the human IgG1 Fc region. In some cases, a linker was placed between the IDS and IgG1 fragments to alleviate any steric hindrance between the two fragments. In all constructs, the signal peptide from the kappa chain V-III, amino acids 1-20 (UniProtKB ID—P01661) was inserted upstream of the fusion to facilitate secretion, and IDS was truncated to consist of amino acids S26-P550 (UniProtKB ID—P22304). The fragment of the human IgG1 Fc region used corresponds to amino acids D104-K330 of the sequence in UniProtKB ID P01857 (positions 221-447, EU numbering, which includes 10 amino acids of the hinge (positions 221-230)). In some embodiments, a second Fc polypeptide derived from human IgG1 residues D104-K330 but lacking the IDS fusion was co-transfected with the IDS-Fc fusion polypeptide in order to generate heterodimeric fusion proteins with one IDS enzyme (a “monozyme”). In some constructs, the IgG1 fragments contained additional mutations to facilitate heterodimerization of the two Fc regions. Control IDS-Fc fusion proteins that lack the mutations that confer TfR binding were designed and constructed analogously, with the difference being that these proteins lacked the mutations that confer TfR binding. As an additional control, we generated IDS (amino acids S26-P550) with a C-terminal hexahistidine tag (SEQ ID NO:203) to facilitate detection and purification.

The TfR-binding IDS-Fc fusion proteins used in the examples are dimers formed by an IDS-Fc fusion polypeptide and a modified Fc polypeptide that binds to TfR. For dimers where the IDS enzyme is linked to the N-terminus of the Fc region, the IDS-Fc fusion polypeptide may have the sequence of any one of SEQ ID NOS:113, 193, and 197. In these sequences, the IDS sequence is underlined and contains a cysteine at position 59 (double underlined) modified to formylglycine. The IDS was joined to the Fc polypeptide by a GGGGS linker (SEQ ID NO:201). A portion of an IgG1 hinge region (DKTHTCPPCP; SEQ ID NO:111) was included at the N-terminus of the Fc polypeptide. The CH2 domain sequence starts at position 541 of SEQ ID NOS:113, 193, and 197.

The IDS-Fc fusion protein ETV:IDS 35.21 is a dimer formed by an IDS-Fc fusion polypeptide having the sequence of any one of SEQ ID NOS:113, 193, and 197 and a modified Fc polypeptide that binds to TfR having the sequence of SEQ ID NO:114. The first 10 amino acids are a portion of an IgG1 hinge region. The CH2 domain sequence starts at position 11 of SEQ ID NO:114.

The IDS-Fc fusion protein ETV:IDS 35.21.17.2 is a dimer formed by an IDS-Fc fusion polypeptide having the sequence of any one of SEQ ID NOS:113, 193, and 197 and a modified Fc polypeptide that binds to TfR having the sequence of SEQ ID NO:190. The first 10 amino acids are a portion of an IgG1 hinge region. The CH2 domain sequence starts at position 11 of SEQ ID NO:190.

The IDS-Fc fusion protein ETV:IDS 35.23.2 is a dimer formed by an IDS-Fc fusion polypeptide having the sequence of any one of SEQ ID NOS:113, 193, and 197 and a modified Fc polypeptide that binds to TfR having the sequence of SEQ ID NO:191. The first 10 amino acids are a portion of an IgG1 hinge region. The CH2 domain sequence starts at position 11 of SEQ ID NO:191.

The IDS-Fc fusion protein ETV:IDS 35.21.17 is a dimer formed by an IDS-Fc fusion polypeptide having the sequence of any one of SEQ ID NOS:113, 193, and 197 and a modified Fc polypeptide that binds to TfR having the sequence of SEQ ID NO:117. The N-terminus of the modified Fc polypeptide may include a portion of an IgG1 hinge region (e.g., SEQ ID NO:111).

### Recombinant Protein Expression and Purification

To express recombinant IDS enzyme fused to an Fc region, ExpiCHO cells (Thermo Fisher Scientific) were transfected with relevant DNA constructs using Expifectamine™ CHO transfection kit according to manufacturer's instructions (Thermo Fisher Scientific). Cells were grown in ExpiCHO™ Expression Medium at 37° C., 6% CO₂ and 120 rpm in an orbital shaker (Infors HT Multitron). In brief, logarithmic growing ExpiCHO™ cells were transfected at 6×10⁶ cells/ml density with 0.8 μg of DNA plasmid per mL of culture volume. After transfection, cells were returned to 37° C. and transfected cultures were supplemented with feed as indicated 18-22 hrs post transfection. Transfected cell culture supernatants were harvested 120 hrs post transfection by centrifugation at 3,500 rpm from 20 mins. Clarified supernatants were filtered (0.22 μM membrane) and stored at 4° C. Expression of an epitope-tagged IDS enzyme (used as a control) was carried out as described above with minor modifications. In brief, an IDS enzyme harboring a C-terminal hexahistidine tag (SEQ ID NO:203) was expressed in ExpiCHO cells.

IDS-Fc fusion proteins with (or without) engineered Fc regions conferring TfR binding were purified from cell culture supernatants using Protein A affinity chromatography. Supernatants were loaded onto a HiTrap MabSelect SuRe Protein A affinity column (GE Healthcare Life Sciences using an Akta Pure System). The column was then washed with >20 column volumes (CVs) of PBS. Bound proteins were eluted using 100 mM citrate/NaOH buffer pH 3.0 containing 150 mM NaCl. Immediately after elution, fractions were neutralized using 1 M arginine-670 mM succinate buffer pH 5.0 (at a 1:5 dilution). Homogeneity of IDS-Fc fusion proteins in eluted fractions was assessed by reducing and non-reducing SDS-PAGE.

To purify hexahistadine-tagged (SEQ ID NO:203) IDS enzyme, transfected supernatants were exhaustively dialyzed against 15 L of 20 mM HEPES pH 7.4 containing 100 mM NaCl overnight. Dialyzed supernatants were bound to a HisTrap column (GE Healthcare Life Sciences using an Akta Pure System). After binding, the column was washed with 20 CV of PBS. Bound proteins were eluted using PBS containing 500 mM imidazole. Homogeneity of IDS enzyme in eluted fractions was assessed by reducing and non-reducing SDS-PAGE. Pooled fractions containing IDS enzyme were diluted 1:10 in 50 mM Tris pH 7.5 and further purified using Q Sepharose High Performance (GE Healthcare). After binding, the column was washed with 10 CV of 50 mM Tris pH 7.5. Bound proteins were eluted using a linear gradient to 50 mM Tris pH 7.5 and 0.5 M NaCl and collected in 1 CV fractions. Fraction purity was assessed by non-reducing SDS-PAGE. Purification yielded homogeneous IDS-Fc fusion proteins and hexahistidine-tagged (SEQ ID NO:203) IDS enzyme.

### Example 8. Effect of Peripheral Administration of ETV:IDS on GAG and Neurofilament Light Chain (Nf-L) in IDS KO x TfR^{muhu} Mice

The effect of weekly IV doses of ETV:IDS on GAG and neurofilament light chain (Nf-L) in IDS KO x TfR^{muhu} mice was examined.

### Materials and Methods

### Animal Care

Mice were housed under a 12-hour light/dark cycle and had access to water and standard rodent diet (LabDiet® #25502, Irradiated) ad libitum.

### Mouse Strains

The IDS KO x TfR^{mu/hu} mice and TfR^{mu/hu} KI mice used in this study are described in Example 1 above. All mice used in this study were males.

### Administration and Sample Collection

8-week old IDS KO; TfR^{mu/hu} KI mice were administered 1 mg/kg, or 3 mg/kg ETV:IDS intravenously (IV) via the tail vein. Eight (8)-week old TfR^{mu/hu} KI mice (IDS WT), injected with vehicle, were used as non-disease controls. The ETV:IDS or vehicle was administered once weekly for a period of 13 weeks. All animals were euthanized 7 days following their last dose.

In-life serum and terminal CSF samples were collected as described in Example 1. Brain and liver tissue samples were also collected as described in Example 1. In addition, urine was collected and chilled immediately prior to termination. Urine samples were then stored in a freezer, set to maintain −60 to −80° C., for urine biomarker analysis.

### Quantification of GAGs

Brain and liver tissues were prepared for quantification of GAGs (e.g. heparan sulfate (HS) and dermatan sulfate (DS)) as described in Example 1. Prior to LCMS assay to quantify GAGs, protein lysates (from tissue) or CSF, urine, or serum were mixed with a combination of Heparinases I, II, III, and Choidriotinase B. The digests were mixed with acetonitrile and analyzed by LCMS. LCMS assay to quantify GAGs was carried out as described in Example 1.

### Methods for CSF and Serum Analysis of Nf-L

Nf-L levels in serum and CSF were measured as described in Example 2.

### Results

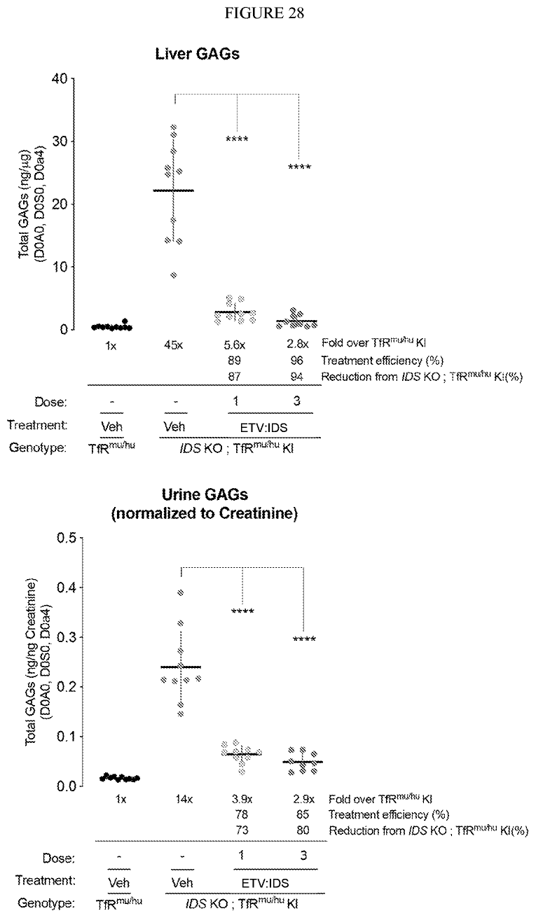

Brain, CSF, liver, and urine GAGs. GAG levels in brain from IDS KO; TfR^{mu/hu} mice were measured 7 days after the last dose of ETV:IDS and compared to vehicle treatment and TfR^{mu/hu} mice. Consistent with earlier results, brain GAG values decreased with increasing doses of ETV:IDS and resulted in a treatment efficiency of 64% and 75% from vehicle treated IDS KO; TfR^{mu/hu} mice for 1 and 3 mg/kg, respectively (data not shown). CSF GAG values also decreased with increasing doses of ETV:IDS and resulted in a treatment efficiency of 60% and 70% from vehicle treated IDS KO; TfR^{mu/hu} mice for 1 and 3 mg/kg, respectively (data not shown). Levels of liver GAGs showed near complete correction at all dose levels of ETV:IDS (treatment efficiency of 98% and 96% from vehicle treated IDS KO; TfR^{mu/hu} mice for 1 and 3 mg/kg, respectively) (FIG. 28). GAG levels in urine were also reduced following 13 weekly treatments with ETV:IDS. The treatment efficiency of creatinine normalized GAG levels were 78% and 85% with 1 and 3 mg/kg respectively (FIG. 28).

CSF neurofilament light chain (Nf-L). As illustrated in FIGS. 26A-26C, elevated levels of Nf-L in the CSF and serum were observed in a mouse model of Hunter syndrome. Weekly doses of ETV:IDS over a 13-week period was capable of reversing these changes, and complete normalization of CSF Nf-L was observed in IDS KO; TfR^{mu/hu} treated with ETV:IDS. The treatment efficiency of Nf-L correction was 82% and 117% at 1 and 3 mg/kg respectively, bringing the levels of Nf-L down to that observed in IDS WT controls (FIG. 27).

### Tables

TABLE 1
Summary of GlcCer levels in brains of IDS
KO mice treated with ETV:IDS (fold over WT)

| | KO + | KO + | KO + |
|---|---|---|---|
| | Vehicle | ETV:IDS | Idursulfase |
| GlcCer (d18:1, 16:0) | 2.2 | 1.2 | 1.8 |
| GlcCer (d18:1, 18:0) | 1.5 | 1.0 | 1.4 |
| GlcCer (d18:2, 18:0) | 1.6 | 0.9 | 1.3 |
| GlcCer (d18:1, 20:0) | 1.6 | 1.1 | 1.4 |
| GlcCer (d18:2, 20:0) | 1.7 | 1.1 | 1.4 |
| GlcCer (d18:1, 22:0) | 2.0 | 1.1 | 1.8 |
| GlcCer (d18:1, 22:1) | 1.2 | 1.1 | 1.1 |
| GlcCer (d18:2, 22:0) | 1.1 | 1.0 | 1.3 |
| GlcCer (d18:1, 24:1) | 1.6 | 1.1 | 1.4 |
| GlcCer (d18:1, 24:0) | 1.9 | 1.1 | 1.6 |

TABLE 2
Lipid levels in brains of IDS KO mice treated with
vehicle, ETV:IDS or Elaprase (fold changes over WT)

| | Fold changes over WT | | | |
|---|---|---|---|---|
| | | 0.9 | 1.1 | |
| | KO + | KO + | KO + | |
| | Vehicle | ETV:IDS | Elaprase | WT |
| PA(16:0_18:1) | 1.01652815 | 0.87805028 | 1.02614953 | 1 |
| PA(18:0_18:1) | 0.9803871 | 0.89061561 | 1.01854752 | 1 |
| PA(18:1_18:1) | 1.01723588 | 0.8753273 | 1.01906474 | 1 |
| PA(18:0_20:4) | 1.05587055 | 0.95439223 | 1.0287576 | 1 |
| PA(18:1_22:6) | 1.03000727 | 0.98349165 | 1.00662459 | 1 |
| PA(18:0_22:6) | 0.99319049 | 0.92980276 | 0.93442667 | 1 |
| PE(P-18:0/18:1) | 0.98195031 | 0.95324202 | 1.02518226 | 1 |
| PE(P-18:0/18:2) | 0.97225052 | 0.89209895 | 0.93871825 | 1 |
| PE(P-16:0/20:4) | 0.99144839 | 0.94499411 | 0.92862372 | 1 |
| PE(P-18:0/20:4) | 0.96754718 | 0.89442024 | 0.91564924 | 1 |
| PE(P-16:0/22:6) | 0.92008888 | 0.85984114 | 0.87469497 | 1 |
| PE(P-18:0/22:6) | 0.99775035 | 0.95455118 | 0.96148973 | 1 |
| Sulfatide C16:0 | 0.90576849 | 0.79127831 | 0.76195962 | 1 |
| Sulfatide C18:0 | 0.81280069 | 0.75779574 | 0.68197925 | 1 |
| Sulfatide C18:0(OH) | 0.76031924 | 0.75628052 | 0.64675377 | 1 |
| Sulfatide C24:0 | 0.75780648 | 0.70713297 | 0.66131545 | 1 |
| Sulfatide C24:0(OH) | 0.78833907 | 0.72508863 | 0.74860619 | 1 |
| Sulfatide C24:1 | 0.821473 | 0.74990649 | 0.75471324 | 1 |
| Sulfatide C24:1(OH) | 0.67258164 | 0.62567179 | 0.57409235 | 1 |
| GM3(d34:1) | 2.09393928 | 1.16672118 | 1.98170536 | 1 |
| GM3(d36:1) | 3.74157091 | 1.08458273 | 2.97575794 | 1 |
| GM3(d38:1) | 5.52033941 | 0.99911522 | 4.11985175 | 1 |
| GM3(d40:1) | 1.42159849 | 1.03491677 | 1.39585927 | 1 |
| GD3(d34:1) | 1.46726848 | 0.96014081 | 1.31353994 | 1 |
| GD3(d36:1) | 1.46699639 | 0.86224602 | 1.21726307 | 1 |
| GD3(d38:1) | 1.53724473 | 0.82158939 | 1.20878056 | 1 |
| GD3(d40:1) | 0.49744039 | 0.55827773 | 0.76669619 | 1 |
| GD3(d42:2) | 1.23998054 | 1.00287447 | 1.21684885 | 1 |
| GD3(d42:1) | 1.37177496 | 1.05759888 | 1.30756718 | 1 |
| GD1a/b(d36:1) | 0.88470817 | 0.82107431 | 0.81837911 | 1 |
| GD1a/b(d38:1) | 0.94221971 | 0.82198461 | 0.86416076 | 1 |
| GT1b(d36:1) | 0.93359111 | 0.81370939 | 0.85646637 | 1 |
| GT1b(d38:1) | 1.01291833 | 1.05855164 | 0.98541269 | 1 |
| LBPA(20:4/20:4) | 0.91555026 | 0.74507558 | 0.88416882 | 1 |
| LBPA(22:6/22:6) | 1.36257845 | 0.96610388 | 1.31658696 | 1 |
| LBPA(18:1/18:1) | 1.69980961 | 0.93424803 | 1.60274415 | 1 |
| Palmitic acid | 0.98744714 | 0.98336985 | 0.9050388 | 1 |
| Palmitoleic acid | 1.0337731 | 1.07564952 | 0.8971913 | 1 |
| Stearic acid | 0.96048221 | 0.92211752 | 0.86237988 | 1 |
| Oleic acid | 1.04267865 | 1.07932738 | 0.94252135 | 1 |
| Linoleic acid | 1.16554943 | 1.23446712 | 1.06289268 | 1 |
| Linolenic acid | 1.09587266 | 1.14758313 | 1.00817176 | 1 |
| Arahidonic acid | 0.96304418 | 0.93138432 | 0.88614686 | 1 |
| EPA | 1.15835012 | 1.09293496 | 1.11325942 | 1 |
| DHA | 1.12990573 | 1.13617975 | 1.05917735 | 1 |
| LPE(P-16:0) | 1.31137183 | 1.23195216 | 1.37622222 | 1 |
| LPE(P-18:0) | 1.30059624 | 1.16733271 | 1.29181659 | 1 |
| LPE(P-18:1) | 1.25418938 | 1.07523775 | 1.2800414 | 1 |
| LPE(16:0) | 1.17836852 | 1.02214629 | 1.18935927 | 1 |
| LPE(18:0) | 1.20717572 | 1.07020161 | 1.19516724 | 1 |
| LPE(18:1) | 1.25977631 | 1.16098986 | 1.34700962 | 1 |
| LPI(16:0) | 1.1754397 | 1.01479906 | 1.20396379 | 1 |
| LPI(18:0) | 1.22645413 | 1.00320543 | 1.22476213 | 1 |
| LPI(20:4) | 1.08781434 | 1.07354529 | 1.1529951 | 1 |
| LPG(16:0) | 1.20599747 | 1.0439152 | 1.21749983 | 1 |
| LPG(18:0) | 1.20955994 | 0.99977385 | 1.15689786 | 1 |
| LPG(18:1) | 1.19938507 | 1.04431583 | 1.21637775 | 1 |
| LPG(20:4) | 1.19976807 | 0.98041911 | 1.16595786 | 1 |
| CL(72:8) | 1.31239964 | 1.17287219 | 1.50060034 | 1 |
| Cholesterol Sulfate | 0.80540874 | 0.94340786 | 0.91029938 | 1 |
| PG(16:0_18:1) | 0.92567469 | 0.83442845 | 0.90850807 | 1 |
| PG(18:0_18:1) | 1.02612596 | 0.96264443 | 0.99514232 | 1 |
| PG(18:1/18:1) | 0.95920271 | 0.89717417 | 0.94675624 | 1 |
| PG(18:0_20:4) | 0.90789033 | 0.82978451 | 0.87514211 | 1 |
| PI(18:0_18:1) | 1.17804687 | 1.06396153 | 1.13113519 | 1 |
| PI(18:1/18:1) | 1.14059806 | 1.00809425 | 1.11351988 | 1 |
| PI(16:0_20:4) | 1.20957205 | 1.10714204 | 1.14720781 | 1 |
| PI(18:0_20:4) | 1.17971223 | 1.09279463 | 1.13849017 | 1 |
| PI(16:0_22:6) | 1.18060688 | 1.0109285 | 1.15474382 | 1 |
| PI(18:0_22:6) | 1.30588528 | 1.1193819 | 1.2830753 | 1 |
| PI(20:4/20:4) | 1.31581237 | 1.26493195 | 1.45542681 | 1 |
| PS(18:0_18:1) | 1.03679611 | 0.90917125 | 0.94646579 | 1 |
| PS(18:0_20:4) | 1.06956884 | 0.94321053 | 0.99958824 | 1 |
| PS(16:0_22:6) | 1.01389572 | 0.87820865 | 0.92402097 | 1 |
| PS(18:1_22:6) | 1.03170698 | 0.93486711 | 0.95345599 | 1 |
| PS(18:0_22:6) | 0.92376879 | 0.84379138 | 0.87852082 | 1 |
| Sphingosine | 1.20956582 | 1.03139557 | 1.07103473 | 1 |
| Sphinganine | 1.12625109 | 0.95945537 | 1.00866548 | 1 |
| Cer(d18:1/16:0) | 1.1898435 | 1.2702808 | 1.09898379 | 1 |
| Cer(d18:1/18:0) | 1.05719808 | 1.04709156 | 0.93672262 | 1 |
| Cer(d18:1/24:0) | 1.07960504 | 0.97523544 | 0.92010478 | 1 |
| Cer(d18:1/24:1) | 0.82995192 | 0.81205788 | 0.87781267 | 1 |
| Cer(d18:0/16:0 | 1.40270677 | 1.30051111 | 1.13522744 | 1 |
| Cer(d18:0/18:0) | 0.98462219 | 0.92661852 | 0.93865067 | 1 |
| Cer(d18:0/24:0) | 0.99180462 | 0.98741525 | 0.97143202 | 1 |
| Cer(d18:0/24:1) | 1.11123432 | 1.04202586 | 1.0700886 | 1 |
| SM(d18:1/16:0) | 1.12700306 | 1.0423671 | 1.07524893 | 1 |
| SM(d18:1/18:0) | 0.92305936 | 0.85541522 | 0.83048724 | 1 |
| SM(d18:1/24:0) | 1.05515306 | 0.88080112 | 1.00315475 | 1 |
| SM(d18:1/24:1) | 1.12213595 | 1.03280034 | 1.03584333 | 1 |
| SM(d18:1/0:0) | 0.91345559 | 0.75717864 | 1.0337506 | 1 |
| LacCer(d18:1/16:0) | 0.89628608 | 0.86982524 | 0.84127411 | 1 |
| LacCer(d18:1/18:0) | 0.85680143 | 0.77685157 | 0.805254 | 1 |
| LacCer(d18:1/24:0) | 0.86945705 | 0.79822554 | 0.78436148 | 1 |
| LacCer(d18:1/24:1) | 1.1604392 | 0.90198172 | 0.9768989 | 1 |
| LPC(16:0) | 1.11016041 | 1.1168508 | 1.2442346 | 1 |
| LPC(18:0) | 1.10803656 | 1.07688874 | 1.1572643 | 1 |
| LPC(18:1) | 1.1154886 | 1.04813535 | 1.24491264 | 1 |
| LPC(20:4) | 0.98466789 | 0.96068998 | 1.1325393 | 1 |
| LPC(22:6) | 0.9658907 | 0.86998883 | 1.06140777 | 1 |
| LPC(26:0) | 0.88498543 | 0.67243113 | 0.9152565 | 1 |
| LPC(24:0) | 1.09363028 | 0.85617717 | 1.07651749 | 1 |
| LPC(26:1) | 1.20767361 | 0.88836639 | 1.16334611 | 1 |
| LPC(24:1) | 1.08223641 | 0.82913148 | 1.09880877 | 1 |
| LPC(16:1) | 1.0717371 | 1.00744658 | 1.19339107 | 1 |
| PC(36:1) | 1.00006753 | 0.93268207 | 0.94070597 | 1 |
| PC(36:2) | 0.99239587 | 0.9360556 | 0.95517289 | 1 |
| PC(36:4) | 0.96248027 | 0.91666785 | 0.9224405 | 1 |
| PC(38:4) | 0.96130284 | 0.92655592 | 0.92290748 | 1 |
| PC(38:6) | 0.96207788 | 0.90828018 | 0.92381277 | 1 |
| PC(40:4) | 0.91798169 | 0.91136589 | 0.86172822 | 1 |
| PC(40:6) | 0.94836904 | 0.90006372 | 0.91057844 | 1 |
| PC(O-18:0/2:0) | 1.14562106 | 1.02053195 | 1.19829164 | 1 |
| PE(36:1) | 1.06114562 | 1.0189909 | 1.05259418 | 1 |
| PE(36:2) | 1.15485876 | 1.04526236 | 1.1343485 | 1 |
| PE(36:4) | 1.04833954 | 1.03567807 | 1.06728264 | 1 |
| PE(38:4) | 1.04079742 | 0.99455675 | 1.0226952 | 1 |
| PE(38:6) | 1.07401418 | 0.99212159 | 1.06265553 | 1 |
| PE(40:4) | 1.04695373 | 1.04931574 | 1.01557279 | 1 |
| PE(40:6) | 1.03972968 | 0.98870268 | 1.04136612 | 1 |
| Cholesterol | 0.91803474 | 0.83017672 | 0.90636493 | 1 |
| CE(16:1) | 0.79707665 | 1.03142836 | 0.94106694 | 1 |
| CE(18:1) | 0.71507121 | 1.0606394 | 0.76736776 | 1 |
| CE(18:2) | 0.86833261 | 0.90755089 | 0.79528551 | 1 |
| CE(20:4) | 1.42343634 | 1.50925476 | 1.39206153 | 1 |
| CE(20:5) | 0.47822541 | 0.98426688 | 0.52619281 | 1 |
| CE(22:6) | 0.46977302 | 1.13646633 | 0.45746034 | 1 |
| TG 52:4/18:1 | 0.9439894 | 3.79244261 | 0.66349662 | 1 |
| TG 52:3/18:1 | 1.08787116 | 3.46853351 | 0.85350626 | 1 |
| TG 54:2/18:0 | 1.07559143 | 1.06097724 | 1.02031321 | 1 |
| TG 52:5/20:4 | 0.95111046 | 0.94243599 | 0.95376133 | 1 |
| TG 54:6/20:4 | 0.95099702 | 1.37444033 | 0.88391879 | 1 |
| TG 54:7/20:4 | 0.97464894 | 1.54022919 | 0.85627423 | 1 |
| TG 56:4/20:4 | 0.73673358 | 0.61495212 | 0.66053781 | 1 |
| TG 56:6/20:4 | 0.9621839 | 0.868923 | 0.9856356 | 1 |
| TG 56:7/20:4 | 0.92092128 | 1.1335638 | 0.87873015 | 1 |
| TG 58:5/20:4 | 0.93020084 | 0.82193456 | 0.94840377 | 1 |
| TG 58:7/20:4 | 0.93722573 | 0.85237293 | 0.89052547 | 1 |
| TG 58:8/22:6 | 1.07576583 | 0.94100942 | 0.99773868 | 1 |
| TG 60:7/22:6 | 0.96749854 | 0.73417824 | 0.82130776 | 1 |
| TG 60:8/22:6 | 1.05266868 | 0.92069031 | 0.9919752 | 1 |
| Sphingosine-1- | 0.81893112 | 0.73370827 | 0.74586929 | 1 |
| phosphate | | | | |
| GB3(d18:1/16:0) | 2.70726534 | 2.32101997 | 1.24086224 | 1 |
| GB3(d18:1/18:0) | 1.72741991 | 0.82100977 | 1.42007533 | 1 |
| GB3(d18:1/24:0) | 1.24531193 | 0.90109689 | 0.98417536 | 1 |
| GB3(d18:1/24:1) | 1.23974776 | 1.99474236 | 1.70175418 | 1 |
| Glu-Cholesterol | 1.16546978 | 1.0592315 | 1.28977423 | 1 |
| Glu-Sitosterol | 0.93789182 | 0.96273937 | 1.05635854 | 1 |
| MG(18:1) | 1.22564038 | 1.32477308 | 1.08297302 | 1 |
| MG(20:4) | 1.37288278 | 0.96352326 | 1.16845401 | 1 |
| MG(18:0) | 0.92851907 | 0.92927545 | 0.90605412 | 1 |
| MG(16:0) | 0.99556967 | 1.03136026 | 0.95593567 | 1 |
| MG(22:6) | 1.33933106 | 1.05594249 | 1.15561146 | 1 |
| DG(18:0_18:1) | 1.0158107 | 0.9205636 | 1.01847638 | 1 |
| DG(18:1/18:1) | 1.07022061 | 0.95162384 | 1.15577943 | 1 |
| DG(16:0_20:4) | 1.05625574 | 0.82770657 | 1.16973462 | 1 |
| DG(18:0_20:4) | 1.06677225 | 0.84833167 | 1.16790474 | 1 |
| DG(18:0_22:6) | 1.15219477 | 0.95279647 | 1.31616862 | 1 |
| DG(18:1_20:4) | 1.03513365 | 0.86622511 | 1.20009423 | 1 |
| GlcCer(d18:1/16:0) | 2.22163513 | 1.19149946 | 1.78189812 | 1 |
| GlcCer(d18:1/18:0) | 1.46205981 | 0.95576955 | 1.36166575 | 1 |
| GlcCer(d18:2/18:0) | 1.57032387 | 0.91791498 | 1.31665568 | 1 |
| GlcCer(d18:1/20:0) | 1.59944265 | 1.10082448 | 1.43078499 | 1 |
| GlcCer(d18:2/20:0) | 1.70565767 | 1.0775347 | 1.39732541 | 1 |
| GlcCer(d18:1/22:0) | 2.02843272 | 1.13859199 | 1.75002022 | 1 |
| GlcCer(d18:1/22:1) | 1.18073058 | 1.0920974 | 1.0967809 | 1 |
| GlcCer(d18:2/22:0) | 1.10416862 | 1.0231995 | 1.30810189 | 1 |
| GlcCer(d18:1/24:1) | 1.63769649 | 1.0627572 | 1.42013123 | 1 |
| GlcCer(d18:1/24:0) | 1.90800362 | 1.10331771 | 1.62785015 | 1 |
| GalCer(d18:1/16:0) | 0.95377573 | 1.03579944 | 0.98731752 | 1 |
| GalCer(d18:1/18:0) | 1.08295723 | 1.0711189 | 1.08097307 | 1 |
| GalCer(d18:2/18:0) | 1.10558046 | 1.07030437 | 1.02805261 | 1 |
| GalCer(d18:1/20:0) | 0.9834055 | 1.01205304 | 0.99950882 | 1 |
| GalCer(d18:2/20:0) | 0.90135811 | 0.91012739 | 0.86083355 | 1 |
| GalCer(d18:1/22:0) | 0.95195268 | 0.96098222 | 0.97624086 | 1 |
| GalCer(d18:1/22:1) | 0.8802212 | 0.95477018 | 0.91442636 | 1 |
| GalCer(d18:2/22:0) | 0.87455475 | 0.84087909 | 0.83798295 | 1 |
| GalCer(d18:1/24:1) | 0.94958635 | 0.91499795 | 0.92923241 | 1 |
| GalCer(d18:1/24:0) | 0.96166861 | 0.90660743 | 0.93744839 | 1 |
| GalCer | 0.8915232 | 0.79920332 | 0.74682448 | 1 |
| alpha(d18:1/16:0) | | | | |
| GluSph | 1.10548038 | 0.94259932 | 0.70868688 | 1 |
| GalSph | 0.9856999 | 0.97263443 | 0.84839691 | 1 |
| 5-HETE | 1.05260938 | 1.24918007 | 0.97156757 | 1 |
| 12-HETE | 1.26557201 | 1.39758942 | 0.90968976 | 1 |
| 15-HETE | 1.38726117 | 1.56716266 | 1.18427095 | 1 |
| 6k-PGF1alpha | 1.11581922 | 1.26819321 | 1.00650322 | 1 |
| TxB2 | 1.661162 | 1.72217815 | 1.45288251 | 1 |
| PGF2alpha | 1.13462868 | 1.35655273 | 1.05179143 | 1 |
| PGE2 | 1.57913809 | 1.51872651 | 1.36194441 | 1 |
| 5,15-DiHETE | 1.47866435 | 1.4028389 | 1.13105795 | 1 |
| 5-iPF2alpha | 1.44800938 | 1.37299339 | 1.42011554 | 1 |
| PGD2 | 1.7946429 | 1.33883673 | 1.4750862 | 1 |
| 9-HOTrE | 1.18369056 | 1.09028544 | 1.13822769 | 1 |
| 9-OxoODE | 1.02194862 | 1.28526776 | 1.00290607 | 1 |
| 13-OxoODE | 0.85385685 | 0.86595781 | 0.84143578 | 1 |
| 9(10)-EpOME | 0.98699842 | 0.95158158 | 1.00759544 | 1 |
| 9-HODE | 1.36011268 | 1.51848008 | 1.16511187 | 1 |
| 13-HODE | 1.14124554 | 1.26747678 | 0.96222687 | 1 |

Informal Sequence Listing

| SEQ ID | | |
|---|---|---|
| NO: | Sequence | Description |
| 1 | APELLGGPSVFLFPPKPKDTLMISRTPEVTCVVVDVSHEDPEVKFNW | Wild-type human Fc |
| | YVDGVEVHNAKTKPREEQYNSTYRVVSVLTVLHQDWLNGKEYKC | sequence |
| | KVSNKALPAPIEKTISKAKGQPREPQVYTLPPSRDELTKNQVSLTCL | positions 231-447 EU |
| | VKGFYPSDIAVEWESNGQPENNYKTTPPVLDSDGSFFLYSKLTVDK | index numbering |
| | SRWQQGNVFSCSVMHEALHNHYTQKSLSLSPGK | |
| 2 | APELLGGPSVFLFPPKPKDTLMISRTPEVTCVVVDVSHEDPEVKFNW | CH2 domain sequence |
| | YVDGVEVHNAKTKPREEQYNSTYRVVSVLTVLHQDWLNGKEYKC | positions 231-340 EU |
| | KVSNKALPAPIEKTISKAK | index numbering |
| 3 | GQPREPQVYTLPPSRDELTKNQVSLTCLVKGFYPSDIAVEWESNGQ | CH3 domain sequence |
| | PENNYKTTPPVLDSDGSFFLYSKLTVDKSRWQQGNVFSCSVMHEAL | Positions 341-447 EU |
| | HNHYTQKSLSLSPGK | index numbering |
| 4 | APELLGGPSVFLFPPKPKDTLMISRTPEVTCVVVDVSHEDPEVKFNW | Clone CH3C.1 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESLGLVWVGYKTTPPVLDSDGSFFLYSKLTVAK | |
| | STWQQGWVFSCSVMHEALHNHYTQKSLSLSPGK | |
| 5 | APELLGGPSVFLFPPKPKDTLMISRTPEVTCVVVDVSHEDPEVKFNW | Clone CH3C.2 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTVWSHYKTTPPVLDSDGSFFLYSKLTVSK | |
| | SEWQQGYVFSCSVMHEALHNHYTQKSLSLSPGK | |
| 6 | APELLGGPSVFLFPPKPKDTLMISRTPEVTCVVVDVSHEDPEVKFNW | Clone CH3C.3 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWSQYKTTPPVLDSDGSFFLYSKLTVEK | |
| | SDWQQGHVFSCSVMHEALHNHYTQKSLSLSPGK | |
| 7 | APELLGGPSVFLFPPKPKDTLMISRTPEVTCVVVDVSHEDPEVKFNW | Clone CH3C.4 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESVGTPWALYKTTPPVLDSDGSFFLYSKLTVLK | |
| | SEWQQGWVFSCSVMHEALHNHYTQKSLSLSPGK | |
| 8 | APELLGGPSVFLFPPKPKDTLMISRTPEVTCVVVDVSHEDPEVKFNW | Clone CH3C.17 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTVWSKYKTTPPVLDSDGSFFLYSKLTVSK | |
| | SEWQQGFVFSCSVMHEALHNHYTQKSLSLSPGK | |
| 9 | APELLGGPSVFLFPPKPKDTLMISRTPEVTCVVVDVSHEDPEVKFNW | Clone CH3C.18 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESLGHVWAVYKTTPPVLDSDGSFFLYSKLTVPK | |
| | STWQQGWVFSCSVMHEALHNHYTQKSLSLSPGK | |
| 10 | APELLGGPSVFLFPPKPKDTLMISRTPEVTCVVVDVSHEDPEVKFNW | Clone CH3C.21 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESLGLVWVGYKTTPPVLDSDGSFFLYSKLTVPK | |
| | STWQQGWVFSCSVMHEALHNHYTQKSLSLSPGK | |
| 11 | APELLGGPSVFLFPPKPKDTLMISRTPEVTCVVVDVSHEDPEVKFNW | Clone CH3C.25 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESMGHVWVGYKTTPPVLDSDGSFFLYSKLTVD | |
| | KSTWQQGWVFSCSVMHEALHNHYTQKSLSLSPGK | |
| 12 | APELLGGPSVFLFPPKPKDTLMISRTPEVTCVVVDVSHEDPEVKFNW | Clone CH3C.34 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESLGLVWVFSKTTPPVLDSDGSFFLYSKLTVPKS | |
| | TWQQGWVFSCSVMHEALHNHYTQKSLSLSPGK | |
| 13 | APELLGGPSVFLFPPKPKDTLMISRTPEVTCVVVDVSHEDPEVKFNW | Clone CH3C.35 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWSSYKTTPPVLDSDGSFFLYSKLTVTKS | |
| | EWQQGFVFSCSVMHEALHNHYTQKSLSLSPGK | |
| 14 | APELLGGPSVFLFPPKPKDTLMISRTPEVTCVVVDVSHEDPEVKFNW | Clone CH3C.44 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWSNYKTTPPVLDSDGSFFLYSKLTVSK | |
| | SEWQQGFVFSCSVMHEALHNHYTQKSLSLSPGK | |
| 15 | APELLGGPSVFLFPPKPKDTLMISRTPEVTCVVVDVSHEDPEVKFNW | Clone CH3C.51 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESLGHVWVGYKTTPPVLDSDGSFFLYSKLTVSK | |
| | SEWQQGWVFSCSVMHEALHNHYTQKSLSLSPGK | |
| 16 | APELLGGPSVFLFPPKPKDTLMISRTPEVTCVVVDVSHEDPEVKFNW | Clone CH3C.3.1-3 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESLGHVWVATKTTPPVLDSDGSFFLYSKLTVPK | |
| | STWQQGWVFSCSVMHEALHNHYTQKSLSLSPGK | |
| 17 | APELLGGPSVFLFPPKPKDTLMISRTPEVTCVVVDVSHEDPEVKFNW | Clone CH3C.3.1-9 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESLGPVWVHTKTTPPVLDSDGSFFLYSKLTVPK | |
| | STWQQGWVFSCSVMHEALHNHYTQKSLSLSPGK | |
| 18 | APELLGGPSVFLFPPKPKDTLMISRTPEVTCVVVDVSHEDPEVKFNW | Clone CH3C.3.2-5 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESLGHVWVDQKTTPPVLDSDGSFFLYSKLTVPK | |
| | STWQQGWVFSCSVMHEALHNHYTQKSLSLSPGK | |
| 19 | APELLGGPSVFLFPPKPKDTLMISRTPEVTCVVVDVSHEDPEVKFNW | Clone CH3C.3.2-19 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESLGHVWVNQKTTPPVLDSDGSFFLYSKLTVPK | |
| | STWQQGWVFSCSVMHEALHNHYTQKSLSLSPGK | |
| 20 | APELLGGPSVFLFPPKPKDTLMISRTPEVTCVVVDVSHEDPEVKFNW | Clone CH3C.3.2-1 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESLGHVWVNFKTTPPVLDSDGSFFLYSKLTVPK | |
| | STWQQGWVFSCSVMHEALHNHYTQKSLSLSPGK | |
| 21 | APELLGGPSVFLFPPKPKDTLMISRTPEVTCVVVDVSHEDPEVKFNW | Clone CH3C.18 variant |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESLGHVWAVYKTTPPVLDSDGSFFLYSKLTVP | |
| | KSTWQQGWVFSCSVMHEALHNHYTQKSLSLSPGK | |
| 22 | APELLGGPSVFLFPPKPKDTLMISRTPEVTCVVVDVSHEDPEVKFNW | Clone CH3C.18 variant |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESLGHVWAVYKTTPPVLDSDGSFFLYSKLTVPK | |
| | STWQQGWVFSCSVMHEALHNHYTQKSLSLSPGK | |
| 23 | APELLGGPSVFLFPPKPKDTLMISRTPEVTCVVVDVSHEDPEVKFNW | Clone CH3C.18 variant |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVYWESLGHVWAVYKTTPPVLDSDGSFFLYSKLTVPK | |
| | STWQQGWVFSCSVMHEALHNHYTQKSLSLSPGK | |
| 24 | APELLGGPSVFLFPPKPKDTLMISRTPEVTCVVVDVSHEDPEVKFNW | Clone CH3C.18 variant |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESLGHVWAVYQTTPPVLDSDGSFFLYSKLTVPK | |
| | STWQQGWVFSCSVMHEALHNHYTQKSLSLSPGK | |
| 25 | APELLGGPSVFLFPPKPKDTLMISRTPEVTCVVVDVSHEDPEVKFNW | Clone CH3C.18 variant |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESLGHVWAVYFTTPPVLDSDGSFFLYSKLTVPK | |
| | STWQQGWVFSCSVMHEALHNHYTQKSLSLSPGK | |
| 26 | APELLGGPSVFLFPPKPKDTLMISRTPEVTCVVVDVSHEDPEVKFNW | Clone CH3C.18 variant |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESLGHVWAVYHTTPPVLDSDGSFFLYSKLTVPK | |
| | STWQQGWVFSCSVMHEALHNHYTQKSLSLSPGK | |
| 27 | APELLGGPSVFLFPPKPKDTLMISRTPEVTCVVVDVSHEDPEVKFNW | Clone CH3C.35.13 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESLGHVWAVYKTTPPVLDSDGSFFLYSKLTVP | |
| | KSTWQQGWVFSCSVMHEALHNHYTQKSLSLSPGK | |
| 28 | APELLGGPSVFLFPPKPKDTLMISRTPEVTCVVVDVSHEDPEVKFNW | Clone CH3C.35.14 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESLGHVWAVYQTTPPVLDSDGSFFLYSKLTVPK | |
| | STWQQGWVFSCSVMHEALHNHYTQKSLSLSPGK | |
| 29 | APELLGGPSVFLFPPKPKDTLMISRTPEVTCVVVDVSHEDPEVKFNW | Clone CH3C.35.15 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESLGHVWAVYQTTPPVLDSDGSFFLYSKLTVP | |
| | KSTWQQGWVFSCSVMHEALHNHYTQKSLSLSPGK | |
| 30 | APELLGGPSVFLFPPKPKDTLMISRTPEVTCVVVDVSHEDPEVKFNW | Clone CH3C.35.16 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESLGHVWVNQKTTPPVLDSDGSFFLYSKLTVP | |
| | KSTWQQGWVFSCSVMHEALHNHYTQKSLSLSPGK | |
| 31 | APELLGGPSVFLFPPKPKDTLMISRTPEVTCVVVDVSHEDPEVKFNW | Clone CH3C.35.17 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESLGHVWVNQQTTPPVLDSDGSFFLYSKLTVPK | |
| | STWQQGWVFSCSVMHEALHNHYTQKSLSLSPGK | |
| 32 | APELLGGPSVFLFPPKPKDTLMISRTPEVTCVVVDVSHEDPEVKFNW | Clone CH3C.35.18 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESLGHVWVNQQTTPPVLDSDGSFFLYSKLTVP | |
| | KSTWQQGWVFSCSVMHEALHNHYTQKSLSLSPGK | |
| 33 | APELLGGPSVFLFPPKPKDTLMISRTPEVTCVVVDVSHEDPEVKFNW | Clone CH3C.35.19 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESYGTEWSSYKTTPPVLDSDGSFFLYSKLTVTK | |
| | SEWQQGFVFSCSVMHEALHNHYTQKSLSLSPGK | |
| 34 | APELLGGPSVFLFPPKPKDTLMISRTPEVTCVVVDVSHEDPEVKFNW | Clone CH3C.35.20 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWSSYKTTPPVLDSDGSFFLYSKLTVTK | |
| | EEWQQGFVFSCSVMHEALHNHYTQKSLSLSPGK | |
| 35 | APELLGGPSVFLFPPKPKDTLMISRTPEVTCVVVDVSHEDPEVKFNW | Clone CH3C.35.21 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESYGTEWSSYKTTPPVLDSDGSFFLYSKLTVTK | |
| | EEWQQGFVFSCSVMHEALHNHYTQKSLSLSPGK | |
| 36 | APELLGGPSVFLFPPKPKDTLMISRTPEVTCVVVDVSHEDPEVKFNW | Clone CH3C.35.22 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESYGTEWSNYKTTPPVLDSDGSFFLYSKLTVTK | |
| | SEWQQGFVFSCSVMHEALHNHYTQKSLSLSPGK | |
| 37 | APELLGGPSVFLFPPKPKDTLMISRTPEVTCVVVDVSHEDPEVKFNW | Clone CH3C.35.23 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWSNYKTTPPVLDSDGSFFLYSKLTVTK | |
| | EEWQQGFVFSCSVMHEALHNHYTQKSLSLSPGK | |
| 38 | APELLGGPSVFLFPPKPKDTLMISRTPEVTCVVVDVSHEDPEVKFNW | Clone CH3C.35.24 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESYGTEWSNYKTTPPVLDSDGSFFLYSKLTVTK | |
| | EEWQQGFVFSCSVMHEALHNHYTQKSLSLSPGK | |
| 39 | APELLGGPSVFLFPPKPKDTLMISRTPEVTCVVVDVSHEDPEVKFNW | Clone CH3C.35.N163 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWSNYKTTPPVLDSDGSFFLYSKLTVTK | |
| | SEWQQGFVFSCSVMHEALHNHYTQKSLSLSPGK | |
| 40 | APELLGGPSVFLFPPKPKDTLMISRTPEVTCVVVDVSHEDPEVKFNW | Clone CH3C.35.K165Q |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWSSYQTTPPVLDSDGSFFLYSKLTVTKS | |
| | EWQQGFVFSCSVMHEALHNHYTQKSLSLSPGK | |
| 41 | APELLGGPSVFLFPPKPKDTLMISRTPEVTCVVVDVSHEDPEVKFNW | Clone CH3C.35.N163. |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | K165Q |
| | VKGFYPSDIAVEWESYGTEWSNYQTTPPVLDSDGSFFLYSKLTVTK | |
| | SEWQQGFVFSCSVMHEALHNHYTQKSLSLSPGK | |
| 42 | APELLGGPSVFLFPPKPKDTLMISRTPEVTCVVVDVSHEDPEVKFNW | Clone CH3C.35.21.1 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWSSYKTTPPVLDSDGSFFLYSKLTVTKS | |
| | EWQQGFVFSCSVMHEALHNHYTQKSLSLSPGK | |
| 43 | APELLGGPSVFLFPPKPKDTLMISRTPEVTCVVVDVSHEDPEVKFNW | Clone CH3C.35.21.2 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWSSYRTTPPVLDSDGSFFLYSKLTVTKS | |
| | EWQQGFVFSCSVMHEALHNHYTQKSLSLSPGK | |
| 44 | APELLGGPSVFLFPPKPKDTLMISRTPEVTCVVVDVSHEDPEVKFNW | Clone CH3C.35.21.3 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWSSYRTTPPVLDSDGSFFLYSKLTVTRE | |
| | EWQQGFVFSCSVMHEALHNHYTQKSLSLSPGK | |
| 45 | APELLGGPSVFLFPPKPKDTLMISRTPEVTCVVVDVSHEDPEVKFNW | Clone CH3C.35.21.4 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWSSYRTTPPVLDSDGSFFLYSKLTVTGE | |
| | EWQQGFVFSCSVMHEALHNHYTQKSLSLSPGK | |
| 46 | APELLGGPSVFLFPPKPKDTLMISRTPEVTCVVVDVSHEDPEVKFNW | Clone CH3C.35.21.5 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWSSYRTTPPVLDSDGSFFLYSKLTVTRE | |
| | EWQQGFVFSCWVMHEALHNHYTQKSLSLSPGK | |
| 47 | APELLGGPSVFLFPPKPKDTLMISRTPEVTCVVVDVSHEDPEVKFNW | Clone CH3C.35.21.6 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWSSYRTTPPVLDSDGSFFLYSKLTVTKE | |
| | EWQQGFVFSCWVMHEALHNHYTQKSLSLSPGK | |
| 48 | APELLGGPSVFLFPPKPKDTLMISRTPEVTCVVVDVSHEDPEVKFNW | Clone CH3C.35.21.7 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWSSYRTTPPVLDSDGSFFLYSKLTVTRE | |
| | EWQQGFVFTCWVMHEALHNHYTQKSLSLSPGK | |
| 49 | APELLGGPSVFLFPPKPKDTLMISRTPEVTCVVVDVSHEDPEVKFNW | Clone CH3C.35.21.8 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWSSYRTTPPVLDSDGSFFLYSKLTVTRE | |
| | EWQQGFVFTCGVMHEALHNHYTQKSLSLSPGK | |
| 50 | APELLGGPSVFLFPPKPKDTLMISRTPEVTCVVVDVSHEDPEVKFNW | Clone CH3C.35.21.9 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWSSYRTTPPVLDSDGSFFLYSKLTVTRE | |
| | EWQQGFVFECWVMHEALHNHYTQKSLSLSPGK | |
| 51 | APELLGGPSVFLFPPKPKDTLMISRTPEVTCVVVDVSHEDPEVKFNW | Clone CH3C.35.21.10 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWSSYRTTPPVLDSDGSFFLYSKLTVTRE | |
| | EWQQGFVFKCWVMHEALHNHYTQKSLSLSPGK | |
| 52 | APELLGGPSVFLFPPKPKDTLMISRTPEVTCVVVDVSHEDPEVKFNW | Clone CH3C.35.21.11 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWSSYRTTPPVLDSDGSFFLYSKLTVTPE | |
| | EWQQGFVFKCWVMHEALHNHYTQKSLSLSPGK | |
| 53 | APELLGGPSVFLFPPKPKDTLMISRTPEVTCVVVDVSHEDPEVKFNW | Clone CH3C.35.21.12 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESYGTEWSSYRTTPPVLDSDGSFFLYSKLTVTR | |
| | EEWQQGFVFSCSVMHEALHNHYTQKSLSLSPGK | |
| 54 | APELLGGPSVFLFPPKPKDTLMISRTPEVTCVVVDVSHEDPEVKFNW | Clone CH3C.35.21.13 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESYGTEWSSYRTTPPVLDSDGSFFLYSKLTVTG | |
| | EEWQQGFVFSCSVMHEALHNHYTQKSLSLSPGK | |
| 55 | APELLGGPSVFLFPPKPKDTLMISRTPEVTCVVVDVSHEDPEVKFNW | Clone CH3C.35.21.14 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESYGTEWSSYRTTPPVLDSDGSFFLYSKLTVTR | |
| | EEWQQGFVFTCWVMHEALHNHYTQKSLSLSPGK | |
| 56 | APELLGGPSVFLFPPKPKDTLMISRTPEVTCVVVDVSHEDPEVKFNW | Clone CH3C.35.21.15 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESYGTEWSSYRTTPPVLDSDGSFFLYSKLTVTG | |
| | EEWQQGFVFTCWVMHEALHNHYTQKSLSLSPGK | |
| 57 | APELLGGPSVFLFPPKPKDTLMISRTPEVTCVVVDVSHEDPEVKFNW | Clone CH3C.35.21.16 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESYGTEWSSYRTTPPVLDSDGSFFLYSKLTVTR | |
| | EEWQQGFVFTCGVMHEALHNHYTQKSLSLSPGK | |
| 58 | APELLGGPSVFLFPPKPKDTLMISRTPEVTCVVVDVSHEDPEVKFNW | Clone CH3C.35.21.17 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWSSYKTTPPVLDSDGSFFLYSKLTVTK | |
| | EEWQQGFVFSCSVMHEALHNHYTQKSLSLSPGK | |
| 59 | APELLGGPSVFLFPPKPKDTLMISRTPEVTCVVVDVSHEDPEVKFNW | Clone CH3C.35.21.18 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWSSYRTTPPVLDSDGSFFLYSKLTVTKE | |
| | EWQQGFVFSCSVMHEALHNHYTQKSLSLSPGK | |
| 60 | APELLGGPSVFLFPPKPKDTLMISRTPEVTCVVVDVSHEDPEVKFNW | Clone CH3C.35.20.1 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESFGTEWSSYKTTPPVLDSDGSFFLYSKLTVTKE | |
| | EWQQGFVFSCSVMHEALHNHYTQKSLSLSPGK | |
| 61 | APELLGGPSVFLFPPKPKDTLMISRTPEVTCVVVDVSHEDPEVKFNW | Clone CH3C.35.20.2 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWASYKTTPPVLDSDGSFFLYSKLTVTK | |
| | EEWQQGFVFSCSVMHEALHNHYTQKSLSLSPGK | |
| 62 | APELLGGPSVFLFPPKPKDTLMISRTPEVTCVVVDVSHEDPEVKFNW | Clone CH3C.35.20.3 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWVSYKTTPPVLDSDGSFFLYSKLTVTK | |
| | EEWQQGFVFSCSVMHEALHNHYTQKSLSLSPGK | |
| 63 | APELLGGPSVFLFPPKPKDTLMISRTPEVTCVVVDVSHEDPEVKFNW | Clone CH3C.35.20.4 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWSSYKTTPPVLDSDGSFFLYSKLTVSKE | |
| | EWQQGFVFSCSVMHEALHNHYTQKSLSLSPGK | |
| 64 | APELLGGPSVFLFPPKPKDTLMISRTPEVTCVVVDVSHEDPEVKFNW | Clone CH3C.35.20.5 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESFGTEWASYKTTPPVLDSDGSFFLYSKLTVTK | |
| | EEWQQGFVFSCSVMHEALHNHYTQKSLSLSPGK | |
| 65 | APELLGGPSVFLFPPKPKDTLMISRTPEVTCVVVDVSHEDPEVKFNW | Clone CH3C.35.20.6 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESFGTEWVSYKTTPPVLDSDGSFFLYSKLTVTK | |
| | EEWQQGFVFSCSVMHEALHNHYTQKSLSLSPGK | |
| 66 | APELLGGPSVFLFPPKPKDTLMISRTPEVTCVVVDVSHEDPEVKFNW | Clone CH3C.35.21.a.1 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESFGTEWSSYKTTPPVLDSDGSFFLYSKLTVTK | |
| | EEWQQGFVFSCSVMHEALHNHYTQKSLSLSPGK | |
| 67 | APELLGGPSVFLFPPKPKDTLMISRTPEVTCVVVDVSHEDPEVKFNW | Clone CH3C.35.21.a.2 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESYGTEWASYKTTPPVLDSDGSFFLYSKLTVTK | |
| | EEWQQGFVFSCSVMHEALHNHYTQKSLSLSPGK | |
| 68 | APELLGGPSVFLFPPKPKDTLMISRTPEVTCVVVDVSHEDPEVKFNW | Clone CH3C.35.21.a.3 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESYGTEWVSYKTTPPVLDSDGSFFLYSKLTVTK | |
| | EEWQQGFVFSCSVMHEALHNHYTQKSLSLSPGK | |
| 69 | APELLGGPSVFLFPPKPKDTLMISRTPEVTCVVVDVSHEDPEVKFNW | Clone CH3C.35.21.a.4 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESYGTEWSSYKTTPPVLDSDGSFFLYSKLTVSK | |
| | EEWQQGFVFSCSVMHEALHNHYTQKSLSLSPGK | |
| 70 | APELLGGPSVFLFPPKPKDTLMISRTPEVTCVVVDVSHEDPEVKFNW | Clone CH3C.35.21.a.5 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESFGTEWASYKTTPPVLDSDGSFFLYSKLTVTK | |
| | EEWQQGFVFSCSVMHEALHNHYTQKSLSLSPGK | |
| 71 | APELLGGPSVFLFPPKPKDTLMISRTPEVTCVVVDVSHEDPEVKFNW | Clone CH3C.35.21.a.6 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESFGTEWVSYKTTPPVLDSDGSFFLYSKLTVTK | |
| | EEWQQGFVFSCSVMHEALHNHYTQKSLSLSPGK | |
| 72 | APELLGGPSVFLFPPKPKDTLMISRTPEVTCVVVDVSHEDPEVKFNW | Clone CH3C.35.23.1 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESFGTEWSNYKTTPPVLDSDGSFFLYSKLTVTK | |
| | EEWQQGFVFSCSVMHEALHNHYTQKSLSLSPGK | |
| 73 | APELLGGPSVFLFPPKPKDTLMISRTPEVTCVVVDVSHEDPEVKFNW | Clone CH3C.35.23.2 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWANYKTTPPVLDSDGSFFLYSKLTVTK | |
| | EEWQQGFVFSCSVMHEALHNHYTQKSLSLSPGK | |
| 74 | APELLGGPSVFLFPPKPKDTLMISRTPEVTCVVVDVSHEDPEVKFNW | Clone CH3C.35.23.3 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWVNYKTTPPVLDSDGSFFLYSKLTVTK | |
| | EEWQQGFVFSCSVMHEALHNHYTQKSLSLSPGK | |
| 75 | APELLGGPSVFLFPPKPKDTLMISRTPEVTCVVVDVSHEDPEVKFNW | Clone CH3C.35.23.4 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWSNYKTTPPVLDSDGSFFLYSKLTVSK | |
| | EEWQQGFVFSCSVMHEALHNHYTQKSLSLSPGK | |
| 76 | APELLGGPSVFLFPPKPKDTLMISRTPEVTCVVVDVSHEDPEVKFNW | Clone CH3C.35.23.5 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESFGTEWANYKTTPPVLDSDGSFFLYSKLTVTK | |
| | EEWQQGFVFSCSVMHEALHNHYTQKSLSLSPGK | |
| 77 | APELLGGPSVFLFPPKPKDTLMISRTPEVTCVVVDVSHEDPEVKFNW | Clone CH3C.35.23.6 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESFGTEWVNYKTTPPVLDSDGSFFLYSKLTVTK | |
| | EEWQQGFVFSCSVMHEALHNHYTQKSLSLSPGK | |
| 78 | APELLGGPSVFLFPPKPKDTLMISRTPEVTCVVVDVSHEDPEVKFNW | Clone CH3C.35.24.1 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESFGTEWSNYKTTPPVLDSDGSFFLYSKLTVTK | |
| | EEWQQGFVFSCSVMHEALHNHYTQKSLSLSPGK | |
| 79 | APELLGGPSVFLFPPKPKDTLMISRTPEVTCVVVDVSHEDPEVKFNW | Clone CH3C.35.24.2 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESYGTEWANYKTTPPVLDSDGSFFLYSKLTVT | |
| | KEEWQQGFVFSCSVMHEALHNHYTQKSLSLSPGK | |
| 80 | APELLGGPSVFLFPPKPKDTLMISRTPEVTCVVVDVSHEDPEVKFNW | Clone CH3C.35.24.3 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESYGTEWVNYKTTPPVLDSDGSFFLYSKLTVT | |
| | KEEWQQGFVFSCSVMHEALHNHYTQKSLSLSPGK | |
| 81 | APELLGGPSVFLFPPKPKDTLMISRTPEVTCVVVDVSHEDPEVKFNW | Clone CH3C.35.24.4 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESYGTEWSNYKTTPPVLDSDGSFFLYSKLTVSK | |
| | EEWQQGFVFSCSVMHEALHNHYTQKSLSLSPGK | |
| 82 | APELLGGPSVFLFPPKPKDTLMISRTPEVTCVVVDVSHEDPEVKFNW | Clone CH3C.35.24.5 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESFGTEWANYKTTPPVLDSDGSFFLYSKLTVTK | |
| | EEWQQGFVFSCSVMHEALHNHYTQKSLSLSPGK | |
| 83 | APELLGGPSVFLFPPKPKDTLMISRTPEVTCVVVDVSHEDPEVKFNW | Clone CH3C.35.24.6 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESFGTEWVNYKTTPPVLDSDGSFFLYSKLTVTK | |
| | EEWQQGFVFSCSVMHEALHNHYTQKSLSLSPGK | |
| 84 | APELLGGPSVFLFPPKPKDTLMISRTPEVTCVVVDVSHEDPEVKFNW | Clone CH3C.35.21.17.1 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESFGTEWSSYKTTPPVLDSDGSFFLYSKLTVTKE | |
| | EWQQGFVFSCSVMHEALHNHYTQKSLSLSPGK | |
| 85 | APELLGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWASYKTTPPVLDSDGSFFLYSKLTVTK | |
| | EEWQQGFVFSCSVMHEALHNHYTQKSLSLSPGK | |
| 86 | APELLGGPSVFLFPPKPKDTLMISRTPEVTCVVVDVSHEDPEVKFNW | Clone CH3C.35.21.17.3 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWVSYKTTPPVLDSDGSFFLYSKLTVTK | |
| | EEWQQGFVFSCSVMHEALHNHYTQKSLSLSPGK | |
| 87 | APELLGGPSVFLFPPKPKDTLMISRTPEVTCVVVDVSHEDPEVKFNW | Clone CH3C.35.21.17.4 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWSSYKTTPPVLDSDGSFFLYSKLTVSKE | |
| | EWQQGFVFSCSVMHEALHNHYTQKSLSLSPGK | |
| 88 | APELLGGPSVFLFPPKPKDTLMISRTPEVTCVVVDVSHEDPEVKFNW | Clone CH3C.35.21.17.5 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESFGTEWASYKTTPPVLDSDGSFFLYSKLTVTK | |
| | EEWQQGFVFSCSVMHEALHNHYTQKSLSLSPGK | |
| 89 | APELLGGPSVFLFPPKPKDTLMISRTPEVTCVVVDVSHEDPEVKFNW | Clone CH3C.35.21.17.6 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESFGTEWVSYKTTPPVLDSDGSFFLYSKLTVTK | |
| | EEWQQGFVFSCSVMHEALHNHYTQKSLSLSPGK | |
| 90 | APELLGGPSVFLFPPKPKDTLMISRTPEVTCVVVDVSHEDPEVKFNW | Clone CH3C.35.N390 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWSNYKTTPPVLDSDGSFFLYSKLTVTK | |
| | SEWQQGFVFSCSVMHEALHNHYTQKSLSLSPGK | |
| 91 | MPPPRTGRGLLWLGLVLSSVCVALGSETQANSTTDALNVLLIIVDDL | Full-length human IDS |
| | RPSLGCYGDKLVRSPNIDQLASHSLLFQNAFAQQAVCAPSRVSFLTG | polypeptide sequence |
| | RRPDTTRLYDFNSYWRVHAGNFSTIPQYFKENGYVTMSVGKVFHP | |
| | GISSNHTDDSPYSWSFPPYHPSSEKYENTKTCRGPDGELHANLLCPV | |
| | DVLDVPEGTLPDKQSTEQAIQLLEKMKTSASPFFLAVGYHKPHIPFR | |
| | YPKEFQKLYPLENITLAPDPEVPDGLPPVAYNPWMDIRQREDVQAL | |
| | NISVPYGPIPVDFQRKIRQSYFASVSYLDTQVGRLLSALDDLQLANS | |
| | TIIAFTSDHGWALGEHGEWAKYSNFDVATHVPLIFYVPGRTASLPE | |
| | AGEKLFPYLDPFDSASQLMEPGRQSMDLVELVSLFPTLAGLAGLQV | |
| | PPRCPVPSFHVELCREGKNLLKHFRFRDLEEDPYLPGNPRELIAYSQ | |
| | YPRPSDIPQWNSDKPSLKDIKIMGYSIRTIDYRYTVWVGFNPDEFLA | |
| | NFSDIHAGELYFVDSDPLQDHNMYNDSQGGDLFQLLMP | |
| 92 | TDALNVLLIIVDDLRPSLGCYGDKLVRSPNIDQLASHSLLFQNAFAQ | Mature human IDS |
| | QAVCAPSRVSFLTGRRPDTTRLYDFNSYWRVHAGNFSTIPQYFKEN | polypeptide sequence |
| | GYVTMSVGKVFHPGISSNHTDDSPYSWSFPPYHPSSEKYENTKTCR | |
| | GPDGELHANLLCPVDVLDVPEGTLPDKQSTEQAIQLLEKMKTSASP | |
| | FFLAVGYHKPHIPFRYPKEFQKLYPLENITLAPDPEVPDGLPPVAYN | |
| | PWMDIRQREDVQALNISVPYGPIPVDFQRKIRQSYFASVSYLDTQVG | |
| | RLLSALDDLQLANSTIIAFTSDHGWALGEHGEWAKYSNFDVATHVP | |
| | LIFYVPGRTASLPEAGEKLFPYLDPFDSASQLMEPGRQSMDLVELVS | |
| | LFPTLAGLAGLQVPPRCPVPSFHVELCREGKNLLKHFRFRDLEEDPY | |
| | LPGNPRELIAYSQYPRPSDIPQWNSDKPSLKDIKIMGYSIRTIDYRYT | |
| | VWVGFNPDEFLANFSDIHAGELYFVDSDPLQDHNMYNDSQGGDLF | |
| | QLLMP | |
| 93 | EPKSCDKTHTCPPCP | Human IgG1 hinge |
| | | amino acid sequence |
| 94 | MMDQARSAFSNLFGGEPLSYTRFSLARQVDGDNSHVEMKLAVDEE | Human transferrin |
| | ENADNNTKANVTKPKRCSGSICYGTIAVIVFFLIGFMIGYLGYCKGV | receptor protein 1 |
| | EPKTECERLAGTESPVREEPGEDFPAARRLYWDDLKRKLSEKLDST | (TFR1) |
| | DFTGTIKLLNENSYVPREAGSQKDENLALYVENQFREFKLSKVWRD | |
| | QHFVKIQVKDSAQNSVIIVDKNGRLVYLVENPGGYVAYSKAATVT | |
| | GKLVHANFGTKKDFEDLYTPVNGSIVIVRAGKITFAEKVANAESLN | |
| | AIGVLIYMDQTKFPIVNAELSFFGHAHLGTGDPYTPGFPSFNHTQFPP | |
| | SRSSGLPNIPVQTISRAAAEKLFGNMEGDCPSDWKTDSTCRMVTSES | |
| | KNVKLTVSNVLKEIKILNIFGVIKGFVEPDHYVVVGAQRDAWGPGA | |
| | AKSGVGTALLLKLAQMFSDMVLKDGFQPSRSIIFASWSAGDFGSVG | |
| | ATEWLEGYLSSLHLKAFTYINLDKAVLGTSNFKVSASPLLYTLIEKT | |
| | MQNVKHPVTGQFLYQDSNWASKVEKLTLDNAAFPFLAYSGIPAVS | |
| | FCFCEDTDYPYLGTTMDTYKELIERIPELNKVARAAAEVAGQFVIKL | |
| | THDVELNLDYERYNSQLLSFVRDLNQYRADIKEMGLSLQWLYSAR | |
| | GDFFRATSRLTTDFGNAEKTDRFVMKKLNDRVMRVEYHFLSPYVS | |
| | PKESPFRHVFWGSGSHTLPALLENLKLRKQNNGAFNETLFRNQLAL | |
| | ATWTIQGAANALSGDVWDIDNEF | |
| 95 | APELLGGPSVFLFPPKPKDTLMISRTPEVTCVVVDVSHEDPEVKFNW | Clone CH3C.35.21 with |
| | YVDGVEVHNAKTKPREEQYNSTYRVVSVLTVLHQDWLNGKEYKC | knob mutation |
| | KVSNKALPAPIEKTISKAKGQPREPQVYTLPPSRDELTKNQVSLWCL | |
| | VKGFYPSDIAVWWESYGTEWSSYKTTPPVLDSDGSFFLYSKLTVTK | |
| | EEWQQGFVFSCSVMHEALHNHYTQKSLSLSPGK | |
| 96 | APEAAGGPSVFLFPPKPKDTLMISRTPEVTCVVVDVSHEDPEVKFN | Clone CH3C.35.21 with |
| | WYVDGVEVHNAKTKPREEQYNSTYRVVSVLTVLHQDWLNGKEY | knob and LALA |
| | KCKVSNKALPAPIEKTISKAKGQPREPQVYTLPPSRDELTKNQVSLW | mutations |
| | CLVKGFYPSDIAVWWESYGTEWSSYKTTPPVLDSDGSFFLYSKLTV | |
| | TKEEWQQGFVFSCSVMHEALHNHYTQKSLSLSPGK | |
| 97 | APELLGGPSVFLFPPKPKDTLYITREPEVTCVVVDVSHEDPEVKFNW | Clone CH3C.35.21 with |
| | YVDGVEVHNAKTKPREEQYNSTYRVVSVLTVLHQDWLNGKEYKC | knob and YTE mutations |
| | KVSNKALPAPIEKTISKAKGQPREPQVYTLPPSRDELTKNQVSLWCL | |
| | VKGFYPSDIAVWWESYGTEWSSYKTTPPVLDSDGSFFLYSKLTVTK | |
| | EEWQQGFVFSCSVMHEALHNHYTQKSLSLSPGK | |
| 98 | APEAAGGPSVFLFPPKPKDTLYITREPEVTCVVVDVSHEDPEVKFN | Clone CH3C.35.21 with |
| | WYVDGVEVHNAKTKPREEQYNSTYRVVSVLTVLHQDWLNGKEY | knob, LALA, and YTE |
| | KCKVSNKALPAPIEKTISKAKGQPREPQVYTLPPSRDELTKNQVSLW | mutations |
| | CLVKGFYPSDIAVWWESYGTEWSSYKTTPPVLDSDGSFFLYSKLTV | |
| | TKEEWQQGFVFSCSVMHEALHNHYTQKSLSLSPGK | |
| 99 | APELLGGPSVFLFPPKPKDTLMISRTPEVTCVVVDVSHEDPEVKFNW | Fc sequence with hole |
| | YVDGVEVHNAKTKPREEQYNSTYRVVSVLTVLHQDWLNGKEYKC | mutations |
| | KVSNKALPAPIEKTISKAKGQPREPQVYTLPPSRDELTKNQVSLSCA | |
| | VKGFYPSDIAVEWESNGQPENNYKTTPPVLDSDGSFFLVSKLTVDK | |
| | SRWQQGNVFSCSVMHEALHNHYTQKSLSLSPGK | |
| 100 | APEAAGGPSVFLFPPKPKDTLMISRTPEVTCVVVDVSHEDPEVKFN | Fc sequence with hole |
| | WYVDGVEVHNAKTKPREEQYNSTYRVVSVLTVLHQDWLNGKEY | and LALA mutations |
| | KCKVSNKALPAPIEKTISKAKGQPREPQVYTLPPSRDELTKNQVSLS | |
| | CAVKGFYPSDIAVEWESNGQPENNYKTTPPVLDSDGSFFLVSKLTV | |
| | DKSRWQQGNVFSCSVMHEALHNHYTQKSLSLSPGK | |
| 101 | APELLGGPSVFLFPPKPKDTLYITREPEVTCVVVDVSHEDPEVKFNW | Fc sequence with hole |
| | YVDGVEVHNAKTKPREEQYNSTYRVVSVLTVLHQDWLNGKEYKC | and YTE mutations |
| | KVSNKALPAPIEKTISKAKGQPREPQVYTLPPSRDELTKNQVSLSCA | |
| | VKGFYPSDIAVEWESNGQPENNYKTTPPVLDSDGSFFLVSKLTVDK | |
| | SRWQQGNVFSCSVMHEALHNHYTQKSLSLSPGK | |
| 102 | APEAAGGPSVFLFPPKPKDTLYITREPEVTCVVVDVSHEDPEVKFN | Fc sequence with hole, |
| | WYVDGVEVHNAKTKPREEQYNSTYRVVSVLTVLHQDWLNGKEY | LALA, and YTE |
| | KCKVSNKALPAPIEKTISKAKGQPREPQVYTLPPSRDELTKNQVSLS | mutations |
| | CAVKGFYPSDIAVEWESNGQPENNYKTTPPVLDSDGSFFLVSKLTV | |
| | DKSRWQQGNVFSCSVMHEALHNHYTQKSLSLSPGK | |
| 103 | APELLGGPSVFLFPPKPKDTLMISRTPEVTCVVVDVSHEDPEVKFNW | Clone CH3C.35.21 with |
| | YVDGVEVHNAKTKPREEQYNSTYRVVSVLTVLHQDWLNGKEYKC | hole mutations |
| | KVSNKALPAPIEKTISKAKGQPREPQVYTLPPSRDELTKNQVSLSCA | |
| | VKGFYPSDIAVWWESYGTEWSSYKTTPPVLDSDGSFFLVSKLTVTK | |
| | EEWQQGFVFSCSVMHEALHNHYTQKSLSLSPGK | |
| 104 | APEAAGGPSVFLFPPKPKDTLMISRTPEVTCVVVDVSHEDPEVKFN | Clone CH3C.35.21 with |
| | WYVDGVEVHNAKTKPREEQYNSTYRVVSVLTVLHQDWLNGKEY | hole and LALA |
| | KCKVSNKALPAPIEKTISKAKGQPREPQVYTLPPSRDELTKNQVSLS | mutations |
| | CAVKGFYPSDIAVWWESYGTEWSSYKTTPPVLDSDGSFFLVSKLTV | |
| | TKEEWQQGFVFSCSVMHEALHNHYTQKSLSLSPGK | |
| 105 | APELLGGPSVFLFPPKPKDTLYITREPEVTCVVVDVSHEDPEVKFNW | Clone CH3C.35.21 with |
| | YVDGVEVHNAKTKPREEQYNSTYRVVSVLTVLHQDWLNGKEYKC | hole and YTE mutations |
| | KVSNKALPAPIEKTISKAKGQPREPQVYTLPPSRDELTKNQVSLSCA | |
| | VKGFYPSDIAVWWESYGTEWSSYKTTPPVLDSDGSFFLVSKLTVTK | |
| | EEWQQGFVFSCSVMHEALHNHYTQKSLSLSPGK | |
| 106 | APEAAGGPSVFLFPPKPKDTLYITREPEVTCVVVDVSHEDPEVKFN | Clone CH3C.35.21 with |
| | WYVDGVEVHNAKTKPREEQYNSTYRVVSVLTVLHQDWLNGKEY | hole, LALA, and YTE |
| | KCKVSNKALPAPIEKTISKAKGQPREPQVYTLPPSRDELTKNQVSLS | mutations |
| | CAVKGFYPSDIAVWWESYGTEWSSYKTTPPVLDSDGSFFLVSKLTV | |
| | TKEEWQQGFVFSCSVMHEALHNHYTQKSLSLSPGK | |
| 107 | APELLGGPSVFLFPPKPKDTLMISRTPEVTCVVVDVSHEDPEVKFNW | Fc sequence with knob |
| | YVDGVEVHNAKTKPREEQYNSTYRVVSVLTVLHQDWLNGKEYKC | mutation |
| | KVSNKALPAPIEKTISKAKGQPREPQVYTLPPSRDELTKNQVSLWCL | |
| | VKGFYPSDIAVEWESNGQPENNYKTTPPVLDSDGSFFLYSKLTVDK | |
| | SRWQQGNVFSCSVMHEALHNHYTQKSLSLSPGK | |
| 108 | APEAAGGPSVFLFPPKPKDTLMISRTPEVTCVVVDVSHEDPEVKFN | Fc sequence with knob |
| | WYVDGVEVHNAKTKPREEQYNSTYRVVSVLTVLHQDWLNGKEY | and LALA mutations |
| | KCKVSNKALPAPIEKTISKAKGQPREPQVYTLPPSRDELTKNQVSLW | |
| | CLVKGFYPSDIAVEWESNGQPENNYKTTPPVLDSDGSFFLYSKLTV | |
| | DKSRWQQGNVFSCSVMHEALHNHYTQKSLSLSPGK | |
| 109 | APELLGGPSVFLFPPKPKDTLYITREPEVTCVVVDVSHEDPEVKFNW | Fc sequence with knob |
| | YVDGVEVHNAKTKPREEQYNSTYRVVSVLTVLHQDWLNGKEYKC | and YTE mutations |
| | KVSNKALPAPIEKTISKAKGQPREPQVYTLPPSRDELTKNQVSLWCL | |
| | VKGFYPSDIAVEWESNGQPENNYKTTPPVLDSDGSFFLYSKLTVDK | |
| | SRWQQGNVFSCSVMHEALHNHYTQKSLSLSPGK | |
| 110 | APEAAGGPSVFLFPPKPKDTLYITREPEVTCVVVDVSHEDPEVKFN | Fc sequence with knob, |
| | WYVDGVEVHNAKTKPREEQYNSTYRVVSVLTVLHQDWLNGKEY | LALA, and YTE |
| | KCKVSNKALPAPIEKTISKAKGQPREPQVYTLPPSRDELTKNQVSLW | mutations |
| | CLVKGFYPSDIAVEWESNGQPENNYKTTPPVLDSDGSFFLYSKLTV | |
| | DKSRWQQGNVFSCSVMHEALHNHYTQKSLSLSPGK | |
| 111 | DKTHTCPPCP | Portion of human IgG1 |
| | | hinge sequence |
| 112 | SETQANSTTD ALNVLLIIVD DLRPSLGCYG DKLVRSPNID | IDS sequence |
| | QLASHSLLFQ NAFAQQAVCA PSRVSFLTGR RPDTTRLYDF | (cysteine modified to |
| | NSYWRVHAGN FSTIPQYFKE NGYVTMSVGK VFHPGISSNH | formylglycine double |
| | TDDSPYSWSF PPYHPSSEKY ENTKTCRGPD GELHANLLCP | underlined) |
| | VDVLDVPEGT LPDKQSTEQA IQLLEKMKTS ASPFFLAVGY | |
| | HKPHIPFRYP KEFQKLYPLE NITLAPDPEV PDGLPPVAYN | |
| | PWMDIRQRED VQALNISVPY GPIPVDFQRK IRQSYFASVS | |
| | YLDTQVGRLL SALDDLQLAN STIIAFTSDH GWALGEHGEW | |
| | AKYSNFDVAT HVPLIFYVPG RTASLPEAGE KLFPYLDPFD | |
| | SASQLMEPGR QSMDLVELVS LFPTLAGLAG LQVPPRCPVP | |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMP | |
| 113 | SETQANSTTD ALNVLLIIVD DLRPSLGCYG DKLVRSPNID | IDS-Fc fusion |
| | QLASHSLLFQ NAFAQQAVCA PSRVSFLTGR RPDTTRLYDF | polypeptide with IDS |
| | NSYWRVHAGN FSTIPQYFKE NGYVTMSVGK VFHPGISSNH | sequence underlined |
| | TDDSPYSWSF PPYHPSSEKY ENTKTCRGPD GELHANLLCP | (cysteine modified to |
| | VDVLDVPEGT LPDKQSTEQA IQLLEKMKTS ASPFFLAVGY | formylglycine double |
| | HKPHIPFRYP KEFQKLYPLE NITLAPDPEV PDGLPPVAYN | underlined) and hole and |
| | PWMDIRQRED VQALNISVPY GPIPVDFQRK IRQSYFASVS | LALAmutations |
| | YLDTQVGRLL SALDDLQLAN STIIAFTSDH GWALGEHGEW | |
| | AKYSNFDVAT HVPLIFYVPG RTASLPEAGE KLFPYLDPFD | |
| | SASQLMEPGR QSMDLVELVS LFPTLAGLAG LQVPPRCPVP | |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APEAAGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPGK | |
| 114 | DKTHTCPPCP APEAAGGPSV FLFPPKPKDT LMISRTPEVT | Clone CH3C.35.21 with |
| | CVVVDVSHED PEVKFNWYVD GVEVHNAKTK PREEQYNSTY | knob and LALA |
| | RVVSVLTVLH QDWLNGKEYK CKVSNKALPA PIEKTISKAK | mutations and portion of |
| | GQPREPQVYT LPPSRDELTK NQVSLWCLVK GFYPSDIAVW | human IgG1 hinge |
| | WESYGTEWSS YKTTPPVLDS DGSFFLYSKL TVTKEEWQQG | sequence |
| | FVFSCSVMHE ALHNHYTQKS LSLSPGK | |
| 115 | SETQANSTTD ALNVLLIIVD DLRPSLGCYG DKLVRSPNID | IDS-Fc fusion |
| | QLASHSLLFQ NAFAQQAVCA PSRVSFLTGR RPDTTRLYDF | polypeptide with IDS |
| | NSYWRVHAGN FSTIPQYFKE NGYVTMSVGK VFHPGISSNH | sequence underlined |
| | TDDSPYSWSF PPYHPSSEKY ENTKTCRGPD GELHANLLCP | (cysteine modified to |
| | VDVLDVPEGT LPDKQSTEQA IQLLEKMKTS ASPFFLAVGY | formylglycine double |
| | HKPHIPFRYP KEFQKLYPLE NITLAPDPEV PDGLPPVAYN | underlined) and hole |
| | PWMDIRQRED VQALNISVPY GPIPVDFQRK IRQSYFASVS | mutations |
| | YLDTQVGRLL SALDDLQLAN STIIAFTSDH GWALGEHGEW | |
| | AKYSNFDVAT HVPLIFYVPG RTASLPEAGE KLFPYLDPFD | |
| | SASQLMEPGR QSMDLVELVS LFPTLAGLAG LQVPPRCPVP | |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APELLGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPGK | |
| 116 | SETQANSTTD ALNVLLIIVD DLRPSLGCYG DKLVRSPNID | IDS-Fc fusion |
| | QLASHSLLFQ NAFAQQAVCA PSRVSFLTGR RPDTTRLYDF | polypeptide with IDS |
| | NSYWRVHAGN FSTIPQYFKE NGYVTMSVGK VFHPGISSNH | sequence underlined |
| | TDDSPYSWSF PPYHPSSEKY ENTKTCRGPD GELHANLLCP | (cysteine modified to |
| | VDVLDVPEGT LPDKQSTEQA IQLLEKMKTS ASPFFLAVGY | formylglycine double |
| | HKPHIPFRYP KEFQKLYPLE NITLAPDPEV PDGLPPVAYN | underlined) and knob |
| | PWMDIRQRED VQALNISVPY GPIPVDFQRK IRQSYFASVS | mutation |
| | YLDTQVGRLL SALDDLQLAN STIIAFTSDH GWALGEHGEW | |
| | AKYSNFDVAT HVPLIFYVPG RTASLPEAGE KLFPYLDPFD | |
| | SASQLMEPGR QSMDLVELVS LFPTLAGLAG LQVPPRCPVP | |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APELLGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLWCLVK | |
| | GFYPSDIAVE WESNGQPENN YKTTPPVLDS DGSFFLYSKL | |
| | TVDKSRWQQG NVFSCSVMHE ALHNHYTQKS LSLSPGK | |
| 117 | APEAAGGPSV FLFPPKPKDT LMISRTPEVT CVVVDVSHED | Clone CH3C.35.21.17 |
| | PEVKFNWYVD GVEVHNAKTK PREEQYNSTY RVVSVLTVLH | with knob and LALA |
| | QDWLNGKEYK CKVSNKALPA PIEKTISKAK GQPREPQVYT | mutations |
| | LPPSRDELTK NQVSLWCLVK GFYPSDIAVL WESYGTEWSS | |
| | YKTTPPVLDS DGSFFLYSKL TVTKEEWQQG FVFSCSVMHE | |
| | ALHNHYTQKS LSLSPGK | |
| 118 | APELLGGPSVFLFPPKPKDTLMISRTPEVTCVVVDVSHEDPEVKFNW | Clone CH3C.35.20.1 |
| | YVDGVEVHNAKTKPREEQYNSTYRVVSVLTVLHQDWLNGKEYKC | with knob mutation |
| | KVSNKALPAPIEKTISKAKGQPREPQVYTLPPSRDELTKNQVSLWCL | |
| | VKGFYPSDIAVEWESFGTEWSSYKTTPPVLDSDGSFFLYSKLTVTKE | |
| | EWQQGFVFSCSVMHEALHNHYTQKSLSLSPGK | |
| 119 | APEAAGGPSVFLFPPKPKDTLMISRTPEVTCVVVDVSHEDPEVKFN | Clone CH3C.35.20.1 |
| | WYVDGVEVHNAKTKPREEQYNSTYRVVSVLTVLHQDWLNGKEY | with knob and LALA |
| | KCKVSNKALPAPIEKTISKAKGQPREPQVYTLPPSRDELTKNQVSLW | mutations |
| | CLVKGFYPSDIAVEWESFGTEWSSYKTTPPVLDSDGSFFLYSKLTVT | |
| | KEEWQQGFVFSCSVMHEALHNHYTQKSLSLSPGK | |
| 120 | APEAAGGPSVFLFPPKPKDTLMISRTPEVTCVVVDVSHEDPEVKFN | Clone CH3C.35.20.1 |
| | WYVDGVEVHNAKTKPREEQYNSTYRVVSVLTVLHQDWLNGKEY | with knob and LALAPG |
| | KCKVSNKALGAPIEKTISKAKGQPREPQVYTLPPSRDELTKNQVSL | mutations |
| | WCLVKGFYPSDIAVEWESFGTEWSSYKTTPPVLDSDGSFFLYSKLT | |
| | VTKEEWQQGFVFSCSVMHEALHNHYTQKSLSLSPGK | |
| 121 | APELLGGPSVFLFPPKPKDTLYITREPEVTCVVVDVSHEDPEVKFNW | Clone CH3C.35.20.1 |
| | YVDGVEVHNAKTKPREEQYNSTYRVVSVLTVLHQDWLNGKEYKC | with knob and YTE |
| | KVSNKALPAPIEKTISKAKGQPREPQVYTLPPSRDELTKNQVSLWCL | mutations |
| | VKGFYPSDIAVEWESFGTEWSSYKTTPPVLDSDGSFFLYSKLTVTKE | |
| | EWQQGFVFSCSVMHEALHNHYTQKSLSLSPGK | |
| 122 | APEAAGGPSVFLFPPKPKDTLYITREPEVTCVVVDVSHEDPEVKFN | Clone CH3C.35.20.1 |
| | WYVDGVEVHNAKTKPREEQYNSTYRVVSVLTVLHQDWLNGKEY | with knob, LALA, and |
| | KCKVSNKALPAPIEKTISKAKGQPREPQVYTLPPSRDELTKNQVSLW | YTEmutations |
| | CLVKGFYPSDIAVEWESFGTEWSSYKTTPPVLDSDGSFFLYSKLTVT | |
| | KEEWQQGFVFSCSVMHEALHNHYTQKSLSLSPGK | |
| 123 | APEAAGGPSVFLFPPKPKDTLYITREPEVTCVVVDVSHEDPEVKFN | Clone CH3C.35.20.1 |
| | WYVDGVEVHNAKTKPREEQYNSTYRVVSVLTVLHQDWLNGKEY | with knob, LALAPG, |
| | KCKVSNKALGAPIEKTISKAKGQPREPQVYTLPPSRDELTKNQVSL | and YTEmutations |
| | WCLVKGFYPSDIAVEWESFGTEWSSYKTTPPVLDSDGSFFLYSKLT | |
| | VTKEEWQQGFVFSCSVMHEALHNHYTQKSLSLSPGK | |
| 124 | APELLGGPSVFLFPPKPKDTLMISRTPEVTCVVVDVSHEDPEVKFNW | Clone CH3C.35.20.1 |
| | YVDGVEVHNAKTKPREEQYNSTYRVVSVLTVLHQDWLNGKEYKC | with hole mutations |
| | KVSNKALPAPIEKTISKAKGQPREPQVYTLPPSRDELTKNQVSLSCA | |
| | VKGFYPSDIAVEWESFGTEWSSYKTTPPVLDSDGSFFLVSKLTVTKE | |
| | EWQQGFVFSCSVMHEALHNHYTQKSLSLSPGK | |
| 125 | APEAAGGPSVFLFPPKPKDTLMISRTPEVTCVVVDVSHEDPEVKFN | Clone CH3C.35.20.1 |
| | WYVDGVEVHNAKTKPREEQYNSTYRVVSVLTVLHQDWLNGKEY | with hole and LALA |
| | KCKVSNKALPAPIEKTISKAKGQPREPQVYTLPPSRDELTKNQVSLS | mutations |
| | CAVKGFYPSDIAVEWESFGTEWSSYKTTPPVLDSDGSFFLVSKLTVT | |
| | KEEWQQGFVFSCSVMHEALHNHYTQKSLSLSPGK | |
| 126 | APEAAGGPSVFLFPPKPKDTLMISRTPEVTCVVVDVSHEDPEVKFN | Clone CH3C.35.20.1 |
| | WYVDGVEVHNAKTKPREEQYNSTYRVVSVLTVLHQDWLNGKEY | with hole and LALAPG |
| | KCKVSNKALGAPIEKTISKAKGQPREPQVYTLPPSRDELTKNQVSLS | mutations |
| | CAVKGFYPSDIAVEWESFGTEWSSYKTTPPVLDSDGSFFLVSKLTVT | |
| | KEEWQQGFVFSCSVMHEALHNHYTQKSLSLSPGK | |
| 127 | APELLGGPSVFLFPPKPKDTLYITREPEVTCVVVDVSHEDPEVKFNW | Clone CH3C.35.20.1 |
| | YVDGVEVHNAKTKPREEQYNSTYRVVSVLTVLHQDWLNGKEYKC | with hole and YTE |
| | KVSNKALPAPIEKTISKAKGQPREPQVYTLPPSRDELTKNQVSLSCA | mutations |
| | VKGFYPSDIAVEWESFGTEWSSYKTTPPVLDSDGSFFLVSKLTVTKE | |
| | EWQQGFVFSCSVMHEALHNHYTQKSLSLSPGK | |
| 128 | APEAAGGPSVFLFPPKPKDTLYITREPEVTCVVVDVSHEDPEVKFN | Clone CH3C.35.20.1 |
| | WYVDGVEVHNAKTKPREEQYNSTYRVVSVLTVLHQDWLNGKEY | with hole, LALA, and |
| | KCKVSNKALPAPIEKTISKAKGQPREPQVYTLPPSRDELTKNQVSLS | YTEmutations |
| | CAVKGFYPSDIAVEWESFGTEWSSYKTTPPVLDSDGSFFLVSKLTVT | |
| | KEEWQQGFVFSCSVMHEALHNHYTQKSLSLSPGK | |
| 129 | APEAAGGPSVFLFPPKPKDTLYITREPEVTCVVVDVSHEDPEVKFN | Clone CH3C.35.20.1 |
| | WYVDGVEVHNAKTKPREEQYNSTYRVVSVLTVLHQDWLNGKEY | with hole, LALAPG, and |
| | KCKVSNKALGAPIEKTISKAKGQPREPQVYTLPPSRDELTKNQVSLS | YTEmutations |
| | CAVKGFYPSDIAVEWESFGTEWSSYKTTPPVLDSDGSFFLVSKLTVT | |
| | KEEWQQGFVFSCSVMHEALHNHYTQKSLSLSPGK | |
| 130 | APELLGGPSVFLFPPKPKDTLMISRTPEVTCVVVDVSHEDPEVKFNW | Clone CH3C.35.23.2 |
| | YVDGVEVHNAKTKPREEQYNSTYRVVSVLTVLHQDWLNGKEYKC | with knob mutation |
| | KVSNKALPAPIEKTISKAKGQPREPQVYTLPPSRDELTKNQVSLWCL | |
| | VKGFYPSDIAVEWESYGTEWANYKTTPPVLDSDGSFFLYSKLTVTK | |
| | EEWQQGFVFSCSVMHEALHNHYTQKSLSLSPGK | |
| 131 | APEAAGGPSVFLFPPKPKDTLMISRTPEVTCVVVDVSHEDPEVKFN | Clone CH3C.35.23.2 |
| | WYVDGVEVHNAKTKPREEQYNSTYRVVSVLTVLHQDWLNGKEY | with knob and LALA |
| | KCKVSNKALPAPIEKTISKAKGQPREPQVYTLPPSRDELTKNQVSLW | mutations |
| | CLVKGFYPSDIAVEWESYGTEWANYKTTPPVLDSDGSFFLYSKLTV | |
| | TKEEWQQGFVFSCSVMHEALHNHYTQKSLSLSPGK | |
| 132 | APEAAGGPSVFLFPPKPKDTLMISRTPEVTCVVVDVSHEDPEVKFN | Clone CH3C.35.23.2 |
| | WYVDGVEVHNAKTKPREEQYNSTYRVVSVLTVLHQDWLNGKEY | with knob and LALAPG |
| | KCKVSNKALGAPIEKTISKAKGQPREPQVYTLPPSRDELTKNQVSL | mutations |
| | WCLVKGFYPSDIAVEWESYGTEWANYKTTPPVLDSDGSFFLYSKLT | |
| | VTKEEWQQGFVFSCSVMHEALHNHYTQKSLSLSPGK | |
| 133 | APELLGGPSVFLFPPKPKDTLYITREPEVTCVVVDVSHEDPEVKFNW | Clone CH3C.35.23.2 |
| | YVDGVEVHNAKTKPREEQYNSTYRVVSVLTVLHQDWLNGKEYKC | with knob and YTE |
| | KVSNKALPAPIEKTISKAKGQPREPQVYTLPPSRDELTKNQVSLWCL | mutations |
| | VKGFYPSDIAVEWESYGTEWANYKTTPPVLDSDGSFFLYSKLTVTK | |
| | EEWQQGFVFSCSVMHEALHNHYTQKSLSLSPGK | |
| 134 | APEAAGGPSVFLFPPKPKDTLYITREPEVTCVVVDVSHEDPEVKFN | Clone CH3C.35.23.2 |
| | WYVDGVEVHNAKTKPREEQYNSTYRVVSVLTVLHQDWLNGKEY | with knob, LALA, and |
| | KCKVSNKALPAPIEKTISKAKGQPREPQVYTLPPSRDELTKNQVSLW | YTE mutations |
| | CLVKGFYPSDIAVEWESYGTEWANYKTTPPVLDSDGSFFLYSKLTV | |
| | TKEEWQQGFVFSCSVMHEALHNHYTQKSLSLSPGK | |
| 135 | APEAAGGPSVFLFPPKPKDTLYITREPEVTCVVVDVSHEDPEVKFN | Clone CH3C.35.23.2 |
| | WYVDGVEVHNAKTKPREEQYNSTYRVVSVLTVLHQDWLNGKEY | with knob, LALAPG, |
| | KCKVSNKALGAPIEKTISKAKGQPREPQVYTLPPSRDELTKNQVSL | and YTE mutations |
| | WCLVKGFYPSDIAVEWESYGTEWANYKTTPPVLDSDGSFFLYSKLT | |
| | VTKEEWQQGFVFSCSVMHEALHNHYTQKSLSLSPGK | |
| 136 | APELLGGPSVFLFPPKPKDTLMISRTPEVTCVVVDVSHEDPEVKFNW | Clone CH3C.35.23.2 |
| | YVDGVEVHNAKTKPREEQYNSTYRVVSVLTVLHQDWLNGKEYKC | with hole mutations |
| | KVSNKALPAPIEKTISKAKGQPREPQVYTLPPSRDELTKNQVSLSCA | |
| | VKGFYPSDIAVEWESYGTEWANYKTTPPVLDSDGSFFLVSKLTVTK | |
| | EEWQQGFVFSCSVMHEALHNHYTQKSLSLSPGK | |
| 137 | APEAAGGPSVFLFPPKPKDTLMISRTPEVTCVVVDVSHEDPEVKFN | Clone CH3C.35.23.2 |
| | WYVDGVEVHNAKTKPREEQYNSTYRVVSVLTVLHQDWLNGKEY | with hole and LALA |
| | KCKVSNKALPAPIEKTISKAKGQPREPQVYTLPPSRDELTKNQVSLS | mutations |
| | CAVKGFYPSDIAVEWESYGTEWANYKTTPPVLDSDGSFFLVSKLTV | |
| | TKEEWQQGFVFSCSVMHEALHNHYTQKSLSLSPGK | |
| 138 | APEAAGGPSVFLFPPKPKDTLMISRTPEVTCVVVDVSHEDPEVKFN | Clone CH3C.35.23.2 |
| | WYVDGVEVHNAKTKPREEQYNSTYRVVSVLTVLHQDWLNGKEY | with hole and LALAPG |
| | KCKVSNKALGAPIEKTISKAKGQPREPQVYTLPPSRDELTKNQVSLS | mutations |
| | CAVKGFYPSDIAVEWESYGTEWANYKTTPPVLDSDGSFFLVSKLTV | |
| | TKEEWQQGFVFSCSVMHEALHNHYTQKSLSLSPGK | |
| 139 | APELLGGPSVFLFPPKPKDTLYITREPEVTCVVVDVSHEDPEVKFNW | Clone CH3C.35.23.2 |
| | YVDGVEVHNAKTKPREEQYNSTYRVVSVLTVLHQDWLNGKEYKC | with hole and YTE |
| | KVSNKALPAPIEKTISKAKGQPREPQVYTLPPSRDELTKNQVSLSCA | mutations |
| | VKGFYPSDIAVEWESYGTEWANYKTTPPVLDSDGSFFLVSKLTVTK | |
| | EEWQQGFVFSCSVMHEALHNHYTQKSLSLSPGK | |
| 140 | APEAAGGPSVFLFPPKPKDTLYITREPEVTCVVVDVSHEDPEVKFN | Clone CH3C.35.23.2 |
| | WYVDGVEVHNAKTKPREEQYNSTYRVVSVLTVLHQDWLNGKEY | with hole, LALA, and |
| | KCKVSNKALPAPIEKTISKAKGQPREPQVYTLPPSRDELTKNQVSLS | YTE mutations |
| | CAVKGFYPSDIAVEWESYGTEWANYKTTPPVLDSDGSFFLVSKLTV | |
| | TKEEWQQGFVFSCSVMHEALHNHYTQKSLSLSPGK | |
| 141 | APEAAGGPSVFLFPPKPKDTLYITREPEVTCVVVDVSHEDPEVKFN | Clone CH3C.35.23.2 |
| | WYVDGVEVHNAKTKPREEQYNSTYRVVSVLTVLHQDWLNGKEY | with hole, LALAPG, and |
| | KCKVSNKALGAPIEKTISKAKGQPREPQVYTLPPSRDELTKNQVSLS | YTE mutations |
| | CAVKGFYPSDIAVEWESYGTEWANYKTTPPVLDSDGSFFLVSKLTV | |
| | TKEEWQQGFVFSCSVMHEALHNHYTQKSLSLSPGK | |
| 142 | APELLGGPSVFLFPPKPKDTLMISRTPEVTCVVVDVSHEDPEVKFNW | Clone CH3C.35.23.3 |
| | YVDGVEVHNAKTKPREEQYNSTYRVVSVLTVLHQDWLNGKEYKC | with knob mutation |
| | KVSNKALPAPIEKTISKAKGQPREPQVYTLPPSRDELTKNQVSLWCL | |
| | VKGFYPSDIAVEWESYGTEWVNYKTTPPVLDSDGSFFLYSKLTVTK | |
| | EEWQQGFVFSCSVMHEALHNHYTQKSLSLSPGK | |
| 143 | APEAAGGPSVFLFPPKPKDTLMISRTPEVTCVVVDVSHEDPEVKFN | Clone CH3C.35.23.3 |
| | WYVDGVEVHNAKTKPREEQYNSTYRVVSVLTVLHQDWLNGKEY | with knob and LALA |
| | KCKVSNKALPAPIEKTISKAKGQPREPQVYTLPPSRDELTKNQVSLW | mutations |
| | CLVKGFYPSDIAVEWESYGTEWVNYKTTPPVLDSDGSFFLYSKLTV | |
| | TKEEWQQGFVFSCSVMHEALHNHYTQKSLSLSPGK | |
| 144 | APEAAGGPSVFLFPPKPKDTLMISRTPEVTCVVVDVSHEDPEVKFN | Clone CH3C.35.23.3 |
| | WYVDGVEVHNAKTKPREEQYNSTYRVVSVLTVLHQDWLNGKEY | with knob and LALAPG |
| | KCKVSNKALGAPIEKTISKAKGQPREPQVYTLPPSRDELTKNQVSL | mutations |
| | WCLVKGFYPSDIAVEWESYGTEWVNYKTTPPVLDSDGSFFLYSKLT | |
| | VTKEEWQQGFVFSCSVMHEALHNHYTQKSLSLSPGK | |
| 145 | APELLGGPSVFLFPPKPKDTLYITREPEVTCVVVDVSHEDPEVKFNW | Clone CH3C.35.23.3 |
| | YVDGVEVHNAKTKPREEQYNSTYRVVSVLTVLHQDWLNGKEYKC | with knob and YTE |
| | KVSNKALPAPIEKTISKAKGQPREPQVYTLPPSRDELTKNQVSLWCL | mutations |
| | VKGFYPSDIAVEWESYGTEWVNYKTTPPVLDSDGSFFLYSKLTVTK | |
| | EEWQQGFVFSCSVMHEALHNHYTQKSLSLSPGK | |
| 146 | APEAAGGPSVFLFPPKPKDTLYITREPEVTCVVVDVSHEDPEVKFN | Clone CH3C.35.23.3 |
| | WYVDGVEVHNAKTKPREEQYNSTYRVVSVLTVLHQDWLNGKEY | with knob, LALA, and |
| | KCKVSNKALPAPIEKTISKAKGQPREPQVYTLPPSRDELTKNQVSLW | YTE mutations |
| | CLVKGFYPSDIAVEWESYGTEWVNYKTTPPVLDSDGSFFLYSKLTV | |
| | TKEEWQQGFVFSCSVMHEALHNHYTQKSLSLSPGK | |
| 147 | APEAAGGPSVFLFPPKPKDTLYITREPEVTCVVVDVSHEDPEVKFN | Clone CH3C.35.23.3 |
| | WYVDGVEVHNAKTKPREEQYNSTYRVVSVLTVLHQDWLNGKEY | with knob, LALAPG, |
| | KCKVSNKALGAPIEKTISKAKGQPREPQVYTLPPSRDELTKNQVSL | and YTE mutations |
| | WCLVKGFYPSDIAVEWESYGTEWVNYKTTPPVLDSDGSFFLYSKLT | |
| | VTKEEWQQGFVFSCSVMHEALHNHYTQKSLSLSPGK | |
| 148 | APELLGGPSVFLFPPKPKDTLMISRTPEVTCVVVDVSHEDPEVKFNW | Clone CH3C.35.23.3 |
| | YVDGVEVHNAKTKPREEQYNSTYRVVSVLTVLHQDWLNGKEYKC | with hole mutations |
| | KVSNKALPAPIEKTISKAKGQPREPQVYTLPPSRDELTKNQVSLSCA | |
| | VKGFYPSDIAVEWESYGTEWVNYKTTPPVLDSDGSFFLVSKLTVTK | |
| | EEWQQGFVFSCSVMHEALHNHYTQKSLSLSPGK | |
| 149 | APEAAGGPSVFLFPPKPKDTLMISRTPEVTCVVVDVSHEDPEVKFN | Clone CH3C.35.23.3 |
| | WYVDGVEVHNAKTKPREEQYNSTYRVVSVLTVLHQDWLNGKEY | with hole and LALA |
| | KCKVSNKALPAPIEKTISKAKGQPREPQVYTLPPSRDELTKNQVSLS | mutations |
| | CAVKGFYPSDIAVEWESYGTEWVNYKTTPPVLDSDGSFFLVSKLTV | |
| | TKEEWQQGFVFSCSVMHEALHNHYTQKSLSLSPGK | |
| 150 | APEAAGGPSVFLFPPKPKDTLMISRTPEVTCVVVDVSHEDPEVKFN | Clone CH3C.35.23.3 |
| | WYVDGVEVHNAKTKPREEQYNSTYRVVSVLTVLHQDWLNGKEY | with hole and LALAPG |
| | KCKVSNKALGAPIEKTISKAKGQPREPQVYTLPPSRDELTKNQVSLS | mutations |
| | CAVKGFYPSDIAVEWESYGTEWVNYKTTPPVLDSDGSFFLVSKLTV | |
| | TKEEWQQGFVFSCSVMHEALHNHYTQKSLSLSPGK | |
| 151 | APELLGGPSVFLFPPKPKDTLYITREPEVTCVVVDVSHEDPEVKFNW | Clone CH3C.35.23.3 |
| | YVDGVEVHNAKTKPREEQYNSTYRVVSVLTVLHQDWLNGKEYKC | with hole and YTE |
| | KVSNKALPAPIEKTISKAKGQPREPQVYTLPPSRDELTKNQVSLSCA | mutations |
| | VKGFYPSDIAVEWESYGTEWVNYKTTPPVLDSDGSFFLVSKLTVTK | |
| | EEWQQGFVFSCSVMHEALHNHYTQKSLSLSPGK | |
| 152 | APEAAGGPSVFLFPPKPKDTLYITREPEVTCVVVDVSHEDPEVKFN | Clone CH3C.35.23.3 |
| | WYVDGVEVHNAKTKPREEQYNSTYRVVSVLTVLHQDWLNGKEY | with hole, LALA, and |
| | KCKVSNKALPAPIEKTISKAKGQPREPQVYTLPPSRDELTKNQVSLS | YTE mutations |
| | CAVKGFYPSDIAVEWESYGTEWVNYKTTPPVLDSDGSFFLVSKLTV | |
| | TKEEWQQGFVFSCSVMHEALHNHYTQKSLSLSPGK | |
| 153 | APEAAGGPSVFLFPPKPKDTLYITREPEVTCVVVDVSHEDPEVKFN | Clone CH3C.35.23.3 |
| | WYVDGVEVHNAKTKPREEQYNSTYRVVSVLTVLHQDWLNGKEY | with hole, LALAPG, and |
| | KCKVSNKALGAPIEKTISKAKGQPREPQVYTLPPSRDELTKNQVSLS | YTE mutations |
| | CAVKGFYPSDIAVEWESYGTEWVNYKTTPPVLDSDGSFFLVSKLTV | |
| | TKEEWQQGFVFSCSVMHEALHNHYTQKSLSLSPGK | |
| 154 | APELLGGPSVFLFPPKPKDTLMISRTPEVTCVVVDVSHEDPEVKFNW | Clone CH3C.35.23.4 |
| | YVDGVEVHNAKTKPREEQYNSTYRVVSVLTVLHQDWLNGKEYKC | with knob mutation |
| | KVSNKALPAPIEKTISKAKGQPREPQVYTLPPSRDELTKNQVSLWCL | |
| | VKGFYPSDIAVEWESYGTEWSNYKTTPPVLDSDGSFFLYSKLTVSK | |
| | EEWQQGFVFSCSVMHEALHNHYTQKSLSLSPGK | |
| 155 | APEAAGGPSVFLFPPKPKDTLMISRTPEVTCVVVDVSHEDPEVKFN | Clone CH3C.35.23.4 |
| | WYVDGVEVHNAKTKPREEQYNSTYRVVSVLTVLHQDWLNGKEY | with knob and LALA |
| | KCKVSNKALPAPIEKTISKAKGQPREPQVYTLPPSRDELTKNQVSLW | mutations |
| | CLVKGFYPSDIAVEWESYGTEWSNYKTTPPVLDSDGSFFLYSKLTV | |
| | SKEEWQQGFVFSCSVMHEALHNHYTQKSLSLSPGK | |
| 156 | APEAAGGPSVFLFPPKPKDTLMISRTPEVTCVVVDVSHEDPEVKFN | Clone CH3C.35.23.4 |
| | WYVDGVEVHNAKTKPREEQYNSTYRVVSVLTVLHQDWLNGKEY | with knob and LALAPG |
| | KCKVSNKALGAPIEKTISKAKGQPREPQVYTLPPSRDELTKNQVSL | mutations |
| | WCLVKGFYPSDIAVEWESYGTEWSNYKTTPPVLDSDGSFFLYSKLT | |
| | VSKEEWQQGFVFSCSVMHEALHNHYTQKSLSLSPGK | |
| 157 | APELLGGPSVFLFPPKPKDTLYITREPEVTCVVVDVSHEDPEVKFNW | Clone CH3C.35.23.4 |
| | YVDGVEVHNAKTKPREEQYNSTYRVVSVLTVLHQDWLNGKEYKC | with knob and YTE |
| | KVSNKALPAPIEKTISKAKGQPREPQVYTLPPSRDELTKNQVSLWCL | mutations |
| | VKGFYPSDIAVEWESYGTEWSNYKTTPPVLDSDGSFFLYSKLTVSK | |
| | EEWQQGFVFSCSVMHEALHNHYTQKSLSLSPGK | |
| 158 | APEAAGGPSVFLFPPKPKDTLYITREPEVTCVVVDVSHEDPEVKFN | Clone CH3C.35.23.4 |
| | WYVDGVEVHNAKTKPREEQYNSTYRVVSVLTVLHQDWLNGKEY | with knob, LALA, and |
| | KCKVSNKALPAPIEKTISKAKGQPREPQVYTLPPSRDELTKNQVSLW | YTEmutations |
| | CLVKGFYPSDIAVEWESYGTEWSNYKTTPPVLDSDGSFFLYSKLTV | |
| | SKEEWQQGFVFSCSVMHEALHNHYTQKSLSLSPGK | |
| 159 | APEAAGGPSVFLFPPKPKDTLYITREPEVTCVVVDVSHEDPEVKFN | Clone CH3C.35.23.4 |
| | WYVDGVEVHNAKTKPREEQYNSTYRVVSVLTVLHQDWLNGKEY | with knob, LALAPG, |
| | KCKVSNKALGAPIEKTISKAKGQPREPQVYTLPPSRDELTKNQVSL | and YTEmutations |
| | WCLVKGFYPSDIAVEWESYGTEWSNYKTTPPVLDSDGSFFLYSKLT | |
| | VSKEEWQQGFVFSCSVMHEALHNHYTQKSLSLSPGK | |
| 160 | APELLGGPSVFLFPPKPKDTLMISRTPEVTCVVVDVSHEDPEVKFNW | Clone CH3C.35.23.4 |
| | YVDGVEVHNAKTKPREEQYNSTYRVVSVLTVLHQDWLNGKEYKC | with hole mutations |
| | KVSNKALPAPIEKTISKAKGQPREPQVYTLPPSRDELTKNQVSLSCA | |
| | VKGFYPSDIAVEWESYGTEWSNYKTTPPVLDSDGSFFLVSKLTVSK | |
| | EEWQQGFVFSCSVMHEALHNHYTQKSLSLSPGK | |
| 161 | APEAAGGPSVFLFPPKPKDTLMISRTPEVTCVVVDVSHEDPEVKFN | Clone CH3C.35.23.4 |
| | WYVDGVEVHNAKTKPREEQYNSTYRVVSVLTVLHQDWLNGKEY | with hole and LALA |
| | KCKVSNKALPAPIEKTISKAKGQPREPQVYTLPPSRDELTKNQVSLS | mutations |
| | CAVKGFYPSDIAVEWESYGTEWSNYKTTPPVLDSDGSFFLVSKLTV | |
| | SKEEWQQGFVFSCSVMHEALHNHYTQKSLSLSPGK | |
| 162 | APEAAGGPSVFLFPPKPKDTLMISRTPEVTCVVVDVSHEDPEVKFN | Clone CH3C.35.23.4 |
| | WYVDGVEVHNAKTKPREEQYNSTYRVVSVLTVLHQDWLNGKEY | with hole and LALAPG |
| | KCKVSNKALGAPIEKTISKAKGQPREPQVYTLPPSRDELTKNQVSLS | mutations |
| | CAVKGFYPSDIAVEWESYGTEWSNYKTTPPVLDSDGSFFLVSKLTV | |
| | SKEEWQQGFVFSCSVMHEALHNHYTQKSLSLSPGK | |
| 163 | APELLGGPSVFLFPPKPKDTLYITREPEVTCVVVDVSHEDPEVKFNW | Clone CH3C.35.23.4 |
| | YVDGVEVHNAKTKPREEQYNSTYRVVSVLTVLHQDWLNGKEYKC | with hole and YTE |
| | KVSNKALPAPIEKTISKAKGQPREPQVYTLPPSRDELTKNQVSLSCA | mutations |
| | VKGFYPSDIAVEWESYGTEWSNYKTTPPVLDSDGSFFLVSKLTVSK | |
| | EEWQQGFVFSCSVMHEALHNHYTQKSLSLSPGK | |
| 164 | APEAAGGPSVFLFPPKPKDTLYITREPEVTCVVVDVSHEDPEVKFN | Clone CH3C.35.23.4 |
| | WYVDGVEVHNAKTKPREEQYNSTYRVVSVLTVLHQDWLNGKEY | with hole, LALA, and |
| | KCKVSNKALPAPIEKTISKAKGQPREPQVYTLPPSRDELTKNQVSLS | YTE mutations |
| | CAVKGFYPSDIAVEWESYGTEWSNYKTTPPVLDSDGSFFLVSKLTV | |
| | SKEEWQQGFVFSCSVMHEALHNHYTQKSLSLSPGK | |
| 165 | APEAAGGPSVFLFPPKPKDTLYITREPEVTCVVVDVSHEDPEVKFN | Clone CH3C.35.23.4 |
| | WYVDGVEVHNAKTKPREEQYNSTYRVVSVLTVLHQDWLNGKEY | with hole, LALAPG, and |
| | KCKVSNKALGAPIEKTISKAKGQPREPQVYTLPPSRDELTKNQVSLS | YTE mutations |
| | CAVKGFYPSDIAVEWESYGTEWSNYKTTPPVLDSDGSFFLVSKLTV | |
| | SKEEWQQGFVFSCSVMHEALHNHYTQKSLSLSPGK | |
| 166 | APELLGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKC | with knob mutation |
| | KVSNKALPAPIEKTISKAKGQPREPQVYTLPPSRDELTKNQVSLWCL | |
| | VKGFYPSDIAVLWESYGTEWASYKTTPPVLDSDGSFFLYSKLTVTK | |
| | EEWQQGFVFSCSVMHEALHNHYTQKSLSLSPGK | |
| 167 | APEAAGGPSVFLFPPKPKDTLMISRTPEVTCVVVDVSHEDPEVKFN | Clone CH3C.35.21.17.2 |
| | WYVDGVEVHNAKTKPREEQYNSTYRVVSVLTVLHQDWLNGKEY | with knob and LALA |
| | KCKVSNKALPAPIEKTISKAKGQPREPQVYTLPPSRDELTKNQVSLW | mutations |
| | CLVKGFYPSDIAVLWESYGTEWASYKTTPPVLDSDGSFFLYSKLTV | |
| | TKEEWQQGFVFSCSVMHEALHNHYTQKSLSLSPGK | |
| 168 | APEAAGGPSVFLFPPKPKDTLMISRTPEVTCVVVDVSHEDPEVKFN | Clone CH3C.35.21.17.2 |
| | WYVDGVEVHNAKTKPREEQYNSTYRVVSVLTVLHQDWLNGKEY | with knob and LALAPG |
| | KCKVSNKALGAPIEKTISKAKGQPREPQVYTLPPSRDELTKNQVSL | mutations |
| | WCLVKGFYPSDIAVLWESYGTEWASYKTTPPVLDSDGSFFLYSKLT | |
| | VTKEEWQQGFVFSCSVMHEALHNHYTQKSLSLSPGK | |
| 169 | APELLGGPSVFLFPPKPKDTLYITREPEVTCVVVDVSHEDPEVKFNW | Clone CH3C.35.21.17.2 |
| | YVDGVEVHNAKTKPREEQYNSTYRVVSVLTVLHQDWLNGKEYKC | with knob and YTE |
| | KVSNKALPAPIEKTISKAKGQPREPQVYTLPPSRDELTKNQVSLWCL | mutations |
| | VKGFYPSDIAVLWESYGTEWASYKTTPPVLDSDGSFFLYSKLTVTK | |
| | EEWQQGFVFSCSVMHEALHNHYTQKSLSLSPGK | |
| 170 | APEAAGGPSVFLFPPKPKDTLYITREPEVTCVVVDVSHEDPEVKFN | Clone CH3C.35.21.17.2 |
| | WYVDGVEVHNAKTKPREEQYNSTYRVVSVLTVLHQDWLNGKEY | with knob, LALA, and |
| | KCKVSNKALPAPIEKTISKAKGQPREPQVYTLPPSRDELTKNQVSLW | YTE mutations |
| | CLVKGFYPSDIAVLWESYGTEWASYKTTPPVLDSDGSFFLYSKLTV | |
| | TKEEWQQGFVFSCSVMHEALHNHYTQKSLSLSPGK | |
| 171 | APEAAGGPSVFLFPPKPKDTLYITREPEVTCVVVDVSHEDPEVKFN | Clone CH3C.35.21.17.2 |
| | WYVDGVEVHNAKTKPREEQYNSTYRVVSVLTVLHQDWLNGKEY | with knob, LALAPG, |
| | KCKVSNKALGAPIEKTISKAKGQPREPQVYTLPPSRDELTKNQVSL | and YTE mutations |
| | WCLVKGFYPSDIAVLWESYGTEWASYKTTPPVLDSDGSFFLYSKLT | |
| | VTKEEWQQGFVFSCSVMHEALHNHYTQKSLSLSPGK | |
| 172 | APELLGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKC | with hole mutations |
| | KVSNKALPAPIEKTISKAKGQPREPQVYTLPPSRDELTKNQVSLSCA | |
| | VKGFYPSDIAVLWESYGTEWASYKTTPPVLDSDGSFFLVSKLTVTK | |
| | EEWQQGFVFSCSVMHEALHNHYTQKSLSLSPGK | |
| 173 | APEAAGGPSVFLFPPKPKDTLMISRTPEVTCVVVDVSHEDPEVKFN | Clone CH3C.35.21.17.2 |
| | WYVDGVEVHNAKTKPREEQYNSTYRVVSVLTVLHQDWLNGKEY | with hole and LALA |
| | KCKVSNKALPAPIEKTISKAKGQPREPQVYTLPPSRDELTKNQVSLS | mutations |
| | CAVKGFYPSDIAVLWESYGTEWASYKTTPPVLDSDGSFFLVSKLTV | |
| | TKEEWQQGFVFSCSVMHEALHNHYTQKSLSLSPGK | |
| 174 | APEAAGGPSVFLFPPKPKDTLMISRTPEVTCVVVDVSHEDPEVKFN | Clone CH3C.35.21.17.2 |
| | WYVDGVEVHNAKTKPREEQYNSTYRVVSVLTVLHQDWLNGKEY | with hole and LALAPG |
| | KCKVSNKALGAPIEKTISKAKGQPREPQVYTLPPSRDELTKNQVSLS | mutations |
| | CAVKGFYPSDIAVLWESYGTEWASYKTTPPVLDSDGSFFLVSKLTV | |
| | TKEEWQQGFVFSCSVMHEALHNHYTQKSLSLSPGK | |
| 175 | APELLGGPSVFLFPPKPKDTLYITREPEVTCVVVDVSHEDPEVKFNW | Clone CH3C.35.21.17.2 |
| | YVDGVEVHNAKTKPREEQYNSTYRVVSVLTVLHQDWLNGKEYKC | with hole and YTE |
| | KVSNKALPAPIEKTISKAKGQPREPQVYTLPPSRDELTKNQVSLSCA | mutations |
| | VKGFYPSDIAVLWESYGTEWASYKTTPPVLDSDGSFFLVSKLTVTK | |
| | EEWQQGFVFSCSVMHEALHNHYTQKSLSLSPGK | |
| 176 | APEAAGGPSVFLFPPKPKDTLYITREPEVTCVVVDVSHEDPEVKFN | Clone CH3C.35.21.17.2 |
| | WYVDGVEVHNAKTKPREEQYNSTYRVVSVLTVLHQDWLNGKEY | with hole, LALA, and |
| | KCKVSNKALPAPIEKTISKAKGQPREPQVYTLPPSRDELTKNQVSLS | YTE mutations |
| | CAVKGFYPSDIAVLWESYGTEWASYKTTPPVLDSDGSFFLVSKLTV | |
| | TKEEWQQGFVFSCSVMHEALHNHYTQKSLSLSPGK | |
| 177 | APEAAGGPSVFLFPPKPKDTLYITREPEVTCVVVDVSHEDPEVKFN | Clone CH3C.35.21.17.2 |
| | WYVDGVEVHNAKTKPREEQYNSTYRVVSVLTVLHQDWLNGKEY | with hole, LALAPG, and |
| | KCKVSNKALGAPIEKTISKAKGQPREPQVYTLPPSRDELTKNQVSLS | YTE mutations |
| | CAVKGFYPSDIAVLWESYGTEWASYKTTPPVLDSDGSFFLVSKLTV | |
| | TKEEWQQGFVFSCSVMHEALHNHYTQKSLSLSPGK | |
| 178 | APELLGGPSVFLFPPKPKDTLMISRTPEVTCVVVDVSHEDPEVKFNW | Clone CH3C.35.23 with |
| | YVDGVEVHNAKTKPREEQYNSTYRVVSVLTVLHQDWLNGKEYKC | knob mutation |
| | KVSNKALPAPIEKTISKAKGQPREPQVYTLPPSRDELTKNQVSLWCL | |
| | VKGFYPSDIAVEWESYGTEWSNYKTTPPVLDSDGSFFLYSKLTVTK | |
| | EEWQQGFVFSCSVMHEALHNHYTQKSLSLSPGK | |
| 179 | APEAAGGPSVFLFPPKPKDTLMISRTPEVTCVVVDVSHEDPEVKFN | Clone CH3C.35.23 with |
| | WYVDGVEVHNAKTKPREEQYNSTYRVVSVLTVLHQDWLNGKEY | knob and LALA |
| | KCKVSNKALPAPIEKTISKAKGQPREPQVYTLPPSRDELTKNQVSLW | mutations |
| | CLVKGFYPSDIAVEWESYGTEWSNYKTTPPVLDSDGSFFLYSKLTV | |
| | TKEEWQQGFVFSCSVMHEALHNHYTQKSLSLSPGK | |
| 180 | APEAAGGPSVFLFPPKPKDTLMISRTPEVTCVVVDVSHEDPEVKFN | Clone CH3C.35.23 with |
| | WYVDGVEVHNAKTKPREEQYNSTYRVVSVLTVLHQDWLNGKEY | knob and LALAPG |
| | KCKVSNKALGAPIEKTISKAKGQPREPQVYTLPPSRDELTKNQVSL | mutations |
| | WCLVKGFYPSDIAVEWESYGTEWSNYKTTPPVLDSDGSFFLYSKLT | |
| | VTKEEWQQGFVFSCSVMHEALHNHYTQKSLSLSPGK | |
| 181 | APELLGGPSVFLFPPKPKDTLYITREPEVTCVVVDVSHEDPEVKFNW | Clone CH3C.35.23 with |
| | YVDGVEVHNAKTKPREEQYNSTYRVVSVLTVLHQDWLNGKEYKC | knob and YTE mutations |
| | KVSNKALPAPIEKTISKAKGQPREPQVYTLPPSRDELTKNQVSLWCL | |
| | VKGFYPSDIAVEWESYGTEWSNYKTTPPVLDSDGSFFLYSKLTVTK | |
| | EEWQQGFVFSCSVMHEALHNHYTQKSLSLSPGK | |
| 182 | APEAAGGPSVFLFPPKPKDTLYITREPEVTCVVVDVSHEDPEVKFN | Clone CH3C.35.23 with |
| | WYVDGVEVHNAKTKPREEQYNSTYRVVSVLTVLHQDWLNGKEY | knob, LALA, and YTE |
| | KCKVSNKALPAPIEKTISKAKGQPREPQVYTLPPSRDELTKNQVSLW | mutations |
| | CLVKGFYPSDIAVEWESYGTEWSNYKTTPPVLDSDGSFFLYSKLTV | |
| | TKEEWQQGFVFSCSVMHEALHNHYTQKSLSLSPGK | |
| 183 | APEAAGGPSVFLFPPKPKDTLYITREPEVTCVVVDVSHEDPEVKFN | Clone CH3C.35.23 with |
| | WYVDGVEVHNAKTKPREEQYNSTYRVVSVLTVLHQDWLNGKEY | knob, LALAPG, and |
| | KCKVSNKALGAPIEKTISKAKGQPREPQVYTLPPSRDELTKNQVSL | YTE mutations |
| | WCLVKGFYPSDIAVEWESYGTEWSNYKTTPPVLDSDGSFFLYSKLT | |
| | VTKEEWQQGFVFSCSVMHEALHNHYTQKSLSLSPGK | |
| 184 | APELLGGPSVFLFPPKPKDTLMISRTPEVTCVVVDVSHEDPEVKFNW | Clone CH3C.35.23 with |
| | YVDGVEVHNAKTKPREEQYNSTYRVVSVLTVLHQDWLNGKEYKC | hole mutations |
| | KVSNKALPAPIEKTISKAKGQPREPQVYTLPPSRDELTKNQVSLSCA | |
| | VKGFYPSDIAVEWESYGTEWSNYKTTPPVLDSDGSFFLVSKLTVTK | |
| | EEWQQGFVFSCSVMHEALHNHYTQKSLSLSPGK | |
| 185 | APEAAGGPSVFLFPPKPKDTLMISRTPEVTCVVVDVSHEDPEVKFN | Clone CH3C.35.23 with |
| | WYVDGVEVHNAKTKPREEQYNSTYRVVSVLTVLHQDWLNGKEY | hole and LALA |
| | KCKVSNKALPAPIEKTISKAKGQPREPQVYTLPPSRDELTKNQVSLS | mutations |
| | CAVKGFYPSDIAVEWESYGTEWSNYKTTPPVLDSDGSFFLVSKLTV | |
| | TKEEWQQGFVFSCSVMHEALHNHYTQKSLSLSPGK | |
| 186 | APEAAGGPSVFLFPPKPKDTLMISRTPEVTCVVVDVSHEDPEVKFN | Clone CH3C.35.23 with |
| | WYVDGVEVHNAKTKPREEQYNSTYRVVSVLTVLHQDWLNGKEY | hole and LALAPG |
| | KCKVSNKALGAPIEKTISKAKGQPREPQVYTLPPSRDELTKNQVSLS | mutations |
| | CAVKGFYPSDIAVEWESYGTEWSNYKTTPPVLDSDGSFFLVSKLTV | |
| | TKEEWQQGFVFSCSVMHEALHNHYTQKSLSLSPGK | |
| 187 | APELLGGPSVFLFPPKPKDTLYITREPEVTCVVVDVSHEDPEVKFNW | Clone CH3C.35.23 with |
| | YVDGVEVHNAKTKPREEQYNSTYRVVSVLTVLHQDWLNGKEYKC | hole and YTE mutations |
| | KVSNKALPAPIEKTISKAKGQPREPQVYTLPPSRDELTKNQVSLSCA | |
| | VKGFYPSDIAVEWESYGTEWSNYKTTPPVLDSDGSFFLVSKLTVTK | |
| | EEWQQGFVFSCSVMHEALHNHYTQKSLSLSPGK | |
| 188 | APEAAGGPSVFLFPPKPKDTLYITREPEVTCVVVDVSHEDPEVKFN | Clone CH3C.35.23 with |
| | WYVDGVEVHNAKTKPREEQYNSTYRVVSVLTVLHQDWLNGKEY | hole, LALA, and YTE |
| | KCKVSNKALPAPIEKTISKAKGQPREPQVYTLPPSRDELTKNQVSLS | mutations |
| | CAVKGFYPSDIAVEWESYGTEWSNYKTTPPVLDSDGSFFLVSKLTV | |
| | TKEEWQQGFVFSCSVMHEALHNHYTQKSLSLSPGK | |
| 189 | APEAAGGPSVFLFPPKPKDTLYITREPEVTCVVVDVSHEDPEVKFN | Clone CH3C.35.23 with |
| | WYVDGVEVHNAKTKPREEQYNSTYRVVSVLTVLHQDWLNGKEY | hole, LALAPG, and |
| | KCKVSNKALGAPIEKTISKAKGQPREPQVYTLPPSRDELTKNQVSLS | YTE mutations |
| | CAVKGFYPSDIAVEWESYGTEWSNYKTTPPVLDSDGSFFLVSKLTV | |
| | TKEEWQQGFVFSCSVMHEALHNHYTQKSLSLSPGK | |
| 190 | DKTHTCPPCPAPEAAGGPSVFLFPPKPKDTLMISRTPEVTCVVVDVS | Clone CH3C.35.21.17.2 |
| | HEDPEVKFNWYVDGVEVHNAKTKPREEQYNSTYRVVSVLTVLHQ | with knob and LALA |
| | DWLNGKEYKCKVSNKALPAPIEKTISKAKGQPREPQVYTLPPSRDE | mutations and portion of |
| | LTKNQVSLWCLVKGFYPSDIAVLWESYGTEWASYKTTPPVLDSDG | human IgG1 hinge |
| | SFFLYSKLTVTKEEWQQGFVFSCSVMHEALHNHYTQKSLSLSPGK | sequence |
| 191 | DKTHTCPPCPAPEAAGGPSVFLFPPKPKDTLMISRTPEVTCVVVDVS | Clone CH3C.35.23.2 |
| | HEDPEVKFNWYVDGVEVHNAKTKPREEQYNSTYRVVSVLTVLHQ | with knob and LALA |
| | DWLNGKEYKCKVSNKALPAPIEKTISKAKGQPREPQVYTLPPSRDE | mutations and portion of |
| | LTKNQVSLWCLVKGFYPSDIAVEWESYGTEWANYKTTPPVLDSDG | human IgG1 hinge |
| | SFFLYSKLTVTKEEWQQGFVFSCSVMHEALHNHYTQKSLSLSPGK | sequence |
| 192 | SETQANSTTD ALNVLLIIVD DLRPSLGCYG DKLVRSPNID | IDS sequence |
| | QLASHSLLFQ NAFAQQAVCA PSRVSFLTGR RPDTTRLYDF | |
| | NSYWRVHAGN FSTIPQYFKE NGYVTMSVGK VFHPGISSNH | |
| | TDDSPYSWSF PPYHPSSEKY ENTKTCRGPD GELHANLLCP | |
| | VDVLDVPEGT LPDKQSTEQA IQLLEKMKTS ASPFFLAVGY | |
| | HKPHIPFRYP KEFQKLYPLE NITLAPDPEV PDGLPPVAYN | |
| | PWMDIRQRED VQALNISVPY GPIPVDFQRK IRQSYFASVS | |
| | YLDTQVGRLL SALDDLQLAN STIIAFTSDH GWALGEHGEW | |
| | AKYSNFDVAT HVPLIFYVPG RTASLPEAGE KLFPYLDPFD | |
| | SASQLMEPGR QSMDLVELVS LFPTLAGLAG LQVPPRCPVP | |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMP | |
| 193 | SETQANSTTD ALNVLLIIVD DLRPSLGCYG DKLVRSPNID | IDS-Fc fusion |
| | QLASHSLLFQ NAFAQQAVCA PSRVSFLTGR RPDTTRLYDF | polypeptide with IDS |
| | NSYWRVHAGN FSTIPQYFKE NGYVTMSVGK VFHPGISSNH | sequence underlined and |
| | TDDSPYSWSF PPYHPSSEKY ENTKTCRGPD GELHANLLCP | hole and LALA |
| | VDVLDVPEGT LPDKQSTEQA IQLLEKMKTS ASPFFLAVGY | mutations |
| | HKPHIPFRYP KEFQKLYPLE NITLAPDPEV PDGLPPVAYN | |
| | PWMDIRQRED VQALNISVPY GPIPVDFQRK IRQSYFASVS | |
| | YLDTQVGRLL SALDDLQLAN STIIAFTSDH GWALGEHGEW | |
| | AKYSNFDVAT HVPLIFYVPG RTASLPEAGE KLFPYLDPFD | |
| | SASQLMEPGR QSMDLVELVS LFPTLAGLAG LQVPPRCPVP | |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APEAAGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPGK | |
| 194 | SETQANSTTD ALNVLLIIVD DLRPSLGCYG DKLVRSPNID | IDS-Fc fusion |
| | QLASHSLLFQ NAFAQQAVCA PSRVSFLTGR RPDTTRLYDF | polypeptide with IDS |
| | NSYWRVHAGN FSTIPQYFKE NGYVTMSVGK VFHPGISSNH | sequence underlined and |
| | TDDSPYSWSF PPYHPSSEKY ENTKTCRGPD GELHANLLCP | hole mutations |
| | VDVLDVPEGT LPDKQSTEQA IQLLEKMKTS ASPFFLAVGY | |
| | HKPHIPFRYP KEFQKLYPLE NITLAPDPEV PDGLPPVAYN | |
| | PWMDIRQRED VQALNISVPY GPIPVDFQRK IRQSYFASVS | |
| | YLDTQVGRLL SALDDLQLAN STIIAFTSDH GWALGEHGEW | |
| | AKYSNFDVAT HVPLIFYVPG RTASLPEAGE KLFPYLDPFD | |
| | SASQLMEPGR QSMDLVELVS LFPTLAGLAG LQVPPRCPVP | |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APELLGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPGK | |
| 195 | SETQANSTTD ALNVLLIIVD DLRPSLGCYG DKLVRSPNID | IDS-Fc fusion |
| | QLASHSLLFQ NAFAQQAVCA PSRVSFLTGR RPDTTRLYDF | polypeptide with IDS |
| | NSYWRVHAGN FSTIPQYFKE NGYVTMSVGK VFHPGISSNH | sequence underlined and |
| | TDDSPYSWSF PPYHPSSEKY ENTKTCRGPD GELHANLLCP | knob mutation |
| | VDVLDVPEGT LPDKQSTEQA IQLLEKMKTS ASPFFLAVGY | |
| | HKPHIPFRYP KEFQKLYPLE NITLAPDPEV PDGLPPVAYN | |
| | PWMDIRQRED VQALNISVPY GPIPVDFQRK IRQSYFASVS | |
| | YLDTQVGRLL SALDDLQLAN STIIAFTSDH GWALGEHGEW | |
| | AKYSNFDVAT HVPLIFYVPG RTASLPEAGE KLFPYLDPFD | |
| | SASQLMEPGR QSMDLVELVS LFPTLAGLAG LQVPPRCPVP | |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APELLGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLWCLVK | |
| | GFYPSDIAVE WESNGQPENN YKTTPPVLDS DGSFFLYSKL | |
| | TVDKSRWQQG NVFSCSVMHE ALHNHYTQKS LSLSPGK | |
| 196 | SETQANSTTD ALNVLLIIVD DLRPSLGCYG DKLVRSPNID | IDS sequence |
| | QLASHSLLFQ NAFAQQAVfGA PSRVSFLTGR RPDTTRLYDF | (formylglycine residue |
| | NSYWRVHAGN FSTIPQYFKE NGYVTMSVGK VFHPGISSNH | “fG” double underlined) |
| | TDDSPYSWSF PPYHPSSEKY ENTKTCRGPD GELHANLLCP | |
| | VDVLDVPEGT LPDKQSTEQA IQLLEKMKTS ASPFFLAVGY | |
| | HKPHIPFRYP KEFQKLYPLE NITLAPDPEV PDGLPPVAYN | |
| | PWMDIRQRED VQALNISVPY GPIPVDFQRK IRQSYFASVS | |
| | YLDTQVGRLL SALDDLQLAN STIIAFTSDH GWALGEHGEW | |
| | AKYSNFDVAT HVPLIFYVPG RTASLPEAGE KLFPYLDPFD | |
| | SASQLMEPGR QSMDLVELVS LFPTLAGLAG LQVPPRCPVP | |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMP | |
| 197 | SETQANSTTD ALNVLLIIVD DLRPSLGCYG DKLVRSPNID | IDS-Fc fusion |
| | QLASHSLLFQ NAFAQQAVfGA PSRVSFLTGR RPDTTRLYDF | polypeptide with IDS |
| | NSYWRVHAGN FSTIPQYFKE NGYVTMSVGK VFHPGISSNH | sequence underlined |
| | TDDSPYSWSF PPYHPSSEKY ENTKTCRGPD GELHANLLCP | (formylglycine residue |
| | VDVLDVPEGT LPDKQSTEQA IQLLEKMKTS ASPFFLAVGY | “fG” double underlined) |
| | HKPHIPFRYP KEFQKLYPLE NITLAPDPEV PDGLPPVAYN | and hole and LALA |
| | PWMDIRQRED VQALNISVPY GPIPVDFQRK IRQSYFASVS | mutations |
| | YLDTQVGRLL SALDDLQLAN STIIAFTSDH GWALGEHGEW | |
| | AKYSNFDVAT HVPLIFYVPG RTASLPEAGE KLFPYLDPFD | |
| | SASQLMEPGR QSMDLVELVS LFPTLAGLAG LQVPPRCPVP | |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APEAAGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPGK | |
| 198 | SETQANSTTD ALNVLLIIVD DLRPSLGCYG DKLVRSPNID | IDS-Fc fusion |
| | QLASHSLLFQ NAFAQQAVfGA PSRVSFLTGR RPDTTRLYDF | polypeptide with IDS |
| | NSYWRVHAGN FSTIPQYFKE NGYVTMSVGK VFHPGISSNH | sequence underlined |
| | TDDSPYSWSF PPYHPSSEKY ENTKTCRGPD GELHANLLCP | (formylglycine residue |
| | VDVLDVPEGT LPDKQSTEQA IQLLEKMKTS ASPFFLAVGY | “fG” double underlined) |
| | HKPHIPFRYP KEFQKLYPLE NITLAPDPEV PDGLPPVAYN | and hole mutations |
| | PWMDIRQRED VQALNISVPY GPIPVDFQRK IRQSYFASVS | |
| | YLDTQVGRLL SALDDLQLAN STIIAFTSDH GWALGEHGEW | |
| | AKYSNFDVAT HVPLIFYVPG RTASLPEAGE KLFPYLDPFD | |
| | SASQLMEPGR QSMDLVELVS LFPTLAGLAG LQVPPRCPVP | |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APELLGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPGK | |
| 199 | SETQANSTTD ALNVLLIIVD DLRPSLGCYG DKLVRSPNID | IDS-Fc fusion |
| | QLASHSLLFQ NAFAQQAVfGA PSRVSFLTGR RPDTTRLYDF | polypeptide with IDS |
| | NSYWRVHAGN FSTIPQYFKE NGYVTMSVGK VFHPGISSNH | sequence underlined |
| | TDDSPYSWSF PPYHPSSEKY ENTKTCRGPD GELHANLLCP | (formylglycine residue |
| | VDVLDVPEGT LPDKQSTEQA IQLLEKMKTS ASPFFLAVGY | “fG” double underlined) |
| | HKPHIPFRYP KEFQKLYPLE NITLAPDPEV PDGLPPVAYN | and knob mutation |
| | PWMDIRQRED VQALNISVPY GPIPVDFQRK IRQSYFASVS | |
| | YLDTQVGRLL SALDDLQLAN STIIAFTSDH GWALGEHGEW | |
| | AKYSNFDVAT HVPLIFYVPG RTASLPEAGE KLFPYLDPFD | |
| | SASQLMEPGR QSMDLVELVS LFPTLAGLAG LQVPPRCPVP | |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APELLGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLWCLVK | |
| | GFYPSDIAVE WESNGQPENN YKTTPPVLDS DGSFFLYSKL | |
| | TVDKSRWQQG NVFSCSVMHE ALHNHYTQKS LSLSPGK | |
| 200 | NSVIIVDKNGRLVYLVENPGGYVAYSKAATVTGKLVHANFGTKKD | Human TfR apical |
| | FEDLYTPVNGSIVIVRAGKITFAEKVANAESLNAIGVLIYMDQTKFPI | domain |
| | VNAELSFFGHAHLGTGDPYTPGFPSFNHTQFPPSRSSGLPNIPVQTIS | |
| | RAAAEKLFGNMEGDCPSDWKTDSTCRMVTSESKNVKLTVS | |
| 201 | GGGGS | Glycine-rich linker |
| 202 | GGGGSGGGGS | Glycine-rich linker |
| 203 | HHHHHH | Hexahistidine tag |
| 204 | YxTEWSS | Library motif |

All publications, patents, and patent documents are incorporated by reference herein, as though individually incorporated by reference. The present disclosure has been described with reference to various specific and preferred embodiments and techniques. However, it should be understood that many variations and modifications may be made while remaining within the spirit and scope of the invention.

What is claimed is:

1. A method of detecting one or more biomarkers in a subject having a lysosomal storage disorder (LSD), the method comprising:
1) measuring the concentration of a combination of two or more lipids in a sample from the subject, wherein the combination of lipids is selected from the group consisting of:
a) a bis(monoacylglycero)phosphate (BMP);
b) a GM2 ganglioside and/or a GM3 ganglioside;
c) a GD3 ganglioside;
d) a GD1a/b ganglioside; and
e) a glucosylceramide (GlcCer);
2) measuring the concentration of GlcCer in a sample from the subject, provided the LSD is a mucopolysaccharidosis (MPS) disorder;
3) measuring the concentration of neurofilament light chain (Nf-L) in a sample from the subject; and/or
4) measuring the concentration of soluble triggering receptor expressed on myeloid cells 2 (sTREM2) in a sample from the subject.

2. A method of evaluating the efficacy of a treatment in a subject having an LSD, the method comprising:
1) measuring the concentration of a combination of two or more lipids in a sample obtained from the subject after administration of the treatment, wherein the combination of lipids is selected from the group consisting of:
a) a BMP;
b) a GM2 ganglioside and/or a GM3 ganglioside;
c) a GD3;
d) a GD1a/b; and
e) a GlcCer;
2) measuring the concentration of GlcCer in a sample obtained from the subject after administration of the treatment, provided the LSD is an MPS disorder;
3) measuring the concentration of Nf-L in a sample obtained from the subject after administration of the treatment; and/or
4) measuring the concentration of sTREM2 in a sample obtained from the subject after administration of the treatment;
wherein a decrease in the concentration of the selected lipid(s)/protein(s) in the sample obtained from the subject after administration of the treatment as compared to the concentration of the lipid(s)/protein(s) in a sample obtained from the subject prior to administration of the treatment correlates with treatment efficacy.

3. The method of claim 1 or 2, further comprising administering an LSD treatment to the subject.

4. The method of any one of claims 1-3, further comprising adjusting a treatment regimen for the subject.

5. A method for treating an LSD in a subject, the method comprising:
1) administering an LSD treatment to the subject;
2) measuring the concentration of:
a) a combination of two or more lipids in a sample from the subject, wherein the combination of lipids is selected from the group consisting of:
i) a BMP;
ii) a GM2 ganglioside and/or a GM3 ganglioside;
iii) a GD3;
iv) a GD1a/b; and
v) a GlcCer;
b) GlcCer in a sample from the subject, provided the LSD is an MPS disorder;
c) Nf-L in a sample from the subject; and/or
d) sTREM2 in a sample from the subject; and
3) adjusting the dosage of the LSD treatment based on the concentration of the selected lipid(s)/protein(s) in the sample from the subject as compared to a control value.

6. The method of any one of claims 1-5, comprising measuring the concentration of a combination of two or more lipids.

7. The method of any one of claims 1-5, comprising measuring the concentration of sTREM2.

8. The method of any one of claims 1-5, comprising measuring the concentration of Nf-L.

9. The method of any one of claims 1-5, comprising measuring the concentration of GlcCer, wherein the LSD is an MPS disorder.

10. The method of any one of claims 1-5, comprising measuring the concentration of one or more lipids and the concentration of sTREM2.

11. The method of any one of claims 1-5, comprising measuring the concentration of one or more lipids and the concentration of Nf-L.

12. The method of any one of claims 1-6, wherein the combination comprises a BMP.

13. The method of any one of claims 1-6, wherein the combination comprises a GlcCer.

14. The method of any one of claims 1-6, wherein the combination comprises a GD3.

15. The method of any one of claims 1-6, wherein the combination comprises a GD1a/b.

16. The method of any one of claims 1-6, wherein the combination comprises a GM2.

17. The method of any one of claims 1-6, wherein the combination comprises a GM3.

18. The method of any one of claims 1-6, wherein the combination comprises: a BMP and a GlcCer; a BMP and a GD3; a BMP and a GD1a/b; a BMP and a GM2; a BMP and a GM3; a GlcCer and a GD3; a GlcCer and a GD1a/b; a GlcCer and a GM2; a GlcCer and a GM3; a GD3 and a GD1a/b; a GD3 and a GM2; a GD3 and a GM3; a GD1a/b and a GM2; a GD1a/b and a GM3; a BMP, a GlcCer and a GD3; a BMP, a GlcCer and a GD1a/b; a BMP, a GlcCer and a GM2; a BMP, a GlcCer and a GM3; a BMP, a GD3 and a GD1a/b; a BMP, a GD3 and a GM2; a BMP, a GD3 and a GM3; a BMP, a GD1a/b and a GM2; a BMP, a GD1a/b and a GM3; a BMP, a GM2 and a GM3; a GlcCer, a GD3 and a GD1a/b; a GlcCer, a GD3 and a GM2; a GlcCer, a GD3 and a GM3; a GlcCer, a GD1a/b and a GM2; a GlcCer, a GD1a/b and a GM3; a GlcCer, a GM2 and a GM3; a GD3, a GD1a/b and a GM2; a GD3, a GD1a/b and a GM3; a GD3, GM2 and a GM3; a GD1a/b, a GM2 and a GM3; a BMP, a GlcCer, a GD3 and a GD1a/b; a BMP, a GlcCer, a GD3 and a GM2; a BMP, a GlcCer, a GD3 and a GM3; a BMP, a GlcCer, a GD1a/b and GM2; a BMP, a GlcCer, a GD1a/b and GM3; a BMP, a GlcCer, a GM2 and GM3; a BMP, a GD3, a GD1a/b and a GM2; a BMP, a GD3, a GD1a/b and a GM3; a BMP, a GD3, a GM2 and a GM3; a BMP, a GD1a/b, a GM2 and a GM3; a GlcCer, a GD3, a GD1a/b and a GM2; a GlcCer, a GD3, a GD1a/b and a GM3; a GlcCer, a GD3, a GM2 and a GM3; a GlcCer, a GD1a/b, a GM2 and a GM3; a GD3, a GD1a/b, a GM2 and a GM3; a BMP, a GlcCer, a GD3, a GD1a/b and a GM2; a BMP, a GlcCer, a GD3, a GD1a/b and a GM3; a BMP, a GD3, a GD1a/b, a GM2 and a GM3; a BMP, a GlcCer, a GD3, a GM2 and a GM3; a BMP, a GlcCer, a GD1a/b, a GM2 and a GM3; a GlcCer, a GD3, a GD1a/b, a GM2 and a GM3; or a BMP, a GlcCer, a GD3, a GD1/b, a GM2 and a GM3.

19. The method of claim any one of claims 1-18, wherein the LSD is an MPS disorder.

20. The method of claim 19, wherein the MPS disorder is Hunter's syndrome.

21. The method of any one of claims 2-20, wherein the LSD treatment comprises haematopoietic stem cell transplantation (HSCT), enzyme replacement therapy (ERT), substrate reduction therapy, chaperone therapy and/or gene therapy.

22. The method of claim 21, wherein the LSD treatment comprises an ERT targeted to the brain.

23. The method of claim 22, wherein the LSD treatment is a protein comprising:
(a) a first Fc polypeptide that is linked to an enzyme replacement therapy (ERT) enzyme, an ERT enzyme variant, or a catalytically active fragment thereof; and
(b) a second Fc polypeptide that forms an Fc dimer with the first Fc polypeptide,
wherein the first Fc polypeptide and/or the second Fc polypeptide does not include an immunoglobulin heavy and/or light chain variable region sequence or an antigen-binding portion thereof.

24. The method of claim 23, wherein the ERT enzyme is iduronate 2-sulfatase (IDS), an IDS variant, or a catalytically active fragment thereof.

25. The method of claim 23, wherein the first Fc polypeptide comprises the amino acid sequence of any one of SEQ ID NOS:113, 193, and 197, and the second Fc polypeptide comprises the amino acid sequence SEQ ID NO:114.

26. The method of claim 23, wherein the first Fc polypeptide comprises the amino acid sequence of any one of SEQ ID NOS:113, 193, and 197, and the second Fc polypeptide comprises the amino acid sequence of SEQ ID NO:131.

27. The method of claim 23, wherein the first Fc polypeptide comprises the amino acid sequence of any one of SEQ ID NOS:113, 193, and 197, and the second Fc polypeptide comprises the amino acid sequence SEQ ID NO:167.

28. The method of claim 23, wherein the first Fc polypeptide comprises the amino acid sequence of any one of SEQ ID NOS:113, 193, and 197, and the second Fc polypeptide comprises the amino acid sequence SEQ ID NO:190.

29. The method of claim 23, wherein the first Fc polypeptide comprises the amino acid sequence of any one of SEQ ID NOS:113, 193, and 197, and the second Fc polypeptide comprises the amino acid sequence SEQ ID NO:191.

30. The method of claim 23, wherein the first Fc polypeptide comprises the amino acid sequence of any one of SEQ ID NOS:113, 193, and 197, and the second Fc polypeptide comprises the amino acid sequence SEQ ID NO:117.

31. A method of screening a test agent for activity as an LSD treatment, the method comprising:
1) contacting a cell with the test agent, wherein the cell has impaired lysosomal storage; and
2) measuring the concentration of:
a) a combination of two or more lipids in the cell, wherein the combination of lipids is selected from the group consisting of:
i) a BMP;
ii) a GM2 ganglioside and/or a GM3 ganglioside;
iii) a GD3;
iv) a GD1a/b; and
v) a GlcCer;
b) GlcCer in the cell, provided the test agent is screened for activity as an MPS treatment;
c) Nf-L in the cell; and/or
d) sTREM2 in the cell;
wherein a decrease in the concentration of the selected lipid(s)/protein(s) in the cell as compared to the concentration of corresponding lipid(s)/protein(s) in a control cell indicates the test agent has activity as an LSD treatment.

32. The method of claim 31, comprising measuring the concentration of sTREM2.

33. The method of claim 31, comprising measuring the concentration of Nf-L.

34. The method of claim 31, comprising measuring the concentration of GlcCer.

35. The method of claim 31, comprising measuring the concentration of a combination of two or more lipids.

36. The method of claim 31, comprising measuring the concentration of one or more lipids and the concentration of sTREM2.

37. The method of claim 31, comprising measuring the concentration of one or more lipids and the concentration of Nf-L.

38. The method of any one of claims 31-37, wherein the cell is a brain cell.

39. A corrected CNS cell comprising reduced accumulation of a metabolic species, wherein a CNS cell comprising a deficiency in a lysosomal enzyme that causes accumulation of the metabolic species within the cell was contacted with a protein comprising:
(i) a first Fc polypeptide linked to the lysosomal enzyme; and
(ii) a second Fc polypeptide that forms an Fc dimer with the first Fc polypeptide, wherein the protein is capable of binding to the transferrin receptor (TfR),
to provide the corrected CNS cell comprising the reduced accumulation of the metabolic species.

40. The CNS cell of claim 39, wherein the protein binds to TfR with an affinity of from about 50 nM to about 350 nM.

41. The CNS cell of claim 39 or 40, wherein the enzyme is iduronate 2-sulfatase (IDS), or an enzymatically active variant thereof.

42. The CNS cell of any one of claims 39-41, wherein the metabolic species is a glycosaminoglycan (GAG) and/or a lysosomal lipid selected from the group consisting of: a ganglioside, a glucosylceramide, a galactosylceramide, and a BMP.

43. The CNS cell of any one of claims 39-42, wherein the CNS cell is selected from the group consisting of: a neuron, an astrocyte, and a microglial cell.

44. A method of sorting populations of CNS cells from a tissue sample, comprising:
(a) contacting the tissue sample with a neuronal marker primary antibody, an astrocyte marker primary antibody, a microglial marker primary antibody, an endothelial marker primary antibody, and an oligodendrocyte marker primary antibody, wherein each primary antibody is uniquely labeled, to provide a labeled tissue sample; and
(b) sorting the cells in the labeled tissue sample by flow cytometry,
wherein the method provides distinct cell populations of neurons, astrocytes, and microglial cells.

45. The method of claim 44, wherein the neuronal marker primary antibody is an anti-Thy1 antibody.

46. The method of claim 44 or 45, wherein the microglial marker primary antibody is an anti-CD11b antibody.

47. The method of any one of claims 44-46, wherein the astrocyte marker primary antibody is selected from the group consisting of: an anti-EAAT2 antibody and an anti-astrocyte cell surface antigen-2 (ACSA-2) antibody.

48. The method of any one of claims 44-47, wherein the endothelial marker primary antibody is an anti-CD31 antibody.

49. The method of any one of claims 44-48, wherein the oligodendrocyte marker primary antibody is an anti-O1 antibody.

50. The method of any one of claims 44-49, which provides a distinct population of microglial cells comprising less than about 20% non-microglial cells, a distinct population of astrocytes comprising less than about 20% non-astrocytic cells and/or a distinct population of neurons comprising less than about 20% non-neuronal cells.

51. The method of any one of claims 44-50, wherein the microglial cell population is sorted based on the marker profile O1⁻/CD31⁻/CD11b⁺; the astrocyte population is sorted based on the marker profile O1⁻/CD31⁻/Thy1⁻/EAAT2⁺ or O1⁻/CD31⁻/Thy1⁻/ACSA-2⁺; and/or the neuron population is sorted based on the marker profile O1⁻/CD31⁻/Thy1⁺/EAAT2⁻ or O1⁻/CD31⁻/Thy1⁺/ACSA-2⁻.

52. The method of any one of claims 44-51, wherein the enriched cell populations are analyzed for quantification of sTREM2, Nf-L, a metabolic species and/or a nucleic acid species.

53. The method of claim 52, wherein the metabolic species is a glycosaminoglycan (GAG) species or a lipid species is selected from the group consisting of: a ganglioside, a glucosylceramide, a galactosylceramide, and a BMP.

54. The method of any one of claims 44-53, wherein the enriched cell populations are analyzed for quantification of an administered therapeutic agent.

55. The method of claim 54, wherein the administered therapeutic agent is ETV:IDS.
